(19) United States
(12) Patent Application Publication
(10) Pub. No.: US 2007/0129364 A1
(43) Pub. Date: Jun. 7, 2007

(54) Pyrrolopyridine kinase inhibiting compounds

(76) Inventors: Han-Qing Dong, Farmingdale, NY (US); Kenneth Foreman, Farmingdale, NY (US); An-Hu Li, Farmingdale, NY (US); Mark Joseph Mulvihill, Farmingdale, NY (US); Bijoy Panicker, Farmingdale, NY (US); Arno G. Steinig, Farmingdale, NY (US); Kathryn M. Stolz, Farmingdale, NY (US); Qinghua Weng, Farmingdale, NY (US); Meizhong Jin, Farmingdale, NY (US); Brian Volk, Farmingdale, NY (US); Jing Wang, Farmingdale, NY (US); Ti Wang, Farmingdale, NY (US); James D. Beard, Farmingdale, NY (US)

Correspondence Address: OSI PHARMACEUTICALS, INC., 41 PINELAWN ROAD, MELVILLE, NY, 11747, US

(21) Appl. No.: 11/634,003

(22) Filed: Dec. 5, 2006

Related U.S. Application Data
(60) Provisional application No. 60/748,110, filed on Dec. 7, 2005.
(60) Provisional application No. 60/860,531.

(51) Int. Cl. A61K 31/5377; A61K 31/496; A61K 31/4745; C07D 471/02

(52) U.S. Cl. 514/232.8; 544/126; 544/362; 546/113; 514/253.04; 514/300

(57) ABSTRACT

Compounds represented by Formula (I): 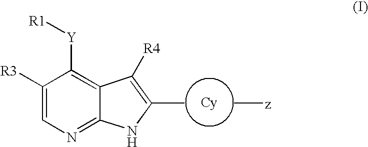 or stereoisomers or pharmaceutically acceptable salts thereof, are inhibitors of least one of the Abl, Aurora-A, Blk, c-Raf, cSRC, Src, PRK2, FGFR3, Flt3, Lck, Mek1, PDK-1, GSK3β, EGFR, p70S6K, BMX, SGK, CaMKII, Tie-2, IGF-1R, Ron, Met, and KDR kinases in animals, including humans, for the treatment and/or prevention of various diseases and conditions such as cancer.

This application claims the benefit of U.S. Patent Application No. 60/748,110 filed Dec. 07, 2005 and 60/860,531 filed Nov. 22, 2006.

### BACKGROUND OF THE INVENTION

The present invention is directed to novel pyrrolopyridine compounds, their salts, and compositions comprising them. In particular, the present invention is directed to novel substituted pyrrolopyridine compounds that inhibit the activity of at least one of the Ab1, Aurora-A, Blk, c-Raf, cSRC, Src, PRK2, FGFR3, Flt3, Lck, Mek1, PDK-1, GSK3β, EGFR, p70S6K, BMX, SGK, CaMKII, Tie-2, IGF-1R, Ron, Met, and KDR kinases in animals, including humans, for the treatment and/or prevention of various diseases and conditions such as cancer.

Cells may migrate and divide inappropriately if the signals for division or motility cannot be stopped. This might occur if the complex system of control proteins and messengers, which signal changes in the actin system, goes awry. One such control factor is the proto-oncogene protein Ab1, a tyrosine kinase. It is implicated in cancer, including leukemia. Accordingly, it is desirable to identify inhibitors of Ab1.

The Aurora kinase family is one regulator of chromosome segregation—regulating the structure and function of centrosomes and mitotic spindle. One member, the Aurora-A kinase, has been shown to play a role in tumorigenesis—being located at a chromosomal hot-spot, 20q13, frequently amplified in a variety of human cancers such as those of colon, ovary, breast and pancreas. It appears that overexpression of Aurora-A kinase alone is sufficient to cause aneupoidy in normal diploid epithelial cells. Over-expression of Aurora-A kinase in NIH3T3 cells results in centrosome aneupoidy. Thus, it is desirable to identify inhibitors of Aurora-A.

Shortly after birth, mice expressing an activated, mutant form of Blk form massive, monoclonal lymphomas and die. Thus, it is likely that Blk regulates cell proliferation. Further, experiments with Blk antisense appear to implicate Blk kinase with growth inhibition and apoptosis. (X.Yao and D. W. Scott, Proc. Nat. Acad. Sci., 90:7946-7950(1993). Thus, it is desirable to identify inhibitors of Blk.

C-Raf is an extracellular signal-regulated kinase and a downstream effector of Ras. It functions to suppress apoptosis and regulates cell differentiation. Accordingly, over-expression can lead to unwarranted suppression of apoptosis and unchecked cell differentiation. Thus, it is desirable to identify inhibitors of c-Raf.

The cytoplasmic tyrosine kinase cSRC, or c-Src, is involved in the signal transduction pathway and is elevated in breast cancer cell lines. Similarly, Src is involved in the regulation of cell growth and transformation. Thus over-expression of Src or cSRC can lead to excess proliferation. Thus, it is desirable to identify inhibitors of Src or c-SRC.

The Protein Kinase c-Related Kinase 2, or PRK2, mediates cytoskeletal organization. It has been implicated in promoting the PDK1-dependent activation of Akt, thereby regulating cell-cycle progression, cell growth, cell survival, cell motility and adhesion, translation of mRNA into protein, and angiogenesis. Thus, it is desirable to identify inhibitors of PRK2.

FGFR3 and Tie-2 are receptor tyrosine kinases that are believed to be important mediators of tumor angiogenesis. For example, FGFR3 mutations are often seen in bladder cancer cells. Tie-2 is a protein receptor found on cells lining blood vessels. When activated by growth factors secreted by tumor cells, Tie2 triggers vessel cell walls to part and grow new capillaries. Thus, it is desirable to identify inhibitors of FGFR3 or Tie-2.

Flt3, also known as “vascular endothelial cell growth factor receptor 3” or VEGFR-3, is believed to assist in vascular development important to angiogenesis. Thus, it is desirable to identify inhibitors of Flt3.

Lck, along with fyn, is an Src kinase implicated in cancer, including breast and colon cancer. Accordingly, it is desirable to identify inhibitors of Lck.

Mek1 is a kinase in the Ras pathway strongly implicated in many cancers, including breast, colon, and ovarian cancer. Thus, it is desirable to identify inhibitors of Mek1.

PDK-1 is a kinase that activates the PI3K/PKB signalling pathway, which is often uncoupled and separate from the EGFR pathway. In particular, a PDK-1 phosphorylating step is essential to activation of PKB (D. R. Alessi et al., Curr. Biol., 7:261-269(1997)). Additionally, PDK-1 activates other oncogene kinases such as PKA, ribosomal p90 S6 kinase (RSK), p70 S6 kinase (S6K), serum and glucocorticoid activated kinase (SGK), PKC-related kinase-2 (PRK-2) and MSK-1 (R. M. Biondi et al., Biochem. J., 372:1-13 (2003)). Thus, inhibition of PDK-1 can be multiply effective in treatment of cancer and tumors, including glioblastoma, melanoma, prostate, endometrial carcinoma, breast, ovarian, and non-small cell lung cancer (NSCLC), because PDK-1 regulates several oncogenic pathways. Accordingly, it is desirable to identify compounds that inhibit PDK-1.

GSK3β kinase is believed to play a strong part in cancers such as breast, ovarian, pancreatic, and prostate cancer. Thus, it is desirable to identify compounds that inhibit GSK3β.

Cell division involves signalling pathways from the cell exterior and interior. Signals travel the pathways and regulate the various activities of cell cycle control genes. Cancer cells have mis-regulation of such signal pathways and control genes—thereby leading to inappropriate or uncontrolled cell division. Over-expression of oncogenes (proteins that signal cells to proliferate) is one such mis-regulation. The Epidermal Growth Factor Receptor (EGFR) is one such oncogene, which is over-expressed in cancers such as brain, breast, gastrointestinal, lung, ovary and prostate cancers. There are selective EGFR inhibitors being investigated for use against cancer. For example, the 4-anilinoquinazoline compound Tarceva™ inhibits only EGFR kinase with high potency, although it can inhibit the signal transduction of other receptor kinases that probably heterodimerize with the EGFR. Nevertheless, other compounds that inhibit EGFR remain needed.

The serine-threonine kinase p70S6K is at the end of one pathway that controls cell growth and is frequently activated in many tumors, including uterine, adenocarcinoma, myeloma, and prostate cancers. Thus, it is desirable to identify compounds that inhibit p70S6K.

BMX is a tyrosine kinase involved in interleukin-6 induced differentiation of prostate cancer cells. It plays a role in EGF-induced apoptosis of breast cancer cells, and is expressed in granocytes and myoloid leukemias, as well as other cancers. Thus, it is desirable to identify compounds that inhibit BMX.

The serum and glucocorticoid-induced protein kinase (“SGK”) is a downstream target in the PI3K/Akt pathway, believed to play a part in cancers such as breast and prostate cancer. Thus, it is desirable to identify compounds that inhibit SGK.

Ca^{2+/}calmodulin-dependent protein kinase II (“CaMKII”) indirectly modulates Fas-mediated signalling in glioma. Therefore inhibition of CaMK II may be effective in the treatment of glioma. See, Bao Feng Yang et al., J. Biological Chemistry, 278:7043-7050 (2003). Thus, it is desirable to identify compounds that inhibit CaMKII.

Endothelial-cell specific receptor protein tyrosine kinases such as KDR and Tie-2 mediate the angiogenic process, and are thus involved in supporting the progression of cancers and other diseases involving inappropriate vascularization (e.g., diabetic retinopathy, choroidal neovascularization due to age-related macular degeneration, psoriasis, arthritis, retinopathy of prematurity, infantile hemangiomas). Thus, it is desirable to identify compounds that inhibit KDR.

RON (recepteur d'origine natais) is a receptor tyrosine kinase that is part of the MET proto-oncogene family. Inhibition of RON has been shown to lead to a decrease in proliferation, induction of apoptosis and affects cell metastasis. Inhibition of the closely related MET family member can cause a decrease in cell motility, proliferation and metastasis. Thus, it is desirable to identify inhibitors of RON and/or it related family MET.

IGF-1R (type 1 insulin-like growth factor receptor) performs important roles in cell division, development, and metabolism, and in its activated state, plays a role in oncogenesis and suppression of apoptosis. IGF-1R is known to be overexpressed in a number of cancer cell lines (IGF-1R overexpression is linked to acromegaly and to cancer of the prostate). By contrast, down-regulation of IGF-1R expression has been shown to result in the inhibition of tumorigenesis and an increased apoptosis of tumor cells. Thus, it is desirable to identify compounds that inhibit IGF-1R.

International Patent Publication No. WO 04/009600 describes 1-heterocyclyalkyl-3-sulfonylazaindole or azaindazole derivatives as 5-hydroxytryptamine-6 ligands. International Patent Publication No. WO 03/101990 describes 1-(aminoalkyl)-3-sulfonylazaindoles as 5-hydroxytryptamine-6 ligands.

International Patent Publication No. WO 05/062795 describes crystal structures of c-Ret kinase domain and surrogates for the design and synthesis of azaindole modulators. International Patent Publication No. WO 04/099205 describes azaindole compounds as Janus Kinase 3 (JAK3 kinase) inhibitors, and their preparation, intermediates, and pharmaceutical compositions. International Patent Publication No. WO 04/032874 describes the preparation of azaindole derivatives as inhibitors of p38 kinase.

U.S. Pat. Nos. 6,232,320 and 6,579,882, International Patent Publications No. WO 00/75145 and 99/62908 describe cell adhesion inhibiting antiinflammatory compounds.

International Patent Publication No. WO 00/44728 and U.S. Pat. Nos. 6,541,481 and 6,284,764 describe substituted bicyclic derivatives useful as anticancer agents.

International Patent Publication No. WO 99/07703 and U.S. Pat. No. 6,187,778 describe 4-aminopyrrole (3,2-d) pyrimidines as neuropeptide Y receptor antagonists. Japanese Patent No. 3119758 describes preparation and formulation of azaindoles as ulcer inhibitors. International Patent Publications No. WO 01/47922, WO 03/000688, and U.S. Pat. Nos. 6,770,643 and 6,897,207 describe the preparation of azaindoles as protein kinase inhibitors.

Although the anticancer compounds described above have made a significant contribution to the art, there is a continuing need to improve anticancer pharmaceuticals with better selectivity or potency, reduced toxicity, or fewer side effects.

### SUMMARY OF THE INVENTION

Compounds represented by Formula (I): 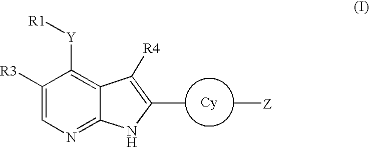
or stereoisomers or pharmaceutically acceptable salts thereof, are inhibitors of least one of the Ab1, Aurora-A, Blk, c-Raf, cSRC, Src, PRK2, FGFR3, Flt3, Lck, Mek1, PDK-1, GSK3β, EGFR, p70S6K, BMX, SGK, CaMKII, Tie-2, Ron, Met, IGF-1R, and KDR kinases in animals, including humans, for the treatment and/or prevention of various diseases and conditions such as cancer

### DETAILED DESCRIPTION OF THE INVENTION

The present invention relates to compounds of Formula I:

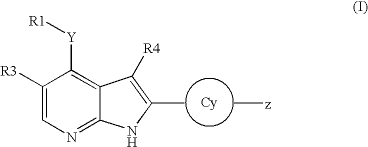

or a pharmaceutically acceptable salt thereof, wherein

Cy is

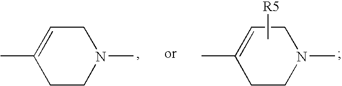

Z is hetaryl, —C₀₋₆alkyl, —C₂₋₆alkyl-O—C₁₋₆alkyl-, —C₀₋₆alkyl-(heterocyclyl), —C₀₋₆alkyl-(hetaryl), —C(O)—C₀₋₆alkyl, —C(O)-C₀₋₆alkyl-O—C₀₋₆alkyl, —C(O)—C₀₋₆alkyl-O—C₁₋₆alkyl-O—C₀₋₆alkyl, —C(O)—C₀₋₆alkyl-N(C₀₋₆alkyl)(C₀₋₆alkyl), —C(O)—C₀₋₆alkyl-N(C₀₋₆alkyl)(aryl), —C(O)—C₀₋₆alkyl-N(C₀₋₆alkyl)(hetaryl), —C(O)—C₀₋₆alkyl-N(C₀₋₆alkyl)(heterocyclyl), —C(O)—C₀₋₆alkyl-N(C₀₋₆alkyl)(cycloalkyl), —C(O)—C₀₋₆alkyl-(heterocyclyl), —C(O)—C₀₋₆alkyl-(heterocyclyl)—C(O)—C₀₋₆alkyl, —C(O)—C₀₋₆alkyl-(hetaryl), —S(O)₂—C₀₋₆alkyl, —S(O)₂—N(C₀₋₆alkyl)(C₀₋₆alkyl), or —S(O)₂—(hetaryl), wherein any of the alkyl, heterocyclyl, or hetaryl optionally is substituted with 1-6 independent halo, OH, —C₀₋₆alkyl-O—C₀₋₆alkyl, —C₀₋₆alkyl-N(C₀₋₆alkyl)(C₀₋₆alkyl), —C(O)—CO₀₋₆alkyl-N(C₀₋₆alkyl)(C₀₋₆alkyl), —C(O)—C₀₋₆alkyl-(heterocyclyl), or —C₀₋₆alkyl; 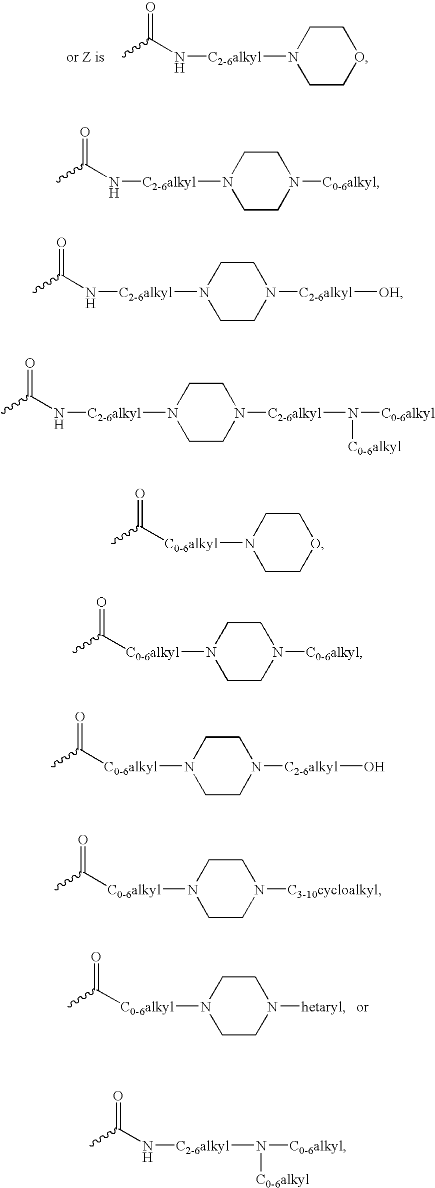
in which the wavy bond is the point of attachment, any of which except at the piperazine or morpholine moieties optionally is substituted with 1-6 independent halo, CN, OH, —C₀₋₆alkyl—O—C₀₋₆alkyl, —C₀₋₆alkyl-N(C₀₋₆alkyl)(C₀₋₆alkyl), —C(O)—C₀₋₆alkyl-N(C₀₋₆alkyl)(C₀₋₆alkyl), —C(O)—C₀₋₆alkyl-(heterocyclyl), or C₀₋₆alkyl substituents, wherein the piperazine or morpholine moieties are optionally substituted with 1-6 C₀₋₆alkyl substituents;

Y is —C(C₀₋₆alkyl)(C₀₋₆alkyl)-, —N(C₀₋₆alkyl)-, —N(C₀₋₆alkyl)—C₁₋₆alkyl-, O, S, >N-C₂₋₆alkyl-N—(C₀₋₆alkyl)(C₀₋₆alkyl), >N—C₂₋₆alkyl-O—C₀₋₆alkyl, >N—C₁₋₆alkyl-C(O)—NH-C₀₋₆alkyl, >N—C₂₋₆alkyl-N—C(O)—C₁₋₆alkyl, or a bond;

R1 is aryl, hetaryl, or heterocyclyl, optionally substituted with 1-6 independent halo, —CN, —OH, —C₀₋₆alkyl, —C₃₋₀cycloalkyl, -haloC₁₋₆alkyl, —C₂₋₆alkynyl, —N(C₀₋₆alkyl)(C₀₋₆alkyl), —C(O)—C₀₋₆alkyl-N(C₀₋₆alkyl)(C₀₋₆alkyl), —C(O)—C₀₋₆alkyl-(heterocyclyl), —C₁₋₆alkyl-C(O)—C₀₋₆alkyl-N(C₀₋₆alkyl)(C₀₋₆alkyl), —O—C₀₋₆alkyl-(heterocyclyl), —C₀₋₆alkyl-O—C₀₋₆alkyl, —alkyl-N(C₀₋₆alkyl)(C₀₋₆alkyl), —O—C₀₋₆alkyl-(hetaryl), —S(O)₂—N(C₀₋₆alkyl)(C₀₋₆alkyl), aryl, hetaryl, or heterocyclyl substituents, or optionally substituted with an oxo (═O) using a bond from the aryl, hetaryl, or heterocyclyl ring, and wherein any of the substituents optionally further is substituted with 1-6 independent halo, CN, OH, —C₀₋₆alkyl-O—C₀₋₆alkyl, —C₀₋₆alkyl-N(C₀₋₆alkyl)(C₀₋₆alkyl), —C(O)—C₀₋₆alkyl-N(C₀₋₆alkyl)(C₀₋₆alkyl), —C(O)—C₀₋₆alkyl-(heterocyclyl), or C₀₋₆alkyl;

R3 is hydrogen, C₀₋₆-alkyl, —C₀₋₆alkyl-O-C₀₋₆alkyl, halogen, azido, wherein any of the alkyl groups can optionally be substituted by halogen;

R4 is hydrogen, C₀₋₆alkyl, halogen, cyano, —S—C₁₋₆alkyl, —C₀₋₆alkyl-N(C₀₋₆alkyl)(C₀₋₆alkyl), N(C₀₋₆alkyl)(aryl), N(C₀₋₆alkyl)(hetaryl), N(C₀₋₆alkyl)(heterocyclyl), N(C₀₋₆alkyl)(cycloalkyl), —C₀₋₆alkyl-O—C₀₋₆alkyl, —C₀₋₆alkyl-O-aryl, —C₀₋₆alkyl-O-hetaryl, —C₀₋₆alkyl-O-cycloalkyl, —C₀₋₆alkyl-S(O)₀₋₂—C₀₋₆alkyl, —C₀₋₆alkyl-S(O)₀₋₂-aryl, —C₀₋₆alkyl-S(O)₀₋₂-hetaryl, —C₀₋₆alkyl-S(O)₀₋₂-cycloalkyl, aryl, hetaryl, cycloalkyl, heterocyclyl, wherein any of the alkyl, aryl, cycloalkyl or hetaryl groups can optionally be substituted with 1-6 independent halogen, CN, OH, —C₀₋₆alkyl-O—C₀₋₆alkyl, —C₀₋₆alkyl-N(C₀₋₆alkyl)(C₀₋₆alkyl), —C(O)—C₀₋₆alkyl-(heterocyclyl), —C(O)—C₀₋₆alkyl-N(C₀₋₆-alkyl)(C₀₋₆alkyl), —C(O)—C₀₋₆alkyl-N(C₀₋₆alkyl)(aryl), —C(O)—CO₀₋₆alkyl-N(C₀₋₆alkyl)(hetaryl), —C(O)—C₀₋₆alkyl-N(C₀₋₆alkyl)(heterocyclyl), —C(O)—C₀₋₆alkyl-N(C₀₋₆alkyl)(cycloalkyl), or C₀₋₆alkyl; and

R5 is hydrogen, C₀₋₆alkyl-C₀₋₆alkyl-O—C₀₋₆alkyl, or —C₀₋₆alkyl-N(C₀₋₆alkyl)(C₀₋₆alkyl), wherein any of the alkyl groups can optionally be substituted by halogen.

It is preferred that R3 is H.

It is preferred that R4 and R5 are H.

It is preferred that Cy is

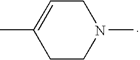

It is preferred that Y is —N(C₀₋₆alkyl)-.

Examples of R1 include, but are not limited to, the following groups, wherein the wavy bond is connected to Y:

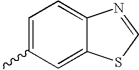

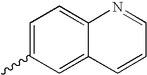

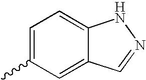

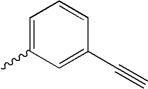

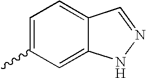

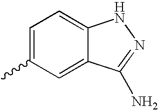

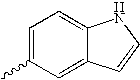

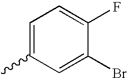

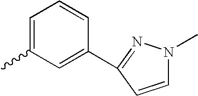

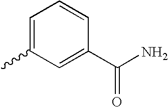

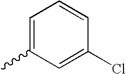

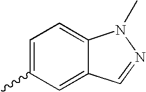

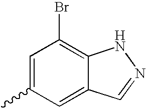

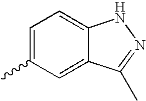

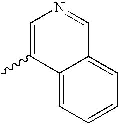

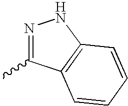

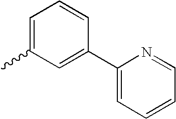

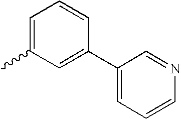

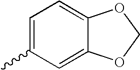

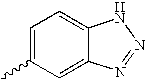

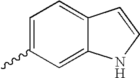

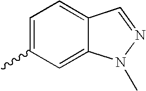

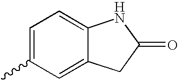

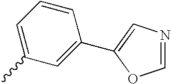

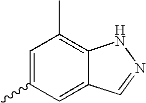

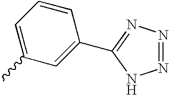

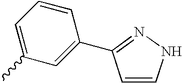

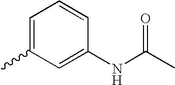

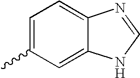

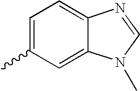

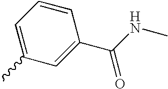

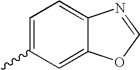

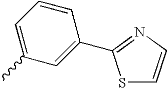

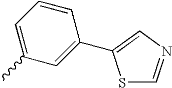

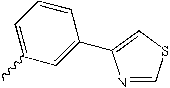

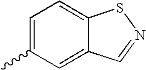

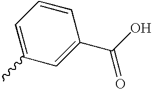

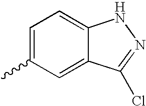

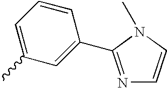

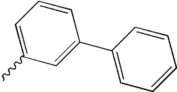

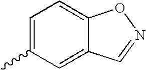

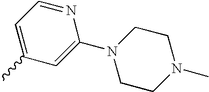

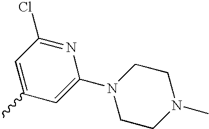

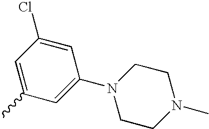

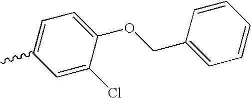

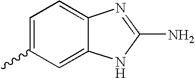

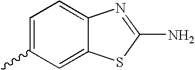

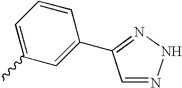

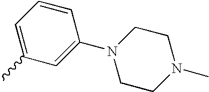

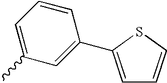

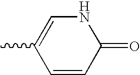

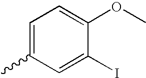

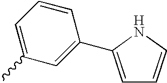

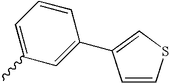

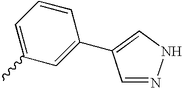

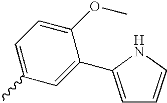

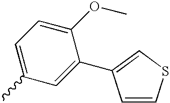

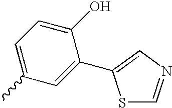

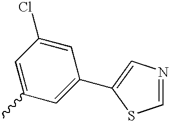

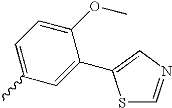

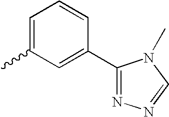

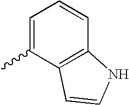

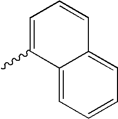

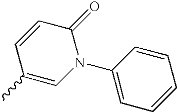

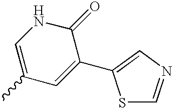

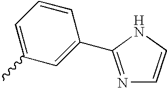

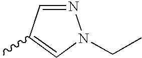

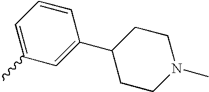

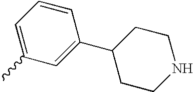

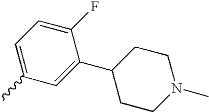

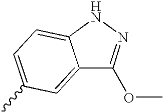

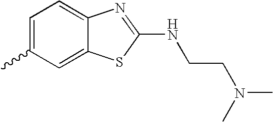

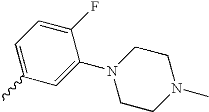

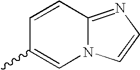

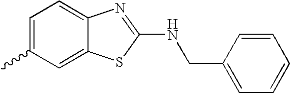

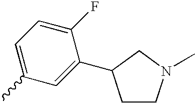

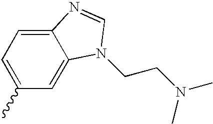

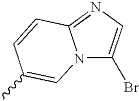

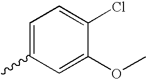

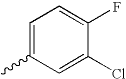

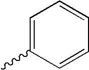

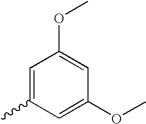

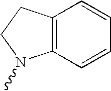

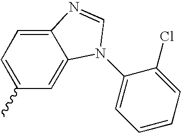

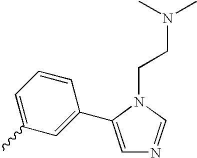

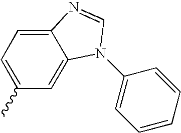

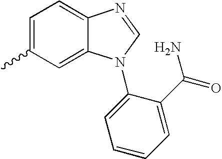

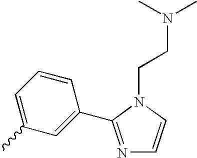

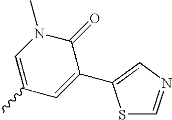

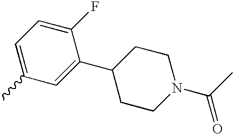

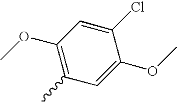

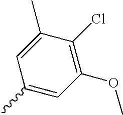

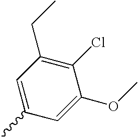

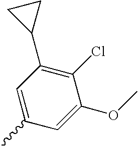

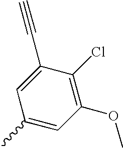

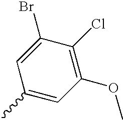

Examples of Z include, but are not limited to, the following groups, wherein the dotted line is connected to Cy:

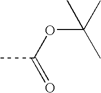

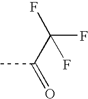

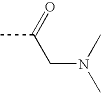

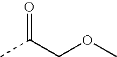

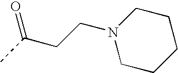

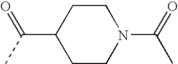

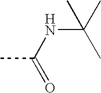

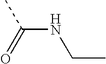

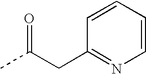

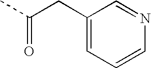

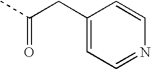

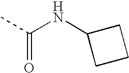

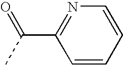

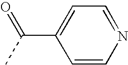

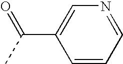

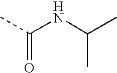

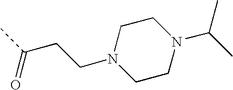

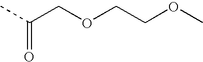

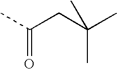

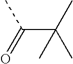

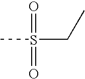

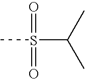

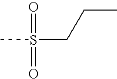

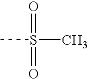

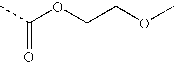

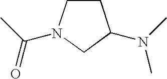

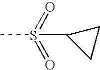

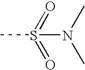

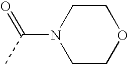

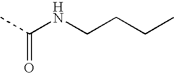

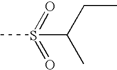

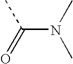

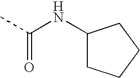

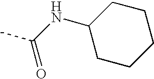

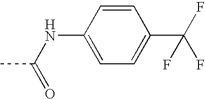

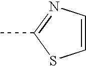

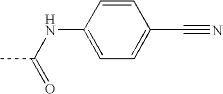

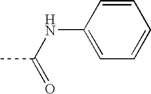

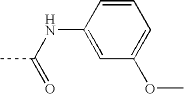

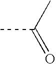

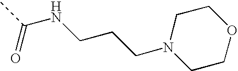

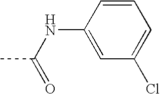

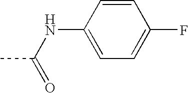

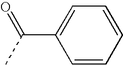

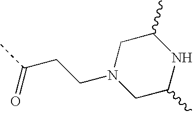

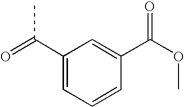

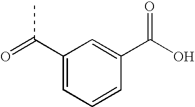

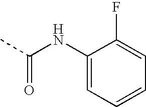

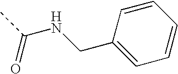

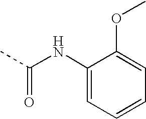

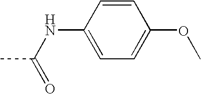

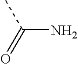

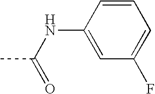

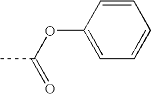

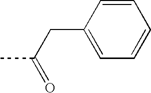

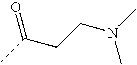

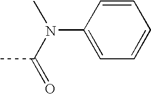

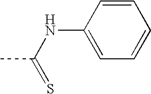

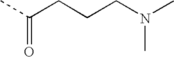

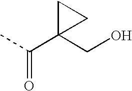

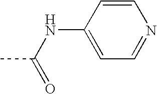

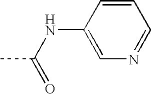

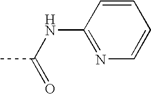

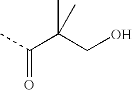

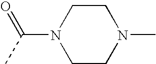

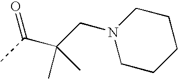

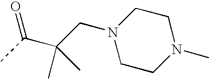

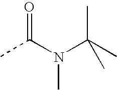

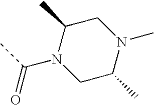

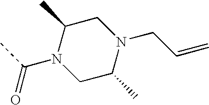

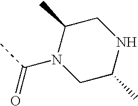

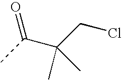

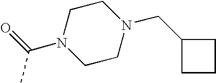

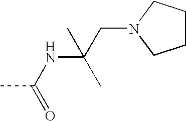

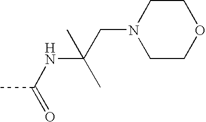

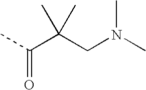

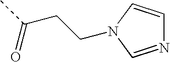

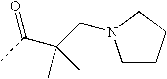

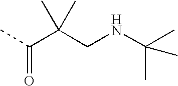

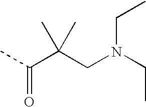

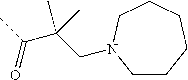

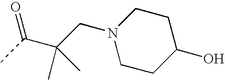

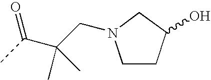

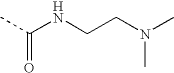

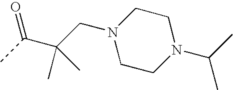

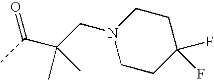

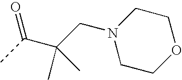

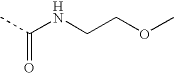

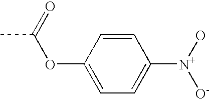

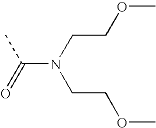

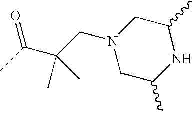

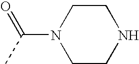

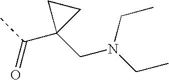

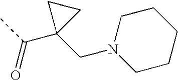

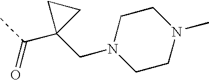

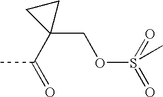

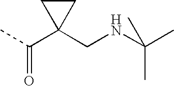

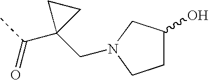

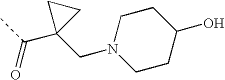

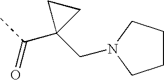

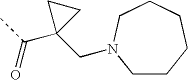

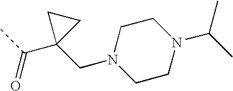

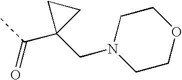

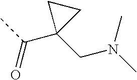

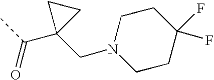

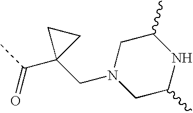

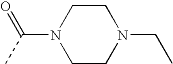

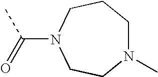

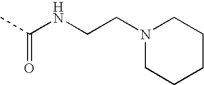

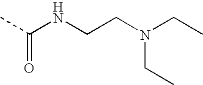

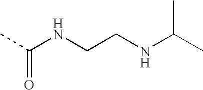

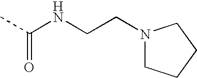

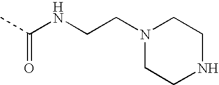

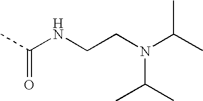

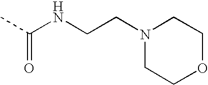

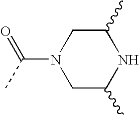

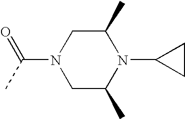

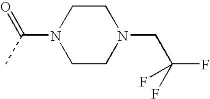

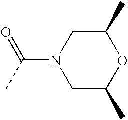

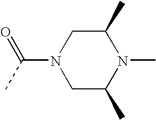

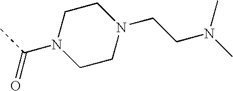

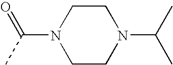

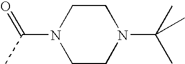

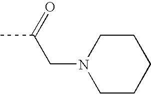

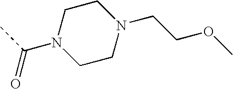

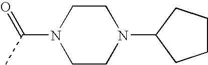

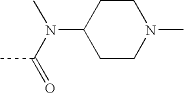

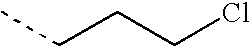

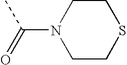

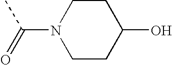

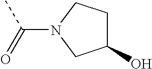

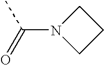

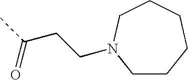

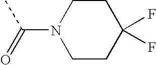

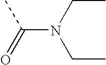

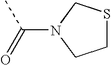

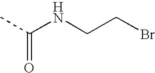

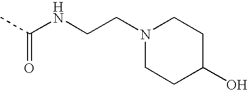

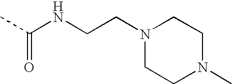

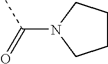

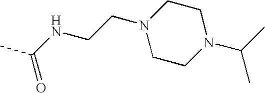

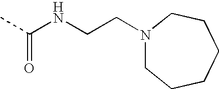

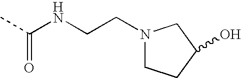

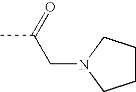

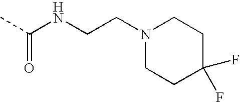

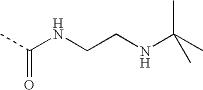

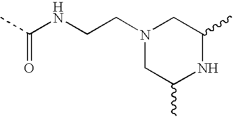

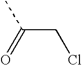

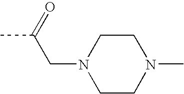

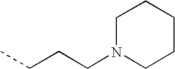

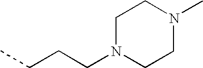

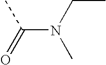

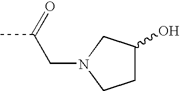

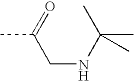

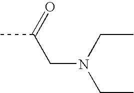

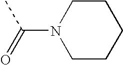

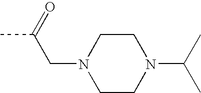

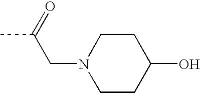

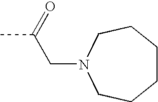

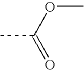

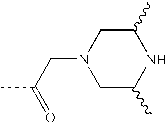

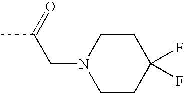

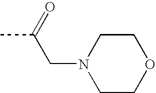

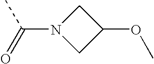

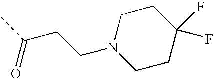

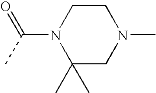

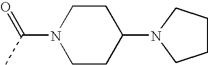

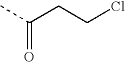

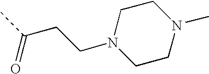

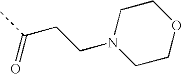

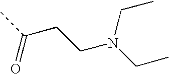

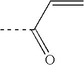

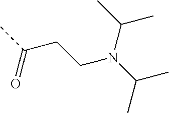

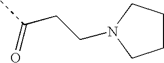

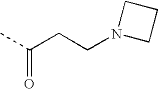

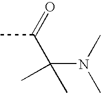

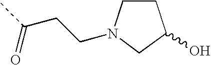

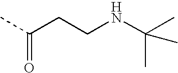

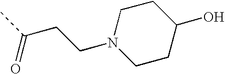

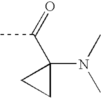

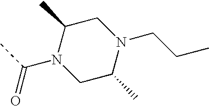

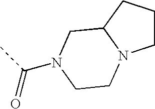

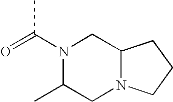

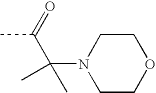

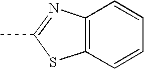

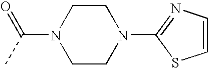

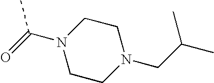

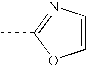

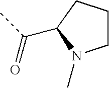

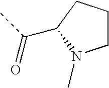

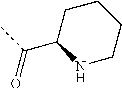

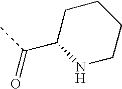

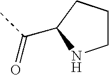

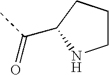

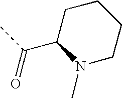

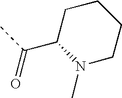

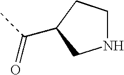

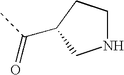

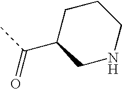

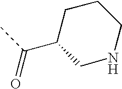

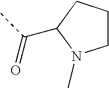

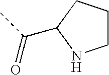

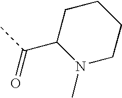

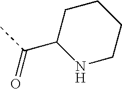

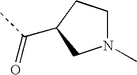

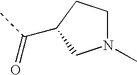

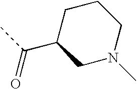

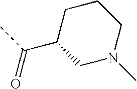

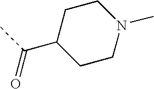

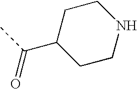

The molecular weight of the compounds of Formula (I) is preferably less than 800, more preferably less than 600.

In the first aspect, the present invention is directed to a compound represented by Formula (I), or a pharmaceutically acceptable salt thereof, wherein R3 is hydrogen and the other variables are as described above.

In an embodiment of the first aspect, the present invention is directed to a compound represented by Formula (I), or a pharmaceutically acceptable salt thereof, wherein R3 is hydrogen, Cy is 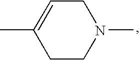
and the other variables are as described above.

In another embodiment of the first aspect, the present invention is directed to a compound represented by Formula (I), or a pharmaceutically acceptable salt thereof, wherein R3 is hydrogen, Cy is 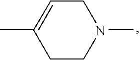
Y is —N(C₀₋₆alkyl)-, and the other variables are as described above.

In a further embodiment of the first aspect, the present invention is directed to a compound represented by Formula (I), or a pharmaceutically acceptable salt thereof, wherein R3, R4, and R5 are hydrogen, Cy is 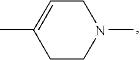
Y is —N(C₀₋₆alkyl)-, and the other variables are as described above.

In yet another embodiment of the first aspect, the present invention is directed to a compound represented by Formula (I), or a pharmaceutically acceptable salt thereof, wherein R3, R4, and R5 are hydrogen, Cy is 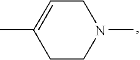
Y is —N(C₀₋₆alkyl)-, Z is —CO₂tBu, —CONHtBu, —CON(H₃)₂, 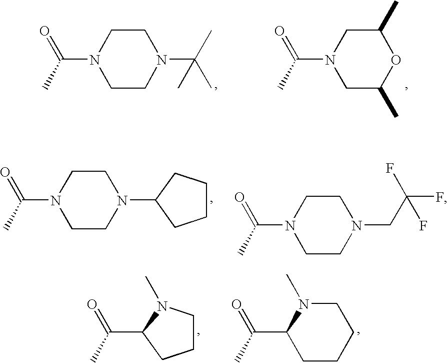
2-thiazolyl, and the other variables are as described above.

In a second aspect, the present invention is directed to a compound represented by: 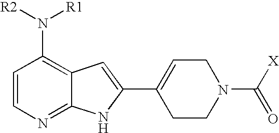
wherein R2 is —C₀₋₆alkyl, —C₂₋₆alkyl-N—(C₀₋₆alkyl)(C₀₋₆alkyl), —C₂₋₆alkyl-O—-C₀₋₆alkyl, —C₁₋₆alkyl-C(O)—NH—C₀₋₆alkyl, or —C₂₋₆alkyl-N—C(O)—C₁₋₆alkyl;
wherein X is —OtBu, —NHtBu, —N(CH₃)₂, or

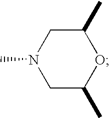

wherein R1 is selected from the following table:

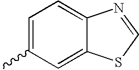

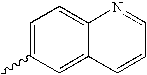

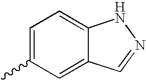

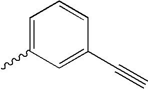

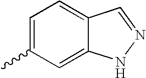

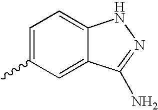

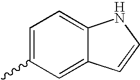

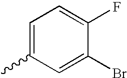

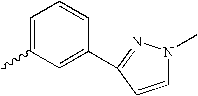

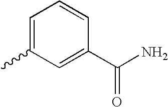

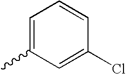

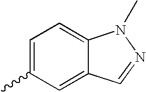

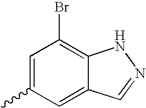

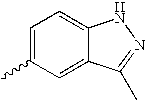

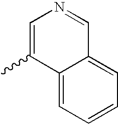

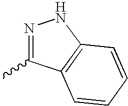

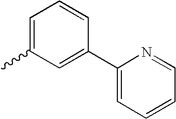

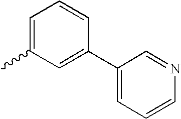

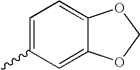

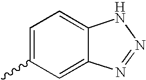

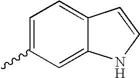

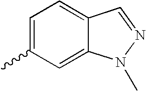

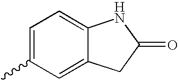

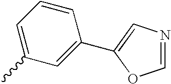

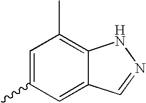

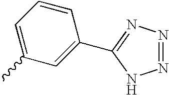

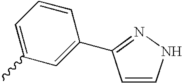

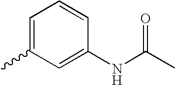

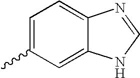

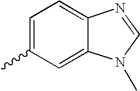

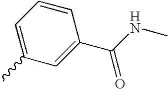

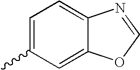

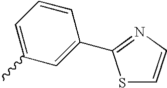

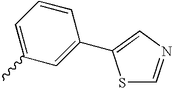

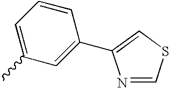

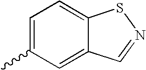

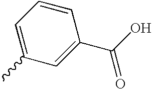

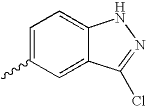

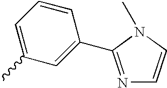

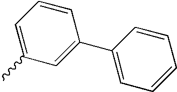

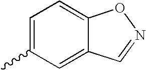

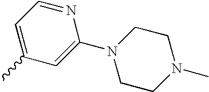

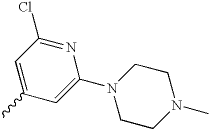

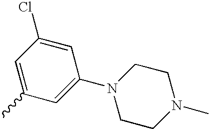

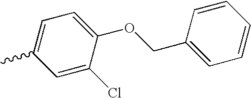

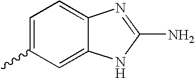

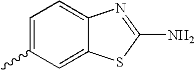

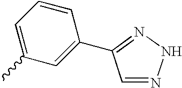

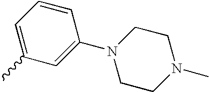

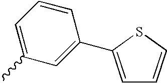

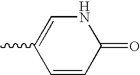

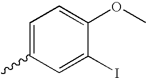

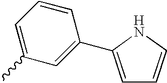

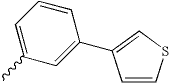

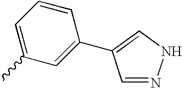

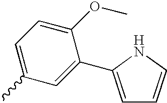

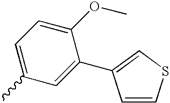

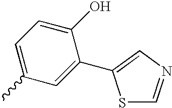

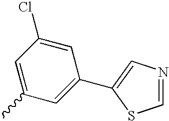

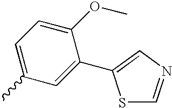

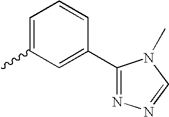

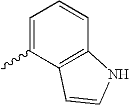

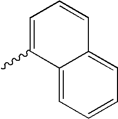

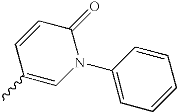

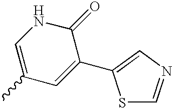

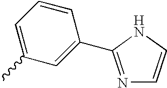

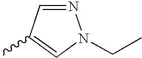

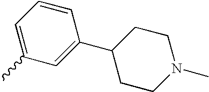

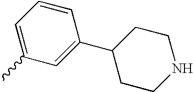

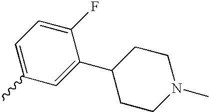

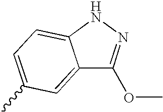

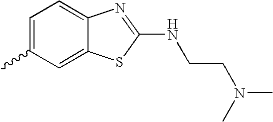

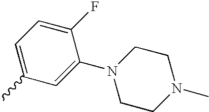

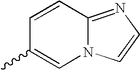

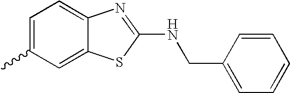

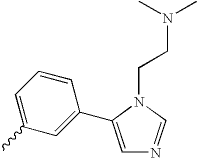

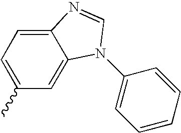

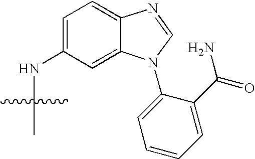

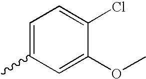

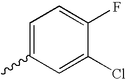

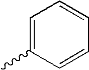

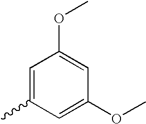

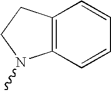

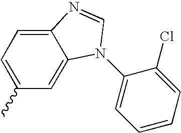

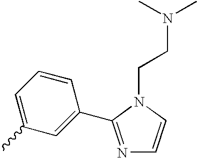

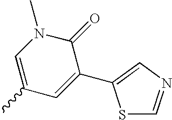

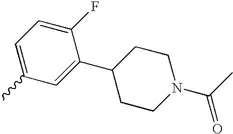

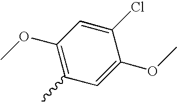

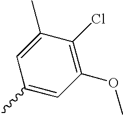

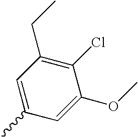

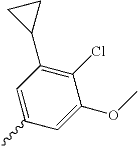

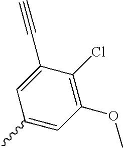

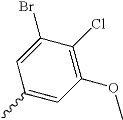

or a stereoisomer, or a pharmaceutically acceptable salt thereof.

In a third aspect, the present invention is directed to a compound represented by: 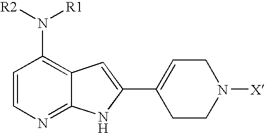
wherein R2 is —C₀₋₆alkyl, —C₂₋₆alkyl-N—(C₀₋₆alkyl)(C₀₋₆alkyl), —₂₋₆alkyl-O—C₀₋₆alkyl, —C₁₆alkyl-C(O)—NH—C₀₋₆alkyl, or —C₂₋₆alkyl-N—C(O)—C₁₋₆alkyl;
wherein X′ is optionally substituted heteroaryl,

wherein R1 is selected from the following table:

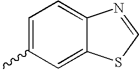

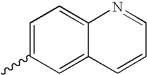

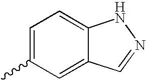

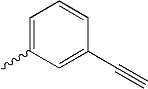

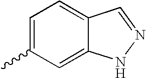

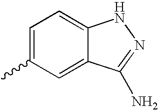

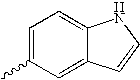

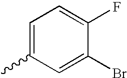

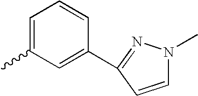

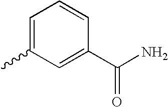

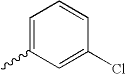

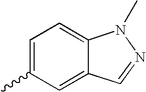

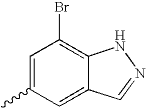

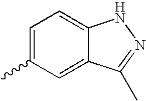

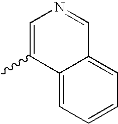

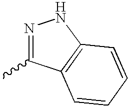

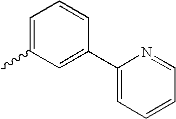

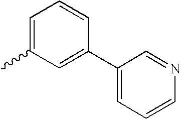

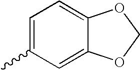

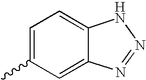

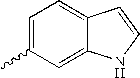

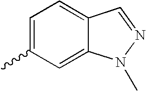

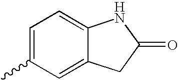

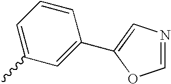

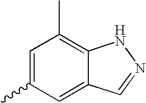

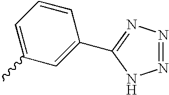

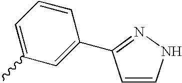

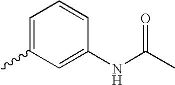

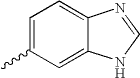

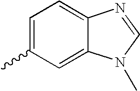

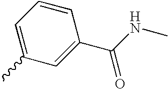

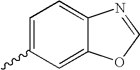

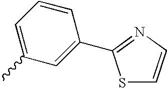

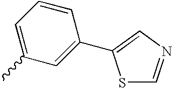

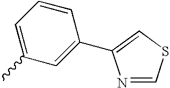

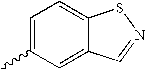

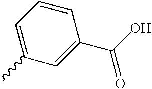

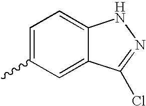

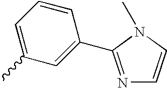

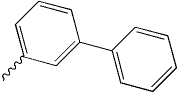

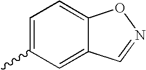

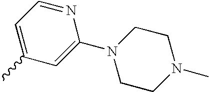

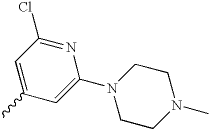

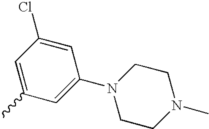

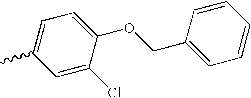

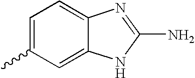

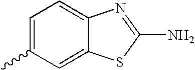

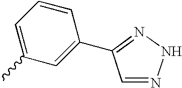

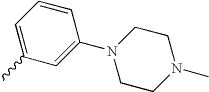

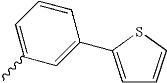

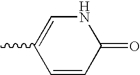

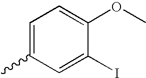

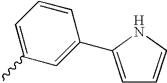

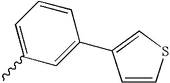

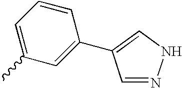

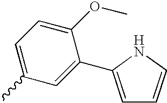

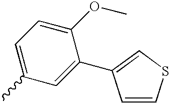

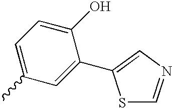

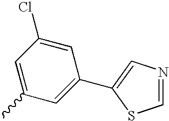

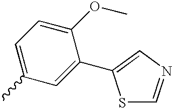

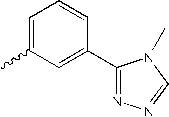

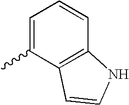

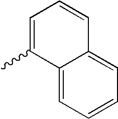

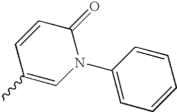

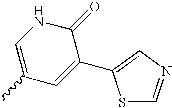

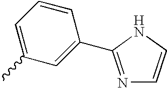

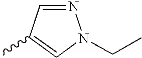

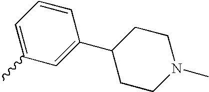

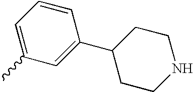

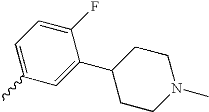

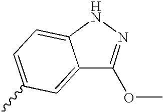

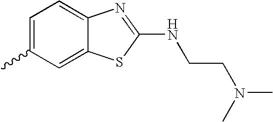

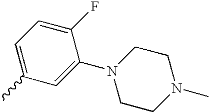

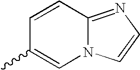

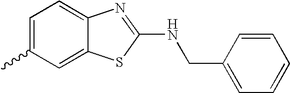

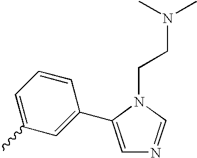

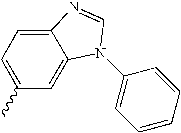

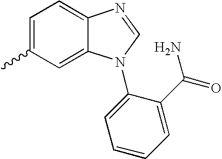

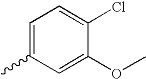

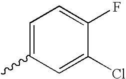

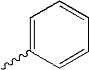

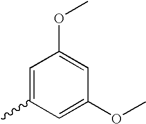

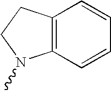

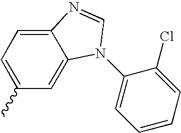

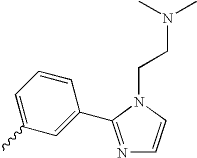

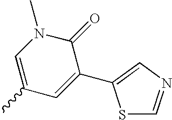

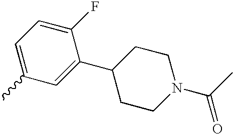

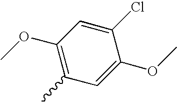

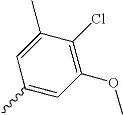

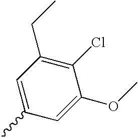

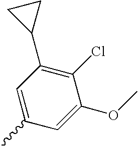

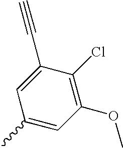

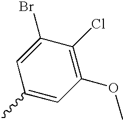

or a stereoisomer, or a pharmaceutically acceptable salt thereof.

In a fourth aspect, the present invention is directed to a compound represented by Formula (I), or a pharmaceutically acceptable salt thereof, wherein Y is —O—, and the other variables are as described above.

In a fifth aspect, the present invention is directed to a compound represented by Formula (I), or a pharmaceutically acceptable salt thereof, wherein R3 is halogen and the other variables are as described above.

In an embodiment of the fifth aspect, the present invention is directed to a compound represented by Formula (I), or a pharmaceutically acceptable salt thereof, wherein R3 is halogen, Cy is 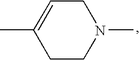
and the other variables are as described above.

In another embodiment of the fifth aspect, the present invention is directed to a compound represented by Formula (I), or a pharmaceutically acceptable salt thereof, wherein R3 is halogen, Cy is 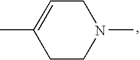
Y is —N(C₀₋₆alkyl)-, and the other variables are as described above.

In yet another embodiment of the fifth aspect, the present invention is directed to a compound represented by Formula (I), or a pharmaceutically acceptable salt thereof, wherein R3 is halogen, R4 and R5 are hydrogen, Cy is 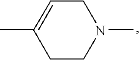
Y is —N(C₀₋₆alkyl)-, and the other variables are as described above.

The compounds of the present invention include

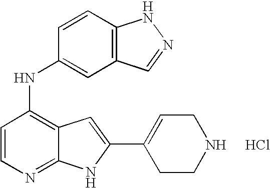

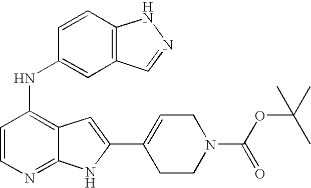

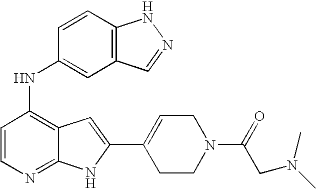

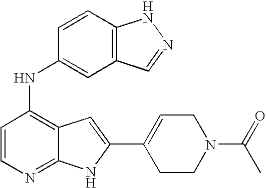

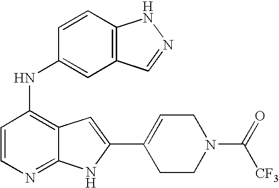

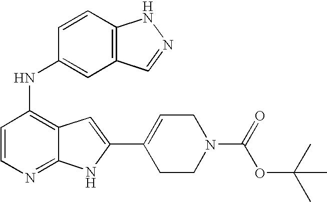

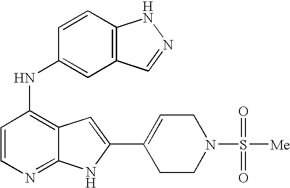

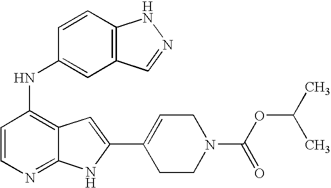

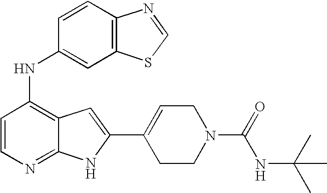

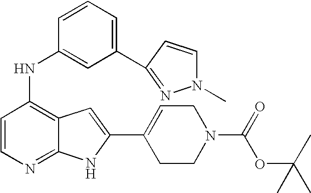

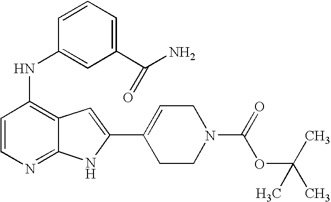

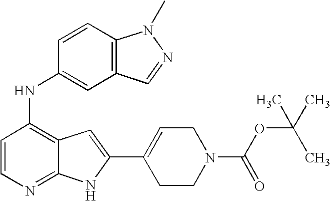

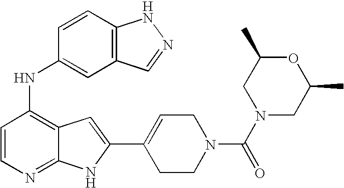

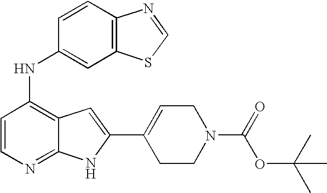

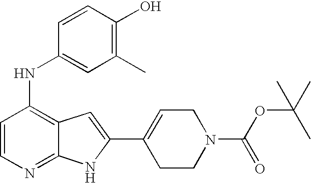

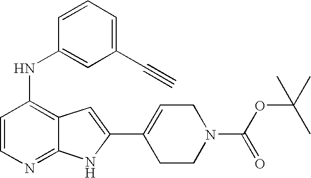

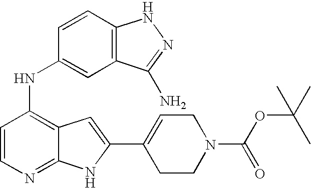

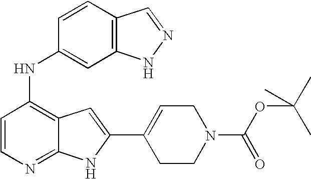

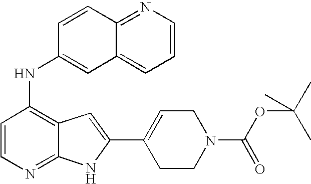

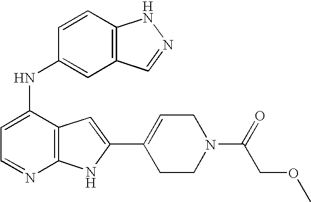

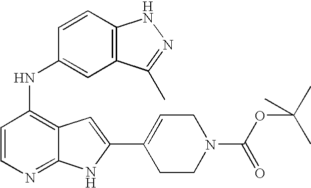

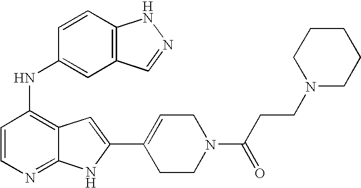

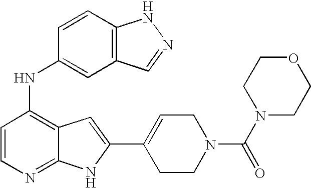

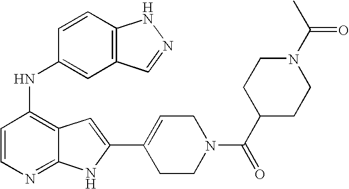

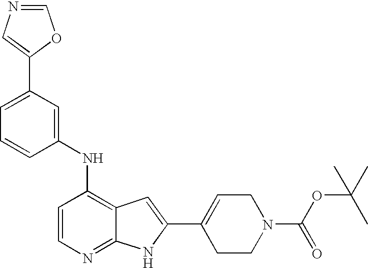

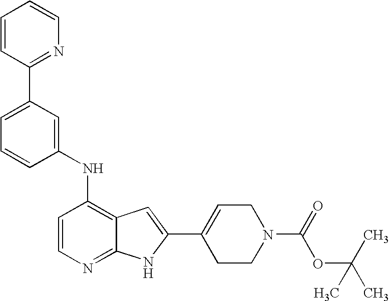

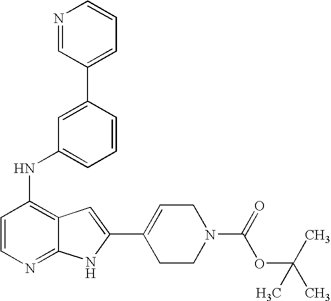

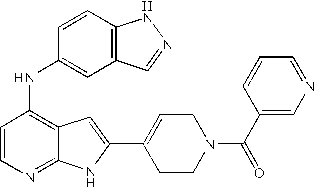

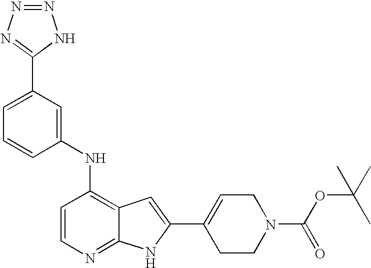

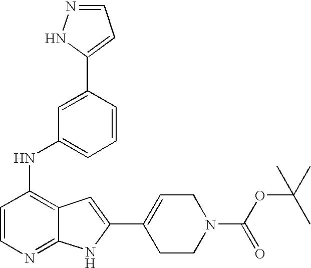

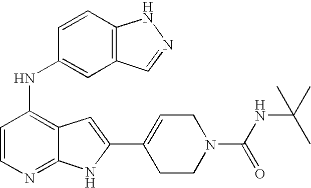

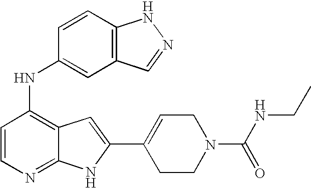

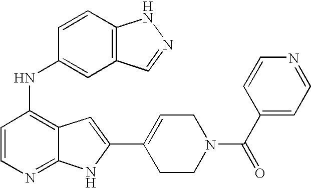

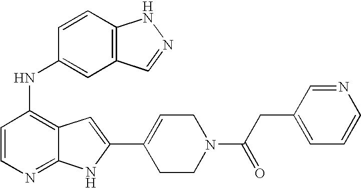

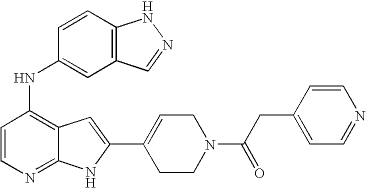

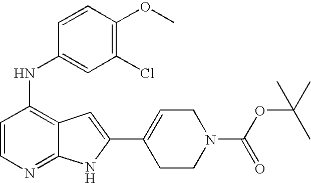

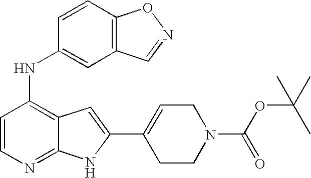

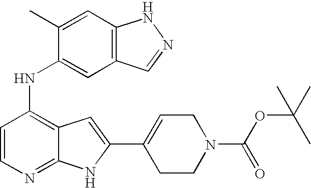

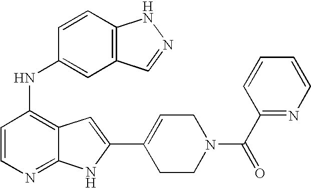

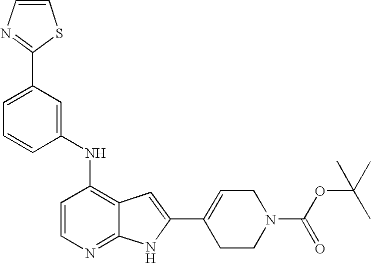

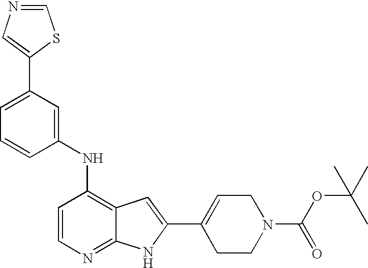

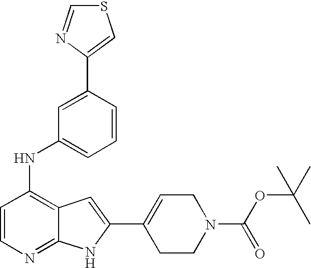

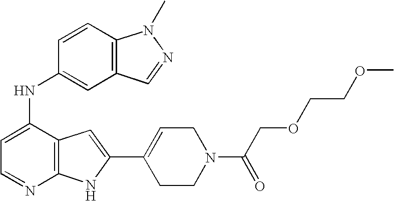

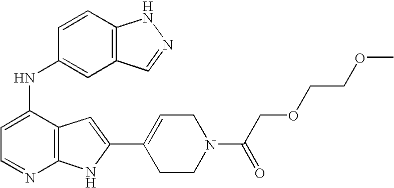

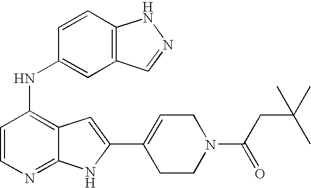

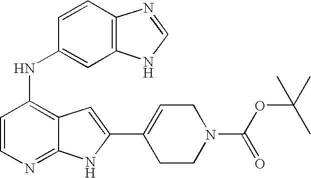

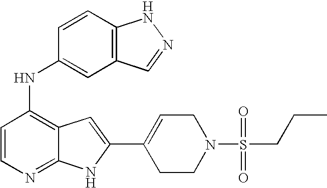

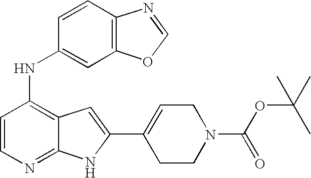

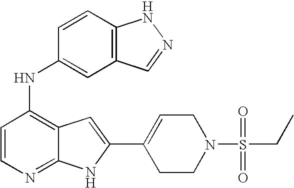

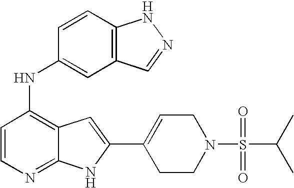

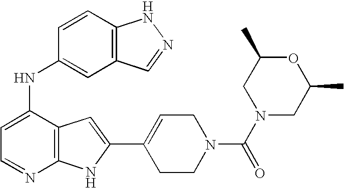

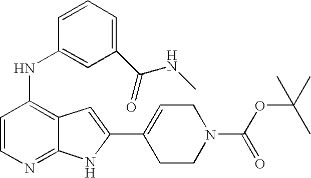

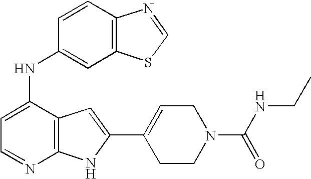

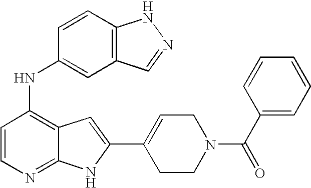

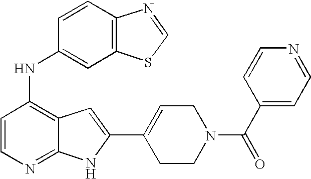

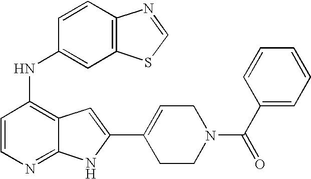

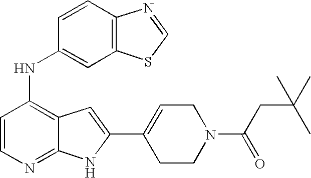

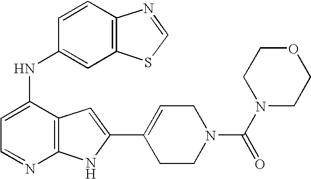

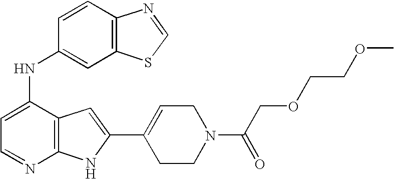

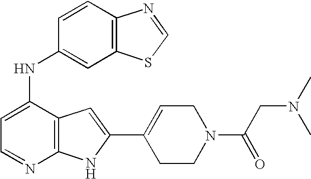

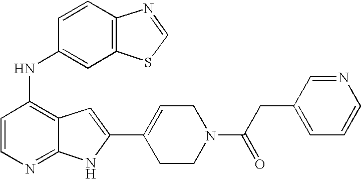

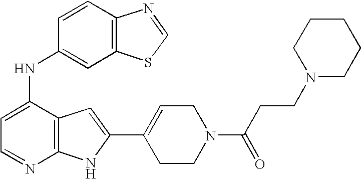

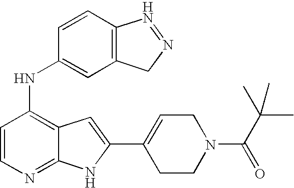

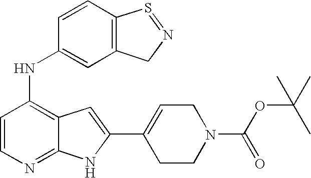

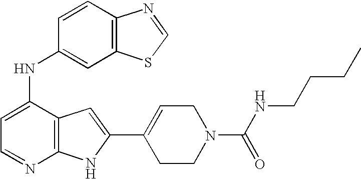

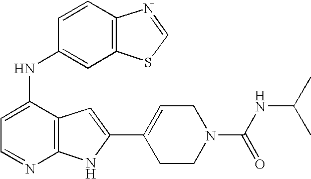

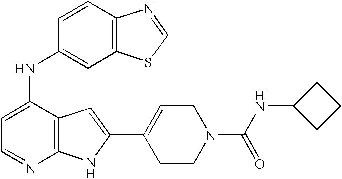

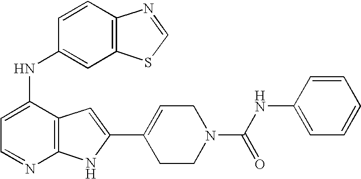

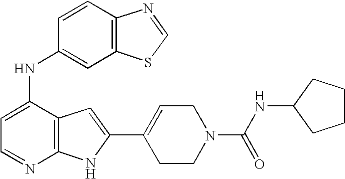

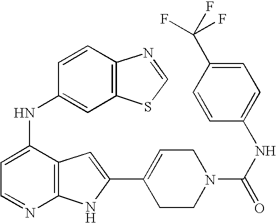

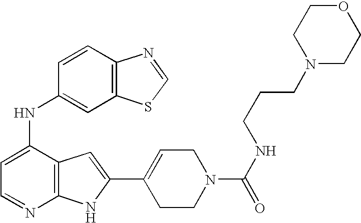

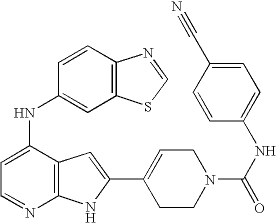

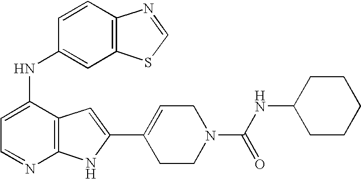

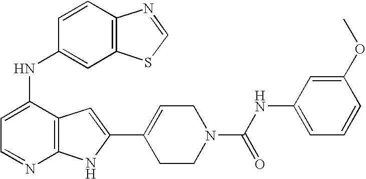

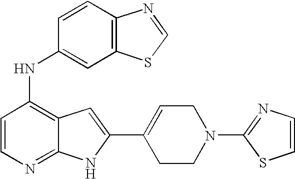

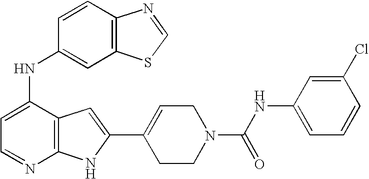

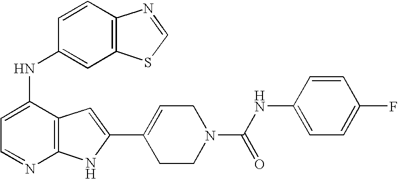

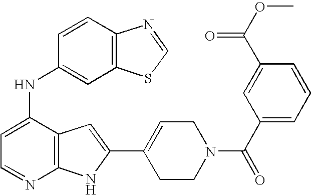

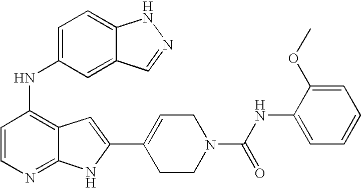

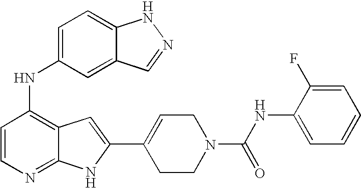

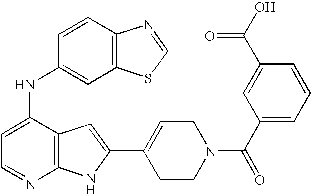

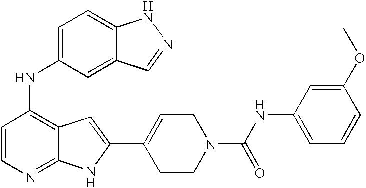

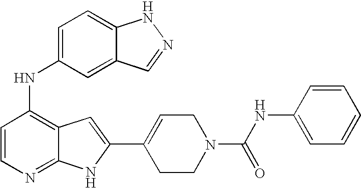

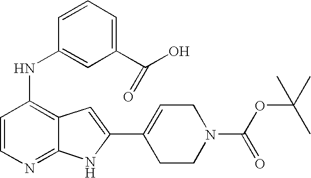

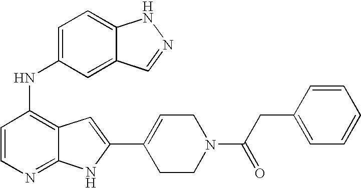

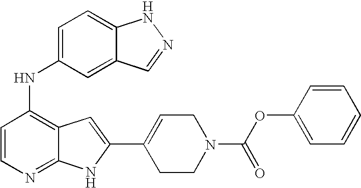

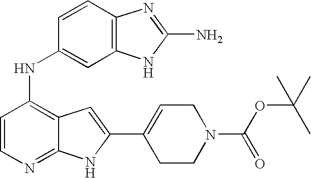

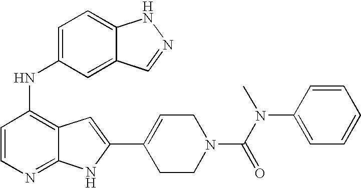

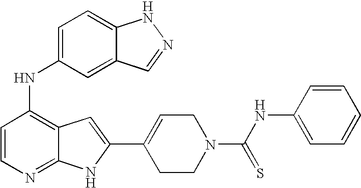

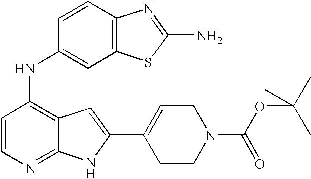

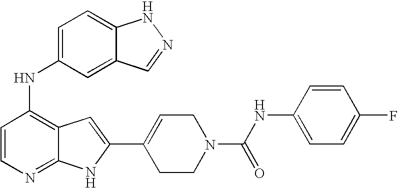

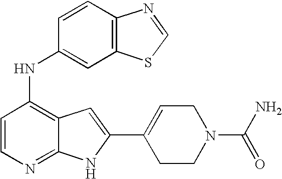

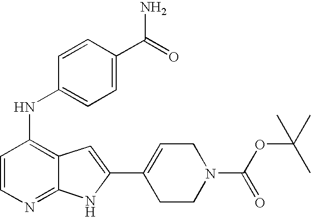

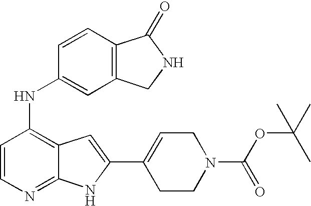

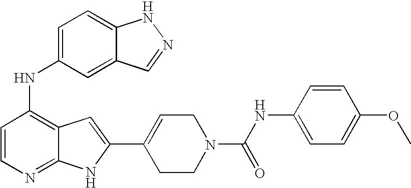

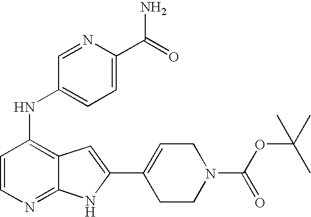

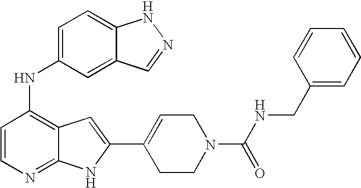

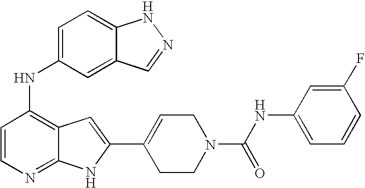

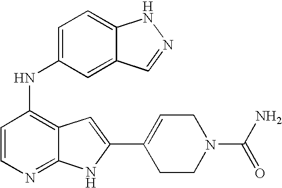

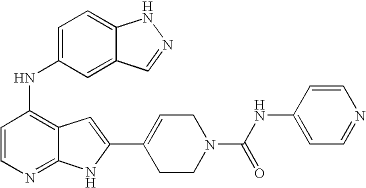

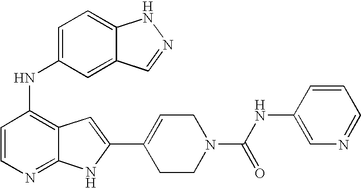

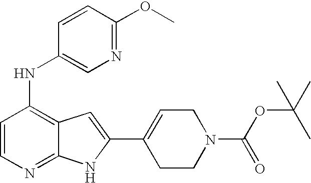

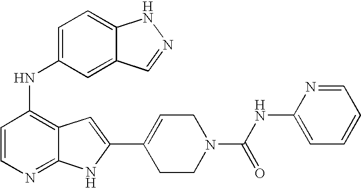

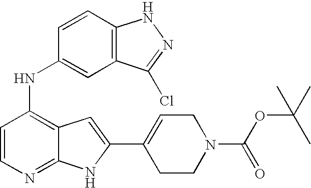

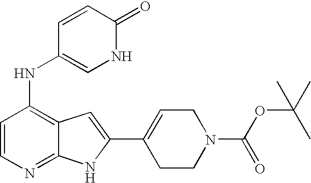

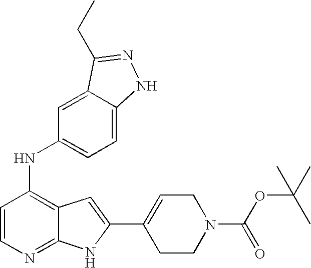

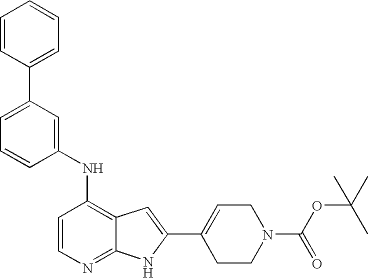

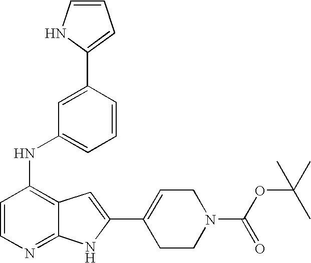

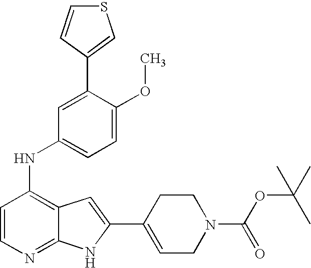

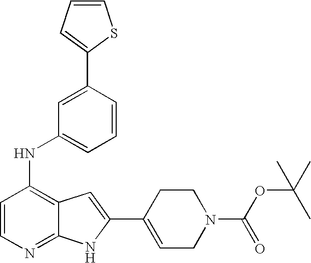

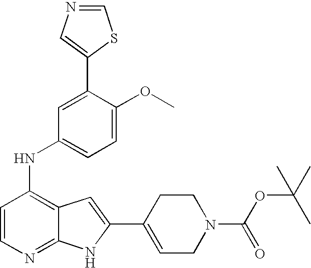

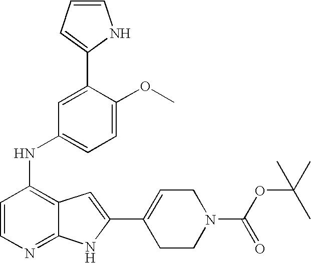

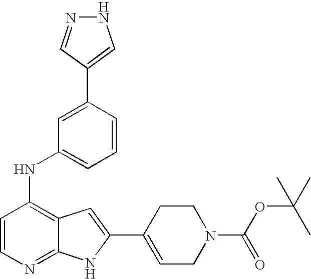

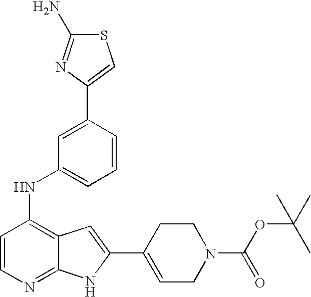

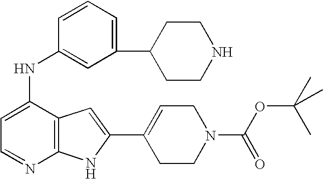

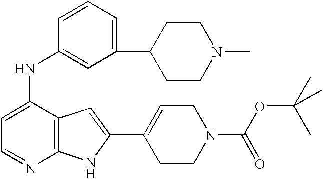

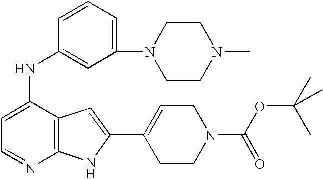

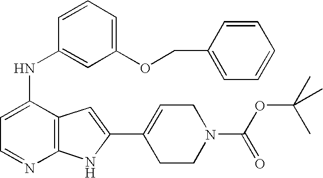

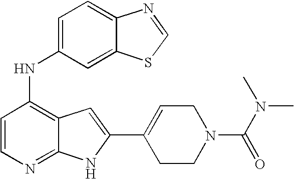

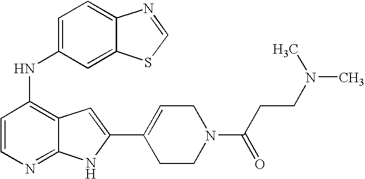

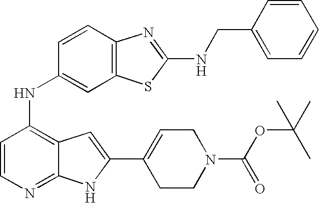

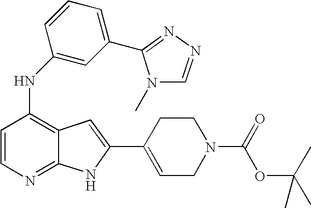

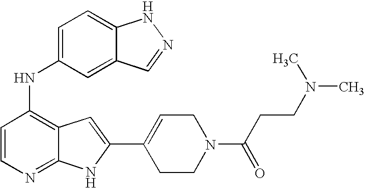

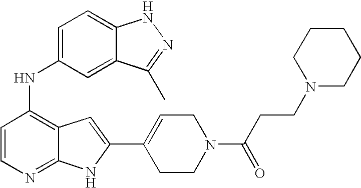

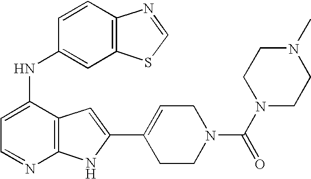

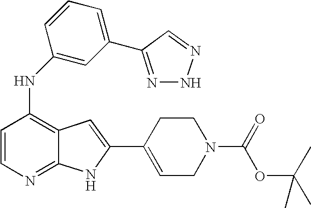

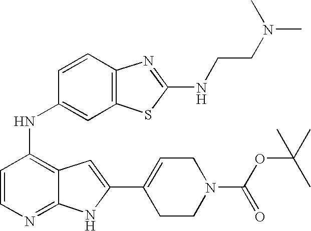

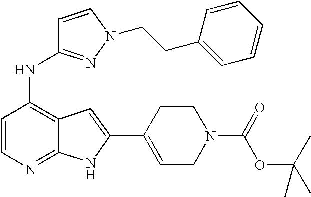

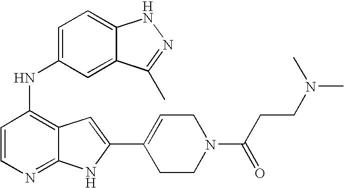

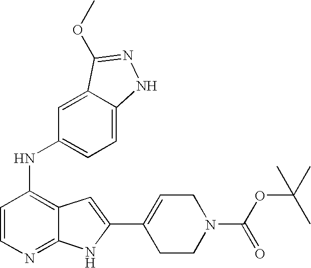

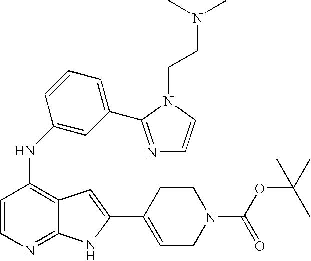

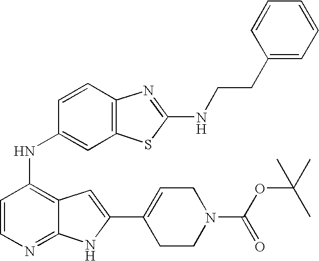

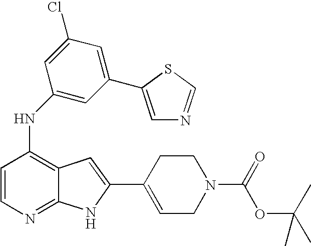

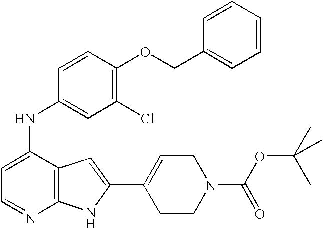

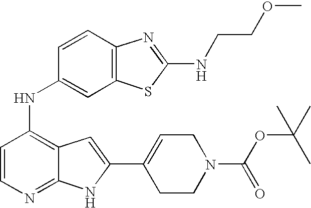

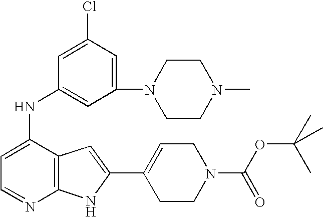

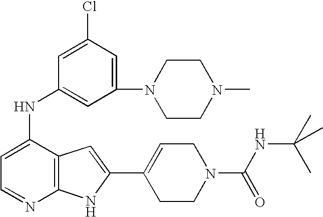

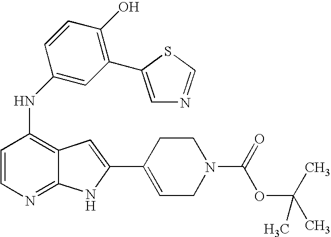

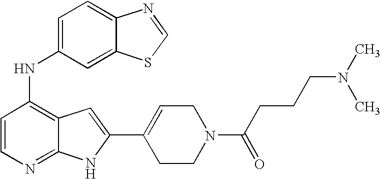

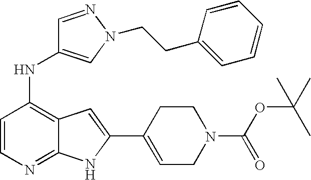

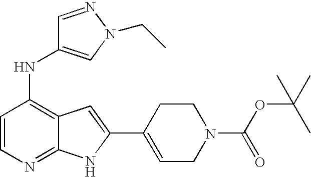

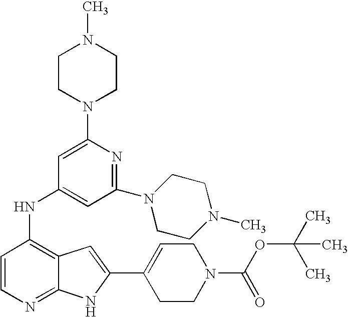

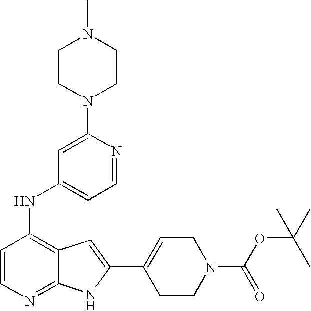

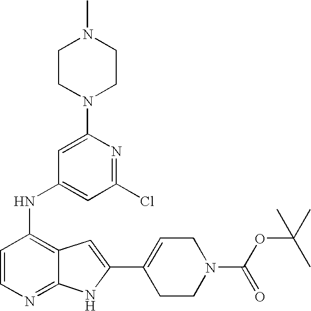

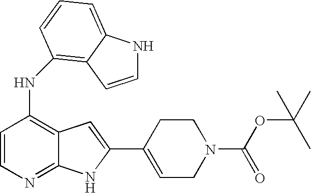

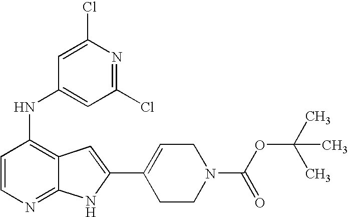

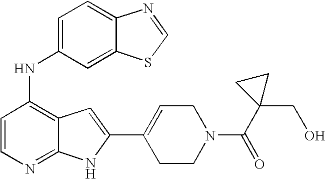

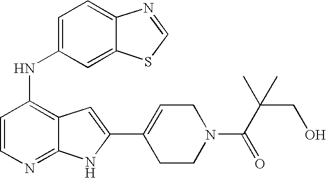

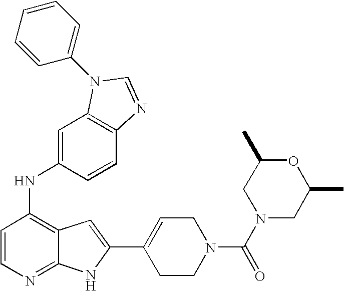

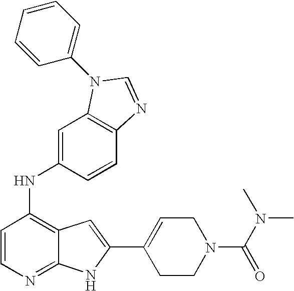

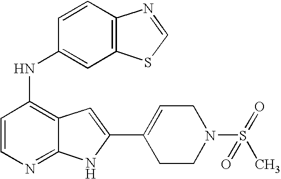

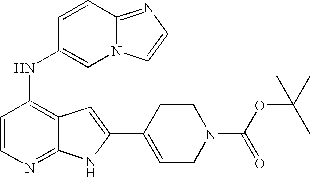

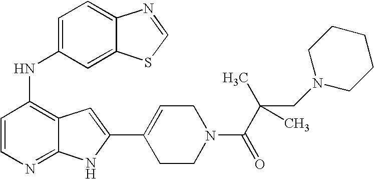

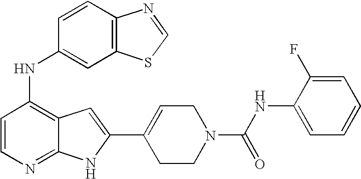

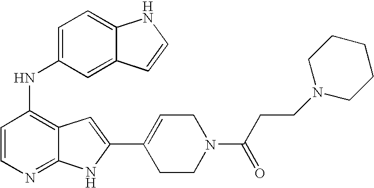

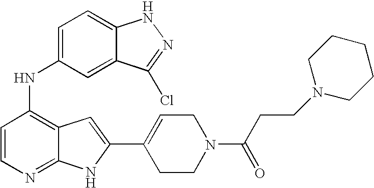

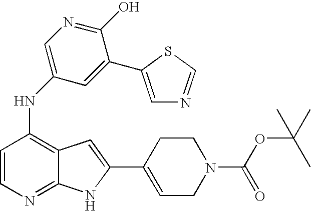

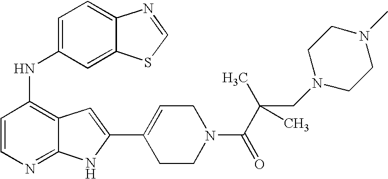

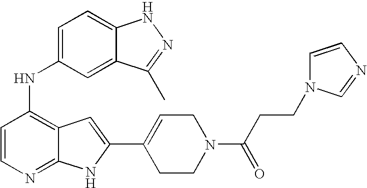

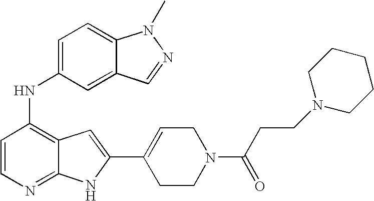

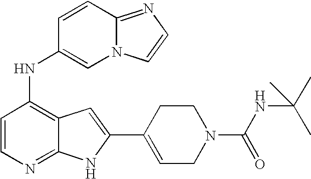

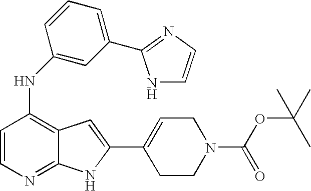

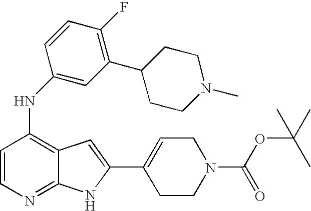

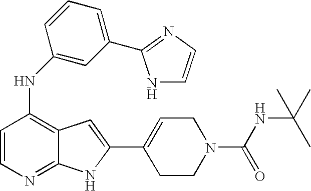

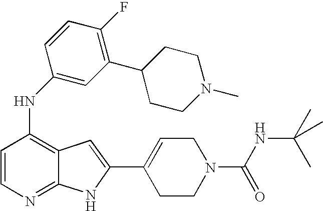

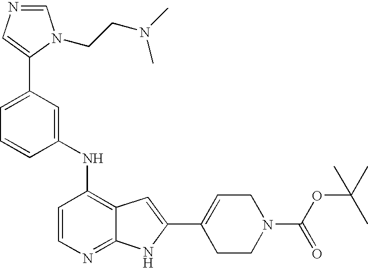

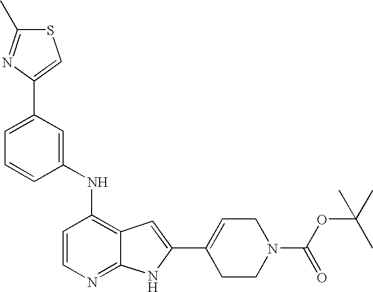

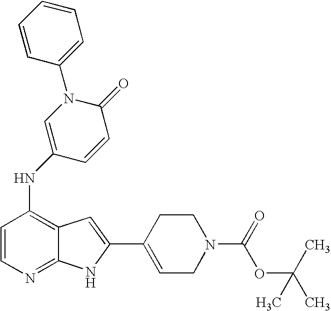

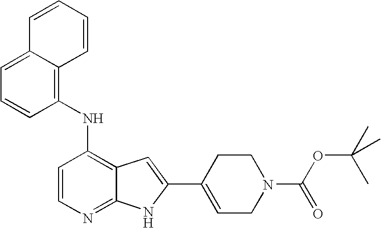

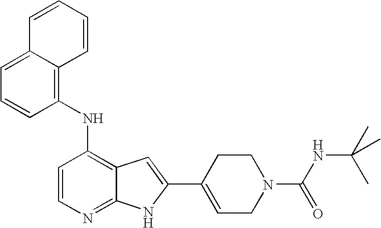

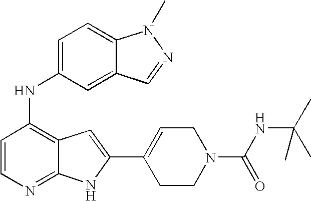

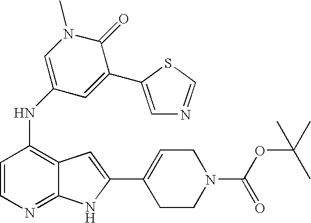

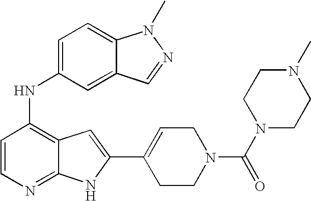

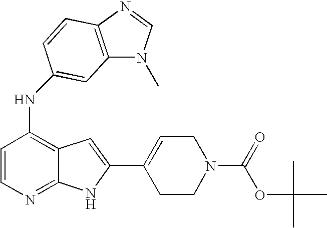

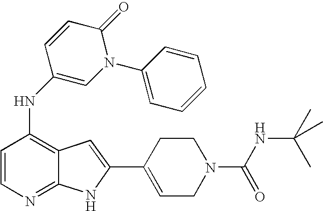

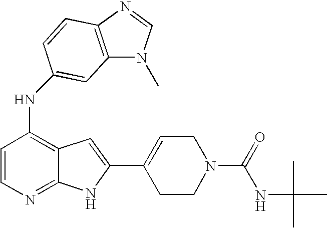

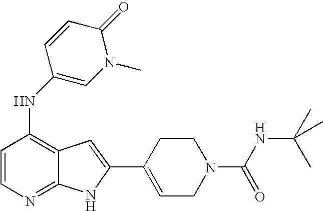

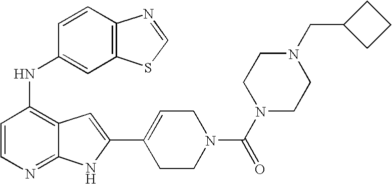

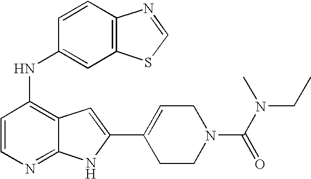

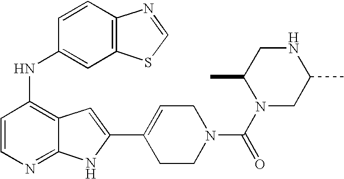

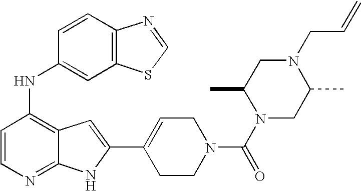

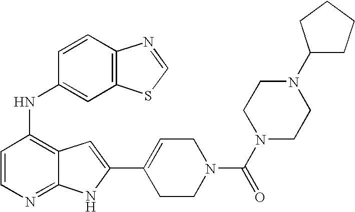

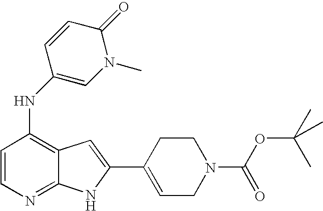

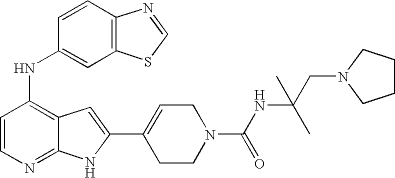

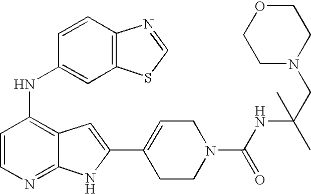

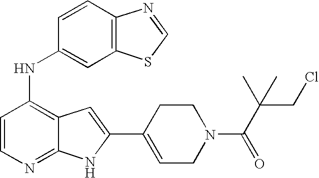

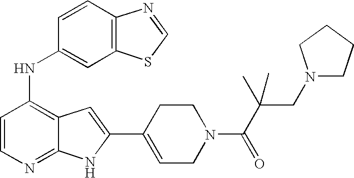

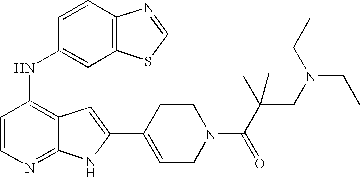

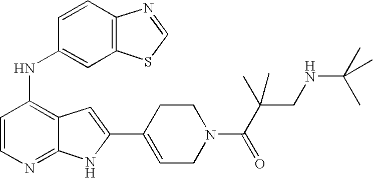

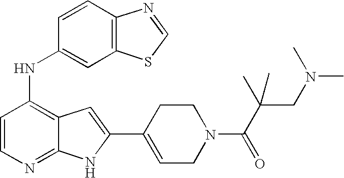

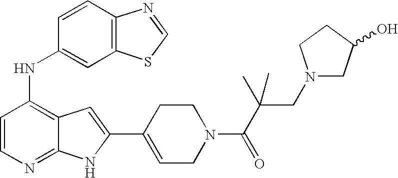

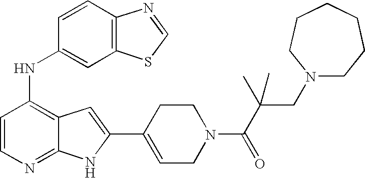

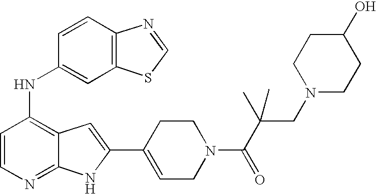

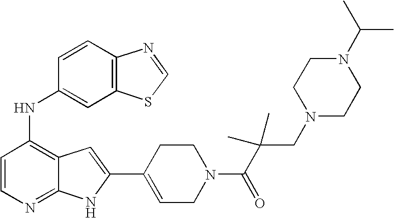

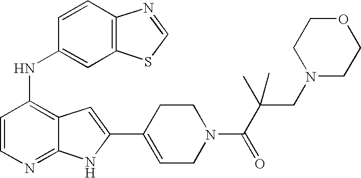

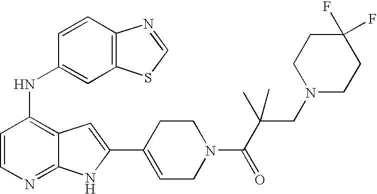

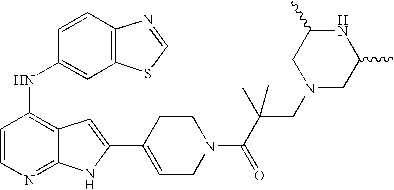

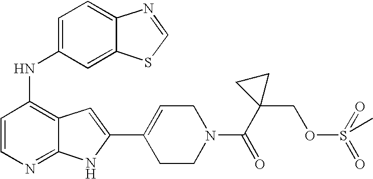

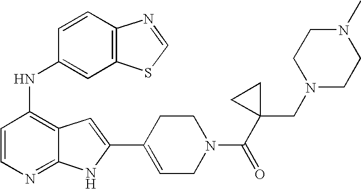

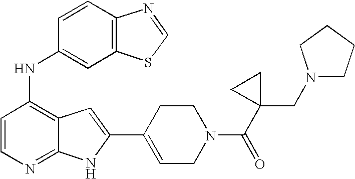

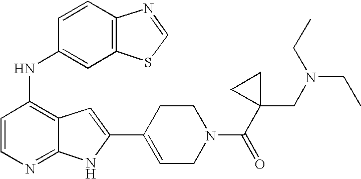

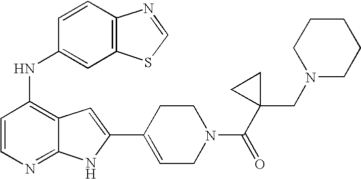

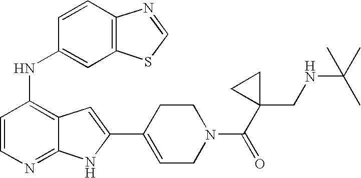

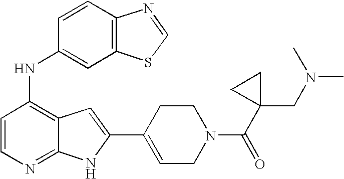

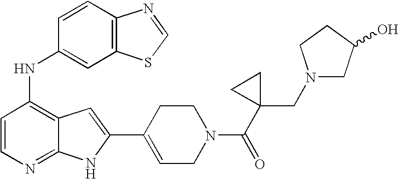

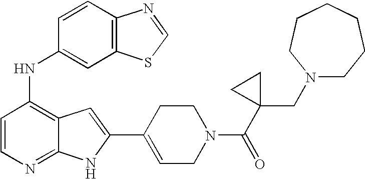

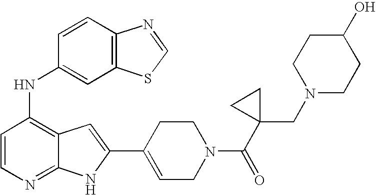

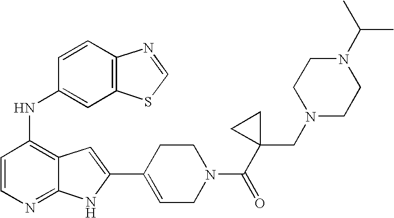

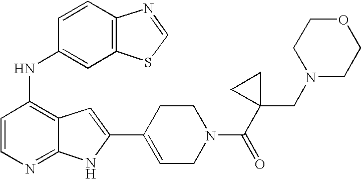

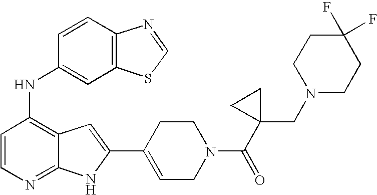

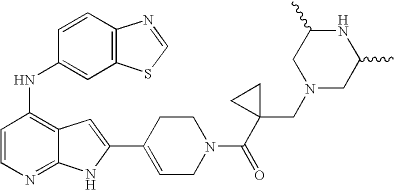

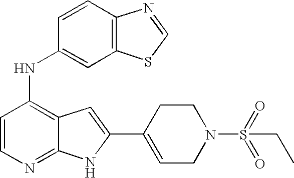

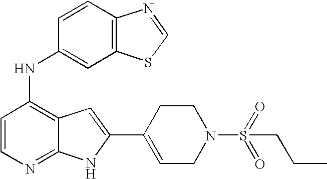

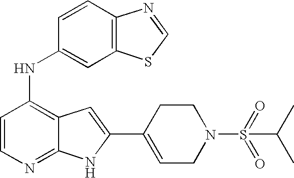

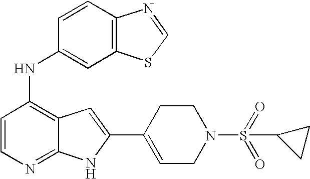

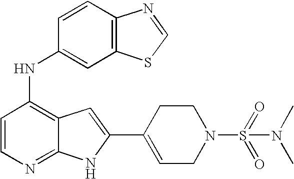

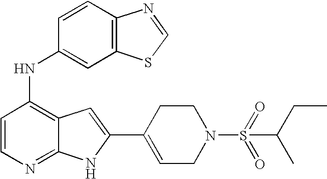

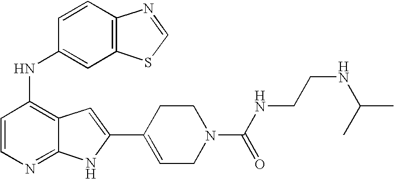

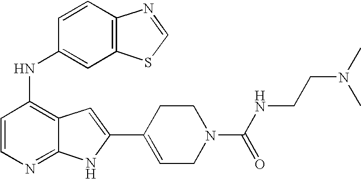

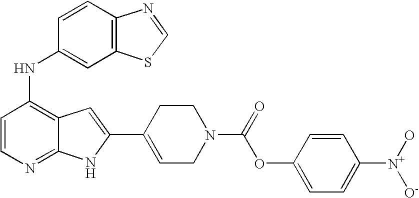

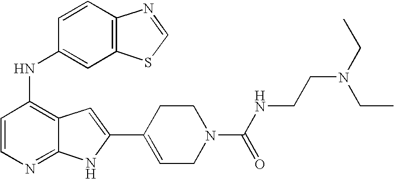

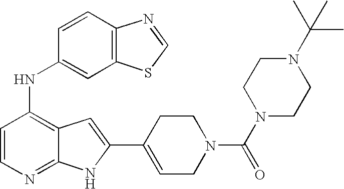

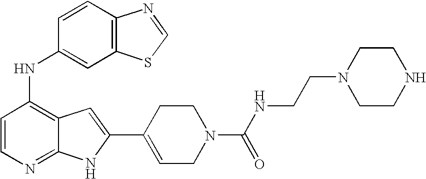

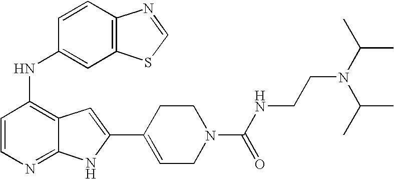

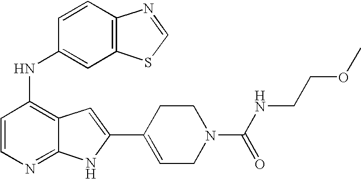

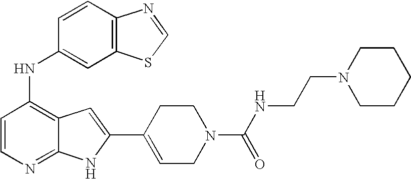

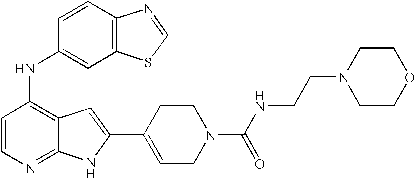

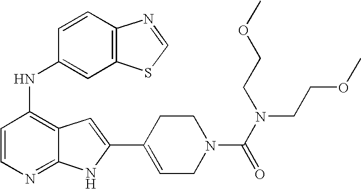

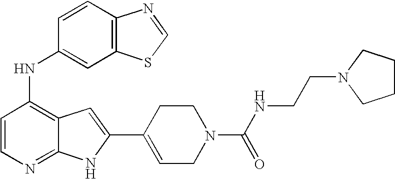

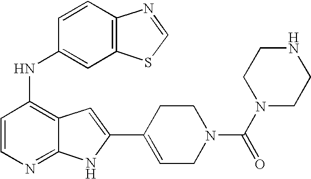

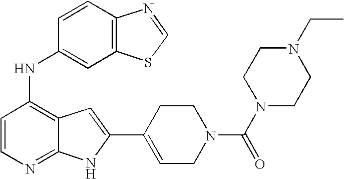

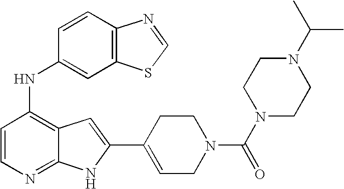

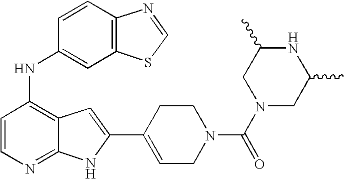

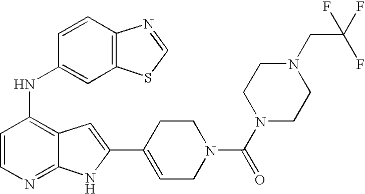

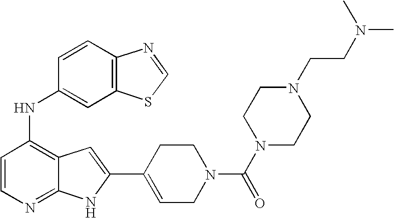

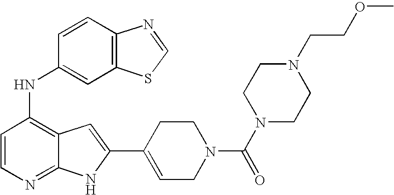

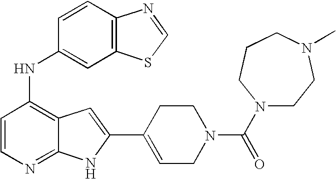

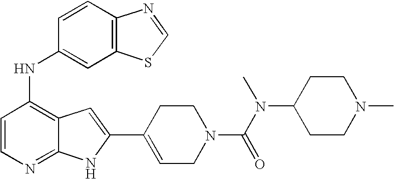

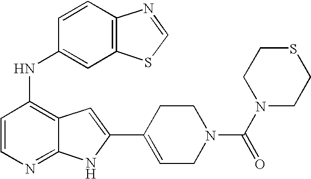

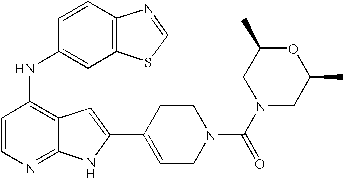

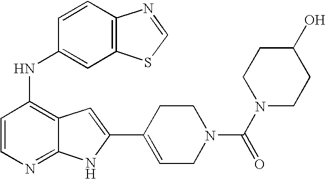

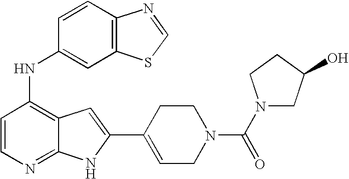

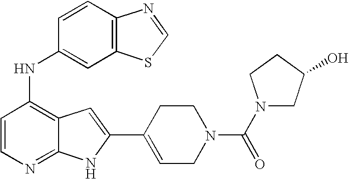

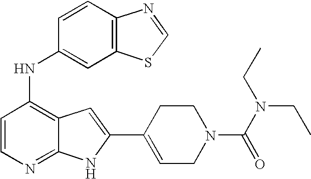

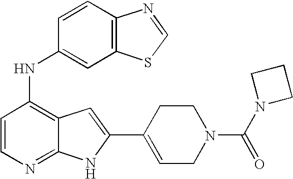

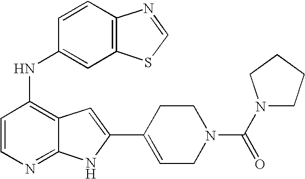

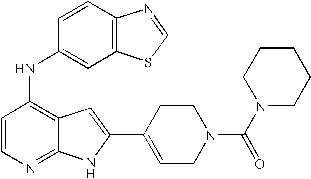

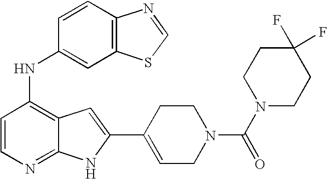

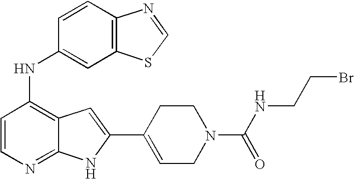

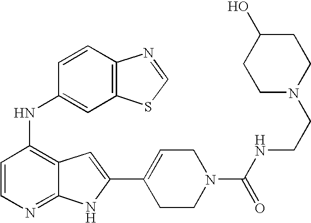

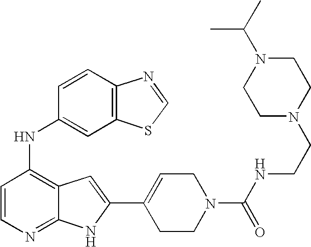

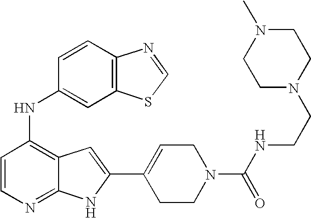

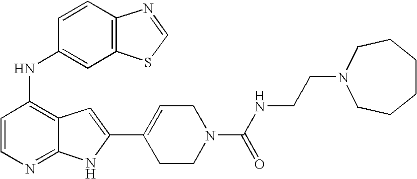

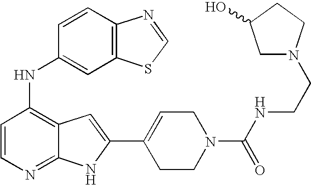

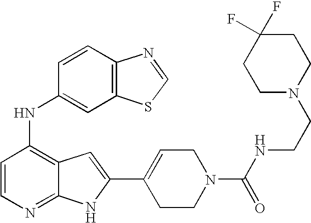

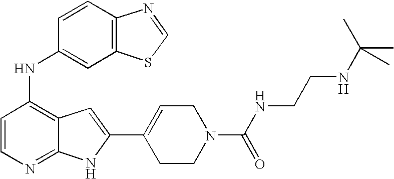

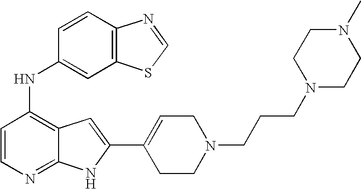

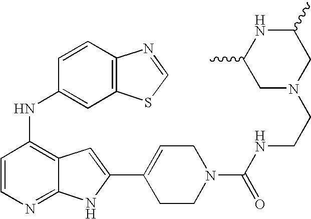

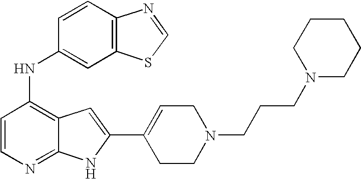

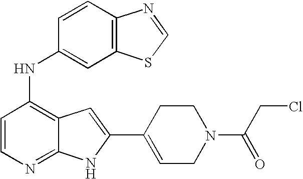

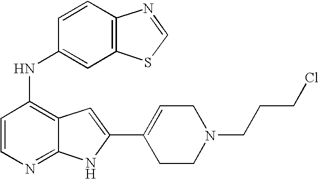

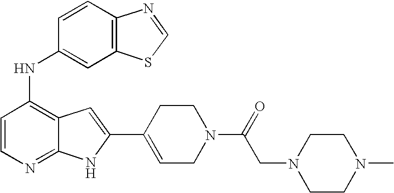

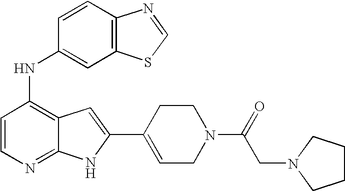

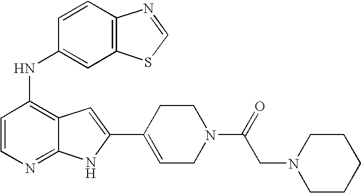

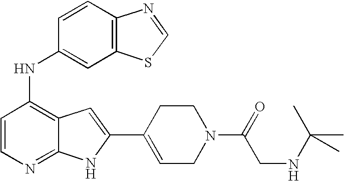

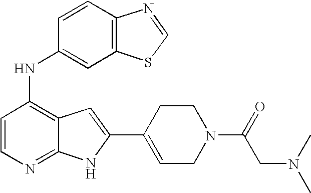

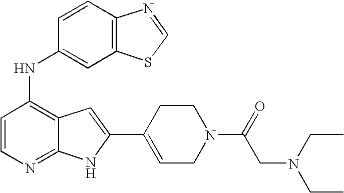

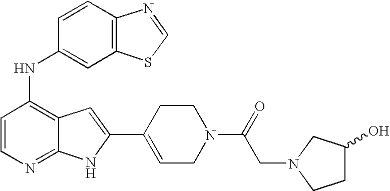

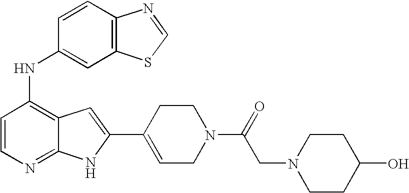

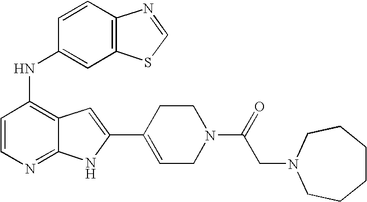

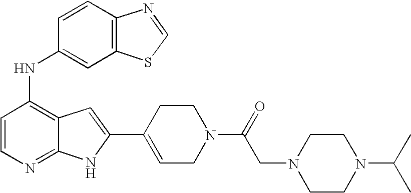

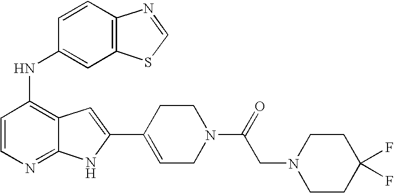

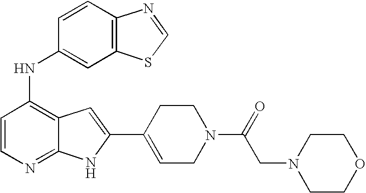

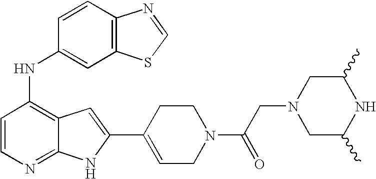

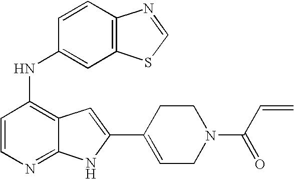

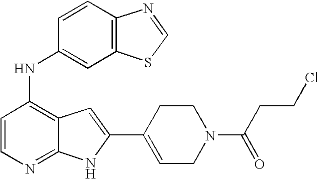

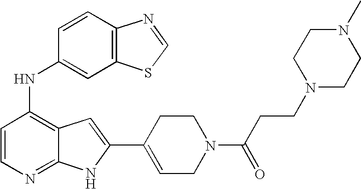

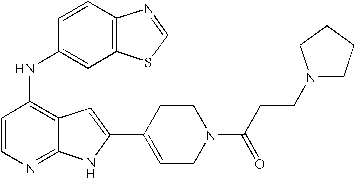

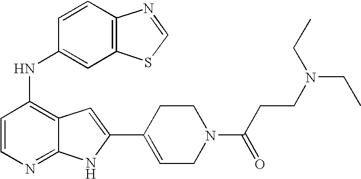

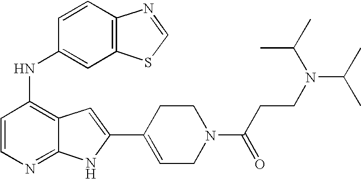

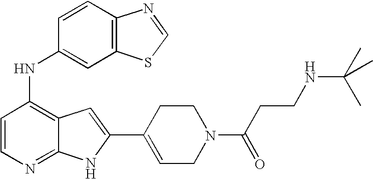

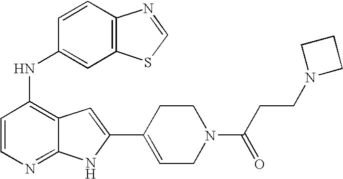

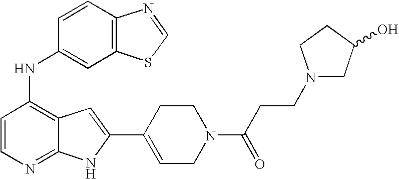

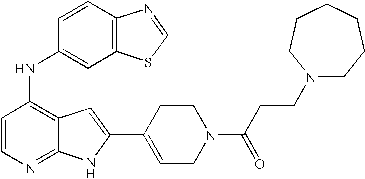

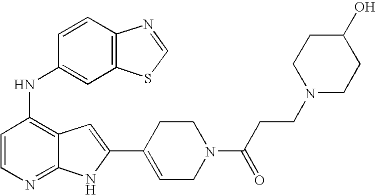

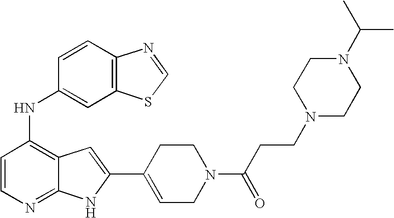

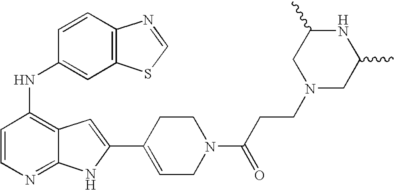

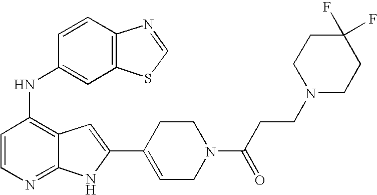

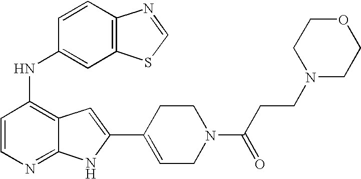

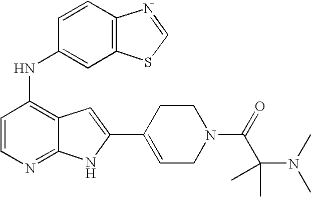

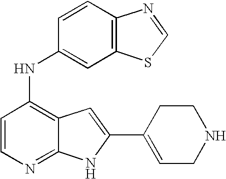

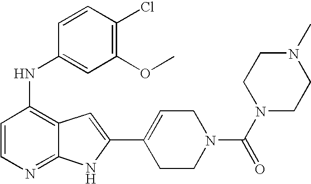

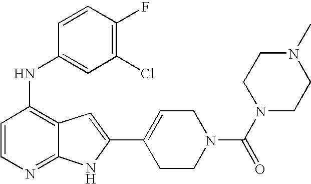

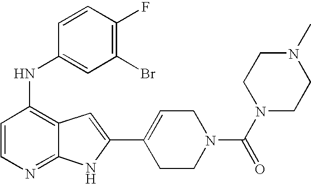

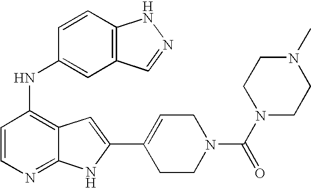

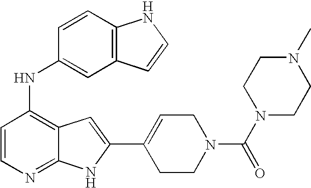

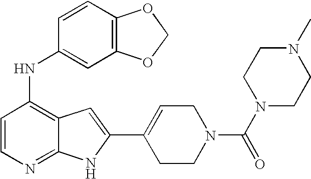

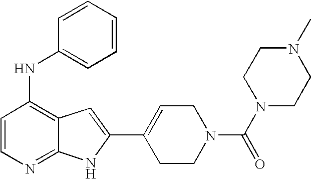

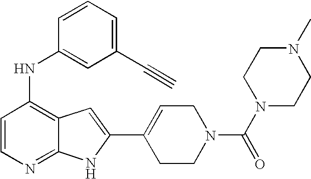

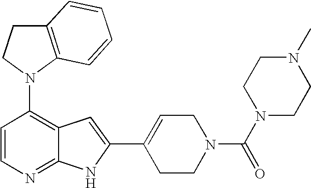

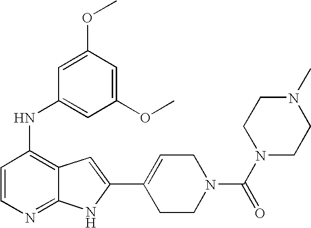

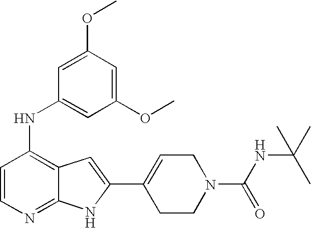

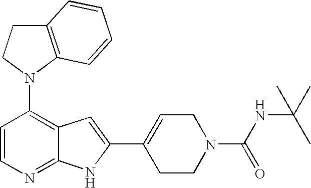

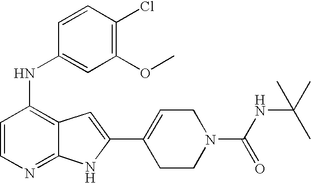

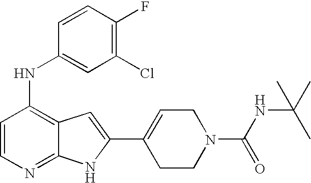

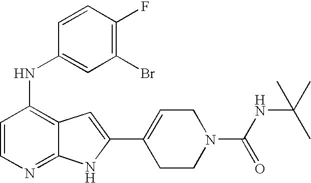

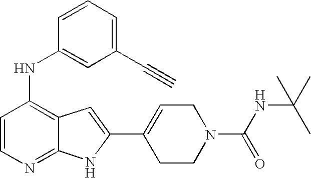

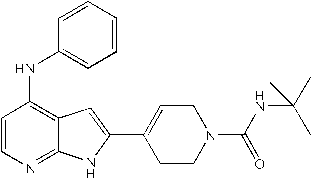

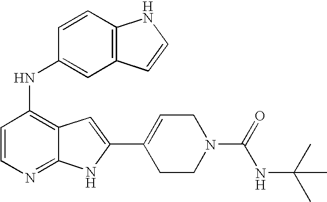

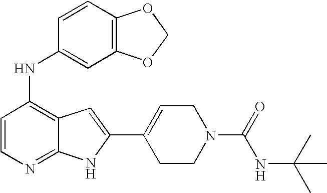

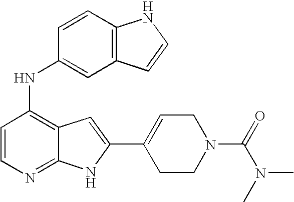

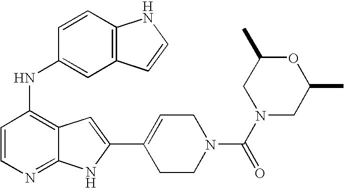

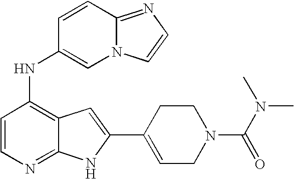

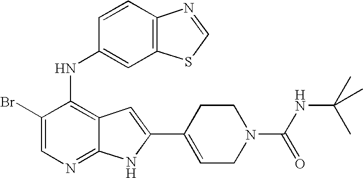

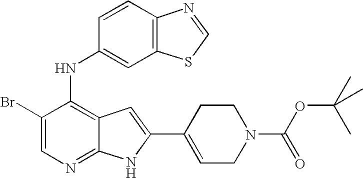

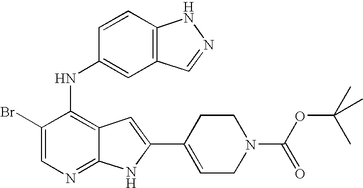

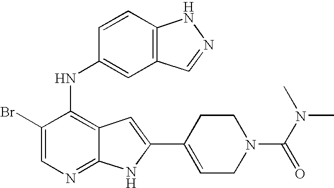

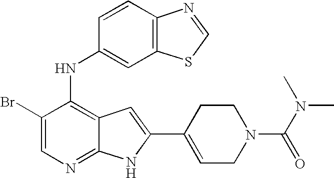

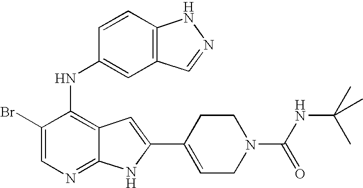

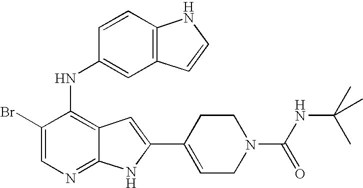

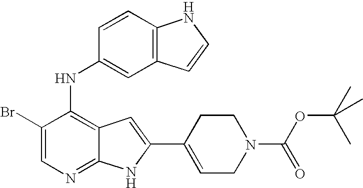

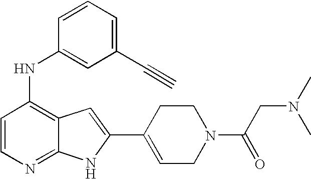

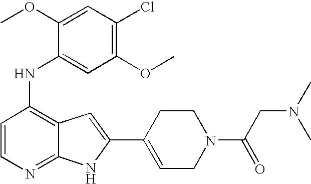

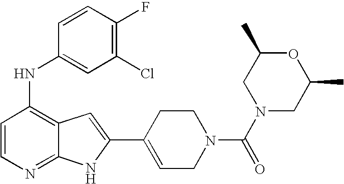

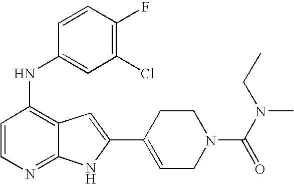

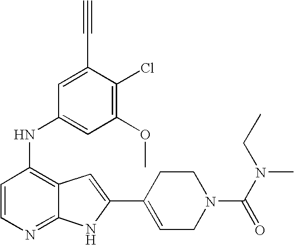

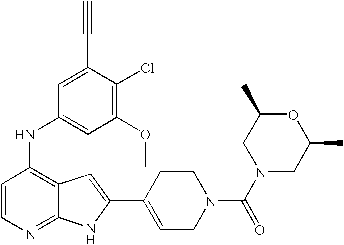

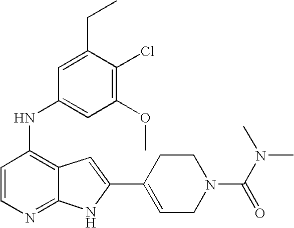

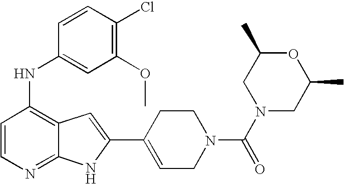

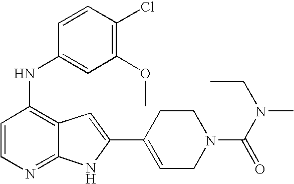

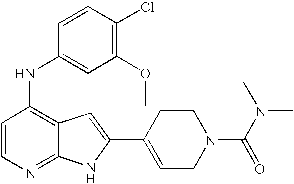

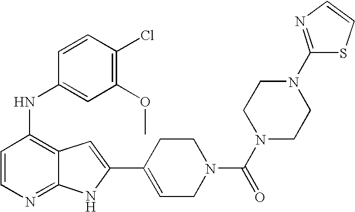

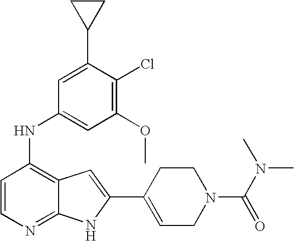

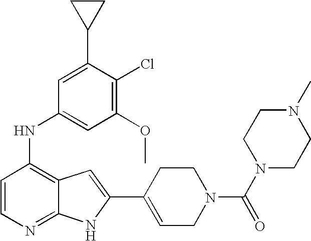

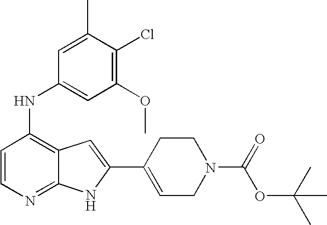

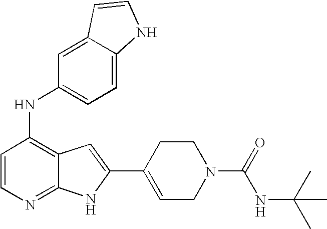

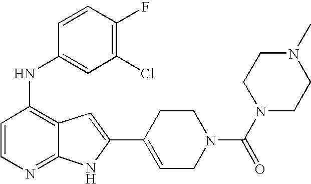

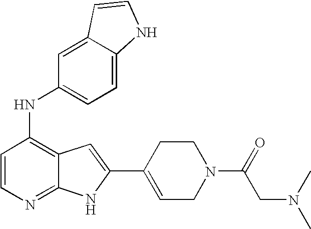

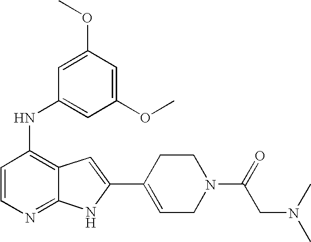

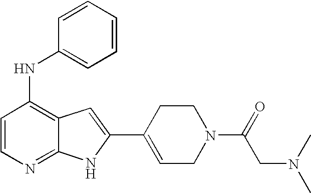

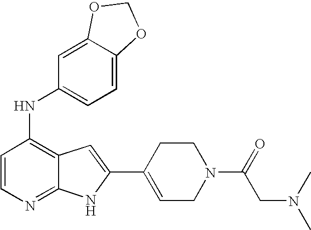

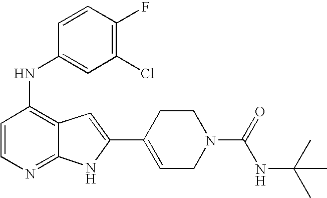

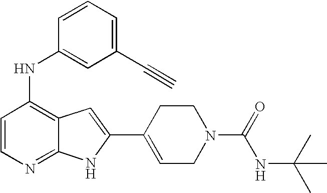

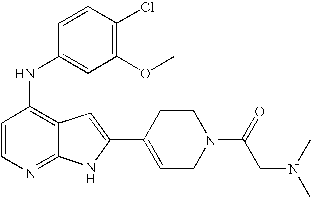

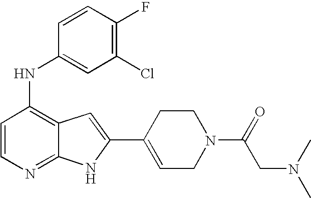

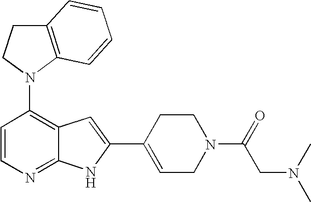

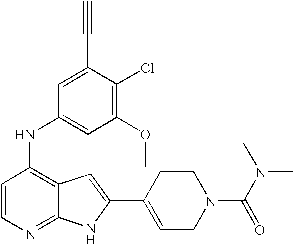

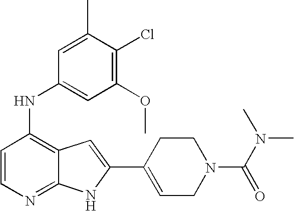

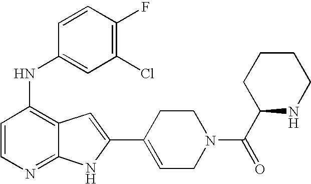

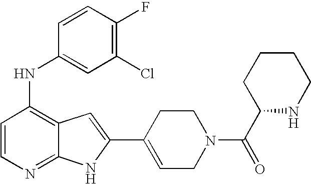

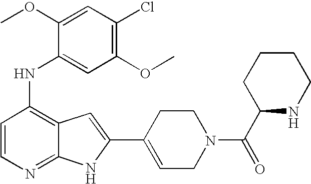

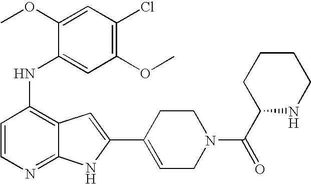

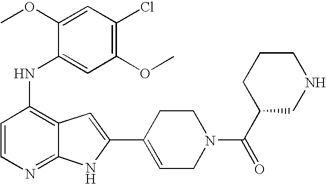

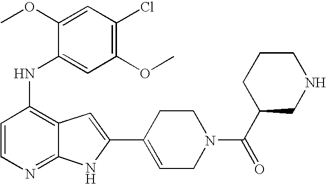

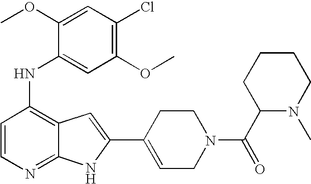

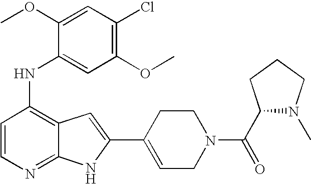

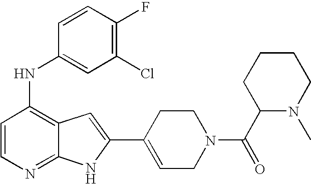

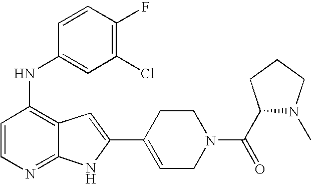

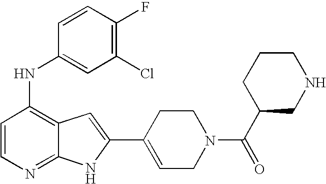

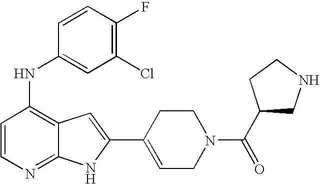

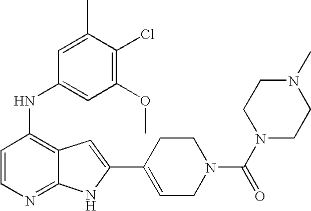

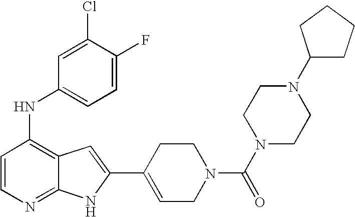

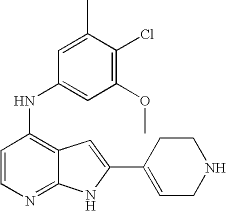

While the preferred groups for each variable have generally been listed above separately for each variable, preferred compounds of this invention include those in which several or each variable in Formula (I) is selected from the preferred, more preferred, most preferred, especially or particularly listed groups for each variable. Therefore, this invention is intended to include all combinations of preferred, more preferred, most preferred, especially and particularly listed groups.

The compounds of the present invention include:

4-[4-(4-Fluoro-3thiazol-5-ylphenylamino)-1H-pyrrolo[2,3-b]pyridin-2-yl]-3,6-dihydro-2H-pyridine-1-carboxylic acid tert-butyl ester;

4-[4-(4-Fluoro-3-thiazol-5-ylphenylamino)-1H-pyrrolo[2,3-b]pyridin-2-yl]-3,6-dihydro-2H-pyridine-1-carboxylic acid tert-butylamide;

4-{4-[4-Fluoro-3-(4-methylpiperazin-1-yl)-phenylamino]-1H-pyrrolo[2,3-b]pyridin-2-yl}-3,6dihydro-2H-pyridine-1-carboxylic acid tert-butyl ester;

4-{4-[4-Fluoro-3(4-methylpiperazin-1-yl)-phenylamino]-1H-pyrrolo[2,3-b]pyridin-2-yl}-3,6-dihydro-2H-pyridine-1-carboxylic acid tert-butylamide;

4-{4-[4-Fluoro-3-(1-methylazetidin-3-ylmethyl)-phenylamino]-1H-pyrrolo[2,3-b]pyridin-2-yl}-3,6-dihydro-2H-pyridine-1-carboxylic acid tert-butyl ester;

4-{4-[4-Fluoro-3(1-methylazetidin-3-ylmethyl)-phenylamino]-1H-pyrrolo[2,3-b]pyridin-2-yl}-3,6-dihydro-2H-pyridine-1-carboxylic acid tert-butylamide;

4-{4-[4-Fluoro-3(1-methylazetidin-3-yloxy)-phenylamino]-1H-pyrrolo[2,3-b]pyridin-2-yl}-6-dihydro-2H-pyridine-1-carboxylic acid tert-butyl ester;

4-{4-[4-Fluoro-3-(1-methylazetidin-3-yloxy)-phenylamino]-1H-pyrrolo[2,3-b]pyridin-2-yl}-3,6-dihydro-2H-pyridine-1-carboxylic acid tert-butyl ester;

4-{4-[4-Fluoro-3-(4-methylpiperazin-1-ylmethyl)-phenylamino]-1H-pyrrolo[2,3-b]pyridin-2-yl}-3,6-dihydro-2H-pyridine-1-carboxylic acid tert-butyl ester;

4-{4-[4-Fluoro-3-(4-methylpiperazin-1-ylmethyl)-phenylamino]-1H-pyrrolo[2,3-b]pyridin-2-yl}-3,6-dihydro-2H-pyridine-1-carboxylic acid tert-butylamide;

4-{4-[4-Fluoro-3-(1-methyl-1,2,3,6-tetrahydropyridin-4-yl)-phenylamino]-1H-pyrrolo[2,3-b]pyridin-2-yl}-3,6-dihydro-2H-pyridine-1-carboxylic acid tert-butyl ester;

4-{4-[4-Fluoro-3-(1-methyl-1,2,3,6-tetrahydropyridin-4-yl)-phenylamino]-1H-pyrrolo[2,3-b]pyridin-2-yl}-3,6-dihydro-2H-pyridine-1-carboxylic acid tert-butyl amide;

4-{4-[4-Fluoro-3-(1-methyl-2,5-dihydro-1H-pyrrol-3-yl)-phenylamino]-1H-pyrrolo[2,3-b]pyridin-2-yl}-3,6-dihydro-2H-pyridine-1-carboxylic acid tert-butyl ester;

4-{4-[4-Fluoro-3-(1-methyl-2,5-dihydro-1H-pyrrol-3-yl)-phenylamino]-1H-pyrrolo[2,3-b]pyridin-2-yl}-3,6-dihydro-2H-pyridine-1-carboxylic acid tert-butyl amide;

(S)-4-{4-[4-Fluoro-3-(1-methylpyrrolidin-3-yl)-phenylamino]-1H-pyrrolo[2,3-b]pyridin-2-yl}-3,6-dihydro-2H-pyridine-1-carboxylic acid tert-butyl ester;

(S)4-{4-[4-Fluoro-3-(1-methylpyrrolidin-3-yl)-phenylamino]-1H-pyrrolo[2,3-b]pyridin-2-yl}-3,6-dihydro-2H-pyridine-1-carboxylic acid tert-butyl amide;

(R)4-{4-[4-Fluoro-3-(1-methylpyrrolidin-3-yl)-phenylamino]-1H-pyrrolo[2,3-b]pyridin-2-yl}-3,6-dihydro-2H-pyridine-1-carboxylic acid tert-butyl ester;

(R)4-{4-[4-Fluoro-3-(1-methylpyrrolidin-3-yl)-phenylamino]-1H-pyrrolo[2,3]pyridin-2-yl}-3,6-dihydro-2H-pyridine-1-carboxylic acid tert-butyl amide;

{4-[4-(Benzothiazol-6-ylamino)-1H-pyrrolo[2,3-b]pyridin-2-yl]-3,6-dihydro-2H-pyridin-1-yl}-(2,2,4-trimethylpiperazin-1-yl)-methanone; {4-[4-(Benzothiazol-6-ylamino)-1H-pyrrolo[2,3-b]pyridin-2-yl]-3,6-dihydro-2H-pyridin-1-yl}(2,4,5-trimethylpiperazin-1-yl)-methanone;

{4-[4{(Benzothiazol-6-ylamino)-1H-pyrrolo[2,3-b]pyridin-2-yl]-3,6-dihydro-2H-pyridin-1-yl}-3,4,5-trimethylpiperazin-1-yl)-methanone;

{4-[4(Benzothiazol-6-ylamino)-1H-pyrrolo[2,3-b]pyridin-2-yl]-3,6-dihydro-2H-pyridin-1-yl}-[4-(2,2,2-trifluoroethyl)-piperazin-1-yl]-methanone; {4-[4(Benzothiazol-6-ylamino)-1H-pyrrolo[2,3-b]pyridin-2-yl]-3,6-dihydro-2H-pyridin-1-yl}-(4-tert-butylpiperazin-1-yl)-methanone;

Benzothiazol-6-yl-[6-(3,6-dihydro-2H-[1,2′]bipyridinyl-4-yl)-1H-pyrrolo[2,3-b]pyridin-2-yl]-amine;

Benzothiazol-6-yl-[6-(1-thiazol-2-yl-1,2,3,6-tetrahydropyridin-4-yl)-1H-pyrrolo[2,3-b]pyridin-2-yl]-amine;

Benzothiazol-6-yl-[6-(1-oxazol-2-yl-1,2,3,6-tetrahydropyridin-4-yl)-1H-pyrrolo[2,3-b]pyridin-2-yl]-amine;

4-[4-(3-Phenyl-3H-benzimidazol-5-ylamino)-1H-pyrrolo[2,3-b]pyridin-2-yl]-3,6-dihydro-2H-pyridine-1-carboxylic acid tert-butyl ester;

4-[4-(3-Phenyl-3H-benzimidazol-5-ylamino)-1H-pyrrolo[2,3-b]pyridin-2-yl]-3,6-dihydro-2H-pyridine-1-carboxylic acid tert-butylamide;

4-{4-[3-(2-Carbamoylphenyl)-3H-benzimidazol-5-ylamino]-1H-pyrrolo[2,3-b]pyridin-2-yl}-3,6-dihydro-2H-pyridine-1-carboxylic acid tert-butylamide;

4-{4-[3-(2-Carbamoylphenyl)-3H-benzimidazol-5-ylamino]-1H-pyrrolo[2,3-b]pyridin-2-yl}3,6-dihydro-2H-pyridine-1-carboxylic acid tert-butyl ester;

4-{4-[3-(2-Aminoethyl)-3H-benzimidazol-5-ylamino]-1H-pyrrolo[2,3-b]pyridin-2-yl}-3,6-dihydro-2H-pyridine-1-carboxylic acid tert-butylamide;

4-{4-[3-(2-Aminoethyl)-3H-benzimidazol-5-ylamino]-1H-pyrrolo[2,3-b]pyridin-2-yl}-3,6-dihydro-2H-pyridine-1-carboxylic acid tert-butyl ester;

4-{4-[3-(2-Dimethylaminoethyl)-3H-benzimidazol-5-ylamino]-1H-pyrrolo[2,3-b]pyridin-2-yl}-3,6-dihydro-2H-pyridine-1-carboxylic acid tert-butylamide;

4-{4-[3-(2-Dimethylaminoethyl)-3H-benzimidazol-5-ylamino]-1H-pyrrolo[2,3-b]pyridin-2-yl}-3,6-dihydro-2H-pyridine-1-carboxylic acid tert-butyl ester;

4-{4-[3-(2-Acetylaminoethyl)-3H-benzimidazol-5-ylamino]-1H-pyrrolo[2,3-b]pyridin-2-yl}-3,6-dihydro-2H-pyridine-1-carboxylic acid tert-butylamide;

4-{4-[3-(2-Acetylaminoethyl)-3H-benzimidazol-5-ylamino]-1H-pyrrolo[2,3-b]pyridin-2-yl}-3,6-dihydro-2H-pyridine-1-carboxylic acid tert-butyl ester;

4-[4-(Imidazo[1,2-a]pyridin-6-ylamino)-1H-pyrrolo[2,3-b]pyridin-2-yl]-3,6-dihydro-2H-pyridine-1-carboxylic acid tert-butylamide;

4-[4-(Imidazo[1,2-a]pyridin-6-ylamino)-1H-pyrrolo[2,3-b]pyridin-2-yl]-3,6-dihydro-2H-pyridine-1-carboxylic acid tert-butyl ester;

4-([4-3-Methylimidazo[1,2-a]pyridin-6-ylamino)-1H-pyrrolo[2,3-b]pyridin-2-yl]-3,6-dihydro-2H-pyridine-1-carboxylic acid tert-butylamide;

4-[4-(3-Methylimidazo[1,2-a]pyridin-6-ylamino)-1H-pyrrolo[2,3-b]pyridin-2-yl]-3,6-dihydro-2H-pyridine-1-carboxylic acid tert-butyl ester;

4-[4-3-Phenylimidazo[1,2-a]pyridin-6-ylamino)-1H-pyrrolo[2,3-b]pyridin-2-yl]-3,6-dihydro-2H-pyridine-1-carboxylic acid tert-butylamide;

4-[4(3-Phenylimidazo[1,2-a]pyridin-6-ylamino)-1H-pyrrolo[2,3-b]pyridin-2-yl]-3,6-dihydro-2H-pyridine-1-carboxylic acid tert-butyl ester;

4-{4-[3-(2-Carbamoylphenyl)-imidazo[1,2-a]pyridin-6-ylamino]-1H-pyrrolo[2,3-b]pyridin-2-yl}-3,6-dihydro-2H-pyridine-1-carboxylic acid tert-butylamide;

4-{4-[3-(2-Carbamoylphenyl)-imidazo[1,2-a]pyridin-6-ylamino]-1H-pyrrolo[2,3-b]pyridin-2-yl}-3,6-dihydro-2H-pyridine-1-carboxylic acid tert-butyl ester;

4-{4-[3-(2-Dimethylaminoethyl)-imidazo[1,2-a]pyridin-6-ylamino]-1H-pyrrolo[2,3-b]pyridin-2-yl}-3,6-dihydro-2H-pyridine-1-carboxylic acid tert-butylamide;

4-{4-[3-(2-Dimethylaminoethyl)-imidazo[1,2-a]pyridin-6-ylamino]-1H-pyrrolo[2,3-b]pyridin-2-yl}-3,6-dihydro-2H-pyridine-1-carboxylic acid tert-butyl ester;

4-{4-[3-(2-Acetylaminoethyl)-imidazo[1,2-a]pyridin-6-ylamino]-1H-pyrrolo[2,3-b]pyridin-2-yl}-3,6-dihydro-2H-pyridine-1-carboxylic acid tert-butylamide;

4-{4-[3-(2-Acetylaminoethyl)-imidazo[1,2-a]pyridin-6-ylamino]-1H-pyrrolo[2,3-b]pyridin-2-yl}-3,6-dihydro-2H-pyridine-1-carboxylic acid tert-butyl ester;

4-[4-(3-Dimethylaminomethylimidazo[1,2-a]pyridin-6-ylamino)-1H-pyrrolo[2,3-b]pyridin-2-yl]-3,6-dihydro-2H-pyridine-1-carboxylic acid tert-butylamide;

4-[4-(3-Dimethylaminomethylimidazo[1,2-a]pyridin-6-ylamino)-1H-pyrrolo[2,3-b]pyridin-2-yl]-3,6-dihydro-2H-pyridine-1-carboxylic acid tert-butyl ester;

4-[4-(7-Aminomethyl-1H-indazol-5-ylamino)-1H-pyrrolo[2,3-b]pyridin-2-yl]-3,6-dihydro-2H-pyridine-1-carboxylic acid tert-butylamide;

4-[4-(7-Aminomethyl-1H-indazol-5-ylamino)-1H-pyrrolo[2,3-b]pyridin-2-yl]-3,6-dihydro-2H-pyridine-1-carboxylic acid tert-butyl ester;

4-[4-(7-Aminomethyl-1-methyl-1H-indazol-5-ylamino)-1H-pyrrolo[2,3-b]pyridin-2-yl]-3,6-dihydro-2H-pyridine-1-carboxylic acid tert-butylamide;

4-[4-(7-Aminomethyl-1-methyl-1H-indazol-5-ylamino)-1H-pyrrolo[2,3-b]pyridin-2-yl]-3,6-dihydro-2H-pyridine-1-carboxylic acid tert-butyl ester;

4-[4-(7-Dimethylaminomethyl-1-methyl-1H-indazol-5-ylamino)-1H-pyrrolo[2,3-b]pyridin-2-yl]-3,6-dihydro-2H-pyridine-1-carboxylic acid tert-butylamide;

4-[4-(7-Dimethylaminomethyl-1-methyl-1H-indazol-5-ylamino)-1H-pyrrolo[2,3-b]pyridin-2-yl]-3,6-dihydro-2H-pyridine-1-carboxylic acid tert-butyl ester;

4-[4-(7-Dimethylaminomethyl-1H-indazol-5-ylamino)-1H-pyrrolo[2,3-b]pyridin-2-yl]-3,6-dihydro-2H-pyridine-1-carboxylic acid tert-butylamide;

4-[4-7-Dimethylaminomethyl-1H-indazol-5-ylamino)-1H-pyrrolo[2,3-b]pyridin-2-yl]-3,6-dihydro-2H-pyridine-1-carboxylic acid tert-butyl ester;

4-{4-[7-(2-Dimethylaminoethyl)-1H-indazol-5-ylamino]-1H-pyrrolo[2,3-b]pyridin-2-yl}-3,6-dihydro-2H-pyridine-1-carboxylic acid tert-butylamide;

4-{4-[7-(2-Dimethylaminoethyl)-1H-indazol-5-ylamino]-1H-pyrrolo[2,3-b]pyridin-2-yl}-3,6-dihydro-2H-pyridine-1-carboxylic acid tert-butyl ester;

4-{4-[7-(2-Dimethylaminoethyl)-1-methyl-1H-indazol-5-ylamino]-1H-pyrrolo[2,3-b]pyridin-2-yl}-3,6-dihydro-2H-pyridine-1-carboxylic acid tert-butylamide;

4-{4-[7-(2-Dimethylaminoethyl)-1-methyl-1H-indazol-5-ylamino]-1H-pyrrolo[2,3-b]pyridin-2-yl}-3,6-dihydro-2H-pyridine-1-carboxylic acid tert-butyl ester;

4-[4-(Imidazo[1,5-a]pyridin-6-ylamino)-1H-pyrrolo[2,3-b]pyridin-2-yl]-3,6-dihydro-2H-pyridine-1-carboxylic acid tert-butylamide;

4-[4-(Imidazo[1,5-a]pyridin-6-ylamino)-1H-pyrrolo[2,3-b]pyridin-2-yl]-3,6-dihydro-2H-pyridine-1-carboxylic acid tert-butyl ester;

4-[4-(3-Methylimidazo[1,5-a]pyridin-6-ylamino)-1H-pyrrolo[2,3-b]pyridin-2-yl]-3,6-dihydro-2H-pyridine-1-carboxylic acid tert-butylamide;

4-[4-(3-Methylimidazo[1,5-a]pyridin-6-ylamino)-1H-pyrrolo[2,3-b]pyridin-2-yl]-3,6-dihydro-2H-pyridine-1-carboxylic acid tert-butyl ester;

4-[4-(1-Methylimidazo[1,5-a]pyridin-6-ylamino)-1H-pyrrolo[2,3-b]pyridin-2-yl]-3,6-dihydro-2H-pyridine-1-carboxylic acid tert-butylamide;

4-[4-(1-Methylimidazo[1,5-a]pyridin-6-ylamino)-1H-pyrrolo[2,3-b]pyridin-2-yl]-3,6-dihydro-2H-pyridine-1-carboxylic acid tert-butyl ester;

4-[4-(Imidazo[1,5-a]pyridin-7-ylamino)-1H-pyrrolo[2,3-b]pyridin-2-yl]-3,6-dihydro-2H-pyridine-1-carboxylic acid tert-butylamide;

4-[4-(Imidazo[1,5-a]pyridin-7-ylamino)-1H-pyrrolo[2,3-b]pyridin-2-yl]-3,6-dihydro-2H-pyridine-1-carboxylic acid tert-butyl ester;

4-[4-(3-Methylimidazo[1,5-a]pyridin-7-ylamino)-1H-pyrrolo[2,3-b]pyridin-2-yl]-3,6-dihydro-2H-pyridine-1-carboxylic acid tert-butylamide;

4-[4-(3-Methylimidazo[1,5-a]pyridin-7-ylamino)-1H-pyrrolo[2,3-b]pyridin-2-yl]-3,6-dihydro-2H-pyridine-1-carboxylic acid tert-butyl ester;

4-[4-(1-Methylimidazo[1,5-a]pyridin-7-ylamino)-1H-pyrrolo[2,3-b]pyridin-2-yl]-3,6-dihydro-2H-pyridine-1-carboxylic acid tert-butylamide;

4-[4-(1-Methylimidazo[1,5-a]pyridin-7-ylamino)-1H-pyrrolo[2,3-b]pyridin-2-yl]-3,6-dihydro-2H-pyridine-1-carboxylic acid tert-butyl ester;

4-[4-(Benzothiazol-6-ylamino)-1H-pyrrolo[2,3-b]pyridin-2-yl]-3,6-dihydro-2H-pyridine-1-carboxylic acid (2-hydroxy-1,1-dimethylethyl)-amide;

4-[4(1-Methyl-1H-indazol-5-ylamino)-1H-pyrrolo[2,3-b]pyridin-2-yl]-3,6-dihydro-2H-pyridine-1-carboxylic acid (2-hydroxy-1,1-dimethylethyl)-amide;

4-[4-(Benzothiazol-6-ylamino)-1H-pyrrolo[2,3-b]pyridin-2-yl]-3,6-dihydro-2H-pyridine-1-carboxylic acid (2-methoxy-1,1-dimethylethyl)-amide;

4-[4(1-Methyl-1H-indazol-5-ylamino)-1H-pyrrolo[2,3-b]pyridin-2-yl]-3,6-dihydro-2H-pyridine-1-carboxylic acid (2-methoxy-1,1-dimethylethyl)-amide;

4-[4-(Benzothiazol-6-ylamino)-1H-pyrrolo[2,3-b]pyridin-2-yl]-3,6-dihydro-2H-pyridine-1-carboxylic acid (2-dimethylamino-1,1-dimethylethyl)-amide;

4-[4-(1-Methyl-1H-indazol-5-ylamino)-1H-pyrrolo[2,3-b]pyridin-2-yl]-3,6-dihydro-2H-pyridine-1-carboxylic acid (2-dimethylamino-1,1-dimethylethyl)-amide;

4-[4-(Benzothiazol-6-ylamino)-1H-pyrrolo[2,3-b]pyridin-2-yl]-3,6-dihydro-2H-pyridine-1-carboxylic acid (1,1-dimethyl-2-pyrrolidin-1-ylethyl)-amide;

4-[4-(1-Methyl-1H-indazol-5-ylamino)-1H-pyrrolo[2,3-b]pyridin-2-yl]-3,6-dihydro-2H-pyridine-1-carboxylic acid (1,1-dimethyl-2-pyrrolidin-1-ylethyl)-amide;

4-[4-(Benzothiazol-6-ylamino)-1H-pyrrolo[2,3-b]pyridin-2-yl]-3,6-dihydro-2H-pyridine-1-carboxylic acid (1,1-dimethyl-2-morpholin-4-ylethyl)-amide;

4-[4-(1-Methyl-1H-indazol-5-ylamino)-1H-pyrrolo[2,3-b]pyridin-2-yl]-3,6-dihydro-2H-pyridine-1-carboxylic acid (1,1-dimethyl-2-morpholin-4-ylethyl)-amide;

4-[4-(1H-Indol-5-ylamino)-1H-pyrrolo[2,3-b]pyridin-2-yl]-3,6-dihydro-2H-pyridine-1-carboxylic acid tert-butylamide;

4-[4-(Quinolin-6-ylamino)-1H-pyrrolo[2,3-b]pyridin-2-yl]-3,6-dihydro-2H-pyridine-1-carboxylic acid tert-butylamide;

(Hexahydropyrrolo[1,2-a]pyrazin-2-yl)-{4-[4-(imidazo[1,2-a]pyridin-6-ylamino)-1H-pyrrolo[2,3-b]pyridin-2-yl]-3,6-dihydro-2H-pyridin-1-yl}-methanone;

(Hexahydropyrrolo[1,2-a]pyrazin-2-yl)-{4-[4-(3-methyl-3H-benzoimidazol-5-ylamino)-1H-pyrrolo[2,3-b]pyridin-2-yl]-3,6-dihydro-2H-pyridin-1-yl}-methanone;

(Hexahydropyrrolo[1,2-a]pyrazin-2-yl)-{4-[4-(1-methyl-1H-indazol-5-ylamino)-1H-pyrrolo[2,3-b]pyridin-2-yl]-3,6-dihydro-2H-pyridin-1-yl}-methanone;

4-[4-(Benzothiazol-6-ylamino)-1H-pyrrolo[2,3-b]pyridin-2-yl]-3,6-dihydro-2H-pyridin-1-yl}-(hexahydropyrrolo[1,2-a]pyrazin-2-yl)-methanone;

2-Dimethylamino-1-{4-[4-(3-ethynylphenylamino)-1H-pyrrolo[2,3-b]pyridin-2-yl]-3,6-dihydro-2H-pyridin-1-yl}-ethanone;

1-{4-[4-(4-Chloro-2,5-dimethoxyphenylamino)-1H-pyrrolo[2,3-b]pyridin-2-yl]-3,6-dihydro-2H-pyridin-1-yl}-2-dimethylaminoethanone;

{4-[4-(3-Chloro-4-fluorophenylamino)-1H-pyrrolo[2,3-b]pyridin-2-yl]-3,6-dihydro-2H-pyridin-1-yl}-(cis-2,6-dimethylmorpholin4-yl)-methanone;

b 4-[4-(3-Chloro-4-fluorophenylamino)-1H-pyrrolo[2,3-b]pyridin-2-yl]-3,6-dihydro-2H-pyridine-1-carboxylic acid ethyl-methyl-amide;

4-[4-(4-Chloro-3-ethynyl-5-methoxyphenylamino)-1H-pyrrolo[2,3-b]pyridin-2-yl]-3,6-dihydro-2H-pyridine-1-carboxylic acid ethyl-methyl-amide;

{4-[4-(4-Chloro-3-ethynyl-5-methoxyphenylamino)-1H-pyrrolo[2,3-b]pyridin-2-yl]-3,6-dihydro-2H-pyridin-1-yl}-(cis-2,6-dimethylmorpholin-4-yl)-methanone;

4-[4-(4-Chloro-3-ethyl-5-methoxyphenylamino)-1H-pyrrolo[2,3-b]pyridin-2-yl]-3,6-dihydro-2H-pyridine-1-carboxylic acid dimethylamide;

{4-[4-(4-Chloro-3-methoxyphenylamino)-1H-pyrrolo[2,3-b]pyridin-2-yl]-3,6-dihydro-2H-pyridin-1-yl}-(cis-2,6-dimethylmorpholin-4-yl)-methanone;

4-[4-(4-Chloro-3-methoxyphenylamino)-1H-pyrrolo[2,3-b]pyridin-2-yl]-3,6-dihydro-2H-pyridine-1-carboxylic acid ethyl-methyl-amide;

4-[4-(4-Chloro-3-methoxyphenylamino)-1H-pyrrolo[2,3-b]pyridin-2-yl]-3,6-dihydro-2H-pyridine-1-carboxylic acid dimethylamide;

{4-[4-(4-Chloro-3-methoxyphenylamino)-1H-pyrrolo[2,3-b]pyridin-2-yl]-3,6-dihydro-2H-pyridin-1-yl}-(4-thiazol-2-yl-piperazin-1-yl)-methanone;

4-[4-(4-Chloro-3-cyclopropyl-5-methoxyphenylamino)-1H-pyrrolo[2,3-b]pyridin-2-yl]-3,6-dihydro-2H -pyridine-1-carboxylicacid dimethylamide;

{4-[4-(4-Chloro-3-cyclopropyl-5-methoxyphenylamino)-1H-pyrrolo[2,3-b]pyridin-2-yl]-3,6-dihydro-2H-pyridin-1-yl}-(4-methylpiperazin-1-yl)-methanone;

4-[4-(4-Chloro-3-methoxy-5-methylphenylamino)-1H-pyrrolo[2,3-b]pyridin-2-yl]-3,6-dihydro-2H-pyridine-1-carboxylic acid tert-butyl ester;

4-[4-(1H-Indol-5-ylamino)-1H-pyrrolo[2,3-b]pyridin-2-yl]-3,6-dihydro-2H-pyridine-1-carboxylic acid tert-butylamide;

{4-[4-(1H-Indol-5-ylamino)-1H-pyrrolo[2,3-b]pyridin-2-yl]-3,6-dihydro-2H-pyridin-1-yl}-(4-methylpiperazin-1-yl)-methanone;

{4-[4-(3-Chloro-4-fluorophenylamino)-1H-pyrrolo[2,3-b]pyridin-2-yl]-3,6-dihydro-2H-pyridin-1-yl}-(4-methylpiperazin-1-yl)-methanone;

2-Dimethylamino-1-4-[4-(1H-indol-5-ylamino)-1H-pyrrolo[2,3-b]pyridin-2-yl]-3,6-dihydro-2H-pyridin-1-yl-ethanone;

4-[4-(3,5-Dimethoxyphenylamino)-1H-pyrrolo[2,3-b]pyridin-2-yl]-3,6-dihydro-2H-pyridin-1-yl-2-dimethylaminoethanone;

2-Dimethylamino-1-[4-(4-phenylamino-1H-pyrrolo[2,3-b]pyridin-2-yl)-3,6-dihydro-2H-pyridin-1-yl]-ethanone;

4-[4-(Benzo[1,3]dioxol-5-ylamino)-1H-pyrrolo[2,3-b]pyridin-2-yl]-3,6-dihydro-2H-pyridin-1-yl-(4-methylpiperazin-1-yl)-methanone;

4-[4-(3-Chloro-4-fluorophenylamino)-1H-pyrrolo[2,3-b]pyridin-2-yl]-3,6-dihydro-2H-pyridine-1-carboxylic acid t-butylamide;

4-[4-(3-Ethynylphenylamino)-1H-pyrrolo[2,3-b]pyridin-2-yl]-3,6-dihydro-2H-pyridine-1-carboxylic acid tert-butylamide;

1-4-[4-(4-Chloro-3-methoxyphenylamino)-1H-pyrrolo[2,3-b]pyridin-2-yl]-3,6-dihydro-2H-pyridin-1-yl-2-dimethylaminoethanone;

1-4-[4-(3-Chloro-4-fluorophenylamino)-1H-pyrrolo[2,3-b]pyridin-2-yl]-3,6-dihydro-2H-pyridin-1-yl-2-dimethylaminoethanone;

1-4-[4-(2,3-Dihydroindol-1-yl)-1H-pyrrolo[2,3-b]pyridin-2-yl]-3,6-dihydro-2H-pyridin-1-yl-2-dimethylaminoethanone;

4-[4-(4-Chloro-3-ethynyl-5-methoxyphenylamino)-1H-pyrrolo[2,3-b]pyridin-2-yl]-3,6-dihydro-2H-pyridine-1-carboxylic acid dimethylamide;

4-[4-(4-Chloro-3-methoxy-5-methylphenylamino)-1H-pyrrolo[2,3-b]pyridin-2-yl]-3,6-dihydro-2H-pyridine-1-carboxylic acid dimethylamide;

{4-[4-(3-Chloro-4-fluorophenylamino)-1H-pyrrolo[2,3-b]pyridin-2-yl]-3,6-dihydro-2H-pyridin-1-yl}-(R)-piperidin-2-yl-methanone;

{4-[4-(4-Chloro-2,5-dimethoxyphenylamino)-1H-pyrrolo[2,3-b]pyridin-2-yl]-3,6-dihydro-2H-pyridin-1-yl}-(R)-piperidin-2-yl-methanone;

{4-[4-(4-Chloro-2,5-dimethoxyphenylamino)-1H-pyrrolo[2,3-b]pyridin-2-yl]-3,6-dihydro-2H-pyridin-1-yl}-(S)-piperidin-2-yl-methanone;

{4-[4-(4-Chloro-2,5-dimethoxyphenylamino)-1H-pyrrolo[2,3-b]pyridin-2-yl]-3,6-dihydro-2H-pyridin-1-yl}-(S)-piperidin-3-yl-methanone;

{4-[4-(4-Chloro-2,5-dimethoxyphenylamino)-1H-pyrrolo[2,3-b]pyridin-2-yl]-3,6-dihydro-2H-pyridin-1-yl}-(R)-piperidin-3-yl-methanone;

{4-[4-(4-Chloro-2,5-dimethoxyphenylamino)-1H-pyrrolo[2,3-b]pyridin-2-yl]-3,6-dihydro-2H-pyridin-1-yl}-(1-methylpiperidin-2-yl)-methanone;

{4-[4-(4-Chloro-2,5-dimethoxyphenylamino)-1H-pyrrolo[2,3-b]pyridin-2-yl]-3,6-dihydro-2H-pyridin-1-yl}-((S)-1-methylpyrrolidin-2-yl)-methanone;

{4-[4-(4-Chloro-2,5-dimethoxyphenylamino)-1H-pyrrolo[2,3-b]pyridin-2-yl]-3,6-dihydro-2H-pyridin-1-yl}-((S)-1-methylpyrrolidin-2-yl)-methanone;

{4-[4-(3-Chloro-4-fluorophenylamino)-1H-pyrrolo[2,3-b]pyridin-2-yl]-3,6-dihydro-2H-pyridin-1-yl}-((s)-1-methylpyrrolidin-2-yl)-methanone;

{4-[4-(3-Chloro-4-fluorophenylamino)-1H-pyrrolo[2,3-b]pyridin-2-yl]-3,6-dihydro-2H-pyridin-1-yl}-(S)-piperidin-3-yl-methanone;

{4-[4-(3-Chloro-4-fluorophenylamino)-1H-pyrrolo[2,3-b]pyridin-2-yl]-3,6-dihydro-2H-pyridin-1-yl}-(S)-pyrrolidin-3-yl-methanone;

{4-[4-(4-Chloro-3-methoxy-5-methylphenylamino)-1H-pyrrolo[2,3-b]pyridin-2-yl]-3,6-dihydro-2H-pyridin-1-yl}-(4-methylpiperazin-1-yl)-methanone;

N-(4-Chloro-3-methoxy-5-methylphenyl)-2(1,2,3,6-tetrahydropyridin4-yl)-1H-pyrrolo[2,3-b]pyridin-4-amine.

As used herein, unless stated otherwise, “alkyl” as well as other groups having the prefix “alk” such as, for example, alkoxy, alkanyl, alkenyl, alkynyl, and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains having at least one unsaturated carbon-carbon bond.

As used herein, for example, “C₀₋₄alkyl” is used to mean an alkyl having 0-4 carbons—that is, 0, 1, 2, 3, or 4 carbons in a straight or branched configuration. An alkyl having no carbon is hydrogen when the alkyl is a terminal group. An alkyl having no carbon is a direct bond when the alkyl is a bridging (connecting) group.

As used herein, the “>” symbol in front of a nitrogen atom refers to two bonds not to the same atom (not a double bond to the nitrogen).

The terms “cycloalkyl” and “carbocyclic ring” mean carbocycles containing no heteroatoms, and include mono-, bi-, and tricyclic saturated carbocycles, as well as fused and bridged systems. Such fused ring systems can include one ring that is partially or fully unsaturated, such as a benzene ring, to form fused ring systems, such as benzofused carbocycles. Cycloalkyl includes such fused ring systems as spirofused ring systems. Examples of cycloalkyl and carbocyclic rings include C3-10cycloalkyl groups, particularly C3-8cycloalkyl groups, such as cyclopropyl, cyclobutyl, cyclopentyl, cyclohexyl, and decahydronaphthalene, adamantane, indanyl, 1,2,3,4-tetrahydronaphthalene and the like.

The term “halogen” includes fluorine, chlorine, bromine, and iodine atoms.

The term “carbamoyl” unless specifically described otherwise means —C(O)—NH— or —NH—C(O)—.

The term “aryl” is well known to chemists. The preferred aryl groups are phenyl and naphthyl, more preferably phenyl.

The term “hetaryl” is well known to chemists. The term includes 5- or 6-membered heteroaryl rings containing 1-4 heteroatoms chosen from oxygen, sulfur, and nitrogen in which oxygen and sulfur are not next to each other. Examples of such heteroaryl rings are furyl, thienyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridyl, pyridazinyl, pyrimidinyl, pyrazinyl, and triazinyl. The term “hetaryl” includes hetaryl rings with fused carbocyclic ring systems that are partially or fully unsaturated, such as a benzene ring, to form a benzofused hetaryl. For example, benzimidazole, benzoxazole, benzothiazole, benzofuran, quinoline, isoquinoline, quinoxaline, and the like. The term “hetaryl” also includes fused 5-6, 5-5, 6-6 ring systems, optionally possessing one nitrogen atom at a ring junction. Examples of such hetaryl rings include, but are not limited to, pyrrolopyrimidinyl, imidazo[1,2-a]pyridinyl, imidazo[2,1-b]thiazolyl, imidazo[4,5-b]pyridine, pyrrolo[2,1f][1,2,4]triazinyl, and the like. Hetaryl groups may be attached to other groups through their carbon atoms or the heteroatom(s), if applicable. For example, pyrrole may be connected at the nitrogen atom or at any of the carbon atoms.

Unless otherwise stated, the terms “heterocyclic ring”, “heterocyclyl” and “heterocycle” are equivalent, and include 4-10-membered, e.g. 5-membered, saturated or partially saturated rings containing 1-4 heteroatoms chosen from oxygen, sulfur, and nitrogen. The sulfur and oxygen heteroatoms are not directly attached to one another. Any nitrogen heteroatoms in the ring may optionally be substituted with C₁₋₄alkyl. Examples of heterocyclic rings include azetidine, oxetane, tetrahydrofuran, tetrahydropyran, oxepane, oxocane, thietane, thiazolidine, oxazolidine, oxazetidine, pyrazolidine, isoxazolidine, isothiazolidine, tetrahydrothiophene, tetrahydrothiopyran, thiepane, thiocane, azetidine, pyrrolidine, piperidine, N-methylpiperidine, azepane, 1,4-diazapane, azocane, [1,3]dioxane, oxazolidine, piperazine, homopiperazine, morpholine, thiomorpholine, 1,2,3,6-tetrahydropyridine and the like. Other examples of heterocyclic rings include the oxidized forms of the sulfur-containing rings. Thus, tetrahydrothiophene-1-oxide, tetrahydrothiophene-1,1-dioxide, thiomorpholine-1-oxide, thiomorpholine-1,1-dioxide, tetrahydrothiopyran-1-oxide, tetrahydrothiopyran-1,1-dioxide, thiazolidine-1-oxide, and thiazolidine-1,1-dioxide are also considered to be heterocyclic rings. The term “heterocyclic” also includes fused ring systems and can include a carbocyclic ring that is partially or fully unsaturated, such as a benzene ring, to form benzofused heterocycles. For example, 3,4-dihydro-1,4-benzodioxine, tetrahydroquinoline, tetrahydroisoquinoline, indoline and the like.

Compounds described herein may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above Formula (I) is shown without a definitive stereochemistry at certain positions. The present invention includes all stereoisomers of Formula (I) and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of Formula (I) exists, the present invention includes any possible tautomers and pharmaceutically acceptable salts thereof, and mixtures thereof, except where specifically stated otherwise.

When the compound of Formula (I) and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The invention also encompasses a pharmaceutical composition that is comprised of a compound of Formula (I), or a pharmaceutically acceptable salt thereof, in combination with a pharmaceutically acceptable carrier.

Preferably the composition is comprised of a pharmaceutically acceptable carrier and a non-toxic therapeutically effective amount of a compound of Formula (I), or a pharmaceutically acceptable salt thereof.

Moreover, within this preferred embodiment, the invention encompasses a pharmaceutical composition for the treatment of disease by inhibiting glycogen phosphorylase, resulting in the prophylactic or therapeutic treatment of diabetes, hyperglycemia, hypercholesterolemia, hyperinsulinemia, hyperlipidemia, hypertension, atherosclerosis or tissue ischemia e.g. myocardial ischemia comprising a pharmaceutically acceptable carrier and a non-toxic therapeutically effective amount of compound of Formula (I), or a pharmaceutically acceptable salt thereof.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include arginine, betaine, caffeine, choline, N N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric, and tartaric acids.

Since the compounds of Formula (I) are intended for pharmaceutical use they are preferably provided in substantially pure form, for example at least 60% pure, more suitably at least 75% pure especially at least 98% pure (% are on a weight for weight basis).

The pharmaceutical compositions of the present invention comprise a compound represented by Formula (I), or a pharmaceutically acceptable salt thereof, as an active ingredient, a pharmaceutically acceptable carrier and optionally other therapeutic ingredients or adjuvants. The compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The compositions are preferably suitable for oral administration. The pharmaceutical compositions may be conveniently presented in unit dosage form and prepared by any of the methods well known in the art of pharmacy.

In practice, the compounds of Formula (I), or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, sachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compounds of Formula (I), or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound of Formula (I) or a pharmaceutically acceptable salt thereof. The compounds of Formula (I), or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each sachet or capsule preferably contains from about 0.05 mg to about 5 g of the active ingredient.

For example, a formulation intended for oral administration to humans may contain from about 0.5 mg to about 5 g of active agent, compounded with an appropriate and convenient amount of carrier material, which may vary from about 5 to about 95% of the total composition. Unit dosage forms will generally contain from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound of Formula (I), or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of Formula (I), or a pharmaceutically acceptable salt thereof, may also be prepared in powder or liquid concentrate form.

Generally, dosage levels on the order of 0.01 mg/kg to about 150 mg/kg of body weight per day are useful in the treatment of the above-indicated conditions, or alternatively about 0.5 mg to about 7 g per patient per day. For example, lung cancer may be effectively treated by the administration of from about 0.01 to 50 mg of the compound per kilogram of body weight per day, or alternatively about 0.5 mg to about 3.5 g per patient per day. Similarly, breast cancer may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

The compounds of Formula (I) and pharmaceutically acceptable salts thereof, may be used in the treatment of diseases or conditions in which the Ab1, Aurora-A, Blk, c-Raf, cSRC, Src, PRK2, FGFR3, Flt3, Lck, Mek1, PDK-1, GSK3β, EGFR, p70S6K, BMX, SGK, CaMKII, Tie-2, Ron, Met, IGF-1R, or KDR kinases plays a role.

Thus the invention also provides a method for the treatment of a disease or condition in which the Ab1, Aurora-A, Blk, c-Raf, cSRC, Src, PRK2, FGFR3, Flt3, Lck, Mek1, PDK-1, GSK3β, EGFR, p70S6K, BMX, SGK, CaMKII, Tie-2, Ron, Met, IGF-1R, or KDR kinases plays a role comprising a step of administering to a subject in need thereof an effective amount of a compound of Formula (I), or a pharmaceutically acceptable salt thereof.

Diseases or conditions in which the Ab1, Aurora-A, Blk, c-Raf, cSRC, Src, PRK2, FGFR3, Flt3, Lck, Mek1, PDK-1, GSK3β, EGFR, p70S6K, BMX, SGK, CaMKII, Tie-2, Ron, Met, IGF-1R, or KDR kinases plays a role include lung, breast, prostate, pancreatic, head and neck cancers, as well as leukemia.

The invention also provides a method for the treatment of cancers of the lung, breast, prostate, pancreas, head, neck or blood comprising a step of administering to a subject in need thereof an effective amount of a compound of Formula (I), or a pharmaceutically acceptable salt thereof.

The invention also provides a method for the treatment of lung cancer, breast cancer, prostate, cancer, pancreatic cancer, head cancer, neck cancer, or leukemia in a human demonstrating such cancers comprising a step of administering to a subject in need thereof an effective amount of a compound of Formula (I), or a pharmaceutically acceptable salt thereof.

The invention also provides a method for the treatment of cancers of the lung, breast, prostate, pancreas, head, neck, or blood comprising a step of administering to a patient in need thereof an effective amount of a compound of Formula (I), or a pharmaceutically acceptable salt thereof.

The invention also provides the use of a compound of Formula (I), or a pharmaceutically acceptable salt thereof, in the treatment of a condition as defined above.

The invention also provides the use of a compound of Formula (I), or a pharmaceutically acceptable salt thereof, in the manufacture of a medicament for the treatment of a condition as defined above.

In the methods of the invention the term “treatment” includes both therapeutic and prophylactic treatment.

The compounds of Formula (I), or pharmaceutically acceptable salts thereof, may be administered alone or in combination with one or more other therapeutically active compounds. The other therapeutically active compounds may be for the treatment of the same disease or condition as the compounds of Formula (I) or a different disease or condition. The therapeutically active compounds may be administered simultaneously, sequentially or separately.

The compounds of Formula (I) may be administered with other active compounds for the treatment of cancers of the lung, breast, prostate, pancreas, head, neck, or blood—for example AVASTIN, IRESSA, TARCEVA, ERBITUX, or cisplatin.

The compounds of Formula (I) may also be administered in combination with AVASTIN, IRESSA, TARCEVA, ERBITUX, or cisplatin.

The compounds of Formula (I) may exhibit advantageous properties compared to known kinase inhibitors; for example, the compounds may exhibit improved solubility thus improving absorption properties and bioavailability. Furthermore the compounds of Formula (I) may exhibit further advantageous properties such as reduced inhibition of cytochrome P450 enzymes, meaning that they are less likely to cause adverse drug-rug interactions than known kinase inhibitors.

Experimental

Scheme 1-Scheme 5 and the examples and intermediates to follow serve to demonstrate how to synthesize compounds of this invention, but in no way limit the invention.

The following abbreviations are used:
NMR Nuclear magnetic resonance
LC/MS or LC-MS Liquid chromatography mass spectrometry
LDA Lithium diisopropylamide
DCM Dichloromethane
THF Tetrahydrofuran
MeCN Acetonitrile
DMSO Dimethylsulfoxide
BOC t-butyloxycarbonyl
DMF N,N-dimethylformamide
PS-DIEA Polymer-supported diisopropylethylamine
EDCI or EDC 1-(3-dimethylaminopropyl)-3-ethylcarbodiimide
HOBt 1-hydroxybenzotriazole
DMAP 4-dimethylaminopyridine
TLC Thin layer chromatography
Min or mins minute(s)
Hr, hrs, or h hour(s)
RT, rt, or r.t room temperature
Rt, or t_{R} Retention time
NBS N-bromosuccinimide
TBTU O-(Benzotriazol-1-yl)-N,N,N′N′-tetramethyluronium tetrafluoroborate
DIPEA N,N-diisopropylethylamine
MS mass
HPLC high performance liquid chromatography
   Description of the Chemistry

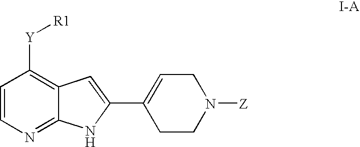

Compound of Formula I-A is equal to compound of Formula I wherein R3, R4, and R5=H. Cy= 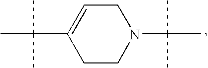
and Y. R1, and Z are as defined above for compounds of Formula I.

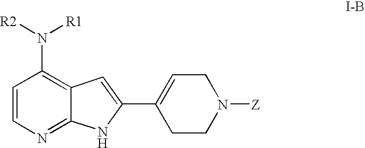

Compound of Formula I-B is equal to compound of Formula I wherein R3, R4, and R5=H, Cy= 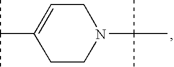
YR1=NR1R2, and R1, R2, and Z are as defined above.

Scheme 1 describes how compounds of Formula I-A may be synthesized.

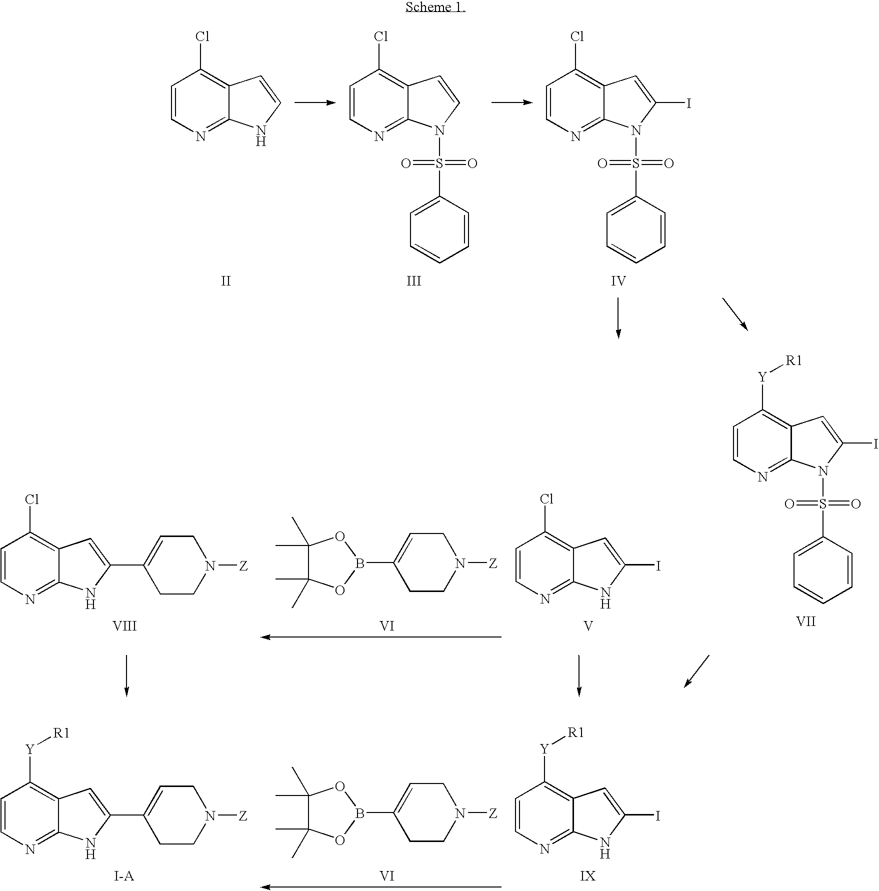

The compound of Formula II can be prepared by methods described in the literature (e.g., J. Phys. Chem. A 2003, 107 (10), 1459-1471; J. Chem. Soc. Perkin 1, 1974, (19), 2270-2274). A benzenesulfonyl group is introduced under typical reaction conditions with typical bases and sulfonylating reagents in typical solvents to give compound of Formula III. Typical reagents and solvents include, but are not limited to, sodium hydride in DMF or THF, alkoxides such as potassium tert-butoxide in THF, a biphasic system consisting of aqueous NaOH and methylene chloride. Typical sulfonylating reagents are, e.g., benzenesulfonyl chloride or the corresponding anhydride. Typical conditions include, but are not limited to, −20° C. to RT, at atmospheric pressure, with equimolar amounts of base and sulfonylating reagent, although larger amounts can be used if desirable. Compounds of Formula III can be iodinated under typical metallation/iodination conditions to yield compounds of Formula IV. Typical conditions include, but are not limited to, adding a lithium amide base, such as LDA or LiTMP, to a cooled (about −78° C. to about 0° C.) solution of compound of Formula III in an ether-type solvent, such as THF, 2-methyl-THF, DME, and the like (optionally containing other solvents such as aliphatic or aromatic hydrocarbons), and reacting the resulting species with an iodine source such as I₂, ICl, or N-iodosuccinimide. Compounds of Formula V can be prepared from compounds of Formula IV by reacting with bases such as NaOH in alcoholic solvents such as MeOH at typical reaction temperatures from about −10° C. to about 40° C. Compounds of Formula VIII can be prepared by palladium-mediated coupling with a boronate of Formula VI under typical Suzuki conditions well known to someone skilled in the art. It will be appreciated that instead of the pinacol boronate shown, other boronate esters or the free boronic acids may also be used. Furthermore, reaction of the corresponding trialkyl tin derivatives of VI (i.e., compounds with, e.g., Bu₃Sn— in place of the pinacolboronate) under typical Stille coupling conditions may also be used to prepare compounds of Formula VIII from compounds of Formula V. Displacement of the chloride of compounds of Formula VIII with HYR1 under typical chloride displacement conditions gives compounds of Formula I-A. Alternatively, the order of steps may be reversed: Compound of Formula V is first reacted with HYR1 under typical chloride displacement conditions to yield compounds of Formula IX, followed by palladium-mediated coupling with a boronate of Formula VI under typical Suzuki conditions as described above to give compounds of Formula I-A.

If deemed advantageous, the removal of the benzesulfonyl group may also be performed after chloride displacement and Suzuki coupling under similar reaction conditions. Someone skilled in the art will realize that other groups may be used in place of the benzenesulfonyl group for the metalation/iodination reaction. Examples include, but are not limited to, toluenesulfonyl, tert-butoxycarbonyl, and tert-butylcarbamoyl. Furthermore, instead of introducing an iodine in the reaction from compound of Formula III to compound of Formula IV one may introduce a bromine using, e.g., bromine, CBr₄, or NBS under otherwise identical conditions and react the resulting compound in the same way as described above.

Further manipulation of the substituents Z may be desirable, and Scheme 2 describes how compounds wherein Z=tert-butoxycarbonyl (Boc) may be used for that purpose.

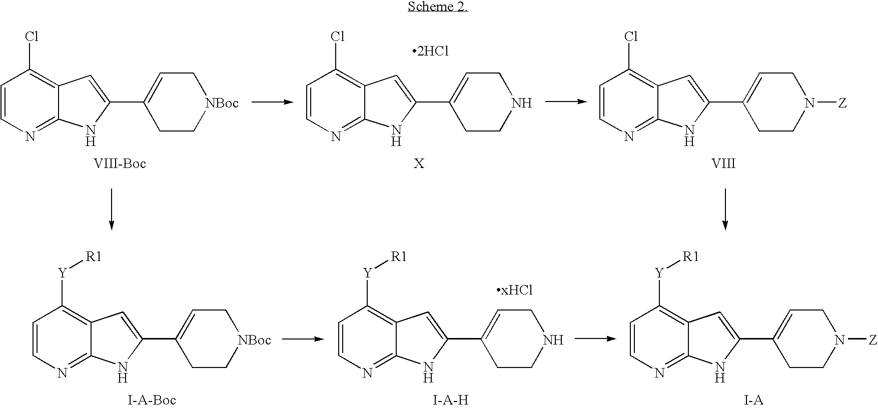

Compound of Formula I-A-Boc can be reacted with HCl in a typical solvent to give the hydrochloride salt of Formula I-A-H. Typical solvents include, but are not limited to, dioxane, MeOH, and water. Compounds of Formula I-A-H can be reacted with acids, anhydrides, acid halids, chloroformates, carbamoyl halides, sulfonyl halides, sulfamoyl halids, sulfonic anhydrides, and the like, under conditions described in the examples to give compounds of Formula I-A. Alternatively, a compound of Formula VIII-Boc can be reacted with HCl as described above to give the hydrochloride salt of Formula X. Introduction of the Z substituents as described above to yield a compound of Formula VIII, followed by chloride displacement with HYR1 gives compounds of Formula I-A.

Someone skilled in the art will realize that acids other than HCl can be used for removal of the Boc group in compounds of Formula VIII-Boc and I-A-Boc.

When HYR1 is equal to HNR1R2, someone skilled in the art will recognize that a variety of typical reaction conditions, typical solvents, and typical additives are available for the conversion of VIII to I-B and of VIII-Boc to I-B-Boc, shown in scheme 3.

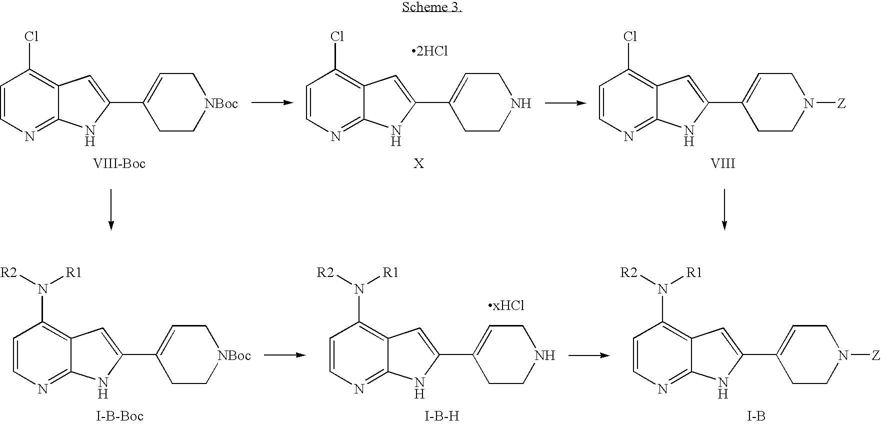

Generally, VIII or VIII-Boc are reacted with HNR1R2 in a suitable solvent. Typical solvents include, but are not limited to, alcohols such as trifluoroethanol (TFE) with additives such as HCl and TFA. The reaction is typically carried out at about 40° C. to about 150° C. If the reaction temperature is higher than the boiling point of the reaction mixture, a pressure reactor should be used. Alternatively, typical transition metal-mediated chloride displacement conditions well known to someone skilled in the art can be used. These conditions typically involve reacting VIII or VIII-Boc with HNR1R2, a transition metal compound, a suitable ligand, and a base in a suitable solvent. Typical solvents include, but are not limited to, dioxane and DMF. Typical catalysts include, but are not limited to, Pd₂dba₃ and palladium acetate. Typical ligands include, but are not limited to, BINAP and dppf. The reaction is typically carried out at about 90° C. to about 150° C.

Someone skilled in the art will recognize that especially when acidic additives are used, the Boc group may be partially or completely removed simultaneously, so that compounds of Formula I-B-H are solely obtained or in a mixture with compounds of Formula I-B-Boc. If one wishes to obtain compounds of Formula I-B-Boc, the reaction mixture containing compounds of Formula I-B-H (either exclusively or as mixture with compounds of Formula I-B-Boc) can directly be treated with a base such as triethylamine or diisopropylethylamine and di-tert-butyldicarbonate without the need for isolation. If one wishes to obtain compounds of Formula I-B-H, a mixture with compounds of Formula I-B-Boc can directly be treated with suitable acids to remove the Boc group completely.

In some cases, compounds of Formula HNR1R2 are commercially available or synthesized according to literature procedures. In cases where neither is available, compounds of Formula HNR1R2 were synthesized via procedures described in the experimental section herein.

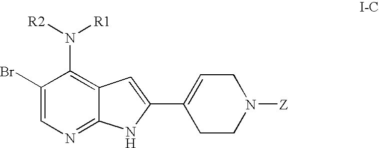

Compound of Formula I-C is equal to compound of Formula I wherein R3=Br, R4 and R5=H, Cy= 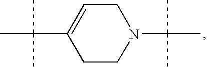
YR1=NR1R2, and R1, R2, and Z are as defined above.

Scheme 4 describes how compounds of Formula I-C may be synthesized.

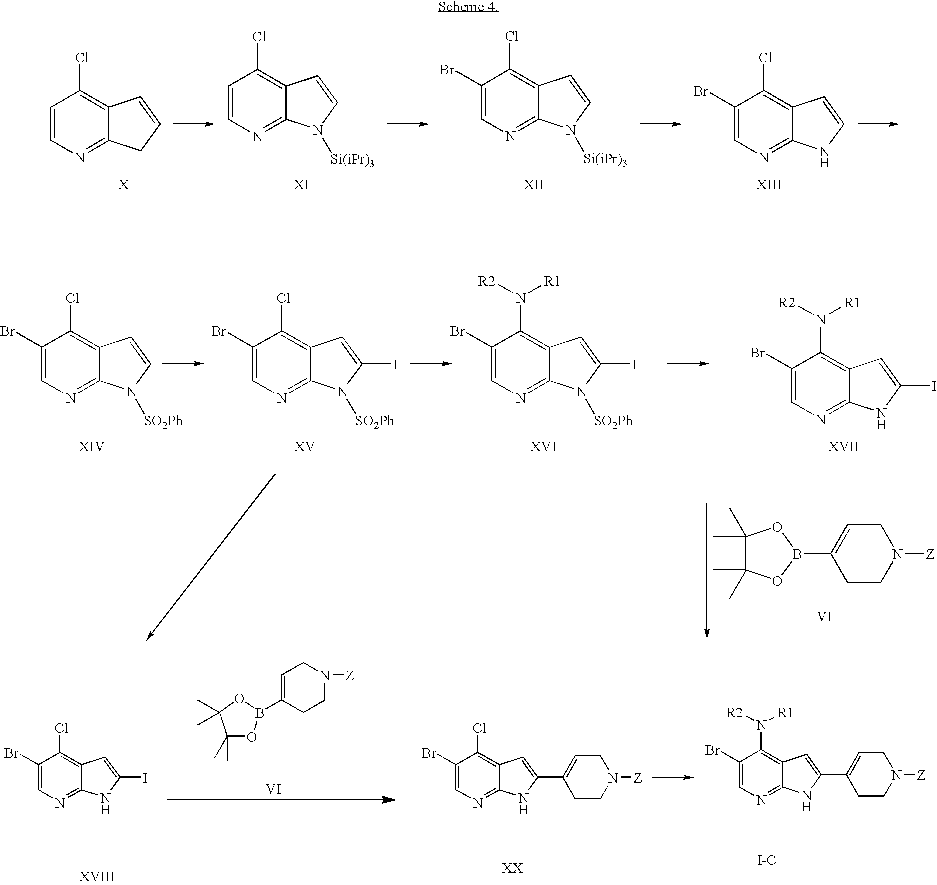

The compound of Formula XII is known in the literature and may be prepared according to a published procedure (Tetrahedron Lett. 2004, 45, 2317-2319), which involves treating a THF solution of the compound of Formula XI with sec-BuLi at about −78° C. and reacting with an electrophilic bromine source, such as carbon tetrabromide. Typical conditions for the removal of the triisopropylsilyl group to obtain compound of Formula XIII include, but are not limited to, treatment with tetrabutylammonium fluoride, or acids such as HCl or H₂SO₄ in alcoholic solvents. A compound of Formula XIV may be obtained from a compound of Formula XIII as described above for the conversion of a compound of Formula II to a compound of Formula III. A compound of Formula XV may be obtained from a compound of Formula XIV as described above for the conversion of a compound of Formula III to a compound of Formula IV. Compounds of Formula XVI can be obtained by reacting compound of Formula XV with HNR1R2 in a typical solvent under typical reaction conditions. Typical solvents include, but are not limited to, alcohols such as trifluoroethanol (TFE) with additives such as HCl and TFA. The reaction is typically carried out at about 40° C. to about 120° C. If the reaction temperature is higher than the boiling point of the reaction mixture, a pressure reactor should be used. The benzenesulfonyl group of compounds of Formula XVI can be removed to give compounds of Formula XVII under conditions described above for the conversion of a compound of Formula IV to a compound of Formula V. Compounds of Formula I-C can then be prepared from compounds of Formula XVII by palladium-mediated coupling with a boronate of Formula VI under typical Suzuki conditions well known to someone skilled in the art. It will be appreciated that instead of the pinacol boronate shown, other boronate esters or the free boronic acids may also be used. Furthermore, reaction of the corresponding trialkyl tin derivatives of VI (i.e., compounds with, e.g., Bu₃Sn— in place of the pinacolboronate) under typical Stille coupling conditions may also be used to prepare compounds of Formula XVII from compounds of Formula XVI. Alternatively, the benzesulfonyl group in compound of Formula XV may be removed first to yield compound of Formula XVIII, followed by coupling with a boronate of Formula VI to give compounds of Formula XX, and chloride displacement with HNR1R2 to give compounds of Formula I-C. X, under conditions described above.

Someone skilled in the art will realize that other groups may be used in place of the benzenesulfonyl group for the metalation/iodination reaction (i.e., XIV→XV). Examples include, but are not limited to, toluenesulfonyl, tert-butoxycarbonyl, and tert-butylcarbamoyl. Furthermore, if Z=Boc, the chloride displacement in compounds of Formula XX under the conditions described above may lead to removal of the Boc group to give a compound of Formula XIX wherein Z=H. Such a compound can be treated with a base such as triethylamine or diisopropylethylamine and di-tert-butyldicarbonate to obtain a compound of Formula XIX wherein Z=Boc, or with other appropriate reagents to introduce the desired Z substituent.

Further manipulation of the substituents Z may be desirable, and Scheme 5 describes how compounds wherein Z=tert-butoxycarbonyl (Boc) may be used for that purpose.

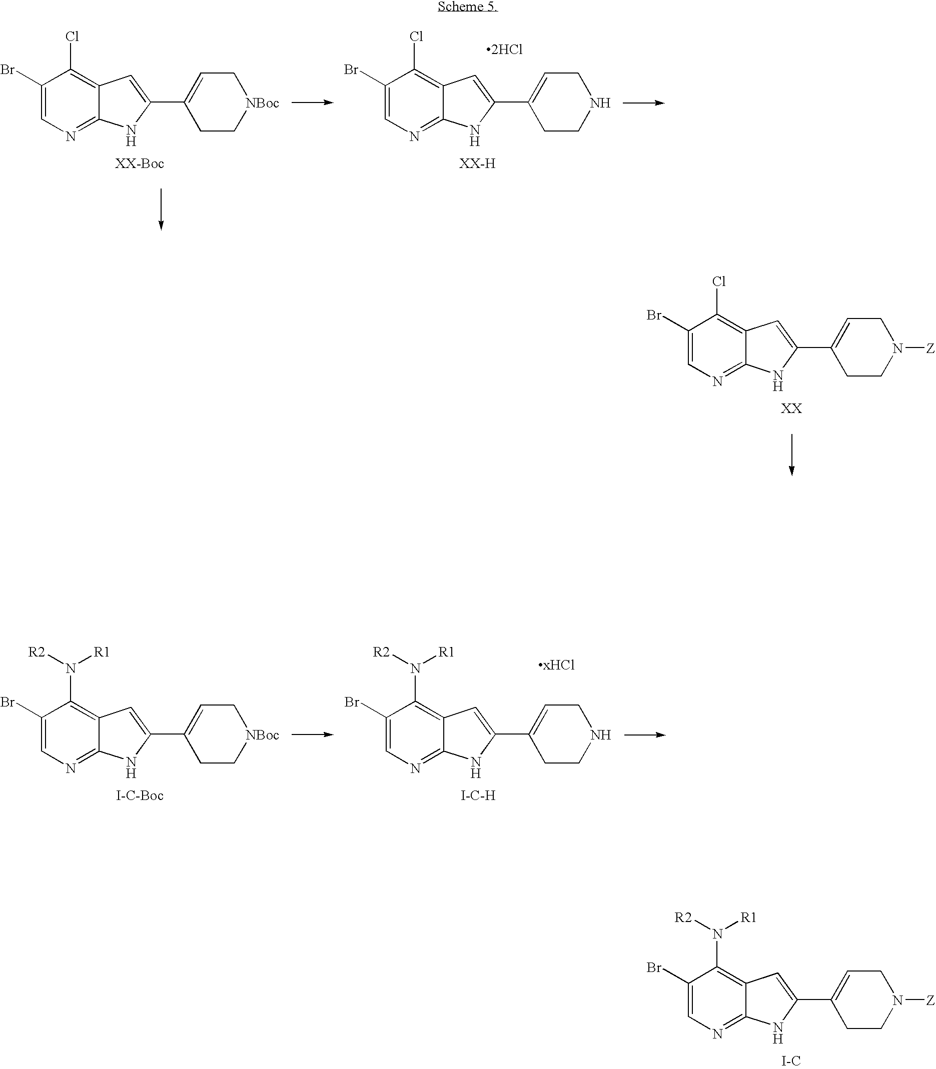

Compounds of Formula XX-Boc or I-C-Boc can be reacted with HCl in a typical solvent to give the hydrochloride salt of Formula XX-H or I-C-H, respectively. Typical solvents include, but are not limited to, dioxane, MeOH, and water. Compounds of Formula XX-H or I-C-H can be reacted with acids, anhydrides, acid halids, chloroformates, carbamoyl halides, sulfonyl halides, sulfamoyl halids, sulfonic anhydrides, and the like, under conditions described in the examples to give compounds of Formula XX or I-C, respectively. Someone skilled in the art will realize that acids other than HCl can be used for removal of the Boc group in compounds of Formula XX-Boc and I-C-Boc.

The methods outlined in Schemes 4 and 5 for compounds of Formula I-C (i.e., wherein R3=Br) can be applied, using no more than ordinary skills, to prepare compounds with other R3 groups within the scope of this invention by using other electrophiles in place of the electrophilic bromine source. Examples include, but are not limited to, N-fluorobenzenesulfonimide for R3=F, hexachloroethane for R3 =Cl, tosyl azide for R3 =N₃, camphorsulfonyloxaziridine or Ti(iOPr)₄/tBuOOLi for R3=OH.

It would be appreciated by those skilled in the art that in some situations, a substituent that is identical or has the same reactivity to a functional group which has been modified in one of the following processes, will have to undergo protection followed by deprotection to afford the desired product and avoid undesired side reactions. Alternatively, another of the processes described within this invention may be employed in order to avoid competing functional groups. Examples of suitable protecting groups and methods for their addition and removal may be found in the following reference: “Protective Groups in Organic Syntheses”, T. W. Greene and P. G. M. Wuts, John Wiley and Sons, 1989.

General Experimental Information:

All melting points were determined with a Mel-Temp II apparatus and are uncorrected. Commercially available anhydrous solvents and HPLC-grade solvents were used without further purification. ¹H NMR and ¹³C NMR spectra were recorded with Varian or Bruker instruments (400 MHz for ¹H, 100.6 MHz for ¹³C) at ambient temperature with TMS or the residual solvent peak as internal standards. The line positions or multiplets are given in ppm (δ) and the coupling constants (J) are given as absolute values in Hertz, while the multiplicities in ¹H NMR spectra are abbreviated as follows: s (singlet), d (doublet), t (triplet), q (quartet), quint (quintet), m (multiplet), m_{c}(centered multiplet), br (broadened), AA′BB′. The signal multiplicities in ¹³C NMR spectra were determined using the DEPT135 pulse sequence and are abbreviated as follows:+(CH or CH₃), −(CH₂), C_{quart}(C). LC/MS analysis was performed using a Gilson 215 autosampler and Gilson 819 autoinjector attached to a Hewlett Packard HP1100 and a Micromass ZQ2000 mass spectrometer. XTERRA MS C18 5 μ 4.6×50 mm columns with detection at 254 nm and electrospray ionization in positive mode were used. For mass-directed purification (MDP), a Waters/MicromassZQ2000 system was used.

The tables below list the mobile phase gradients (solvent A: acetonitrile; solvent B: 0.01% formic acid in HPLC water) and flow rates for the analytical HPLC programs.

| | | | |
|---|---|---|---|
| | | | Flow Rate |
| | | | (mL/min) |
| Time | A % | B % | MicromassZQ |

Polar_5min

| | | | |
|---|---|---|---|
| 0.00 | 5 | 95 | 1.3 |
| 3.00 | 90 | 10 | 1.3 |
| 3.50 | 90 | 10 | 1.3 |
| 4.00 | 5 | 95 | 1.3 |
| 5.00 | 5 | 95 | 1.3 |

Polar_15min

| | | | |
|---|---|---|---|
| 0.00 | 5 | 95 | 1.3 |
| 1.00 | 30 | 70 | 1.3 |
| 7.50 | 90 | 10 | 1.3 |
| 10.00 | 100 | 0 | 1.3 |
| 13.00 | 5 | 95 | |
| 15.00 | 5 | 95 | 1.3 |

Nonpolar_5min

| | | | |
|---|---|---|---|
| 0.00 | 25 | 75 | 1.3 |
| 3.00 | 99 | 1 | 1.3 |
| 3.50 | 99 | 1 | 1.3 |
| 4.00 | 25 | 75 | 1.3 |
| 5.00 | 25 | 75 | 1.3 |

Nonpolar_7min

| | | | |
|---|---|---|---|
| 0.00 | 25 | 75 | 1.3 |
| 4.00 | 100 | 0 | 1.3 |
| 5.50 | 100 | 0 | 1.3 |
| 6.00 | 25 | 75 | 1.3 |
| 7.00 | 25 | 75 | 1.3 |

Nonpolar_15min

| | | | |
|---|---|---|---|
| 0.00 | 15 | 85 | 1.3 |
| 7.50 | 99 | 1 | 1.3 |
| 11.00 | 99 | 1 | 1.3 |
| 12.50 | 15 | 85 | 1.3 |
| 15.00 | 15 | 85 | 1.3 |

Syntheses of Examples and Intermediates

The following EXAMPLES 1-2 are compounds of Formula I wherein R3=Br.

### EXAMPLE 1

### 4-[5-Bromo-4-(1H-indazol-5-ylamino)-1H-pyrrolo[2,3-b]pyridin-2-yl]-3,6-dihydro-2H-pyridine-1-carboxylic acid tert-butyl ester

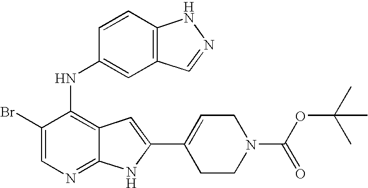

To a mixture of (5-bromo-2-iodo-1H-pyrrolo[2,3-b]pyridin-4-yl)-(1H-indazol-5-yl)- amine (25 mg, 0.069 mmol), potassium carbonate (19 mg, 0.14 mmol), tetrakistriphenylphosphine palladium (10 mg, 0.014 mmol) and 4-(4,4,5,5-tetramethyl-[1,3,2]dioxaborolan-2-yl)-3,6-dihydro-2H-pyridine-1-carboxylic acid tert-butyl ester (21.6 mg, 0.069 mmol) was added degassed DMF (3 mL) and water (0.75 mL) and the mixture was heated to reflux for 5h. Water was added to the reaction and filtered. The precipitate was washed with water and the filtrate was extracted with DCM. The precipitate was dissolved in DCM/MeOH mixture (9:1) and combined with the DCM extract and evaporated. The crude product was purified by preparative TLC using 8% methanol in DCM as eluent to afford the title compound as beige solid. ¹H NMR (400 MHz, CD₃OD): δ=10.32 (s, 1H), 8.11 (s, 1H), 8.02 (s, 1H), 7.58 (d, J=0.8 Hz, 1H), 7.47 (d, J=8.9 Hz, 1H), 7.26 (dd, J=8.8, 1.9 Hz, 1H), 6.69 (s, 1H), 5.95 (bs, 1H), 5.16 (bs, 1H), 3.99-4.03 (m, 2H), 3.42 (t, J=5.4 Hz, 2H), 2.00-2.07 (m, 2H), 1.39 (s, 9H); MS (ES+): m/z 510.89 (100) [MH⁺]; HPLC: t_{R}=2.63 min (ZQ2000, polar_{—}5 min).

### (5-Bromo-2-iodo-1H-pyrrolo[2,3-b]pyridin-4-yl-(1H-indazol-5-yl)amine

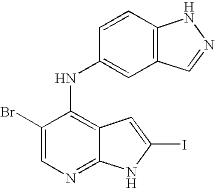

To a slurry of a mixture of 1-benzenesulfonyl-5-bromo-4-chloro-2-iodo-1H-pyrrolo[2,3-b]pyridine and 5-bromo-4-chloro-2-iodo-1-(2-iodobenzenesulfonyl)-1H-pyrrolo[2,3-b]pyridine (560 mg, 1.0 mmol) in trifluoroethanol (6 mL) was added trifluoroacetic acid (0.041 mL, 0.52 mmol) and indazole (266 mg, 2.0 mmol), and the reaction was heated in a sealed tube at 120° C. for 4 days. Additional indazole (67 mg, 0.5 mmol) was added twice to the reaction on the second and third days. The reaction was cooled to RT, diluted with methanol (20 mL), saturated sodium bicarbonate solution was added (1 mL) and evaporated to dryness under reduced pressure. The residue obtained was triturated with methanol:DCM (1:1) mixture and filtered. The filtrate was evaporated and the crude obtained was purified by chromatography on silica gel [Jones Flashmaster, 70g/150 mL cartridge, eluting with DCM:Methanol 100:0→96:4], yielding the product, which was triturated with 4:1 methanol:DCM mixture and filtered. The precipitate obtained was dried under vacuum to afford the title compound as an off-white solid. The low polar fractions from the column were combined, evaporated and the residue was stirred with 3 N NaOH in methanol (2 mL) for 30 min and quenched with saturated ammonium chloride solution (2 mL). Water (10 mL) was added and filtered to afford additional title compound. MS (ES+): m/z 453.63 (100) [MH⁺]; HPLC: t_{R}=2.58 min (ZQ2000, polar_{—}5 min).

### 1-Benzenesulfonyl-5-bromo-4-chloro-2-iodo-1H-pyrrolo[2,3-b]pyridine & 5-Bromo-4-chloro-2-iodo-1-(2-iodobenzenesulfonyl)-1H-pyrrolo [2,3-b]pyridine

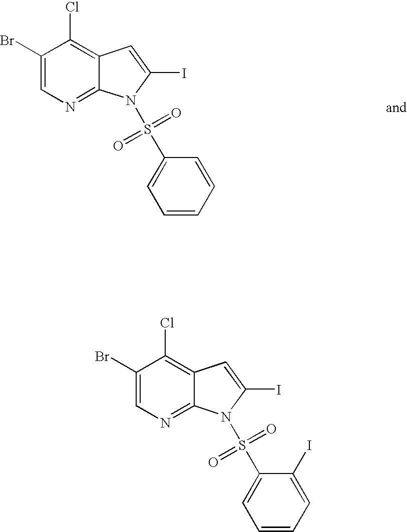

To a solution of 1-benzenesulfonyl-5-bromo-4-chloro-1H-pyrrolo[2,3-b]pyridine (2.585 g, 6.95 mmol) in anhydrous THF (330 mL) at −78° C. was added LDA (11.6 mL, 1.5 M solution, 17.39 mmol), and the mixture was stirred for 30 min. A solution of iodine (4.854 g, 19.12 mmol) in THF (20 mL) was added and stirring was continued for an additional 2h at −78° C. The reaction was quenched with aqueous sodium thiosulfate solution and extracted with DCM (4×80 mL). The combined DCM layer was washed with brine, dried over anhydrous sodium sulfate and evaporated under reduced pressure. The crude product was purified by chromatography on silica gel [Jones Flashmaster, 70g/150 mL cartridge, eluting with hexane:ethyl acetate 100:0→99:2], yielding a mixture of the title compounds. 1-Benzenesulfonyl-5-bromo-4-chloro-2-iodo-1H-pyrrolo[2,3-b]pyridine: MS (ES+): m/z 498.58 (100) [MH⁺], HPLC: t_{R}=7.19 min (ZQ2000, nonpolar_{—}15 min). 5-Bromo4-chloro-2-iodo-1-(2-iodobenzenesulfonyl)-1H-pyrrolo[2,3-b]pyridine: MS (ES+): m/z 624.41 (100) [MH⁺], HPLC: t_{R}=7.58 min (ZQ2000, nonpolar_{—}5 min).

### 1-Benzenesulfonyl-5-bromo4-chloro-1H-pyrrolo[2,3-b]pyridine

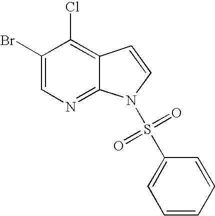

To a solution of 5-bromo4-chloropyrrolopyridine (248 mg, 1.07 mmol) in THF (5 mL) at 0° C. was added sodium hydride (39 mg, 1.6 mmol) and the mixture was stirred for 15 min. Benzenesulfonyl chloride (227 mg, 1.28 mmol) was added and the mixture was allowed to warm to RT and stirred for 4h. Water was added to the reaction mixture and extracted with DCM (3×25 mL). The combined DCM layer was washed with brine, dried over anhydrous sodium sulfate and evaporated under reduced pressure. The crude product obtained was purified by chromatography on silica gel [Jones Flashmaster, 50 g/150 mL cartridge, eluting with hexane:ethylacetate 100:0→95:05], yielding the title compound as colorless solid. ¹H NMR (400 MHz, CDCl₃): δ=8.50 (s, 1H), 8.16-8.19 (m, 2H), 7.77 (d, J=4.0 Hz, 1H), 7.59-7.64 (m, 1H), 7.49-7.53 (m, 2H), 6.69 (d, J=4.0 Hz, 1H); MS (ES+): m/z 372.85 (100) [MH⁺]; HPLC: t_{R}=4.39 min (ZQ2000, nonpolar_{—}7 min).

### 5-Bromo-4-chloro-1H-pyrrolo[2,3-b]pyridine

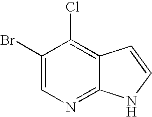

To a solution of 5-bromo4-chloro-1-triisopropylsilanyl-1H-pyrrolo[2,3-b]pyridine (10.1 g, ≈14.0 mmol; crude material prepared according to Tetrahedron Lett., 2004, 45, 2317-2319) in IPA (250 mL) at 0° C. was added 2N H₂SO₄ (25 mL) and the mixture was allowed to warm to RT and stirred overnight. IPA was evaporated at 35° C. and water was added to the residue and neutralized with 2N NaOH. The precipitate formed was filtered, washed with water followed by hexane, and dried under vacuum to yield the title compound as off-white solid. MS (ES+): m/z 233.01 (100) [MH⁺]; HPLC: t_{R}=4.51 min (ZQ2000, polar_{—}15 min).

### EXAMPLE 2

### 4-[4-(Benzothiazol-6-ylamino)5-bromo-1H-pyrrolo[2,3-b]pyridin-2-yl]-3,6-dihydro-2H-pyridine-1-carboxylicacid tert-butyl ester

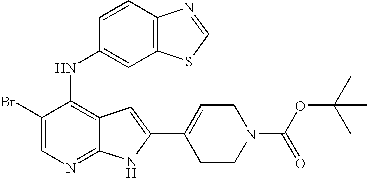

To a mixture (55:45) of benzothiazol-6-yl-[5-bromo-2-(1,2,3,6-tetrahydropyridin-4-yl)-1H-pyrrolo[2,3-b]pyridin-4-yl]-amine and 5-bromo-4-chloro-2-(1,2,3,6-tetrahydropyridin-4-yl)-1H-pyrrolo[2,3-b]pyridine (101 mg, 0.272 mmol) in THF (5 mL) was added triethylamine (83 mg, 0.816 mmol), DMAP (5 mg) and (Boc)₂O (47 mg, 0.215 mmol) and the reaction was stirred overnight at RT under nitrogen atmosphere. The solvent was evaporated and the residue was purified by preparative TLC using 4% methanol in DCM as eluent to afford a mixture of 4-(5-bromo4-chloro-1H-pyrrolo[2,3-b]pyridin-2-yl)-3,6-dihydro-2H-pyridine-1-carboxylic acid tert-butyl ester and the title compound. 4-[4-(Benzothiazol-6-ylamino)-5-bromo-1H-pyrrolo[2,3-b]pyridin-2-yl]-3,6-dihydro-2H-pyridine-1-carboxylicacid tert-butyl ester: ¹H NMR (400 MHz, CDCl₃): δ=8.98 (s, 1H), 8.19 (s, 1H), 8.11 (d, J=8.6 Hz, 1H), 7.75 (d, J=2.0 Hz, 1H), 7.37 (dd, J=8.7,2.1 Hz, 1H), 6.81 (s, 1H), 6.15 (bs, 1H), 5.51 (s, 1H), 4.90-4.15 (m, 2H), 3.54 (t,J=5.3 Hz, 2H), 2.95 (s, 1H), 2.88 (s, 1H), 2.17-2.25 (m, 2H), 1.47 (s, 9H); MS (ES+): m/z 527.84 (100) [MH⁺]; HPLC: t_{R}=3.30 min (ZQ2000, polar_{—}5 min).

### Benzothiazol-6-yl-[5-bromo-2-(1,2,3,6tetrahydropyridin-4-yl)-1H-pyrrolo[2,3-b]pyridin-4-yl]-amine

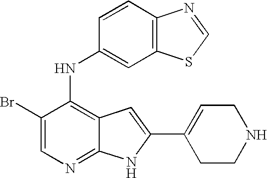

To a slurry of 4-(5-bromo-4-chloro-1H-pyrrolo[2,3-b]pyridin-2-yl)-3,6-dihydro-2H-pyridine-1-carboxylic acid tert-butyl ester (120 mg, 0.29 mmol) in trifluoroethanol (9 mL) were added trifluoroacetic acid (0.112 mL, 1.46 mmol) and 6-benzothiazolamine (58 mg, 1.5 mmol), and the reaction was heated in a sealed tube at 120° C. for 6 days. Additional 6-benzothiazolamine (39 mg, 0.29 mmol) was added twice to the reaction mixture on the third and fifth days. The reaction was cooled to RT, diluted with methanol (20 mL), saturated sodium bicarbonate solution was added (1 mL) and evaporated to dryness under reduced pressure. The residue was triturated with methanol:DCM (1:1) mixture and filtered. The filtrate was evaporated and the crude product was purified by preparative TLC using 25% methanol in DCM to afford a mixture of benzothiazol-6-yl-[5-bromo-2-(1,2,3,6-tetrahydropyridin4-yl)-1H-pyrrolo[2,3-b]pyridin4-yl]-amine and 5-bromo-4chloro-2-(1,2,3,6-tetrahydro-pyridin-4-yl)-1H-pyrrolo[2,3-b]pyridine as beige solid. Benzothiazol-6-yl-[5-bromo-2-(1,2,3,6-tetrahydropyridin-4-yl)-1H-pyrrolo[2,3-b]pyridin-4-yl]-amine: MS (ES+): m/z 427.79 (100) [MH⁺], HPLC: t_{R}=2.12 min (ZQ2000, polar_{—}5 min). 5-Bromo-4-chloro-2-(1,2,3,6-tetrahydropyridin-4-yl)-1H-pyrrolo[2,3-b]pyridine MS (ES+): m/z 313.85 (100) [MH⁺], HPLC: t_{R}=2.30 min (ZQ2000, polar_{—}5min).

### 4-(5-Bromo-4-chloro-1H-pyrrolo[2,3-b]pyridin-2-yl)-3,6-dihydro-2H-pyridine-1-carboxylic acid tert-butyl ester

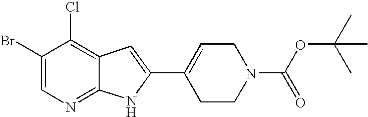

To a mixture of 5-bromo-4-chloro-2-iodo-1H-pyrrolo[2,3-b]pyridine (46 mg, 0.12 mmol), potassium carbonate (36 mg, 0.25 mmol), dichlorobis(triphenylphosphine)palladium (9 mg, 0.01 mmol), and 4-(4,4,5,5-tetramethyl-[1,3,2]dioxaborolan-2-yl)-3,6-dihydro-2H-pyridine-1-carboxylic acid tert-butyl ester (42 mg, 0.13 mmol) were added degassed dioxane (4 mL) and water (1 mL), and the mixture was heated to reflux for 5h. The reaction was evaporated under reduced pressure and the residue was dissolved in DCM and filtered. The DCM filtrate was evaporated and the crude product obtained was purified by chromatography on silica gel [Jones Flashmaster, 20 g/70 mL cartridge, eluting with DCM:methanol 100:0→99.5:0.5], yielding the title compound. MS (ES+): m/z 413.84 (100) [MH⁺]; HPLC: t_{R}=6.95 min (ZQ2000, nonpolar_{—}15 min).

### 5-Bromo-4-chloro-2-iodo-1H-pyrrolo[2,3-b]pyridine

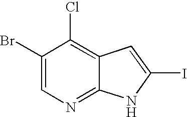

To a solution of a 4:6 mixture of 1-benzenesulfonyl-5-bromo-4-chloro-2-iodo-1H-pyrrolo[2,3-b]pyridine and 5-bromo-4-chloro-2-iodo-1-(2-iodobenzenesulfonyl)-1H-pyrrolo[2,3-b]pyridine (100 mg, 0.178 mmol) in THF (4 mL) was added 3N NaOH in methanol (1 mL), and the mixture was stirred at RT for 30 min. The reaction was quenched with saturated ammonium chloride solution (2 mL), water (5 mL) was added, and the mixture was filtered. The precipitate obtained was washed with water (3×10 mL) followed by hexane (3×10 mL) and dried under vacuum to yield the title compound as white solid. ¹H NMR (400 MHz, CD₃O D): δ=8.21 (s, 1H), 6.74 (s, 1H); MS (ES+): m/z 358.75 (100) [MH⁺]; HPLC: t_{R}=5.68 min (ZQ2000, nonpolar_{—}15 min).

The following EXAMPLES 3-69 are compounds of Formula I wherein R3=H.

### EXAMPLE 3

### 4-[4-(Benzothiazol-6-ylamino)-1H-pyrrolo[2,3-b]pyridin-2-yl]-3,6-dihydro-2H-pyridin-1-ylmorpholin-4-ylmethanone

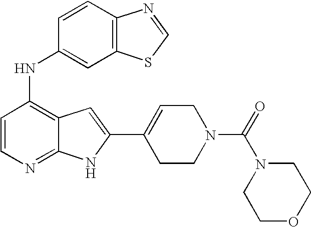

To a mixture of 4-[4-benzothiazol-6-yl-[2-(1,2,3,6-tetrahydropyridin-4-yl)-1H-pyrrolo[2,3-b]pyridin-4-yl]-amine trihydrochloride (150 mg, 0.33 mmol) and N,N-diisopropylethyl amine (64 mg, 0.49 mmol) in dry DMF (2.0 mL) at 0° C. was added 4-chlorocarbonylmorpholine (49.4 mg, 0.33 mmol) and the mixture was allowed to warm to RT and stirred overnight. The reaction was purified by column chromatography over silica gel [Jones FlashMaster, 50 g cartridge, eluting with DCM/methanol] to yield the title compound as yellow solid. ¹H NMR (400 MHz, CDCl₃): δ=11.63 (s, 1H), 9.24 (s, 1H), 8.96 (s, 1H), 8.02-8.07 (m, 2H), 7.92 (d, J=5.6 Hz, 1H), 7.47 (dd, J=8.8, 2.4 Hz, 1H), 6.76 (d, J=5.6 Hz, 1H), 6.64 (s, 1H), 6.40 (bs, 1H), 3.94-3.96 (m, 2H), 3.61 (t, J=4.8 Hz, 4H), 3.41 (t, J=5.6 Hz, 2H), 3.33 (bs, 2H), 3.14-3.18 (m, 4H); MS (ES+): m/z 461.10 (100) [MH⁺]; HPLC: t_{R}=2.01 min (ZQ2000, polar_{—}5 min).

### EXAMPLE 4

### 4-[4-(Benzothiazol-6-ylamino)-1H-pyrrolo[2,3-b]pyridin-2-yl]-3,6-dihydro-2H-pyridine-1-carboxylic acid tert-butylamide

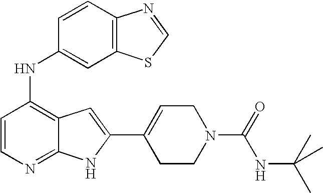

To a mixture of 4-[4-benzothiazol-6-yl-[2-(1,2,3,6-tetrahydropyridin4-yl)-1H-pyrrolo[2,3-b]pyridin-4-yl]-amine trihydrochloride (160 mg, 0.35 mmol) and N,N-diisopropylethyl amine (229 mg, 1.76 mmol) in dry DMF (3.0 mL) at 0° C. was added tert-butyl isocyanate (35 mg, 0.35 mmol) and the mixture was allowed to warm to RT and stirred overnight. DMF was evaporated and the reaction was purified by column chromatography over silica gel [Jones FlashMaster, 50 g cartridge, eluting with DCM/methanol] to yield the title compound as yellow solid. ¹H NMR (400 MHz, CDCl₃): δ=8.99 (s, 1H), 8.05 (d, J=8.8 Hz, 1H), 7.90 (dd, J=6.0, 1.2 Hz, 2H), 7.51 (dd, J=8.8, 2.4 Hz, 1H), 6.81 (d, J=5.6 Hz, 1H), 6.55 (s, 1H), 6.24 (bs, 1H), 5.02 (s, 1H), 4.04-4.06 (m, 2H), 3.61 (t, J=5.6 Hz, 2H), 2.58-2.60 (m, 2H), 1.38 (s, 9H); MS (ES+): m/z 447.14 (100) [MH⁺]; HPLC: t_{R}=2.82 min (ZQ2000, polar_{—}5 min).

### Benzothiazol-6-yl-[2-(1,2,3,6-tetrahydropyridin-4-yl)-1H-pyrrolo[2,3-b]pyridin-4-yl]-amine trihydrochloride

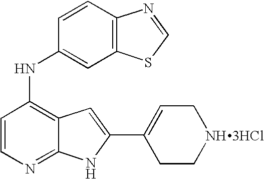

A mixture of 4-[4-(benzothiazol-6-ylamino)-1H-pyrrolo[2,3-b]pyridin-2-yl]-3,6-dihydro-2H-pyridine-1-carboxylic acid tert-butyl ester (175 mg, 0.391 mmol) and 4M hydrogen chloride in 1,4-dioxane (10 mL) was stirred at RT for 3h. TLC indicated the completion of the reaction. The yellow solid formed was filtered washed with hexane and dried under vacuum to yield 4-[4-(benzothiazol-6-ylamino)-[2-(1,2,3,6-tetrahydropyridin-4-yl)-1H-pyrrolo[2,3-b]pyridin-4-yl]-amine trihydrochloride. MS (ES+): m/z 348.12 (100) [MH⁺]; HPLC: t_{R}=0.50 & 1.65 min (ZQ2000, polar_{—}5min).

### EXAMPLE 5

### 4-[4-(Benzothiazol-6-ylamino)-1H-pyrrolo[2,3-b]pyridin-2-yl]-3,6-dihydro-2H-pyridine-1-carboxylic acid tert-butyl ester

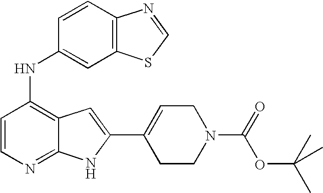

To a mixture of 4-4-chloro-1H-pyrrolo[2,3-b]pyridin-2-yl)-3,6-dihydro-2H-pyridine-1-carboxylic acid tert-butyl ester (834 mg, 2.5 mmol), 1,3-benzothiazol-6-amine (450 mg, 3.0 mmol), palladium acetate (56 mg, 0.25 mmol), (R)-(+)-2,2′-bis(diphenylphosphino)-1,1′-binaphthyl (467 mg, 0.75 mmol) and cesium carbonate (1221 mg, 3.75 mmol) was added degassed dioxane (50 mL) and the reaction was refluxed for 5h. The reaction was evaporated and the residue was purified by column chromatography over silica gel [Jones FlashMaster, 70g cartridge, eluting with methanol in DCM (0→5%)], yielding the title compound. ¹H NMR (400 MHz, CDCl₃): δ=8.97 (s, 1H), 8.00 (d, J=8.8 Hz, 1H), 7.86 (d, J=5.6 Hz, 2H), 7.47 (dd, J=8.8, 1.6 Hz, 1H), 6.76 (d, J=6.0 Hz, 1H), 6.50 (s, 1H), 6.21 (bs, 1H), 4.10-4.12 (m, 2H), 3.62 (t, J=5.6 Hz, 2H), 2.52-2.55 (m, 2H), 1.47 (s, 9H); MS (ES+): m/z 448.08 (100) [MH⁺]; HPLC: t_{R}=2.32 min (ZQ2000, polar_{—}5 min).

### 4-(4-Chloro-1H-pyrrolo[2,3-b]pyridin-2-yl)-3,6-dihydro-2H-pyridine-1-carboxylic acid tert-butyl ester

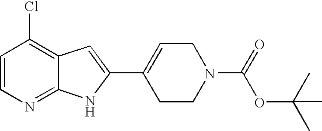

To a mixture of 4-chloro-2-iodo-1H-pyrrolo[2,3-b]pyridine (744 mg, 2.67 mmol), potassium carbonate (738 mg, 5.34 mmol), dichlorobistriphenylphosphine palladium (188 mg, 0.267 mmol) and 4-(4,4,5,5-tetramethyl-[1,3,2]dioxaborolan-2-yl)-3,6-dihydro-2H-pyridine-1-carboxylic acid tert-butyl ester (908 mg, 2.93 mmol) was added degassed dioxane (30 mL) and water (7.5 L) and the mixture was heated to reflux overnight. The reaction was evaporated under reduced pressure, water was added and extracted with DCM. The DCM extract was washed with brine, dried over anhydrous sodium sulphate and evaporated under reduced pressure. The crude product obtained was purified by chromatography on silica gel [Jones FlashMaster, 50 g/150 mL cartridge, eluting with DCM:methanol 100:0→98:2], yielding the title compound. MS (ES+): ml/z 334.10 (100) [MH⁺]; HPLC: t_{R}=3.73 min.

### 4-Chloro-2-iodo-1H-pyrrolo[2,3-b]pyridine

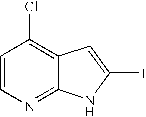

To a solution of 4-chloro-2-iodo-1-(2-iodobenzenesulfonyl)-1H-pyrrolo[2,3-b]pyridine (3.34 g, 6.133 mmol) in THF (30 mL) was added 5M sodium hydroxide in methanol (4 mL, 20 mmol) and the mixture was stirred at RT for 30 min. Water (300 mL) was added followed by saturated aqueous ammonium chloride solution (50 mL) and the precipitate formed was filtered, washed with water and hexane and dried to afford the title compound. MS (ES+): m/z 278.94 (100) [MH⁺]; HPLC: t_{R}=3.26 min.

### 1-Benzenesulfonyl-4-chloro-2-iodo-1H-pyrrolo[2,3-b]pyridine and 4-Chloro-2-iodo-1-(2-iodobenzenesulfonyl)-1H-pyrrolo[2,3-b]pyridine

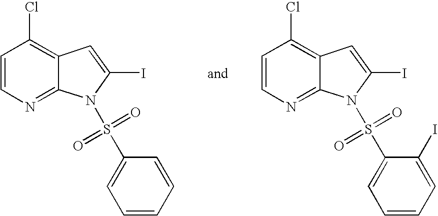

To a solution of 1-benzenesulfonyl-4-chloro-1H-pyrrolo[2,3-b]pyridine (5.000 g, 17.07 mmol) in anhydrous THF (225mL) at −78° C. was added LDA (17 mL, 1.5 M solution, 25.62 mmol) and the mixture was stirred for 30 min. A solution of iodine (8.670 g, 34.15 mmol) in THF (25 mL) was added, and the mixture was stirred for 4h at −78° C. The reaction was quenched with aqueous sodium thiosulfate solution, diluted with DCM (200 mL) and the organic layer separated. The aqueous layer was extracted with DCM. The combined organic layer was washed with brine, dried over anhydrous sodium sulfate and evaporated under reduced pressure. The crude product was purified by chromatography on silica gel [Jones FlashMaster, 100 g cartridge, eluting with DCM], yielding 4-chloro-2-iodo-1-(2-iodobenzenesulfonyl)-1H-pyrrolo[2,3-b]pyridine and 1-benzenesulfonyl-4-chloro-2-iodo-1H-pyrrolo-8 2,3-b]pyridine as a mixture in 1.5:1 ratio. MS (ES+): m/z 544.66 (100) [M⁺]; HPLC: t_{R}=4.01 min (ZQ2000, polar_{—}5 min); MS (ES+): m/z 418.74 (100) [M⁺]; HPLC: t_{R}=3.85 min.

### 1-Benzenesulfonyl-4-chloro-1H-pyrrolo[2,3-b]pyridine

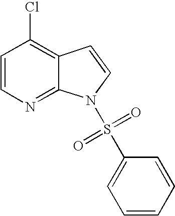

To a solution of 4-chloropyrrolopyridine (5.0 g, 32.76 mmol) in THF (100 mL) at 0° C. was added sodium hydride (1.179 g, 49.15 mmol) and the mixture was stirred for 15 min. Benzenesulfonyl chloride (6.945 g, 39.32 mmol) was added and the mixture was stirred for 4h. The reaction was quenched with saturated ammonium chloride solution and the THF layer was separated. The aqueous layer was extracted with DCM (2×75 mL). The combined organic layer was washed with brine, dried over anhydrous sodium sulfate and evaporated under reduced pressure. The crude product obtained was purified by chromatography on silica gel [Jones FlashMaster, 70 g/150 mL cartridge, eluting with hexane:ethyl acetate 100:0→92:08], yielding the title compound as a colorless solid. MS (ES+): m/z 293.02 (100) [MH⁺]; HPLC: t_{R}=3.56 min (ZQ2000, polar_{—}5 min).

### EXAMPLE 6

### 4-[4-(1H-Indazol-5-ylamino)-1H-pyrrolo[2,3-b]pyridin-2-yl]-3,6-dihydro-2H-pyridine-1-carboxylic acid tert-butylamide

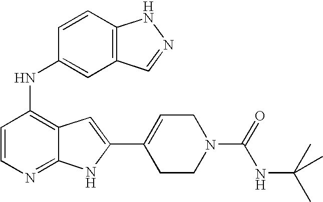

To a mixture of (1H-indazol-5-yl)-[2-(1,2,3,6-tetrahydropyridin-4-yl)-1H-pyrrolo[2,3-b]pyridin4-yl]-amine trihydrochloride (92 mg, 0.21 mmol) and N,N-diisopropylethyl amine (135 mg, 1.04 mmol) in dry DMF (3.0 mL) at 0° C. was added tert-butyl isocyanate (21 mg, 0.21 mmol) and the mixture was allowed to warm to RT and stirred overnight. The reaction was purified by column chromatography over silica gel [Jones FlashMaster, 50 g cartridge, eluting with DCM/methanol] followed by preparative TLC (8% methanol in DCM was used as eluent) to yield the title compound as yellow solid. ¹H NMR (400 MHz, CDCl₃): δ=8.00 (s, 1H), 7.84 (d, J=5.6 Hz, 1H), 7.65 (d, J=0.8 Hz, 1H), 7.54 (d, J=8.8 Hz, 1H), 7.37 (dd, J=4.8, 2.0 Hz, 1H), 6.52 (d, J=5.6 Hz, 1H), 6.41 (s, 11H), 6.17 (bs, 1H), 4.59 (s, 1H), 4.01-4.03 (m, 2H), 3.61 (t, J=5.6 Hz, 2H), 3.29 (bs, 2H), 2.54-2.57 (m, 2H), 1.38 (s, 9H); MS (ES+): m/z 430.21 (100) [MH⁺]; HPLC: t_{R}=2.09 min (ZQ2000, polar_{—}5 min).

### (1H-Indazol-5-yl)-[2-(1,2,3,6-tetrahydropyridin-4-yl)-1H-pyrrolo[2,3-b]pyridin4-yl]-amine trihydrochloride

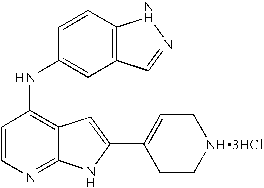

A mixture of 4-[4-(1H-indazol-5-ylamino)-1H-pyrrolo[2,3-b]pyridin-2-yl]-3,6-dihydro-2H-pyridine-1-carboxylic acid tert-butyl ester (101 mg, 0.23 mmol) and 4M hydrogen chloride in 1,4-dioxane (4 mL) was stirred at RT for 3h. The solids formed were filtered, washed with hexane, and dried by azeotropic evaporation using toluene followed by vacuum to yield the title compound. MS (ES+): m/z 331.21 (100) [MH⁺]; HPLC: t_{R}=1.45 & 0.48 min (ZQ2000, polar_{—}5 min).

### EXAMPLE 7

### 4-[4-(1H-Indazol-5-ylamino)-1H-pyrrolo[2,3-b]pyridin-2-yl]-3,6-dihydro-2H-pyridine-1-carboxylic acid tert-butyl ester

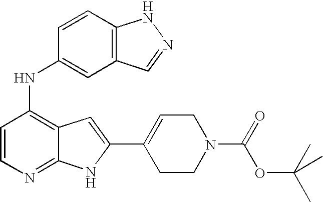

To a mixture of 4-4-chloro-1H-pyrrolo[2,3-b]pyridin-2-yl)-3,6dihydro-2H-pyridine-1-carboxylic acid tert-butyl ester (200 mg, 0.6 mmol), 5-aminoindazole (96 mg, 0.72 mmol), palladium acetate (13.4 mg, 0.06 mmol), (R)-(+)-2,2′-bis(diphenylphosphino)-1,1′-binaphthyl (112 mg, 0.18 mmol) and cesium carbonate (293 mg, 0.9 mmol) was added degassed DMF (7 mL), and the reaction was heated at 150° C. overnight. DMF was evaporated and the residue was purified by column chromatography over silica gel [Jones FlashMaster, 50 g cartridge, eluting with DCM/methanol], followed by a preparative TLC purification using 7% methanol in DCM as eluent, afforded the title compound. ¹H NMR (400 MHz, CDCl₃): δ=7.92 (dd, J=2.4, 1.2 Hz, 1H), 7.75 (t, J=5.6 Hz, 2H), 7.56 (dd, J=5.2, 1.6 Hz, 1H), 7.46 (dd, J=8.8, 3.2 Hz, 1H), 7.25-7.29 (m, 2H), 6.45 (t, J=6.0 Hz, 1H), 6.31 (bs, 1H), 6.14 (bs, 1H), 4.04-4.07 (m, 1H), 3.53-3.60 (m, 2H), 3.44-3.61 (m, 1H), 2.45-2.47 (m, 1H), 1.88-2.12 (m, 1H), 1.41 (s, 9H); MS (ES+): m/z 431.16 (100) [MH⁺]; HPLC: t_{R}=2.25 min (ZQ2000, polar_{—}5min).

### EXAMPLE 8

### {4-[4-(Benzothiazol-6-ylamino)-1H-pyrrolo[2,3-b]pyridin-2-yl]-3,6-dihydro-2H-pyridin-1-yl}-(4-methylpiperazin-1-yl)methanone

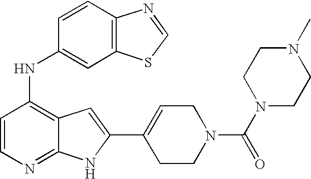

To a mixture of benzothiazol-6-yl-[2-(1,2,3,6-tetrahydropyridin-4-yl)-1H-pyrrolo[2,3-b]pyridin-4-yl]-amine trihydrochloride (51.8 mg, 0.113 mmol) in anhydrous DMF (4 mL), N,N-diisopropylethylamine (100 μL, 0.6 mmol) was added at rt. After cooling to 0° C., 4-methyl-1-piperazinecarbonyl chloride hydrochloride (24.9 mg, 0.148 mmol) in anhydrous DMF (4 mL) was added. The reaction was stirred at 0° C. for 1h, after which it was quenched with MeOH and concentrated in vacuo. The crude was purified by chromatography on silica gel [Jones Flashmaster, 5 g/25 mL, eluting with 10% MeOH:CH₂Cl₂→7N NH₃(MeOH):CH₂Cl₂ 2%→5% ]. Fractions containing product were combined, concentrated in vacuo, and further purified by trituration in CH₂Cl₂, affording the title compound as a white solid. ¹H NMR (400 MHz, DMSO-d₆): δ=2.20 (s, 3H), 2.33 (s, br, 4H), 2.47-2.53 (m, obscured, 2H), 3.17 (s, br, 4H), 3.39 (t, J=5.6 Hz, 2H), 3.92 (d, J=1.6 Hz, 2H), 6.39 (s, br, 1H), 6.62 (d, J=2.0 Hz, 1H), 6.76 (d, J=5.2 Hz, 1H), 7.46 (dd, J=8.8, 2.0 Hz, 1H), 7.91 (d, J=5.2 Hz, 1H), 8.02 (d,J=2.4 Hz, 1H), 8.03 (d, J=9.2 Hz, 1H), 8.88 (s, —NH), 9.22 (s, 1H), 11.55 (d, J=1.2 Hz, —NH); MS (ES+): m/z 474.03 (35) [MH⁺]; HPLC: t_{R}=1.67 min (OpenLynx, polar_{—}5 min).

### EXAMPLE 9

### 4-[4-(Benzothiazol-6-ylamino)-1H-pyrrolo[2,3-b]pyridin-2-yl]-3,6-dihydro-2H-pyridine-1-carboxylic acid dimethylamide

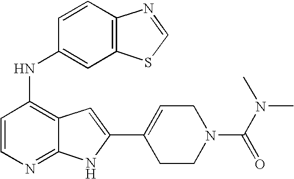

To a suspension of benzothiazol-6-yl-[2-(1,2,3,6-tetrahydropyridin-4-yl)-1H-pyrrolo[2,3-b]pyridin-4-yl]-amine trihydrochloride (98 mg, 0.21 mmol) in anhydrous DMF (6 mL), N,N-diisopropylethylamine (200 μL, 1 mmol) was added at rt. After cooling to 0° C., N,N-dimethylcarbamoyl chloride (33.1 mg, 0.302 mmol) in anhydrous DMF (4 mL) was added. The reaction was stirred at 0° C. for 1h, after which it was quenched with MeOH and concentrated in vacuo. The crude was purified by chromatography on silica gel [0.5″×10″ column, eluting with neat DCM→ MeOH:CH₂Cl₂ 2%→5%→6%]. Fractions containing product were combined and concentrated in vacuo. The residue was dissolved in a 5% solution of MeOH:CH₂Cl₂, washed with water (3×), dried over anhydrous Na₂SO₄, filtered, and concentrated in vacuo, yielding the title compound as a yellow solid. ¹H NMR (400 MHz, DMSO-d₆): δ=2.45-2.54 (m, obscured, 2H), 2.77 (s, 6H), 3.36 (t, J=5.2 Hz, 2H), 3.88 (d, J=2.8 Hz, 2H), 6.40 (s, br, 1H), 6.62 (d, J=1.6 Hz, 1H), 6.76 (d, J=5.6 Hz, 1H), 7.46 (dd, J=8.8,2.0 Hz, 1H), 7.91 (d, J=5.6 Hz, 1H), 8.02 (d, J=2.4 Hz, 1H), 8.03 (d, J=8.8 Hz, 1H), 8.89 (s, —NH), 9.22 (s, 1H), 11.56 (s, —NH); MS (ES+): m/z 419.13 (100) [MH⁺]; HPLC: t_{R}=2.05 min (OpenLynx, polar_{—}5 min).

### EXAMPLE 10

### 4-[4-(Benzothiazol-ylamino)-1H-pyrrolo[2,3-b]pyridin-2-yl]-3,6-dihydro-2H-pyridin-1-yl-(4-cyclopentylpiperazin-1-yl)-methanone.

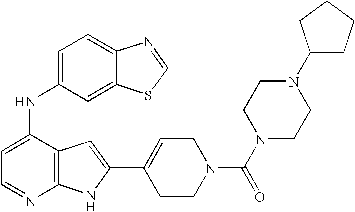

To a suspension of benzothiazol-6-yl-[22-(1,2,3,6-tetrahydropyridin-4-yl)-1H-pyrrolo[2,3-b]pyridin-4-yl]-amine trihydrochloride (80.8 mg, 0.177 mmol) in anhydrous DMF (6 mL), N,N-diisopropylethylamine (200 μL, 0.9 mmol) was added at rt. After cooling to 0° C., 4-cyclopentylpiperazine-1-carbonyl chloride (56.3 mg, 0.26 mmol) in DMF (2mL) was added. The reaction was stirred at 0° C. for 1h, after which it was quenched with 7N solution of NH₃ in MeOH and concentrated in vacuo. The crude was purified by chromatography on silica gel [0.5″×10″ column, eluting with MeOH:CH₂Cl₂ 1%→10%]. Fractions containing product were combined and concentrated in vacuo. The residue was dissolved in CH₂Cl₂, washed with water (2×) and brine (1×), dried over anhydrous Na₂SO₄, filtered, and concentrated in vacuo, affording the title compound as a yellow solid. ¹H NR (400 Hz, DMSO-d₆): δ=1.18-1.39 (m, 3H), 1.39-1.55 (m, 2H), 1.55-1.67 (m, 2H), 1.68-1.84 (m, 2H), 2.41 (s, br, 4H), 2.44-2.55 (m, obscured, 2H), 3.16 (s, br, 4H), 3.39 (t, obscured, J=5.2 Hz, 2H), 3.91 (s, br, 2H), 6.39 (s, br, 1H), 6.62 (d, J=2.0 Hz, 1H), 6.76 (d, J=5.6 Hz, 1H), 7.46 (dd, J=8.8, 2.0 Hz, 1H), 7.91 (d, J=5.6 Hz, 1H), 8.02 (d, J=2.0 Hz, 1H), 8.03 (d, J=9.2 Hz, 1H), 8.88 (s, 1H), 9.22 (s, 1H), 11.54 (s, —NH); MS (ES+): m/z 528.18 (5) [MH⁺]. HPLC: t_{R}=1.77 min (OpenLynx, polar_{—}5 min).

### EXAMPLE 11

General procedure A: To a stirred mixture of 1-[4-(4-chloro-1H-pyrrolo[2,3-b]pyridin-2-yl)-3,6-dihydro-2H-pyridin-1-yl]-2-dimethylaminoethanone (0.09 mmol) and a (substituted)aniline (0.10 mmol) in n-BuOH (0.5 mL) and DMF (0.1 mL) was added AlCl₃ (24 mg, 2 eq). The mixture was heated to 95° C. and stirred at the same temperature for 4-6 hours. Reaction was monitored by TLC and LC-MS. After TLC shows a complete conversion, the mixture was cooled down to room temperature, quenched with NaHCO₃ (sat. aq. solution) and extracted with CHCl₃ (34 times), dried (Na₂SO₄) and evaporated to give crude product, which was then purified by flash chromatography (3% MeOH in DCM) to provide the desired product.

### EXAMPLE 12

### 2-Dimethylamino-1-{4-[4-(3-ethynylphenylamino)-1H-pyrrolo[2,3-b]pyridin-2-yl-3,6-dihydro-2H-pyridin-1-yl}-ethanone

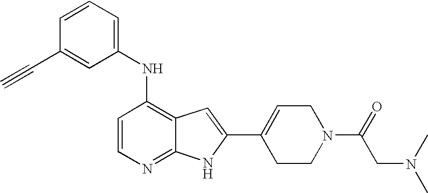

Prepared by General Procedure A. MS (ES+): m/z: 400.20 (MH⁺). HPLC: t_{R}=1.87 min (OpenLynx, polar_{—}5min).

### EXAMPLE 13

### 1-{4-[4-(4-Chloro-2,5-dimethoxyphenylamino)-1H-pyrrolo[2,3-b]pyridin-2-yl]-3,6-dihydro-2H-pyridin-1-yl]-2-dimethylaminoethanone

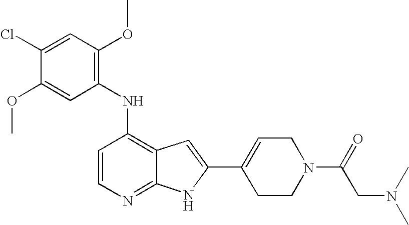

Prepared by General Procedure A. MS (ES+): m/z: 470.03 (MH⁺). HPLC: t_{R}=1.92 min (OpenLynx, polar_{—}5 min).

### EXAMPLE 14

### {4-[4-(3-Chloro-4-fluorophenylamino)-1H-pyrrolo[2,3-b]pyridin-2-yl]-3,6-dihydro-2H-pyridin-1-yl}-(cis-2,6-dimethylmorpholin-4-yl)-methanone

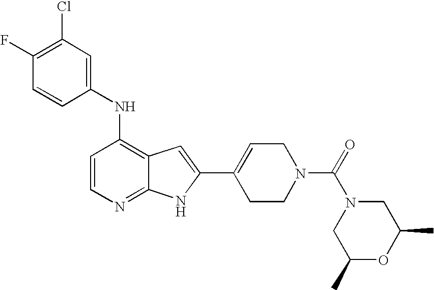

Prepared by General Procedure A. MS (ES+): m/z: 483.99 (MH⁺). HPLC: t_{R}=2.37 min (OpenLynx, polar_{—}5 min).

### EXAMPLE 15

### 4-[4-(3-Chloro-4-fluorophenylamino)-1H-pyrrolo [2,3-b]pyridin-2-yl]-3,6-dihydro-2H-pyridine-1-carboxylic acid ethyl-methyl-amide

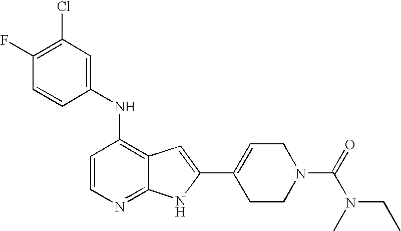

Prepared by General Procedure A. MS (ES+): m/z: 427.95 (MH⁺). HPLC: t_{R}=2.35 min (OpenLynx, polar_{—}5 min).

### EXAMPLE 16

### 4-[4-(4-Chloro-3-ethynyl-5-methoxyphenylamino)-1H-pyrrolo[2,3-b]pyridin-2-yl]-3,6-dihydro-2H-pyridine-1-carboxylic acid ethyl-methyl-amide

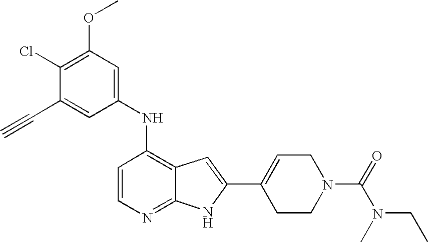

Prepared by General Procedure A. MS (ES+): m/z 464.17 (MH⁺, ³⁵Cl), 466.13 (MH⁺, ³⁷Cl). HPLC: t_{R}=2.51 min (OpenLynx, polar_{—}5 min).

### EXAMPLE 17

### {4-[4-(4-Chloro-3-ethynyl-5-methoxyphenylamino)-1H-pyrrolo[2,3-b]pyridin-2-yl]-3,6-dihydro-2H-pyridin-1-yl}-(cis-2,6-dimethylmorpholin-4-yl)-methanone

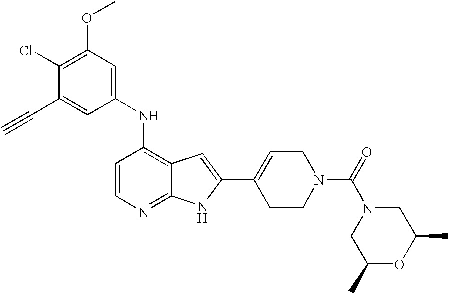

Prepared by General Procedure A. MS (ES+): m/z 520.13 (MH⁺, ³⁵Cl), 522.19 (MH⁺, ³⁷Cl). HPLC: t_{R}=2.53 min (OpenLynx, polar_{—}5 min).

### EXAMPLE 18

### 4-14-(4-Chloro-3ethyl-5-methoxyphenylamino)-1H-pyrrolo[2,3-b]pyridin-2-yl]-3,6-dihydro-2H-pyridine-1-carboxylic acid dimethylamide

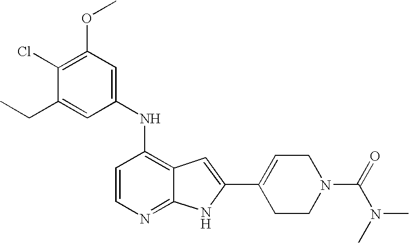

Prepared by General Procedure A. MS (ES+): m/z 454.15 (MH⁺, ³⁵Cl), 456.15 (MH⁺, ³⁷Cl). HPLC: t_{R}=2.53 min (OpenLynx, polar_{—}5min).

### EXAMPLE 19

### {4-[4-(4-Chloro-3-methoxyphenylamino)-1H-pyrrolo[2,3-b]pyridin-2-yl]-3,6-dihydro-2H-pyridin-1-yl}-(cis-2,6-dimethylmorpholin-4-yl)-methanone

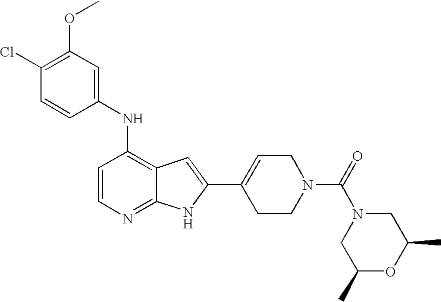

Prepared by General Procedure A. MS (ES+): m/z 496.18 (MH⁺, ³⁵Cl), 498.18 (MH⁺, ³⁷Cl). HPLC: t_{R}=2.43 min (OpenLynx, polar_{—}5 min).

### EXAMPLE 20

### 4-[4-(4-Chloro-3-methoxyphenylamino)-1H-pyrrolo[2,3-b]pyridin-2-yl]-3,6-dihydro-2H-pyridine-1-carboxylic acid ethyl-methyl-amide

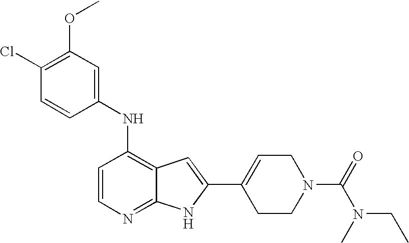

Prepared by General Procedure A. MS (ES+): m/z 440.14 (MH⁺, ³⁵Cl), 442.11 (MH⁺, ³⁷Cl). HPLC: t_{R}=2.44 min (OpenLynx, polar_{—}5 min).

### EXAMPLE 21

### 4-[4-(4-Chloro-3-methoxyphenylamino)-1H-pyrrolo[2,3-b]pyridin-2-yl]-3,6-dihydro-2H-pyridine-1-carboxylic acid dimethylamide

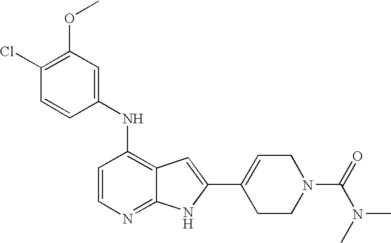

Prepared by General Procedure A. MS (ES+): m/z 425.96 (MH⁺, ³⁵Cl), 427.96 (MH⁺, ³⁷Cl). HPLC : t_{R}=2.26 min (OpenLynx, polar_{—}5 min).

### EXAMPLE 22

### {4-[4-(4-Chloro-3-methoxyphenylamino)-1H-pyrrolo[2,3-b]pyridin-2-yl]-3,6-dihydro-2H-pyridin-1-yl}-(4-thiazol-2-yl-piperazin-1-yl)-methanone

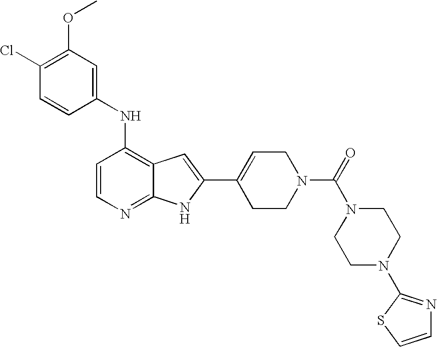

Prepared by General Procedure A. MS (ES+): m/z 549.94 (MH⁺, ³⁵C), 551.86 (MH⁺, ³⁷C). HPLC: t_{R}=2.40 min (OpenLynx, polar_{—}5 min).

### EXAMPLE 23

### 4-[4-(4-Chloro-3-cyclopropyl-5-methoxyphenylamino)-1H-pyrrolo[2,3-b]pyridin-2-yl]-3,6dihydro-2H-pyridine-1-carboxylicacid dimethylamide

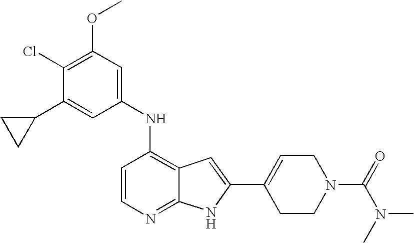

Prepared by General Procedure A. Yield: 35%. ¹H-NMR (CD₃O, 400 MHz): δ=0.62-0.69 (m, 2 H), 0.96-1.07 (m, 2 H), 2.18-2.30 (m, 1H), 2.62 (d,J=1.52 Hz, 3 2 H), 2.88 (s, 6 H), 3.49 (t, J=5.68 Hz, 2H), 3.85 (s, 3H), 4.00 (d, J=3.03 Hz, 2H), 6.27 (br, s, 1 H), 6.49-6.54 (m, 2H), 6.69 (d, J=5.81 Hz, 1 H), 6.82 (d, J=2.27 Hz, 1H), 7.86 (d, J=5.81 Hz, 1 H). MS (ES+): m/z 466.15 (MH⁺, ³⁵Cl), 468.17 (MH⁺, ³⁷Cl). HPLC: t_{R}=2.48 min (OpenLynx, polar_{—}5 min).

### EXAMPLE 24

### {4-[4-(4-Chloro-3-cyclopropyl-5-methoxyphenylamino)-1H-pyrrolo[2,3-b]pyridin-2-yl]-3,6-dihydro-2H-pyridin-1-yl}-(4-methylpiperazin-1-yl)-methanone

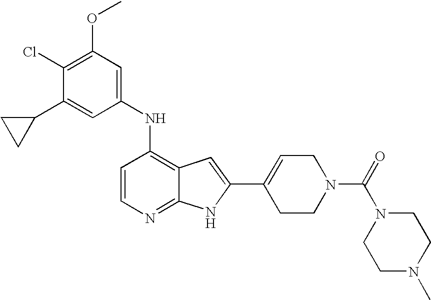

Prepared by General Procedure A. MS (ES+): m/z 521.20 (MH⁺, ³⁵Cl), 523.16 (MH⁺, ³⁷Cl). HPLC: t_{R}=2.08 min (OpenLynx, polar_{—}5 min).

### EXAMPLE 25

General procedure B: To a mixture of 4-chloro-1H-pyrrolo[2,3-b]pyridine compound (0.277 mmol), (R)-+)-2,2′-bis(diphenylphosphino)-1,1′-binaphthyl (52 mg, 0.083 mmol), palladium acetate (6.2 mg, 0.027 mmol), cesium carbonate (136 mg, 0.41 mmol) in anhydrous dioxane (2 mL) under nitrogen atmosphere was added an aniline (0.36 mmol). The reaction was heated to reflux for 4h-3 days. After the reaction was complete, the reaction mixture was cooled to room temperature, methanol:dichloromethane (2:1, 5 mL) was added. The resulting mixture was filtered and the filtrate was evaporated to dryness. The residue was purified by silica chromatography to afford the desired product.

### EXAMPLE 26

### 4-[4-(4-Chloro-3-methoxy-5-methylphenylamino)-1H-pyrrolo[2,3-b]pyridin-2-yl]-3,6-dihydro-2H-pyridine-1-carboxylic acid tert-butyl ester

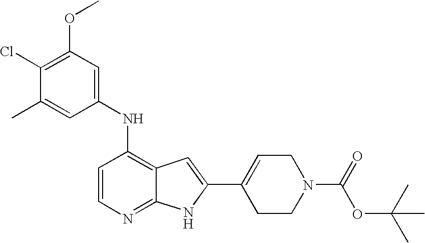

Prepared by General Procedure B. Yield: 89%. m.p.: 224-225° C. ¹H-NMR (300 MHz, DMSO-d6): δ=1.42 (s, 9 H), 2.29 (s, 3 H), 2.40 (s, 2 H), 3.55 (t, J=2 Hz, 2 H), 3.80 (s, 3 H), 4.02 (br s, 2 H), 6.40 (br, s, 1 H), 6.60 (s, 1 H), 6.74 (d, J=6 Hz, 1H), 6.85 (s, 2 H), 7.90 (d, J=6 Hz, 1 H), 8.67 (s, 1 H), 11.53 (br, s, NH). MS (ES+): m/z 469 (MH⁺).

### EXAMPLE 27

### (4-Methylpiperazin-1-yl)-14-(4-phenylamino-1H-pyrrolo[2,3-b]pyridin-2-yl)-3,6-dihydro-2H-pyridin-1-yl]-methanone

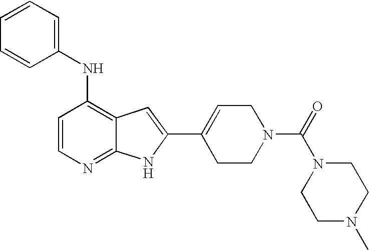

Prepared by General Procedure B. MS (ES+): m/z 417.01 (MH⁺). HPLC: t_{R}=1.66 min (OpenLynx, polar_{—}5 min).

### EXAMPLE 28

### 4-[4-1H-Indol-5-ylamino)-1H-pyrrolo[2,3-b]pyridin-2-yl]-3,6-dihydro-2H-pyridine-1-carboxylic acid tert-butylamide

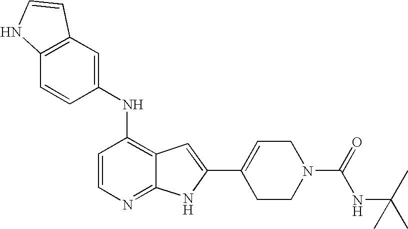

Prepared by General Procedure B. MS (ES+): m/z 428.95 (MH⁺). HPLC: t_{R}=2.30 min (OpenLynx, polar_{—}5 min).

### EXAMPLE 29

### {4-[4-(1H-Indol-5-ylamino)-1H-pyrrolo[2,3-b]pyridin-2-yl]-3,6-dihydro-2H-pyridin-1-yl}-(4-methylpiperazin-1-yl)-methanone

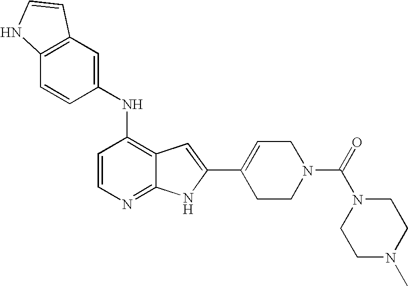

Prepared by General Procedure B. MS (ES+): m/z 455.95 (MH⁺). HPLC: t_{R}=1.80 min (OpenLynx, polar_{—}5 min).

### EXAMPLE 30

### {4-[4-(3-Chloro4-fluorophenylamino)-1H-pyrrolo[2,3-b]pyridin-2-yl]-3,6-dihydro-2H-yridin-1-yl}-(4-methylpiperazin-1-yl)-methanone

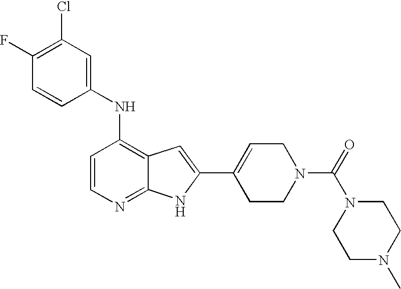

Prepared by General Procedure B. MS (ES+): ml/z 469.23 (MH⁺, ³⁵Cl), 471.17 (MH⁺, ³⁷Cl). HPLC: t_{R}=1.94 min (OpenLynx, polar_{—}5 min).

### EXAMPLE 31

### {4-[4-4-Chloro-3-methoxyphenylamino)-1H-pyrrolo[2,3-b]pyridin-2-yl]-3,6-dihydro-2H-pyridin-1-yl}-(4-methylpiperazin-1-yl)methanone

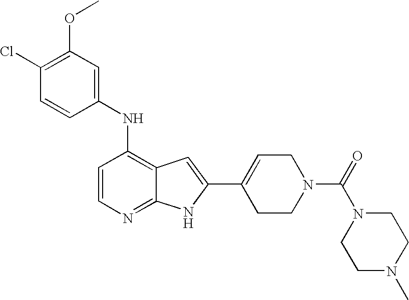

Prepared by General Procedure B. MS (ES+): m/z 481.18 (MH⁺, ³⁵Cl), 483.20 (MH⁺, ³⁷Cl). HPLC: t_{R}=1.95 min (OpenLynx, polar_{—}5 min).

### EXAMPLE 32

### 1-4-[4-(Benzothiazol-6-ylamino)-1H-pyrrolo[2,3-b]pyridin-2-yl]-3,6-dihydro-2H-pyridin-1-yl-2-dimethylaminoethanone

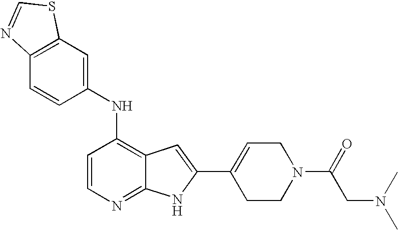

Prepared by General Procedure B. MS (ES+): m/z 433.13 (100) [MH⁺]. HPLC: t_{R}=0.54, 1.73 min (OpenLynx, polar_{—}5 min).

### EXAMPLE 33

### 2-Dimethylamino-1-4-[4-(1H-indol-5-ylamino)-1H-pyrrolo[2,3-b]pyridin-2-yl]-3,6-dihydro-2H-pyridin-1-yl-ethanone

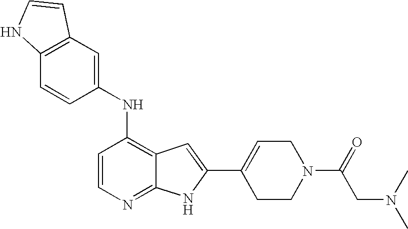

Prepared by General Procedure B. MS (ES+): m/z 414.94 (100) [MH⁺]. HPLC: t_{R}=0.43, 1.64 min (OpenLynx, polar_{—}5 min).

### EXAMPLE 34

### 1-4-[4-(3,5-Dimethoxyphenylamino)-1H-pyrrolo[2,3-b]pyridin-2-yl]-3,6-dihydro-2H-pyridin-1-yl-2-dimethylaminoethanone

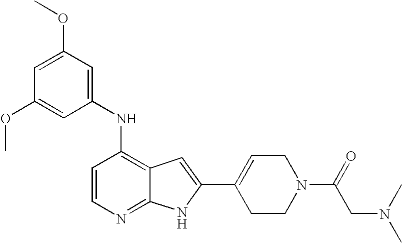

Prepared by General Procedure B. MS (ES+): m/z 435.96 (100) [MH⁺]. HPLC: t_{R}=1.70 min (OpenLynx, polar_{—}5 min).

### EXAMPLE 35

### 2-Dimethylamino-1-[4-(4-phenylamino-1H-pyrrolo 12,3-b]pyridin-2-yl)-3,6-dihydro-2H-pyridin-1-yl]-ethanone

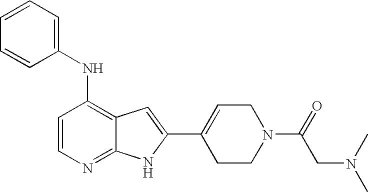

Prepared by General Procedure B. MS (ES+): m/z 375.98 (100) [MH⁺]. HPLC: t_{R}=0.43, 1.61 min (OpenLynx, polar_{—}5 min).

### EXAMPLE 36

### 4-[4-(Benzo[1,3]dioxol-5-ylamino)-1H-pyrrolo[2,3-b]pyridin-2-yl]-3,6-dihydro-2H-pyridin-1-yl-(4-methylpiperazin-1-yl)-methanone

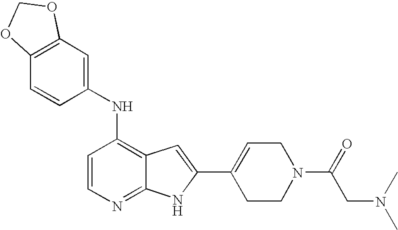

Prepared by General Procedure B. MS (ES+): m/z 460.96 (MH⁺). HPLC: t_{R}=1.75 min (OpenLynx, polar_{—}5 min).

### EXAMPLE 37

### 4-[4-(3-Chloro-4-fluorophenylamino)-1H-pyrrolo[2,3-b]pyridin-2-yl]-3,6-dihydro-2H-pyridine-1-carboxylic acid t-butylamide

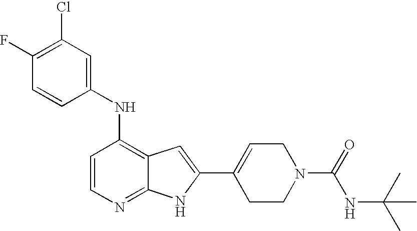

Prepared by General Procedure B. MS (ES+): m/z 441.96 (MH⁺). HPLC: t_{R}=2.41 min (OpenLynx, polar_{—}5 min).

### EXAMPLE 38

### {4-[4-(3-Ethynylphenylamino)-1H-pyrrolo[2,3-b]pyridin-2-yl]-3,6-dihydro-2H-pyridin-1-yl}-(4-methylpiperazin-1-yl)methanone

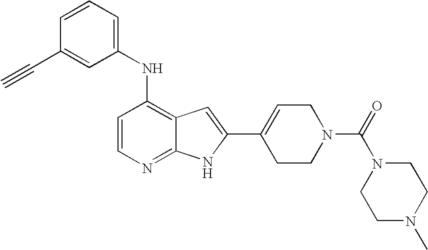

Prepared by General Procedure B. MS (ES+): m/z 441.01, 442.02 [MH⁺]. HPLC: t_{R}=1.80 min (OpenLynx, polar_{—}5 min).

### EXAMPLE 39

### 4-[4-(3-Ethynylphenylamino)-1H-pyrrolo[2,3-b]pyridin-2-yl]-3,6-dihydro-2H-pyridine-1-carboxylic acid tert-butylamide

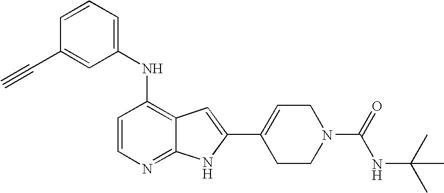

Prepared by General Procedure B. MS (ES+): ml/z 414.27 & 414.29 [MH⁺]. HPLC: t_{R}=2.45 & 3.34 min (OpenLynx, polar_{—}5 min).

### EXAMPLE 40

### {4-[4-(3,5-Dimethoxyphenylamino)-1H-pyrrolo[2,3-b]pyridin-2-yl]-3,6-dihydro-2H-pyridin-1-yl}-(4-methylpiperazin-1-yl)-methanone

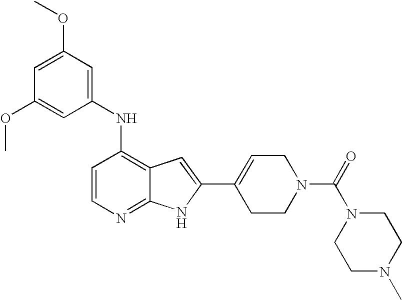

Prepared by General Procedure B. MS (ES+): m/z 476.96 [MH⁺]. HPLC: t_{R}=1.84 min (OpenLynx, polar_min).

### EXAMPLE 41

### {4-[4-(1H-Indazol-5-ylamino)-1H-pyrrolo[2,3-b]pyridin-2-yl]-3,6-dihydro-2H-pyridin-1-yl}-(4-methylpiperazin-1-yl)methanone

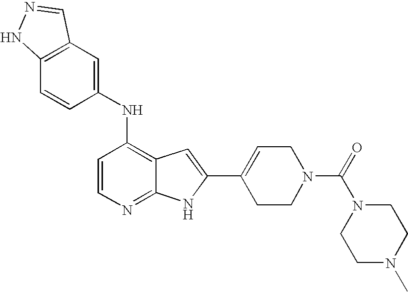

Prepared by General Procedure B. MS (ES+): m/z 456.99 [MH⁺]. HPLC: t_{R}=0.49 & 1.67 min (OpenLynx, polar_{—}5 min).

### EXAMPLE 42

### {4-[4-(2,3-Dihydroindol-1-yl)-1H-pyrrolo[2,3-b]pyridin-2-yl]-3,6-dihydro-2H-pyridin-1-yl}-(4-methylpiperazin-1-yl)methanone

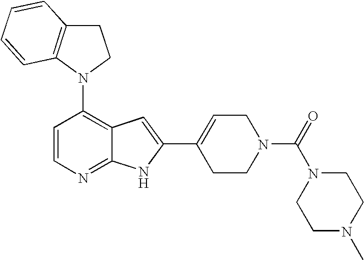

Prepared by General Procedure B. MS (ES+): m/z 443.01 [MH⁺]. HPLC: t_{R}=1.88 min (OpenLynx, polar_{—}5 min).

### EXAMPLE 43

### 1-4-[4-(4-Chloro-3-methoxyphenylamino)-1H-pyrrolo[2,3-b]pyridin-2-yl]-3,6-dihydro-2H-pyridin-1-yl-2-dimethylaminoethanone

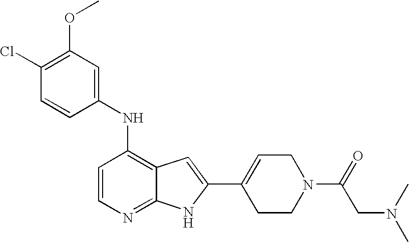

Prepared by General Procedure B. MS (ES+): m/z 440.00 [MH⁺]. HPLC: t_{R}=1.85 min (OpenLynx, polar_{—}5 min).

### EXAMPLE 44

### 1-4-[4-(3-Chloro-4-fluorophenylamino)-1H-pyrrolo[2,3-b]pyridin-2-yl]-3,6-dihydro-2H-pyridin-1-yl-2-dimethylaminoethanone

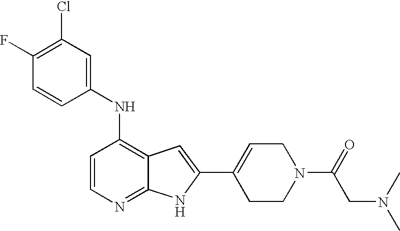

Prepared by General Procedure B. MS (ES+): m/z 428.00 [MH⁺]. HPLC: t_{R}=1.85 min (OpenLynx, polar_{—}5 min).

### EXAMPLE 45

### 1-4-[4-(Benzo[1,3]dioxol-5-ylamino)-1H-pyrrolo[2,3-b]pyridin-2-yl-3,6-dihydro-2H-pyridin-1-yl-2-dimethylaminoethanone

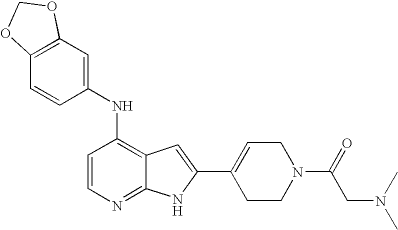

Prepared by General Procedure B. MS (ES+): m/z 420.05 [MH⁺]. HPLC: t_{R}=0.50 & 1.71 min (OpenLynx, polar_{—}5 min).

### EXAMPLE 46

### 1-4-14-(2,3-Dihydroindol-1-yl)-1H-pyrrolo [2,3-b]pyridin-2-yl]-3,6-dihydro-2H-pyridin-1-yl-2-dimethylaminoethanone

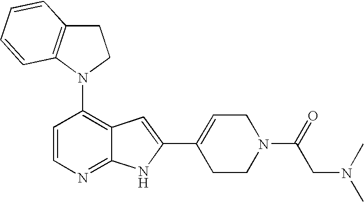

Prepared by General Procedure B. MS (ES+): m/z 401.98 [MH⁺]. HPLC: t_{R}=1.73 min (OpenLynx, polar_{—}5 min).

### EXAMPLE 47

### 2-Dimethylamino-1-4-[4-(1H-indazol-5-ylamino)-1H-pyrrolo[2,3-b]pyridin-2-yl]-3,6-dihydro-2H-pyridin-1-yl-ethanone

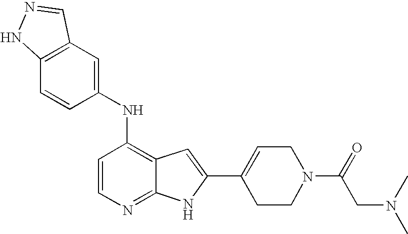

Prepared by General Procedure B. MS (ES+): m/z 415.95 [MH⁺]. HPLC: t_{R}=0.42 & 1.52 min (OpenLynx, polar_{—}5 min).

### EXAMPLE 48

### 4-[4-(4-Chloro-3-ethynyl-5-methoxyphenylamino)-1H-pyrrolo[2,3-b]pyridin-2-yl]-3,6-dihydro-2H-pyridine-1-carboxylic acid dimethylamide

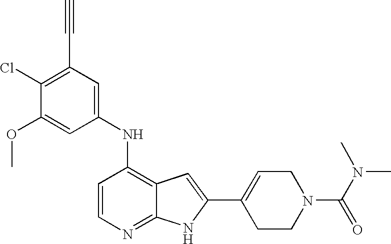

Prepared by General Procedure B. MS (ES+): m/z 450.17 (MH⁺, ³⁵Cl), 452.14 (MH⁺, ³⁷Cl). HPLC: t_{R}=2.49 min (OpenLynx, polar_{—}5 min).

### EXAMPLE 49

### 4-[4-(4-Chloro-3-methoxy-5-methylphenylamino)-1H-pyrrolo[2,3-b]pyridin-2-yl-3,6-dihydro-2H-pyridine-1-carboxylic acid dimethylamide

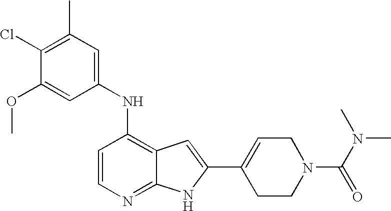

Prepared by General Procedure B. MS (ES+): m/z 440.18 (MH⁺, ³⁵Cl), 442.11 (MH⁺, ³⁷Cl). HPLC: t_{R}=2.53 min (OpenLynx, polar_{—}5 min).

### EXAMPLE 50

### 4-14-(4-Chloro-3-cyclopropyl-5-methoxyphenylamino)-1H-pyrrolo[2,3-b]pyridin-2-yl]-3,6dihydro-2H-pyridine-1-carboxylic acid dimethylamide

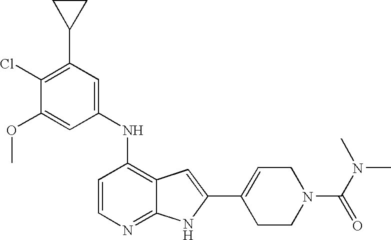

Prepared by General Procedure B. MS (ES+): m/z 466.16 (MH⁺, ³⁵Cl), 468.08 (MH⁺, ³⁷Cl). HPLC: t_{R}=2.51 min (OpenLynx, polar_{—}5 min).

### EXAMPLE 51

General procedure C: A small vial was charged with an amine (0.117 mmol), an acid (0.128 mmol), TBTU (40.0 mg, 0.125 mmol), DIPEA (0.102 mL, 0.583 mmol), DMF (0.5 mL) and a stirring bar. The reaction mixture was allowed to stir at room temperature for 2 h. LC-MS indicated complete conversion of the starting materials. Water (30 mL) was added to the reaction mixture and the precipitate was collected in a sintered glass frit by filtration. The crude product was washed with 3×5 mL H₂O and then dissolved in MeOH/dichloromethane and purified by silica gel chromatography to afford the desired product.

### EXAMPLE 52

### 1-4-[4-(3-Chloro-4-fluorophenylamino)-1H-pyrrolo[2,3-b]pyridin-2-yl]3,6-dihydro-2H-pyridin-1-yl}-(R)-piperidin-2-yl-methanone

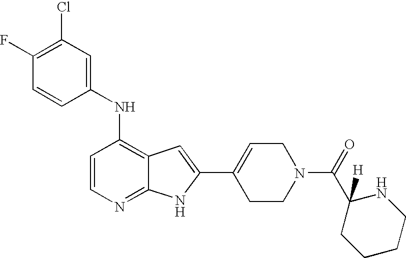

Prepared by General Procedure C. Yield: 28.2%. ¹H-NMR (CDCl₃/MeOD, 400 MHz): δ=7.83 (d, J=5.81 Hz, 1H), 7.33 (dd, J=6.06, 2.28 Hz, 1H), 7.16 (s, 1 H), 7.09 (d, J=8.59 Hz, 1 H), 6.56 (d, J=5.31 Hz, 1 H), 6.46 (d, J=5.31 Hz, 1 H), 6.20-6.12 (m, 1 H), 4.29 (m, 2 H), 4.03 (m, 2 H), 3.80 (m, 2 H), 3.54-3.36 (m, 2 H), 2.97 (m, 2 H), 1.88 (m, 2 H), 1.62 (m, 2 H), 1.75 (m, 1 H). MS (ES+): m/z 454.41 (MH⁺). HPLC: t_{R}=2.03 min (OpenLynx, polar_{—}5 min).

### EXAMPLE 53

### {4-[4-4-Chloro-2,5-dimethoxyphenylamino)-1H-pyrrolo[2,3-b]pyridin-2-yl]-3,6-dihydro-2H-pyridin-1-yl}-(R)-piperidin-2-yl-methanone

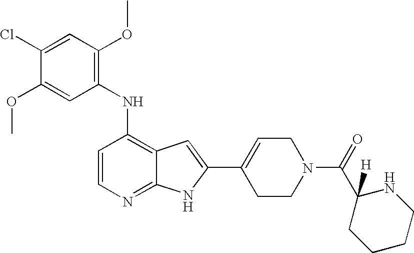

Prepared by General Procedure C. MS (ES+): ml/z 496.10 (MH⁺). HPLC: t_{R}=1.91 min (OpenLynx, polar_{—}5 min).

### EXAMPLE 54

### {4-[4-(4-Chloro-2,5-dimethoxyphenylamino)-1H-pyrrolo[2,3-b]pyridin-2-yl]-3,6-dihydro-2H-pyridin-1-yl}-(S)-piperidin-2-yl-methanone

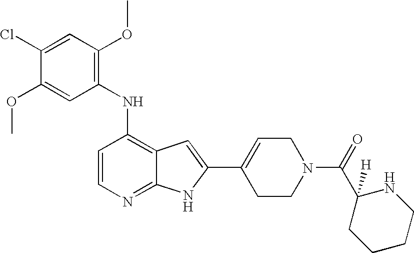

Prepared by General Procedure C. MS (ES+): m/z 496.10 (MH⁺). HPLC: t_{R}=1.91 min (OpenLynx, polar_{—}5 min).

### EXAMPLE 55

### {4-[4-(4-Chloro-2,5-dimethoxyphenylamino)-1H-pyrrolo[2,3-b]pyridin-2-yl]-3,6-dihydro-2H-pyridin-1-yl}-(S)-piperidin-3-yl-methanone

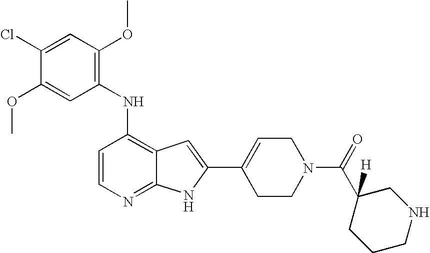

Prepared by General Procedure C. MS (ES+): m/z 496.39 (MH⁺). HPLC: t_{R}=2.01 min (OpenLynx, polar_{—}5 min).

### EXAMPLE 56

### {4-[4-(4-Chloro-2,5-dimethoxyphenylamino)-1H-pyrrolo[2,3-b]pyridin-2-yl]-3,6-dihydro-2H-pyridin-1-yl}-(R)-piperidin-3-yl-methanone

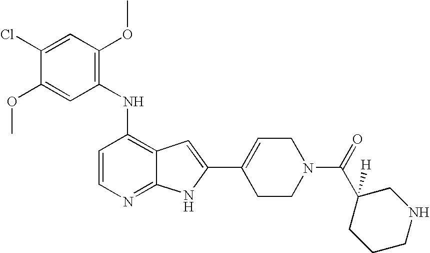

Prepared by General Procedure C. MS (ES+): m/z 496.39 (MH⁺). HPLC: t_{R}=2.01 min (OpenLynx, polar_{—}5 min).

### EXAMPLE 57

### {4-[4-(4-Chloro-2,5-dimethoxyphenylamino)-1H-pyrrolo[2,3-b]pyridin-2-yl]-3,6-dihydro-2H-pyridin-1-yl}-(1-methylpiperidin-2-yl)-methanone

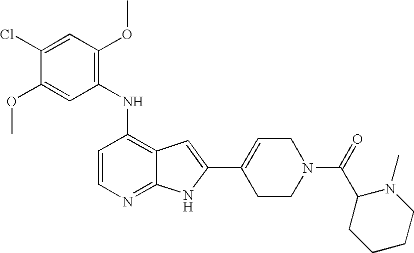

Prepared by General Procedure C. MS (ES+): m/z 510.41 (MH⁺). HPLC: t_{R}=1.96 min (OpenLynx, polar₁₃ 5 min).

### EXAMPLE 58

### {4-[4-(4-Chloro-2,5-dimethoxyphenylamino)-1H-pyrrolo[2,3-b]pyridin-2-yl]-3,6-dihydro-2H-pyridin-1-yl}-(S)-1-methylpyrrolin-2-yl)-methanone

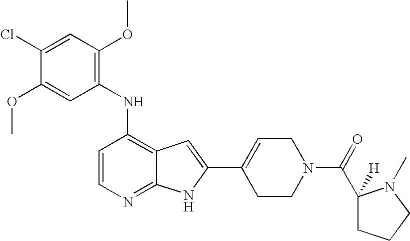

Prepared by General Procedure C. MS (ES+): m/z 496.40 (MH⁺). HPLC: t_{R}=1.94 min (OpenLynx, polar_{—}5 min).

### EXAMPLE 59

### {4-[4-(3-Chloro-4-fluorophenylamino)-1H-pyrrolo[2,3-b]pyridin-2-yl]-3,6-dihydro-2H-pyridin-1-yl}-(1-methylpiperidin-2-yl)-methanone

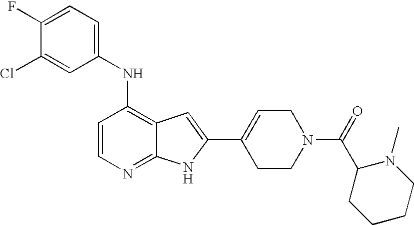

Prepared by General Procedure C. MS (ES+): m/z 468.34 (MH⁺). HPLC: t_{R}=2.03 min (OpenLynx, polar_{—}5 min).

### EXAMPLE 60

### {4-[4-(3-Chloro-4-fluorophenylamino)-1H-pyrrolo[2,3-b]pyridin-2-yl]-3,6-dihydro-2H-pyridin-1-yl}-((S)-1-methylpyrrolidin-2-yl)-methanone

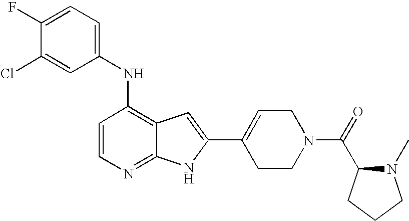

Prepared by General Procedure C. MS (ES+): m/z 454.36 (MH⁺). HPLC: t_{R}=2.04 min (OpenLynx, polar_{—}5 min).

### EXAMPLE 61

### {4-[4-(3-Chloro-4-fluorophenylamino)-1H-pyrrolo[2,3-b]pyridin-2-yl]-3,6-dihydro-2H-pyridin-1-yl}-(S)-piperidin-3-yl-methanone

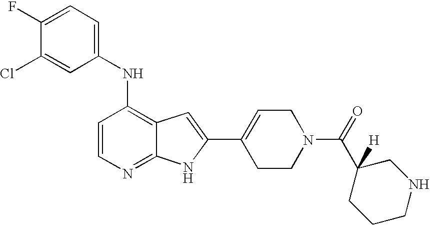

Prepared by General Procedure C. MS (ES+): m/z 454.34 (MH⁺). HPLC: t_{R}=2.03 min (OpenLynx, polar_{—}5 min).

### EXAMPLE 62

### {4-[4-(3-Chloro-4-fluorophenylamino)-1H-pyrrolo[2,3-b]pyridin-2-yl]-3,6-dihydro-2H-pyridin-1-yl}-(S)-pyrrolidin-3-yl-methanone

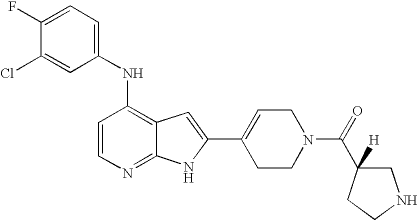

Prepared by General Procedure C. MS (ES+): m/z 440.34 (MH⁺). HPLC: t_{R}=2.00 min (OpenLynx, polar_{—}5 min).

### EXAMPLE 63

General procedure D: A small vial was charged with an amine (0.249 mmol), a carbamoyl chloride (0.251 mmol), DIPEA (3-5 eq), DMF (2.0 mL), and a stirring bar. The reaction mixture was allowed to stir at room temperature for 3 h. LC-MS indicated complete conversion of the starting materials. The reaction mixture was added to water (40 mL) and the precipitate was collected by filtration. The precipitate was redissolved in dichloromethane/MeOH (10:1) and extracted into 4 N HCl. The aqueous solution was isolated and then brought to pH=13 by addition of 3 M NaOH creating a fine precipitate. The free-base product was extracted into Et₂O/MeOH (10:1) and washed with 50 mL H₂O. The organics were isolated and the solvent was removed under reduced pressure. The residue was purified by silica gel chromatography to afford the desired product.

### EXAMPLE 64

### {4-[4-(4-Chloro-3-methoxy-5-methylphenylamino)-1H-pyrrolo[2,3-b]pyridin-2-yl]-3,6-dihydro-2H-pyridin-1-yl)-(4-methylpiperazin-1-yl)methanone

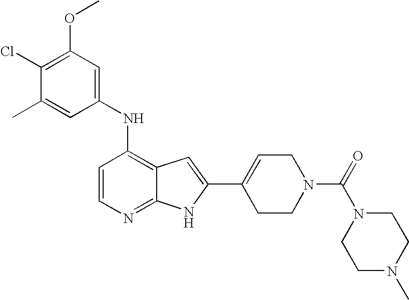

Prepared by General Procedure D. Yield: 25%. ¹H-NMR (CDCl₃, 400 MHz): δ=7.96 (d, J=5.56 Hz, 1 H), 6.79 (d, J=2.02 Hz), 6.74-6.73 (m, 2 H), 6.70 (sb, 1 H), 6.33 (s, 1 H), 4.03 (s, 2 H), 3.85 (s, 3 H), 3.47 (t, J=5.42 Hz, 2 H), 3.34 (m, 4 H), 2.59 (s, 2 H), 2.43 (m, 4H), 2.38 (s, 3H), 2.30 (s, 3H). MS (ES+): m/z 495.44 (MH⁺). HPLC: t_{R}=1.97 min (OpenLynx, polar_{—}5 min).

### EXAMPLE 65

### {4-[4-(3-Chloro-4-fluorophenylamino)-1H-pyrrolo[2,3-b]pyridin-2-yl]-3,6-dihydro-2H-pyridin-1-yl}-(4-cyclopentylpiperazin-1-yl)-methanone

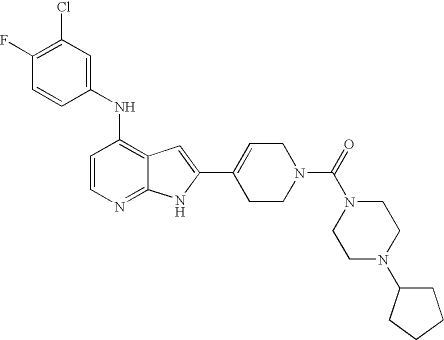

Prepared by General Procedure D. MS (ES+): m/z 523.45 (MH⁺). HPLC: t_{R}=1.95 min (OpenLynx, polar_{—}5 min).

### EXAMPLE 66

### 4-Chloro-2-(1,2,3,6-tetrahydropyridin-4-yl)-1H-pyrrolo[2,3-b]pyridine bis-hydrochloride

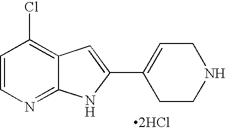

tert-Butyl 4-(4-chloro-1H-pyrrolo[2,3-b]pyridine-2-yl)-3,6-dihydro(2H)-pyridine-1-carboxylate (16 g, 48 mmol) was placed in a three-necked round bottom flask (1 L), equipped with a calcium chloride guard tube and a low temperature thermometer. Dry dichloromethane (250 mL) was added and the mixture was cooled to −5 to 0° C. To the stirring slurry was added 9.9 M HCl in dioxane (73 mL, 718 mmol) at −5 to 0° C. via syringe. Stirring was continued at the same temperature for 2-3 h and then at room temperature overnight. Thin layer chromatography (5% MeOH in dichloromethane) indicated complete conversion of the starting material. The resulting yellow crystals were collected by filtration, washed with hexane and dried in a vacuum oven at 50° C. to give yellow solids (14.2 g, 96.6%). ¹H-NMR (D₂O, 300 MHz): δ=2.26 (d, J=2.1 Hz, 2 H), 2.99 (t, J=6.3 Hz, 2 H), 3.44 (d, J=3.3 Hz, 2 H), 5.93 (br, s, 1 H), 6.2 (s, 1 H), 6.95 (d, J=6.3 Hz, 1 H), 7.65 (d, J=6.3 Hz, 1 H).

### EXAMPLE 67

### [4-(4-Chloro-1H-pyrrolo[2,3-b]pyridin-2-yl)-3,6-dihydro-2H-pyridin-1-yl]-4-methylpiperazin-1-yl)methanone

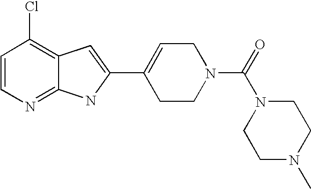

To a mixture of 4-chloro-2-(1,2,3,6-tetrahydropyridin-4-yl)-1H-pyrrolo[2,3-b]pyridine (1.00 g, 0.00326 mol) and DIPEA (2.95 g, 0.0228 mol) in DMF (6 mL) was added 4-methylpiperazine-1-carbonyl chloride hydrochloride (1.30 g, 0.00652 mol). The mixture was stirred at room temperature for 3 h and concentrated under vacuum. The resulting solids were dissolved in dichloromethane and washed with water. The dichloromethane solution was dried over Na₂SO₄, filtered, and concentrated under vacuum to afford 1.0 g desired product (yield: 85%). MS (ES+): m/z 360.12 (MH⁺, ³⁵Cl), 362.08 (MH⁺, ³⁷C). HPLC: t_{R}=2.12 min (OpenLynx, polar_{—}5 min).

### EXAMPLE 68

### 4-(4-Chloro-1H-pyrrolo[2,3-b]pyridin-2-yl)-3,6-dihydro(2H) pyridine1-N,N-dimethylcarboxamide

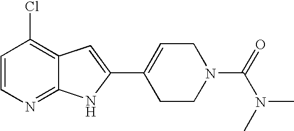

4-Chloro-2-(1,2,3,6-tetrahydropyridin-4-yl)-1H-pyrrolo[2,3-b]pyridine bis-hydrochloride (2 g, 6.56 mmol) was placed in an oven-dried three-necked round bottom flask (250 mL), followed by N,N-diisopropylethylamine (4.24 g, 32.7 mmol) and dry DMF (60 mL). The mixture was stirred for 10 min. at −2 to 0° C. to give a slurry. To the slurry was added N,N-dimethylcarbamyl chloride (0.626 mL, 6.88 mmol) slowly over 10 min and the resulting suspension was stirred at the same temperature for 6 h. At this point, the reaction became homogeneous. Stirring was continued for overnight. TLC (1% MeOH in dichloromethane with one drop of triethylamine) indicated complete conversion of starting material. DMF solvent was removed under high vacuum. To the residue was added ice-cold water. The mixture was stirred for a few minutes and filtered. The product thus obtained was pure and dried in a vacuum oven over P₂O₅ overnight to affod 1.8 g white solid (yield: 90%). m.p.: 190-191° C. ¹H-NMR (CDCl₃, 300 MHz): δ=2.75 (br, s, 2 H), 2.98 (s, 6 H), 3.55 (t, J=6 Hz, 2 H), 4.15 (br, s, 2 H), 6.42 (br, s, 1 H), 6.6 (s, 1 H), 7.2 (br, s, 1 H), 8.2 (br, s, 1 H), 11.8 (br, s, NH). MS: m/z: 305 (M+1).

### EXAMPLE 69

### N-(4Chloro-3-methoxy-5-methylphenyl)-2-(1,2,3,6-tetrahydropyridin-4-yl)-1H-pyrrolo[2,3-b]pyridin-4-amine tri-hydrochloride

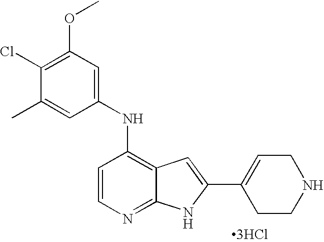

tert-Butyl 4-(4-(4-chloro-3-methoxy-5-methylphenylamino)-1H-pyrrolo[2,3-b]pyridine-2-yl)-3,6-dihydro(2H)pyridine-1-carboxylate (11.26 g, 24.06 mmol) was placed in a three-necked round bottom flask (500 mL), equipped with a low temperature thermometer and a calcium chloride guard tube, and dry dichloromethane (50 mL) was added. The resulting slurry was cooled to −5 to −2° C. and 4 M HCl in dioxane (160 mL, 25 eq) was added through a syringe over a period of 30 min. Initially, the reaction became homogeneous. After stirred at 0° C. for 2 h and at room temperature overnight, the reaction mixture became a thick slurry. Thin layer chromatography (TLC, 10% MeOH in dichloromethane) indicated complete conversion of starting material. The reaction mixture was cooled with ice-water bath, filtered, and washed with dichloromethane followed by hexane. The wet cake was dried over P₂O₅ in a vacuum oven at 50 ° C. overnight to give light yellow crystals of the desired product (9.9 g, yield: 85.6%). m.p.: 276 ° C. (decomposed). ¹H-NMR (D₂O, 300 MHz): δ=1.82 (s, 3 H), 2.65 (br, s, 2 H), 3.47 (br, s, 2 H), 3.51(s, 3 H), 3.91 (br, s, 2 H), 6.19 (s, 1 H), 6.26 (s, 1 H), 6.30 (s, 1 H), 6.37 (s, 1 H), 6.63 (d, J=7 Hz, 1 H), 7.57 (d, J=7 Hz, 1 H). MS: 369 (M+1).

### EXAMPLE 70

### 3-Bromo-4-chloro-5-methoxyphenylamine

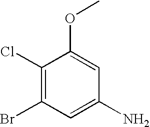

NBS (0.582 g, 3.27 mmol) was added to a solution of 4-chloro-3-methoxyphenylamine (0.468 g, 2.97 mmol) in THF (15 mL) at −78 ° C. The resulting mixture was stirred at −78° C. for 30 min and then at room temperature for 2 h. LC-MS showed the completion of the reaction. After evaporating the solvent, the residue was dissolved in dichloromethane, washed with water, dried over Na₂SO₄, and concentrated to afford the desired product. MS (ES+): m/z 235.99 (MH⁺, ³⁵Cl, ⁷⁹Br), 239.93 (MH⁺, ³⁷Cl, ⁸¹Br). HPLC: t_{R}=3.19 min (OpenLynx, polar_{—}5 min).

### EXAMPLE 71

### 4-Chloro-3-methoxy-5-methylphenylamine

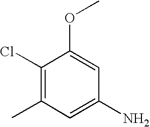

General Procedure E: A solution of 3-bromo-4-chloro-5-methoxyphenylamine (0.300 g, 1.27 mmol), potassium carbonate (0.386 g, 2.79 mol), Pd(PPh₃)₂Cl₂ (0.0445 g, 0.0634 mmol), and methylboronic acid (0.0911 g, 1.52 mmol) in H₂O (1.5 mL) and 1,4-dioxane (6.0 mL) was degassed and then stirred at 100° C. for 4 h. LC-MS showed the completion of the reaction. The reaction mixture was dissolved in dichloromethane, washed with water, dried over Na₂SO₄, filtered, and concentrated under reduced pressure to give the crude product, which was purified by silica gel chromatography (hexanes:EtOAc=2:1) to afford 0.16 g of desired product (yield: 73%). ¹H-NMR (CDCl₃, 400 MHz): δ=2.20 (s, 3 H), 3.75 (s, 3 H), 6.07 (d, J=2.27 Hz, 1 H), 6.13 (d, J=2.02 Hz, 1 H). MS (ES+): m/z 172.14 (MH⁺, ³⁵Cl), 174.08 (MH⁺, ³⁷Cl). HPLC: t_{R}=2.82 min (OpenLynx, polar_{—}5 min).

### EXAMPLE 72

### 4-Chloro-3-ethyl-5-methoxyphenylamine

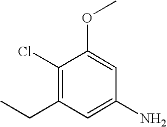

Prepared by General Procedure E. MS (ES+): m/z 186.13 (MH⁺, ³⁵Cl), 188.08 (MH⁺, ³⁷Cl). HPLC: t_{R}=3.09 min (OpenLynx, polar_{—}5 min).

### EXAMPLE 73

### 4-Chloro-3-cyclopropyl-5-methoxy-phenylamine

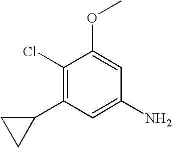

Prepared by General Procedure E. MS (ES+): m/z 198.37 (MH⁺, ³⁵Cl), 200.37 (MH⁺, ³⁷Cl). HPLC: t_{R}=3.07 min (OpenLynx, polar_{—}5 min).

### EXAMPLE 74

### 4-(5-Amino-2-chloro-3-methoxyphenyl)2-methylbut-3-yn-2-ol

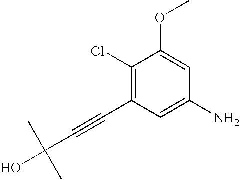

Prepared by General Procedure E. Product directly used in the next step.

### EXAMPLE 75

### 4-Chloro-3-ethynyl-5-methoxyphenylamine

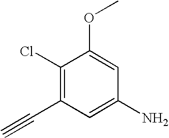

To a solution of 4-(5-amino-2-chloro-3-methoxyphenyl)-2-methylbut-3-yn-2-ol (0.302 g, 1.26 mmol) in toluene (4 mL) and DMF (0.5 mL) was added fine-powdered sodium hydroxide (0.43 g, 11 mmol). The resulting mixture was stirred at 100° C. for 4 h. The reaction mixture was cooled to room temperature, filtered, and purified by silica gel chromatography (hexane/EtOAc=1:1) to afford the desired product, overall yield: 30% (from EXAMPLE 73). MS (ES+): m/z 182.12 (MH⁺, ³⁵Cl), 184.15 (MH⁺, ³⁷Cl). HPLC: t_{R}=2.92 min (OpenLynx, polar_{—}5 min).

In Vitro Activity

All kinases described in the assays below were recombinant and generated at Upstate (Dundee, UK) except for the KDR assay. Assays were run within 15 μM of the apparent Km for ATP where determined, or at 100 μM ATP. For each enzyme, 1U activity is defined as the incorporation of 1 nmol phosphate into the appropriate substrate for a given kinase per minute at 30° C. with a final ATP concentration of 100 μM.

Assay ATP concentrations for individual kinases are included in the text.

Ab1 (human) —45 μM ATP: In a final reaction volume of 25 μL, Ab1 (h) (5-10 mU) is incubated with 8 mM MOPS pH 7.0, 0.2 mM EDTA, 50 μM EAIYAAPFAKKK, 10 mM MgAcetate and [γ-33P-ATP] (specific activity approx. 500 cpm/pmol, concentration as required). The reaction is initiated by the addition of the MgATP mix. After incubation for 40 min at rt, the reaction is stopped by the addition of 5 μL of a 3% phosphoric acid solution. Then, 10 μL of the reaction is spotted onto a P30 filtermat and washed three times for 5min in 75mM phosphoric acid and once in methanol prior to drying and scintillation counting.

Aurora-A (human) —15 μM ATP: In a final reaction volume of 25 μL, Aurora-A (h) (5-10 mU) is incubated with 8mM MOPS pH 7.0, 0.2mM EDTA, 200 μM LRRASLG (Kemptide), 10 mM MgAcetate and [γ-33P-ATP] (specific activity approx. 500 cpm/pmol, concentration as required). The reaction is initiated by the addition of the MgATP mix. After incubation for 40 min at rt, the reaction is stopped by the addition of 5 μL of a 3% phosphoric acid solution. 10 μL of the reaction is then spotted onto a P30 filtermat and washed three times for 5 min in 50 mM phosphoric acid and once in methanol prior to drying and scintillation counting.

Blk (mouse)—120 μM ATP: In a final reaction volume of 25 μL, Blk (m) (5-10 mU) is incubated with 50 mM Tris pH 7.5, 0.1 mM EGTA, 0.1 mM Na₃VO₄, 0.1% β-mercaptoethanol, 0.1 mg/mL poly(Glu, Tyr) 4:1, 10 mM MgAcetate and [γ-33P-ATP] (specific activity approx. 500 cpm/pmol, concentration as required). The reaction is initiated by the addition of the MgATP mix. After incubation for 40min at rt, the reaction is stopped by the addition of 5 μL of a 3% phosphoric acid solution. 10 μL of the reaction is then spotted onto a Filtermat A and washed three times for 5 min in 75 mM phosphoric acid and once in methanol prior to drying and scintillation counting.

Bmx (human)—45 μM ATP: In a final reaction volume of 25 μL, Bmx (h) (5-10 mU) is incubated with 8 mM MOPS pH 7.0, 0.2 mM EDTA, 0.1 mg/mL poly(Glu, Tyr) 4: 1, 10 mM MgAcetate and [γ-33P-ATP] (specific activity approx. 500 cpm/pmol, concentration as required). The reaction is initiated by the addition of the MgATP mix. After incubation for 40 min at rt, the reaction is stopped by the addition of 5 μL of a 3% phosphoric acid solution. 10 μL of the reaction is then spotted onto a Filtermat A and washed three times for 5 min in 75 mM phosphoric acid and once in methanol prior to drying and scintillation counting.

CaMKII (rat)—15 μM ATP: In a final reaction volume of 25 μL, CaMKII (r) (5-10 mU) is incubated with 40 mM HEPES pH 7.4, 5 mM CaCl₂, 30 μg/mL calmodulin, 30 μM KKLNRTLSVA, 10 mM MgAcetate and [γ-33P-ATP] (specific activity approx. 500 cpm/pmol, concentration as required). The reaction is initiated by the addition of the MgATP mix. After incubation for 40 min at rt, the reaction is stopped by the addition of 5 μL of a 3% phosphoric acid solution. 10 L of the reaction is then spotted onto a P30 filtermat and washed three times for 5 min in 75 mM phosphoric acid and once in methanol prior to drying and scintillation counting.

c-RAF (human)—45 μM ATP: In a final reaction volume of 25 μL, c-RAF (h) (5-10 mU) is incubated with 25 mM Tris pH 7.5, 0.02 mM EGTA, 0.66 mg/mL myelin basic protein, 10 mM MgAcetate and [γ-33P-ATP] (specific activity approx. 500 cpm/pmol, concentration as required). The reaction is initiated by the addition of the MgATP mix. After incubation for 40 min at rt, the reaction is stopped by the addition of 5 μL of a 3% phosphoric acid solution. 10 μL of the reaction is then spotted onto a P30 filtermat and washed three times for 5 min in 75 mM phosphoric acid and once in methanol prior to drying and scintillation counting.

cSRC (human)—200 μM ATP: In a final reaction volume of 25 μL, cSRC (h) (5-10 mU) is incubated with 8 mM MOPS pH 7.0, 0.2 mM EDTA, 250 μM KVEKIGEGTYGVVYK (Cdc2 peptide), 10 mM MgAcetate and [γ-33P-ATP] (specific activity approx. 500 cpm/pmol, concentration as required). The reaction is initiated by the addition of the MgATP mix. After incubation for 40 min at rt, the reaction is stopped by the addition of 5 μL of a 3% phosphoric acid solution. 10 μL of the reaction is then spotted onto a P30 filtermat and washed three times for 5 min in 75 mM phosphoric acid and once in methanol prior to drying and scintillation counting.

EGFR (human)—10 μM ATP: In a final reaction volume of 25 μL, EGFR (h) (5-10 mU) is incubated with 8 mM MOPS pH 7.0, 0.2 mM EDTA, 10 mM MnCl₂, 0.1 mg/mL poly(Glu, Tyr) 4:1, 10 mM MgAcetate and [γ-33P-ATP] (specific activity approx. 500 cpm/pmol, concentration as required). The reaction is initiated by the addition of the MgATP mix. After incubation for 40 min at rt, the reaction is stopped by the addition of 5 μL of a 3% phosphoric acid solution. 10 μL of the reaction is then spotted onto a Filtermat A and washed three times for 5 min in 75 mM phosphoric acid and once in methanol prior to drying and scintillation counting.

FGFR3 (human)—15 μM ATP: In a final reaction volume of 25 μL, FGFR3 (h) (5-10 mU) is incubated with 8 mM MOPS pH 7.0, 0.2 mM EDTA, 0.1 mg/mL poly(Glu, Tyr) 4:1, 10 mM MnCl₂, 10 mM MgAcetate and [γ-33P-ATP] (specific activity approx. 500 cpm/pmol, concentration as required). The reaction is initiated by the addition of the MgATP mix. After incubation for 40 min at rt, the reaction is stopped by the addition of 5 μL of a 3% phosphoric acid solution. 10 L of the reaction is then spotted onto a Filtermat A and washed three times for 5 min in 75 mM phosphoric acid and once in methanol prior to drying and scintillation counting.

Flt3 (human)—200 μM ATP: In a final reaction volume of 25 μL Flt3 (h) (5-10 mU) is incubated with 8 mM MOPS pH 7.0, 0.2mM EDTA, 50 μM EAIYAAPFAKKK, 10 mM MgAcetate and [γ-33P-ATP] (specific activity approx. 500 cpm/pmol, concentration as required). The reaction is initiated by the addition of the MgATP mix. After incubation for 40 min at rt, the reaction is stopped by the addition of 5 μL of a 3% phosphoric acid solution. 10 μL of the reaction is then spotted onto a P30 filtermat and washed three times for 5 min in 75 mM phosphoric acid and once in methanol prior to drying and scintillation counting.

GSK3β(human)—15 μM ATP: In a final reaction volume of 251 μL, GSK3β(h) (5-10 mU) is incubated with 8 mM MOPS pH 7.0, 0.2 mM EDTA, 20 μM YRRAAVPPSPSLSRHSSPHQS(p)EDEEE (phospho GS2 peptide), 10 mM MgAcetate and [γ-33P-ATP] (specific activity approx. 500cpm/pmol, concentration as required). The reaction is initiated by the addition of the MgATP mix. After incubation for 40 min at rt, the reaction is stopped by the addition of 5 μL of a 3% phosphoric acid solution. 10 μL of the reaction is then spotted onto a P30 filtermat and washed three times for 5 min in 50 mM phosphoric acid and once in methanol prior to drying and scintillation counting.

Lck (human)—90 μM ATP: In a final reaction volume of 25 μL, Lck (h) (5-10 mU) is incubated with 50 mM Tris pH 7.5, 0.1 mM EGTA, 0.1 mM Na3VO4, 250 μM KVEKIGEGTYGVVYK (Cdc2 peptide), 10 mM MgAcetate and [γ-33P-ATP] (specific activity approx. 500 cpm/pmol, concentration as required). The reaction is initiated by adding the MgATP mix. After incubation for 40 min at rt, the reaction is stopped by adding 5 μL of a 3% phosphoric acid solution. 10 μL of the reaction is then spotted onto a P30 filtermat and washed three times for 5min in 75 mM phosphoric acid and once in methanol prior to drying and scintillation counting.

MEK1 (human)—10 M ATP: In a final reaction volume of 25 μL, MEK1 (h) (1-5 mU) is incubated with 50 mM Tris pH 7.5, 0.2 mM EGTA, 0.1% β-mercaptoethanol, 0.01% Brij-35, 1 μM inactive MAPK2 (m), 10 mM MgAcetate and cold ATP (concentration as required). The reaction is initiated by the addition of the MgATP. After incubation for 40 min at rt, 5 μL of this incubation mix is used to initiate a MAPK2 (m) assay. In a final reaction volume of 25 μL, MAPK2 (h) (5-10 mU) is incubated with 25 mM Tris pH 7.5, 0.02 mM EGTA, 0.33 mg/mL myelin basic protein, 10 mM MgAcetate and [γ-33P-ATP] (specific activity approx. 500 cpm/pmol, concentration as required). The reaction is initiated by the addition of the MgATP mix. After incubation for 40 min at rt, the reaction is stopped by the addition of 5 μL of a 3% phosphoric acid solution. 10 L of the reaction is then spotted onto a P30 filtermat and washed three times for 5 min in 75 mM phosphoric acid and once in methanol prior to drying and scintillation counting.

PDK1 (human)—10 μM ATP: In a final reaction volume of 25 μL, PDK1 (h) (5-10 mU) is incubated with 50 mM Tris pH 7.5, 100 μM KTFCGTPEYLAPEVRREPRILSEEEQEMFRDFDYIADWC (PDKtide), 0.1% β-mercaptoethanol, 10 mM MgAcetate and [γ-33P-ATP] (specific activity approx. 500 cpm/pmol, concentration as required). The reaction is initiated by the addition of the MgATP mix. After incubation for 40 min at rt, the reaction is stopped by the addition of 5 μL of a 3% phosphoric acid solution. 10 μL of the reaction is then spotted onto a P30 filtermat and washed three times for 5 min in 75 mM phosphoric acid and once in methanol prior to drying and scintillation counting.

PRK2 (human)—15 μM ATP: In a final reaction volume of 25 μL, PRK2 (h) (5-10 mU) is incubated with 50 mM Tris pH 7.5, 0.1 mM EGTA, 0.1% β-mercaptoethanol, 30 μM AKRRRLSSLRA, 10 mM MgAcetate and [γ-33P-ATP] (specific activity approx. 500 cpm/pmol, concentration as required). The reaction is initiated by the addition of the MgATP mix. After incubation for 40 min at rt, the reaction is stopped by the addition of 5 μL of a 3% phosphoric acid solution. 10 μL of the reaction is then spotted onto a P30 filtermat and washed three times for 5 min in 75 mM phosphoric acid and once in methanol prior to drying and scintillation counting.

p70S6K (human)—15 μM ATP: In a final reaction volume of 25 μL, p70S6K (h) (5-10 mU) is incubated with 8 mM MOPS pH 7.0, 0.2mM EDTA, 100 μM KKRNRTLTV, 10 mM MgAcetate and [γ-33P-ATP] (specific activity approx. 500 cpm/pmol, concentration as required). The reaction is initiated by the addition of the MgATP mix. After incubation for 40 min at rt, the reaction is stopped by the addition of 5 μL of a 3% phosphoric acid solution. 10 μL of the reaction is then spotted onto a P30 filtermat and washed three times for 5 min in 75 mM phosphoric acid and once in methanol prior to drying and scintillation counting.

SGK (human)—90 μM ATP: In a final reaction volume of 25 μL, SGK (h) (5-10 mU) is incubated with 8 mM MOPS pH 7.0, 0.2 mM EDTA, 30 μM GRPRTSSFAEGKK, 10 mM MgAcetate and [γ-33P-ATP] (specific activity approx. 500 cpm/pmol, concentration as required). The reaction is initiated by the addition of the MgATP mix. After incubation for 40 min at rt, the reaction is stopped by the addition of 5 μL of a 3% phosphoric acid solution. 10 μL of the reaction is then spotted onto a P30 filtermat and washed three times for 5 min in 75 mM phosphoric acid and once in methanol prior to drying and scintillation counting.

Tie2 (human)—200 μM ATP: In a final reaction volume of 25 μL, Tie2 (h) (5-10 mU) is incubated with 8 mM MOPS pH 7.0, 0.2 mM EDTA, 0.5 mM MnCl₂, 0.1 mg/mL poly(Glu, Tyr) 4:1, 10 mM MgAcetate and [γ-33P-ATP] (specific activity approx. 500 cpm/pmol, concentration as required). The reaction is initiated by the addition of the MgATP mix. After incubation for 40 min at rt, the reaction is stopped by the addition of 5 μL of a 3% phosphoric acid solution. 10 μL of the reaction is then spotted onto a Filtermat A and washed three times for 5 min in 75 mM phosphoric acid and once in methanol prior to drying and scintillation counting.

KDR (human)—18 μM ATP: 96-well plates are coated with 0.5 μg/75 μFL/well poly(Glu, Tyr) over night at 37° C. 50 μL per well of 50 mM Hepes, pH7.4, 125 mM NaCl, 24 mM MgCl2, and 18 μM ATP± compounds are added. The reaction is initiated by the addition of 30 μL (5 ng) KDR (Proqinase) diluted in assay buffer. After incubation for 30 min at rt, the plates are washed and phosphor Tyr detected using pY-20 HRP conjugated antibody with subsequent development using ABTS reagent (KPL) and detection by absorbance at 405 nm.

Someone skilled in the art will appreciate that other assay formats may be used in place of those described above. For example, AlphaScreen (Amplified Luminescent Proximity Homogeneous Assay) technology was used with the kinases described below. Assay ATP concentrations for individual kinases are included in the text.

KDR (human)—100 μM ATP: 9 μL of the reaction mix containing ATP at the desired concentration, biotinylated poly(Glu,Tyr) (84 ng/mL) and 0.334 mM vanadate in assay buffer (50 mM HEPES (pH=7.4), 12.5 mM MgCl₂ and 1% glycerol) are added to a well of a 384 well plate along with 1 μl of compound (or vehicle control, usually DMSO). DMSO concentration is controlled at a concentration of 1%. KDR is diluted to the optimized concentration (optimized on a lot-by-lot basis) in an enzyme diluent buffer (50 mM HEPES pH=7.4, 12.5 mM MgCl₂ and 1% glycerol, 0.03% Brij35 and 0.3 mM EGTA). 5μL of this solution are then added to the well, and the complete reaction mixture is incubated for 60 min at RT. In subdued light, 5 μL of PT66 donor and acceptor beads (diluted 1:200 from manufacturers provision in a 25 mM Tris HCl (pH=7.5), 200mM NaCl, 100 mM EDTA, 0.3% BSA buffer) are added to the wells. The plates are then incubated for 4h and read on an AlphaQuest plate reader.

IGF-1R (human)—100 μM ATP: To a well of a 384 well plate are added 9 μL of the reaction mix containing ATP at the desired concentration, biotinylated poly(Glu,Tyr) (84 ng/mL) and 0.334 mM vanadate in assay buffer (50 mM HEPES (pH=7.4), 12.5mM MgCl₂ and 1% glycerol) along with 1 μL of compound (or vehicle control, usually DMSO). DMSO concentration is controlled at a concentration of 1%. IGF-1R is diluted to the optimized concentration (optimized on a lot-by-lot basis) in an enzyme diluent buffer (50 mM HEPES pH=7.4,12.5 mM MgCl₂ and 1% glycerol, 0.03% Brij35, 0.3 mM EGTA, 6 mM DTT, and 0.003% BSA). 5 μL of this solution are then added to the well, and the complete reaction mixture is incubated for 60 min at RT. In subdued light, 5 μL of PT66 donor and acceptor beads (diluted 1:200 from manufacturer's provision in a 25 mM Tris HCl (pH=7.5), 200 mM NaCl, 100 mM EDTA, 0.3% BSA buffer) are added to the wells. The plates are then incubated for 4h and read on an AlphaQuest plate reader.

RON (human)—Kₘ of ATP: RON assay is performed in a 384 well assay containing 200 ng/μL biotinylated poly(Glu,Tyr), 0.334mM vanadate, desired concentration of ATP optimized for the enzyme in assay buffer (50 mM HEPES (pH=7.4),12.5 mM MgCl₂ and 1% glycerol). Desired compound is added in a final concentration of 1%DMSO with control being vehicle of DMSO alone. RON is diluted to the optimized (on a lot-by-lot basis) concentration in an enzyme diluent buffer (50 mM HEPES pH=7.4, 12.5 mM MgCl₂ and 1% glycerol, 0.03% Brij35, 0.3 mM EGTA, 1 mM DTT, and 0.003% BSA). Enzyme is added to initiate the reaction and incubated for 30min at RT. In subdued light, appropriate amount of PT66 donor and acceptor beads (diluted 1:260 from manufacturer's provision in a 25 mM Tris HCl (pH=7.5), 200 mM NaCl, 100 mM EDTA, 0.3% BSA buffer) are added to the wells. The plates, incubated for I h, are read on an AlphaQuest plate reader.

Met (human)—Km of ATP: MET assay is performed in a 384 well assay containing 200 ng/μL biotinylated poly(Glu,Tyr), 0.334 mM vanadate, desired concentration of ATP optimized for the enzyme in assay buffer (50 mM HEPES (pH=7.4), 5 mM MgCl₂, 5 mM MnCl₂, and 1% glycerol). Desired compound is added in a final concentration of 1%DMSO with control being vehicle of DMSO alone. MET is diluted to the optimized concentration (optimized on a lot-by-lot basis) in an enzyme diluent buffer (50 mM Tris pH=7.4, 1% glycerol, 0.03% Brij35, 0.24 mM EGTA, 1 mM DTT, and 0.003% BSA). Enzyme is added to initiate the reaction and incubated for 60 min at RT. In subdued light, appropriate amount of PT66 donor and acceptor beads (diluted 1:260 from manufacturer's provision in a 25 mM Tris HCl (pH=7.5), 400 mM NaCl, 100 mM EDTA, 0.3% BSA buffer) are added to the wells. The plates, incubated for 1h, are read on an AlphaQuest plate reader

EGFR (human)—4 μM ATP: To a well of a 384 well plate are added 1 μL of compound (or vehicle control, usually DMSO; DMSO concentration is controlled at a concentration of 1%), followed by 9 μL of the reaction mix (ATP, at the desired concentration, is added diluted in assay buffer (50 mM HEPES (pH=7.4), 12.5 mM MgCl₂ and 1% glycerol), containing 69.4 mM NaCl, biotinylated poly(Glu,Tyr) (84.5 ng/mL) and 0.334 mM vanadate). EGFR is diluted to the optimized concentration (optimized on a lot-by-lot basis) in an enzyme diluent buffer (50 mM HEPES pH=7.4, 12.5 mM MgCl₂ and 1% glycerol, 0.3% Brij35 and 0.3 mM EGTA) and Stablecoat (SurModics), and DTT is also added for a concentration of 3 mM. 5 μL of this solution are then added to the well, and the complete reaction mixture is incubated for 20 min at RT. In subdued light, 5 μL of PT66 donor and acceptor beads (diluted 1:200 from manufacturers provision in a 25 mM Tris HCl (pH=7.5), 200 mM NaCl, 100 mM EDTA, 0.3% BSA buffer) are added to the wells. The plates are then incubated for 4h and read on an AlphaQuest plate reader.

EGFR (human)—100 μM ATP: To a well of a 384 well plate are added 1 μL of compound (or vehicle control, usually DMSO; DMSO concentration is controlled at a concentration of 1%), followed by 9 μl of the reaction mix (ATP, at the desired concentration, is added diluted in assay buffer (50 mM HEPES (pH=7.4), 12.5mM MgCl₂ and 1% glycerol), containing 69.4 mM NaCl, biotinylated poly(Glu,Tyr) (84.5 ng/mL) and 0.334 mM vanadate). EGFR is diluted to the optimized concentration (optimized on a lot-by-lot basis) in an enzyme diluent buffer (50 mM HEPES pH=7.4, 12.5 mM MgCl₂ and 1% glycerol, 0.3% Brij35 and 0.3 mM EGTA) and Stablecoat (SurModics), and DTT is also added for a concentration of 3 mM. 5 μL of this solution are then added to the well, and the complete reaction mixture is incubated for 60 min at RT. In subdued light, 5 μL of PT66 donor and acceptor beads (diluted 1:200 from manufacturer's provision in a 25 mM Tris HCl (pH=7.5), 200 mM NaCl, 100 mM EDTA, 0.3% BSA buffer) are added to the wells. The plates are then incubated for 4h and read on an AlphaQuest plate reader.

PDK-1 (human)—100 μM ATP: To a well of a 384 well plate are added 1 μL of compound (or vehicle control, usually DMSO; DMSO concentration is controlled at a concentration of 1%), followed by 9 μL of the reaction mix (ATP, at the desired concentration, is added diluted in assay buffer (50 mM Tris pH=7.4, 15 mM MgCl₂, 0.1 mg/mL Bovine gamma globulin, 2 mM DTT) containing biotinylated peptide substrate (83.5 nM)). PDK-1 (obtained from Upstate, 200 ng/μL) is diluted 1:25000 in an enzyme diluent buffer (50 mM Tris pH=7.4, 15 mM MgCl₂, 0.1 mg/ml Bovine gamma globulin, 2 mM DTT). 5 μL of this solution are then added to the well, and the complete reaction mixture is incubated for 2h at RT protected from light. 2.5 μL/well of stop buffer (200 mM EDTA in 20 mM Tris/200 nM NaCl) are added, and the mixture is incubated for 1h at RT protected from light. 2.5 μL/well of antibody/bead complex (antibody diluted 1:1250, donor and acceptor beads diluted 1:200 from manufacturer's provision) are added. The plates are then incubated for 2h at RT protected from light and read on an AlphaQuest plate reader.

PDK-1 (human)—4.5 μM ATP: Same procedure, except for the different ATP concentration.

EXAMPLES 1-69 inhibit at least one of the Ab1, Aurora-A, Blk, c-Raf, cSRC, Src, PRK2, FGFR3, Flt3, Lck, Mek1, PDK-1, GSK3β, EGFR, p70S6K, BMX, SGK, CaMKII, IGF-1R, Tie-2, Ron, Met, and KDR kinases at an IC₅₀ of greater than 50% inhibition at 30 μM. It is advantageous that the measured IC₅₀ be lower than 10 μM. It is still more advantageous for the IC₅₀ to be lower than 5 μM. It is even more advantageous for the IC₅₀ to be lower than 0.5 μM. It is yet more advantageous for the IC₅₀ to be lower than 0.05 μM.

What is claimed is:

1. A compound represented by Formula I: 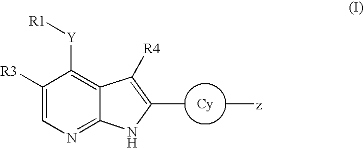
or a pharmaceutically acceptable salt thereof, wherein
Cy is, 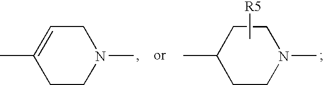
Z is hetaryl, —C₀₋₆alkyl, —C₂₋₆alkyl-O—C₁₋₆alkyl-, —C₀₋₆alkyl-(heterocyclyl), —C₀₋₆alkyl-(hetaryl), —C(O)—C₀₋₆alkyl, —C(O)—C₀₋₆alkyl-O—C₀₋₆alkyl, —C(O)—C₀₋₆alkyl-O—C₁₋₆alkyl-O—CO₀₋₆alkyl, —C(O)—C₀₋₆alkyl-N(C₀₋₆alkyl)(C₀₋₆alkyl), —C(O)—C₀₋₆alkyl-N(C₀₋₆alkyl)(aryl), —C(O)—C₀₋₆alkyl-N(C₀₋₆alkyl)(hetaryl), —C(O)—C₀₋₆alkyl-N(C₀₋₆alkyl)(heterocyclyl), —C(O)—C₀₋₆alkyl-N(C₀₋₆alkyl)(cycloalkyl), —C(O)—C₀₋₆alkyl-(heterocyclyl), —C(O)—C₀₋₆alkyl-(heterocyclyl)—C(O)—C₀₋₆alkyl, —C(O)—C₀₋₆alkyl-(hetaryl), —S(O)₂—C₀₋₆alkyl, —S(O)₂—N(C₀₋₆alkyl)(C₀₋₆alkyl), or —S(O)₂—(hetaryl), wherein any of the alkyl, heterocyclyl, or hetaryl optionally is substituted with 1-6 independent halo, OH, —C₀₋₆alkyl-O—C₀₋₆alkyl, —C₀₋₆alkyl-N(C₀₋₆alkyl)(C₀₋₆alkyl), —C(O)—C₀₋₆alkyl-N(C₀₋₆alkyl)(C₀₋₆alkyl), —C(O)—C₀₋₆alkyl-(heterocyclyl), or —C₀₋₆alkyl; 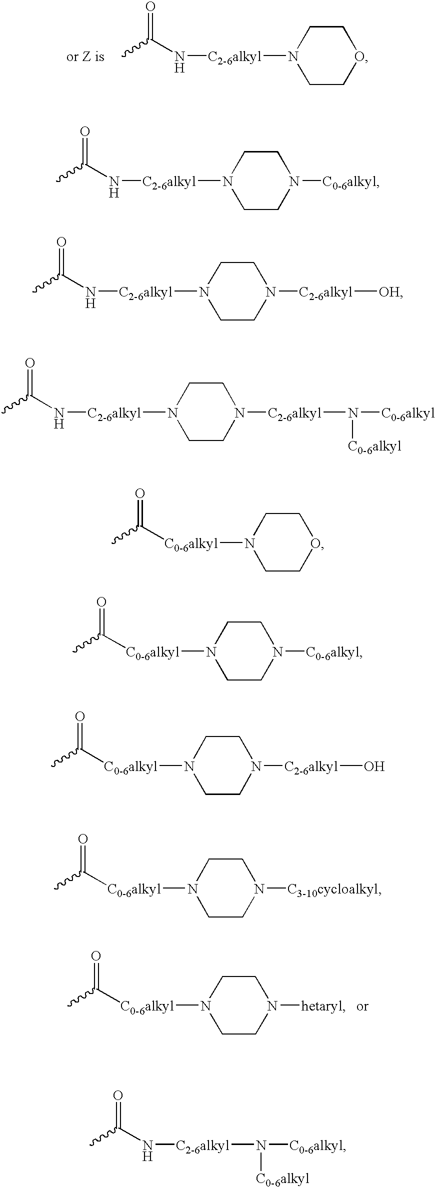
in which the wavy bond is the point of attachment, any of which except at the piperazine or morpholine moieties optionally is substituted with 1-6 independent halo, CN, OH, —C₀₋₆alkyl-O—C₀₋₆alkyl, —C₀₋₆alkyl-N(C₀₋₆alkyl)(C₀₋₆alkyl), —C(O)—-C₀₋₆alkyl-N(C₀₋₆alkyl)(C₀₋₆alkyl), —C(O)—C₀₋₆alkyl-(heterocyclyl), or C₀₋₆alkyl substituents, wherein the piperazine or morpholine moieties are optionally substituted with 1-6 C₀₋₆alkyl substituents;
Y is —C(C₀₋₆alkyl)(C₀₋₆alkyl)-, —N(C₀₋₆alkyl)-, —N(C₀₋₆alkyl)—C₁₋₆alkyl-, O, S, >N—C₂₋₆alkyl-N—(C₀₋₆alkyl)(C₀₋₆alkyl), >N—C₂₋₆alkyl-O—C₀₋₆alkyl, >N—C₁₋₆alkyl-C(O)—NH—C₀₋₆alkyl, >N—C₂₋₆alkyl-N—C(O)—C₁₋₆—alkyl, or a bond;
R1 is aryl, hetaryl, or heterocyclyl, optionally substituted with 1-6 independent halo, —CN, —OH, —C₀₋₆alkyl, —C₃₋₁₀cycloalkyl, -haloC₁₋₆alkyl, —C₂₋₆alkynyl, —N(C₀₋₆alkyl)(C₀₋₆alkyl), —C(O)—C₀₋₆alkyl-N(C₀₋₆alkyl)(C₀₋₆alkyl), —C(O)—C₀₋₆alkyl-(heterocyclyl), —C₁₋₆alkyl-C(O)—C₀₋₆alkyl-N(C₀₋₆alkyl)(C₀₋₆alkyl), —O—C₀₋₆alkyl-(heterocyclyl), —C₀₋₆alkyl-O—C₀₋₆alkyl, —C₀₋₆alkyl-N(C₀₋₆alkyl)(C₀₋₆alkyl), —O—C₀₋₆alkyl-(hetaryl), —S(O)₂—N(C₀₋₆alkyl)(C₀₋₆alkyl), aryl, hetaryl, or heterocyclyl substituents, or optionally substituted with an oxo (═O) using a bond from the aryl, hetaryl, or heterocyclyl ring, and wherein any of the substituents optionally further is substituted with 1-6 independent halo, CN, OH, —C₀₋₆alkyl-O—C₀₋₆alkyl, —C₀₋₆alkyl-N(C₀₋₆alkyl)(C₀₋₆alkyl), —C(O)—C₀₋₆alkyl-N(C₀₋₆alkyl)(C₀₋₆alkyl), —C(O)—C₀₋₆alkyl-(heterocyclyl), or C₀₋₆alkyl;
R3 is hydrogen, C₀₋₆alkyl, —C₀₋₆alkyl-O—C₀₋₆alkyl, halogen, azido, wherein any of the alkyl groups can optionally be substituted by halogen;
R4 is hydrogen, C₀₋₆alkyl, halogen, cyano, —S—C₀₋₆alkyl, —C₀₋₆alkyl-N(C₀₋₆alkyl)(C₀₋₆alkyl), N(C₀₋₆alkyl)(aryl), N(C₀₋₆alkyl)(hetaryl), N(C₀₋₆alkyl)(heterocyclyl), N(C₀₋₆alkyl)(cycloalkyl), —C₀₋₆alkyl-O—C₀₋₆alkyl, —C₀₋₆alkyl-O-aryl, —C₀₋₆alkyl-O-hetaryl, —C₀₋₆alkyl-O-cycloalkyl, —C₀₋₆alkyl-S(O)₀₋₂—C₀₋₆alkyl, —C₀₋₆alkyl-S(O)₀₋₂-aryl, —C₀₋₆alkyl-S(O)₀₋₂-hetaryl, —C₀₋₆alkyl-S(O)₀₋₂-cycloalkyl, aryl, hetaryl, cycloalkyl, heterocyclyl, wherein any of the alkyl, aryl, cycloalkyl or hetaryl groups can optionally be substituted with 1-6 independent halogen, CN, OH, —C₀₋₆alkyl-O—C₀₋₆alkyl, —C₀₋₆alkyl-N(C₀₋₆alkyl)(C₀₋₆alkyl), —C(O)—C₀₋₆alkyl-(heterocyclyl), —C(O)—C₀₋₆alkyl-N(C₀₋₆alkyl)(C₀₋₆alkyl), —C(O)—C₀₋₆alkyl-N(C₀₋₆alkyl)(aryl), —C(O)—C₀₋₆alkyl-N(C₀₋₆alkyl)(hetaryl), —C(O)—C₀₋₆alkyl-N(C₀₋₆alkyl)(heterocyclyl), —C(O)—C₀₋₆alkyl-N(C₀₋₆alkyl)(cycloalkyl), or C₀₋₆alkyl; and
R5 is hydrogen, C₀₋₆alkyl, —C₀₋₆alkyl-O—C₀₋₆alkyl, or —C₀₋₆alkyl-N(C₀₋₆alkyl)(C₀₋₆alkyl), wherein any of the alkyl groups can optionally be substituted by halogen.

2. The compound according to claim 1, or a pharmaceutically acceptable salt thereof, wherein R3 is hydrogen.

3. The compound according to claim 2, or a pharmaceutically acceptable salt thereof, wherein Cy is

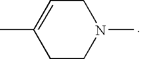

4. The compound according to claim 3, or a pharmaceutically acceptable salt thereof, wherein Y is —N(C₀₋₆alkyl)-.

5. The compound according to claim 1, or a pharmaceutically acceptable salt thereof, wherein
R3, R4, and R5 are hydrogen,
Cy is 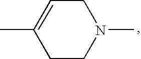
and
Y is —N(C₀₋₆alkyl)-.

6. The compound according to claim 5, or a pharmaceutically acceptable salt thereof, wherein Z is —CO₂tBu, —CONHtBu, —CON(CH₃)₂, 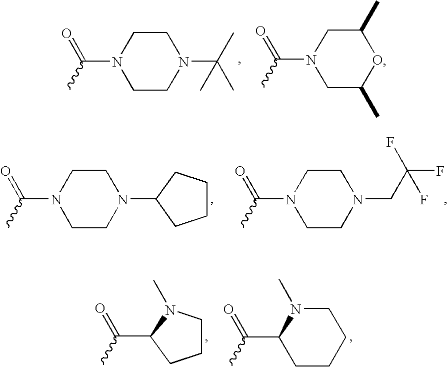
or 2-thiazolyl.

7. The compound according to claim 1, represented by: 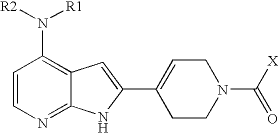
wherein
R2 is —C₀₋₆alkyl, —C₂₋₆alkyl-N—(C₀₋₆alkyl)(C₀₋₆alkyl), —C₂₋₆alkyl-O—C₀₋₆alkyl, —C₁₋₆alkyl-C(O)-NH—C₀₋₆alkyl, or —C₂₋₆alkyl-N—C(O)—C₁₋₆alkyl;
X is —OtBu, —NHtBu, —N(CH₃)₂or 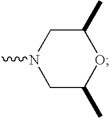
and
R1 is selected from the following table: 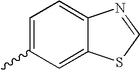 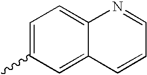 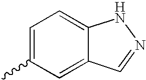 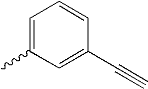 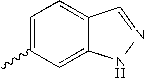 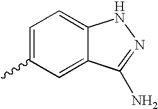 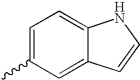 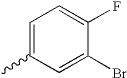 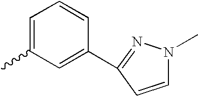 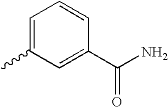 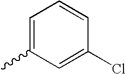 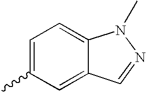 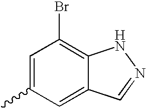 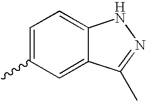 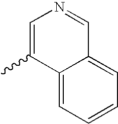 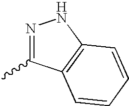 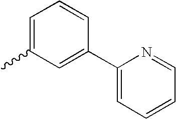 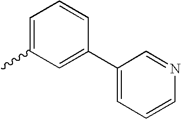 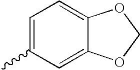 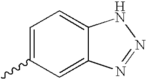 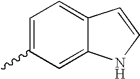 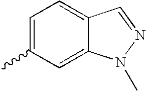 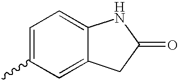 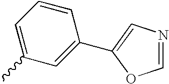 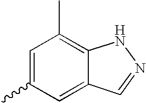 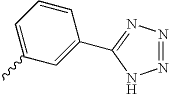 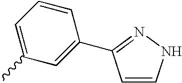 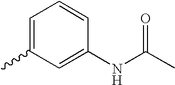 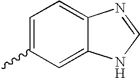 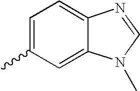 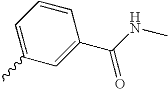 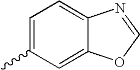 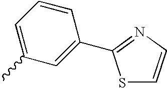 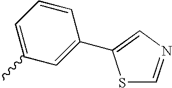 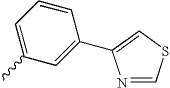 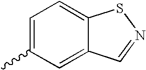 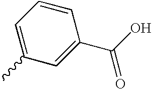 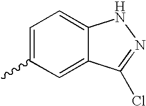 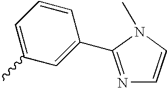 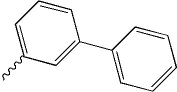 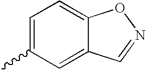 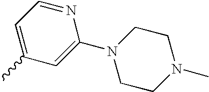 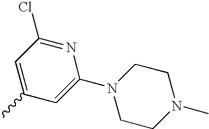 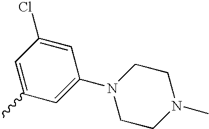 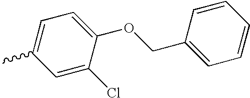 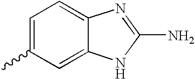 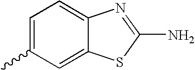 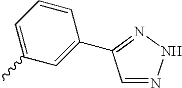 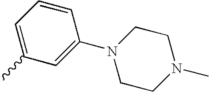 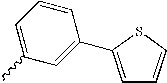 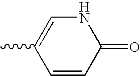 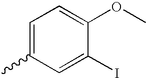 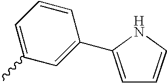 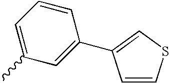 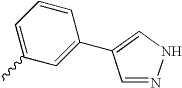 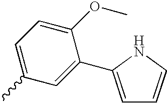 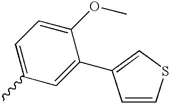 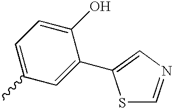 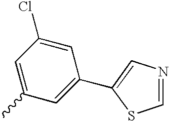 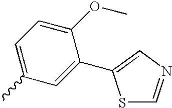 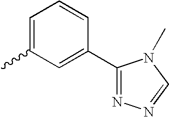 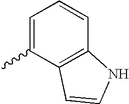 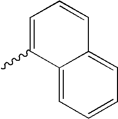 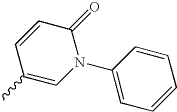 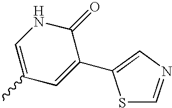 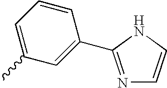 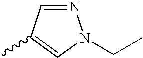 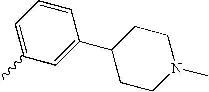 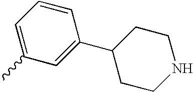 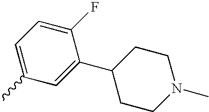 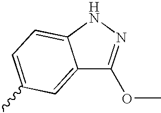 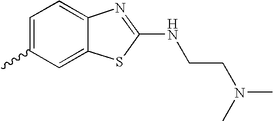 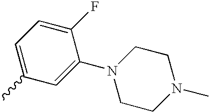 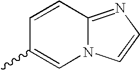 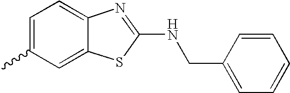 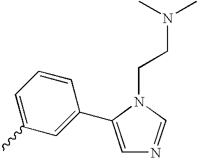 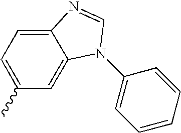 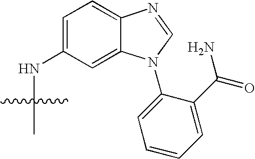 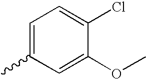 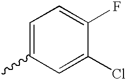 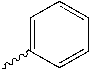 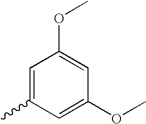 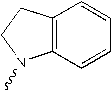 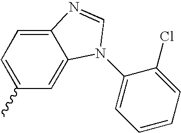 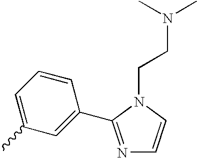 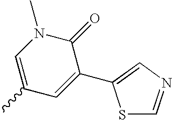 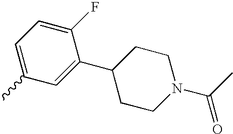 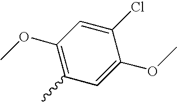 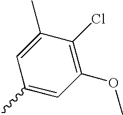 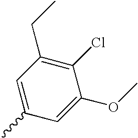 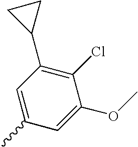 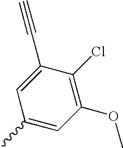 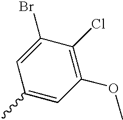
or a stereoisomer, or a pharmaceutically acceptable salt thereof.

8. The compound according to claim 1, represented by: 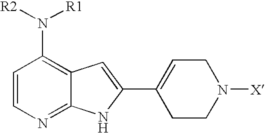
wherein
R2 is —C₀₋₆alkyl, —C₂₋₆alkyl-N—(C₀₋₆alkyl)(C₀₋₆alkyl), —C₂₋₆alkyl-O—C₀₋₆alkyl, —C₁₋₆alkyl-C(O)-NH—C₀₋₆alkyl, or —C₂₋₆alkyl-N—C(O)—C₁₋₆alkyl;
X′ is optionally substituted heteroaryl; and
R1 is selected from the following table: 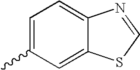 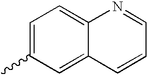 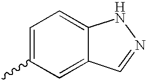 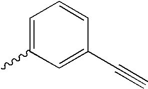 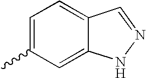 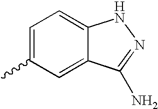 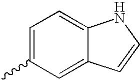 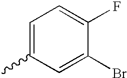 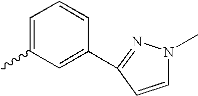 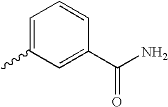 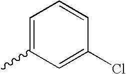 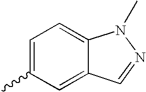 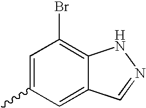 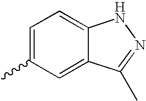 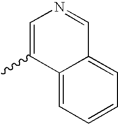 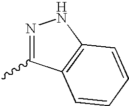 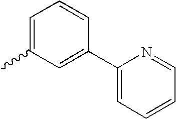 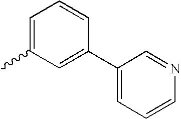 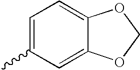 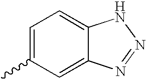 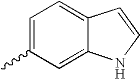 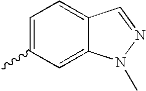 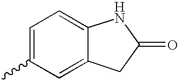 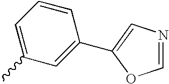 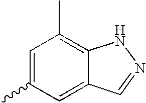 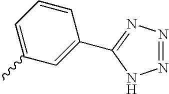 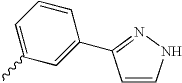 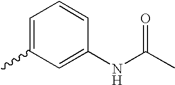 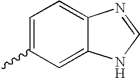 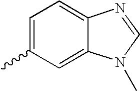 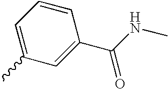 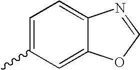 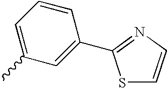 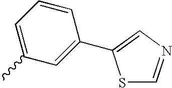 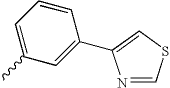 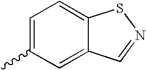 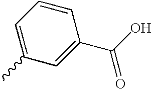 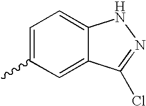 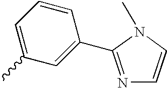 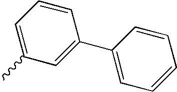 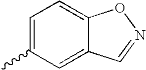 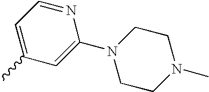 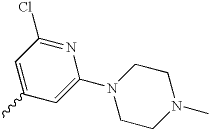 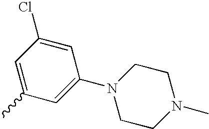 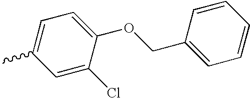 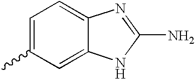 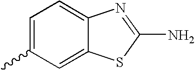 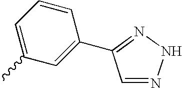 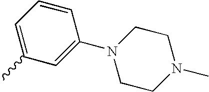 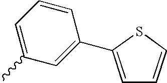 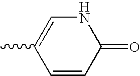 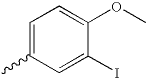 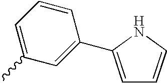 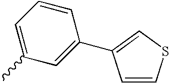 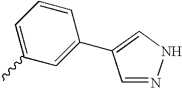 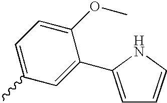 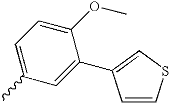 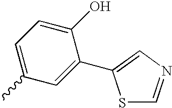 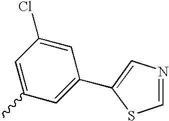 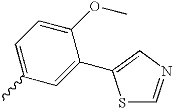 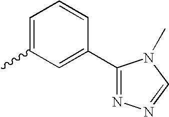 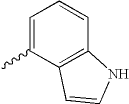 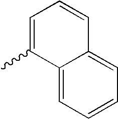 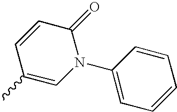 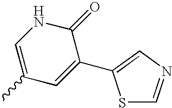 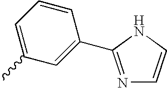 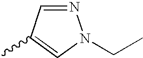 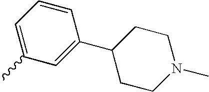 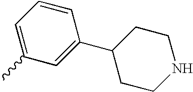 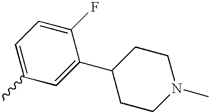 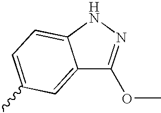 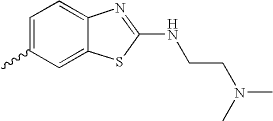 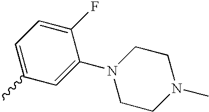 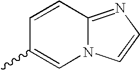 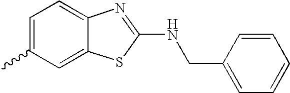 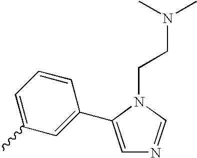 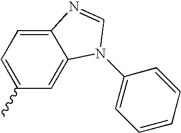 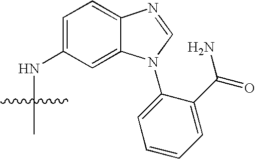 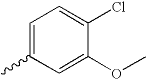 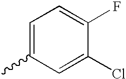 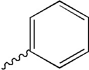 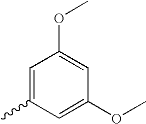 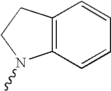 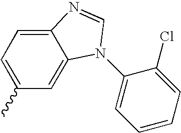 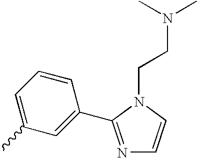 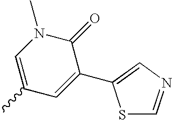 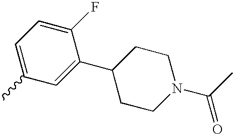 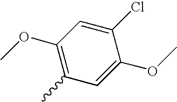 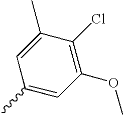 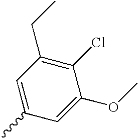 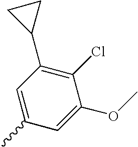 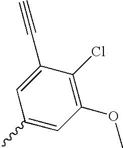 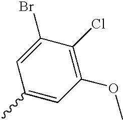
or a stereoisomer, or a pharmaceutically acceptable salt the thereof.

9. The compound according to claim 1, or a pharmaceutically acceptable salt thereof, wherein Y is

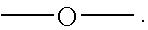

10. The compound according to claim 1, or a pharmaceutically acceptable salt thereof, wherein R3 is halogen.

11. The compound according to claim 10, or a pharmaceutically acceptable salt thereof, wherein Cy is

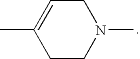

12. The compound according to claim 11, or a pharmaceutically acceptable salt thereof, wherein Y is —N(C₀₋₆alkyl)-.

13. The compound according to claim 10, or a pharmaceutically acceptable salt thereof, wherein R3 is halogen, R4 and R5 are hydrogen, Cy is 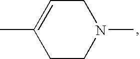
and Y is —N(C₀₋₆alkyl)-.

14. The compound according to claim 1 selected from 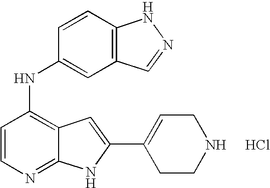 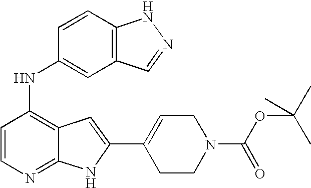 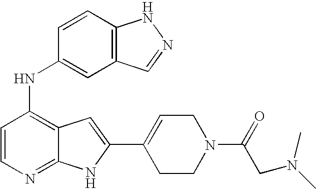 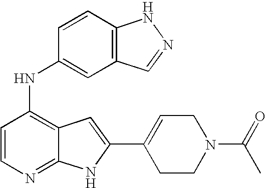 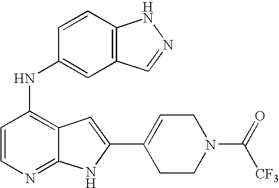 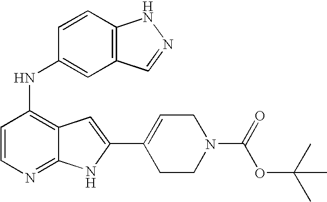 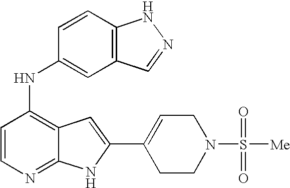 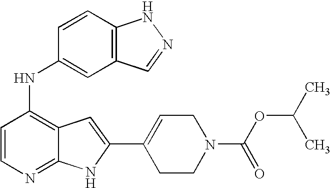 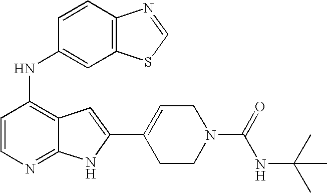 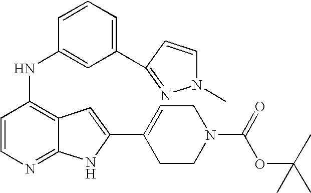 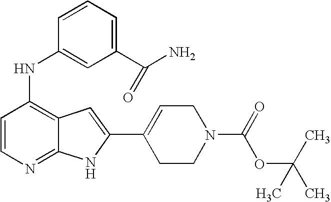 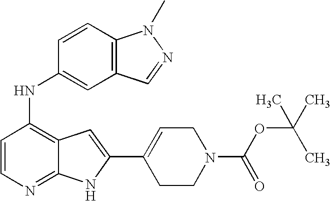 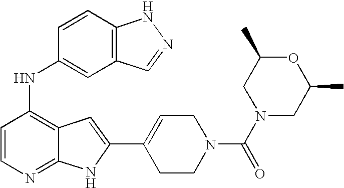 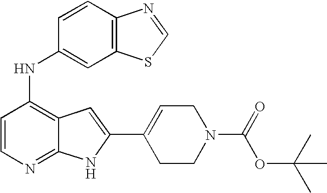 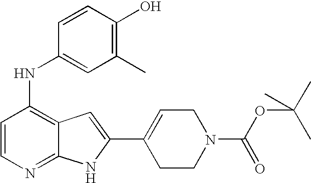 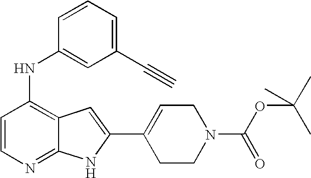 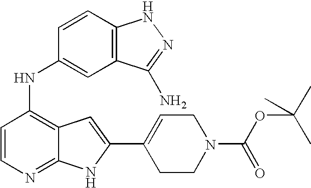 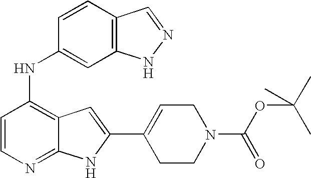 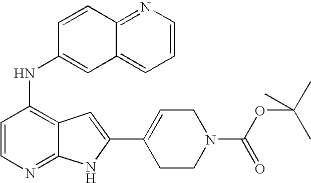 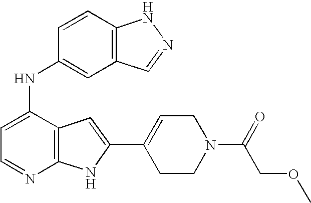 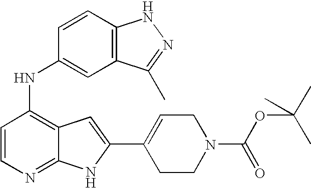 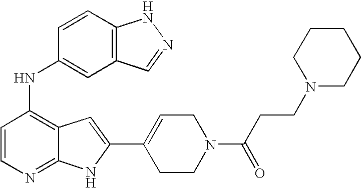 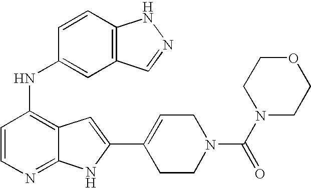 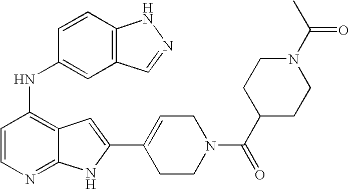 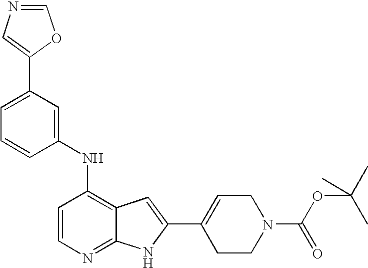 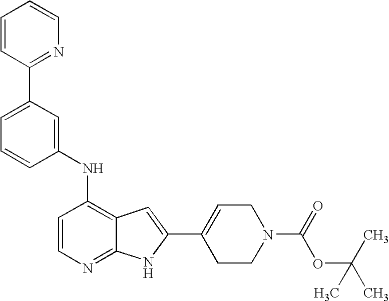 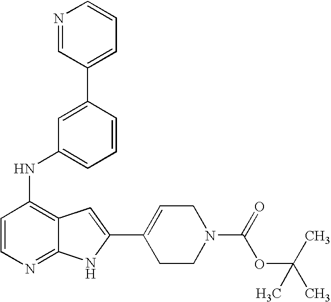 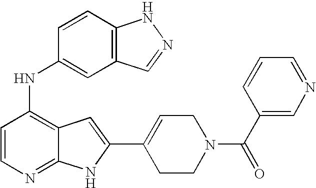 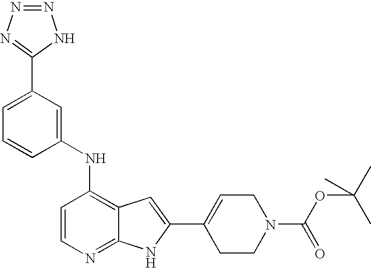 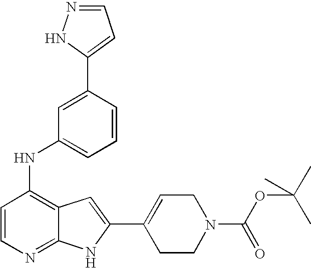 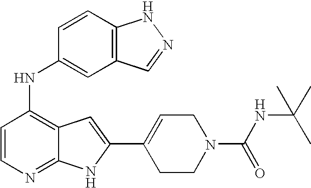 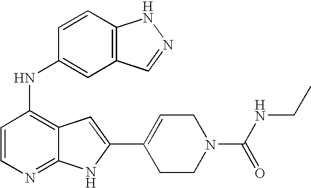 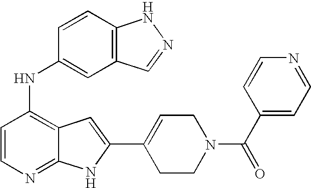 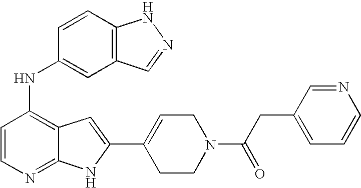 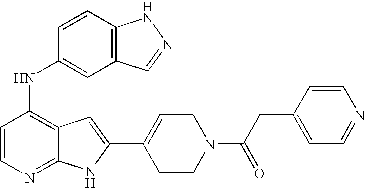 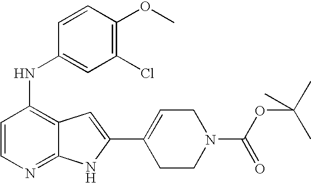 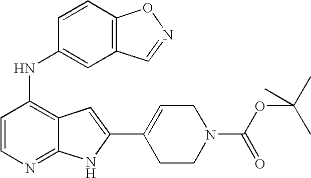 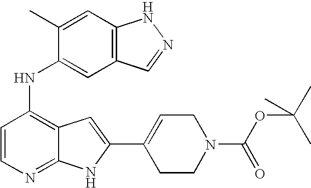 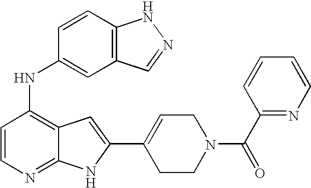 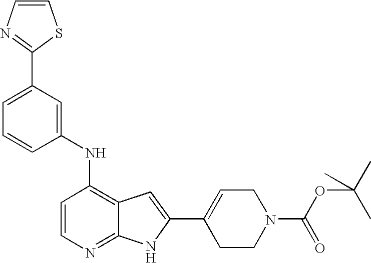 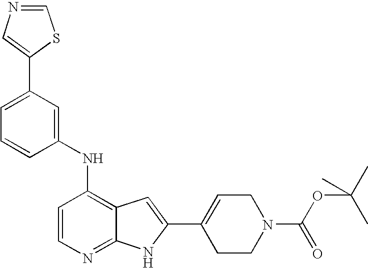 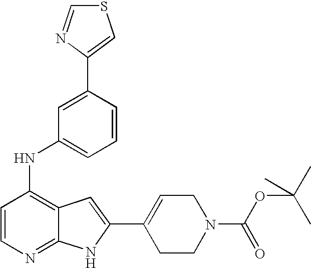 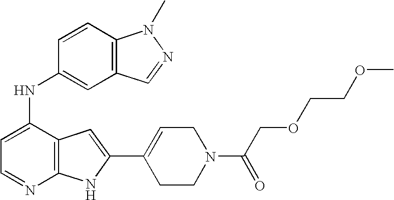 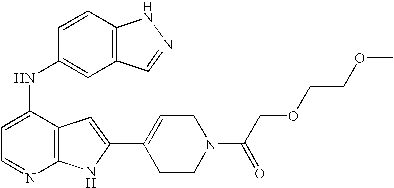 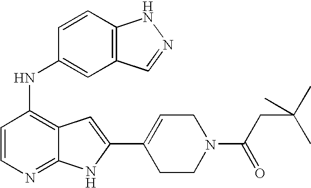 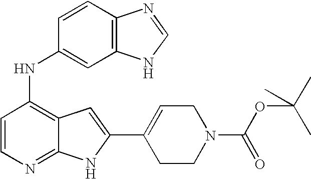 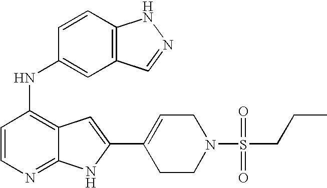 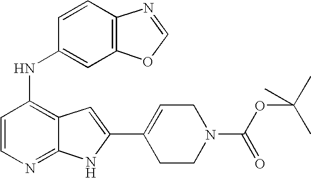 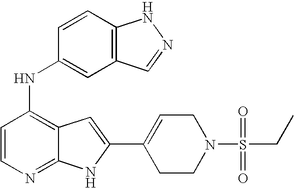 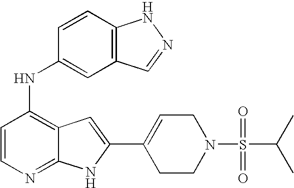 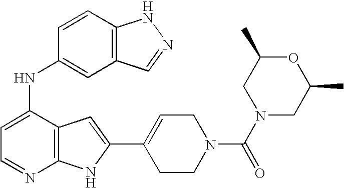 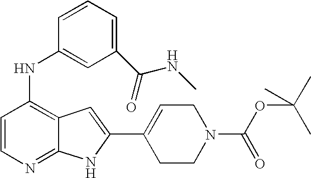 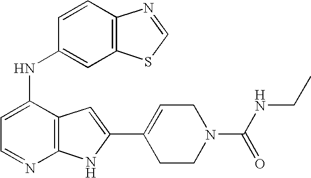 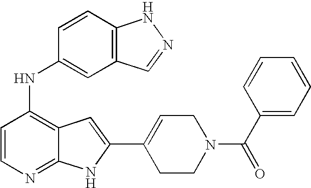 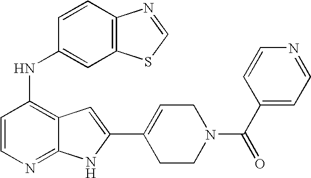 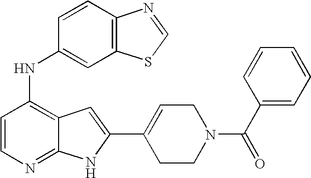 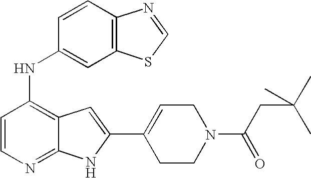 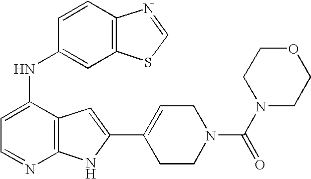 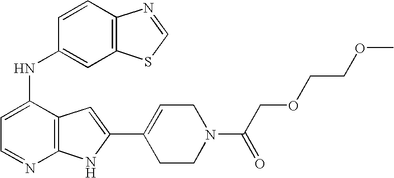 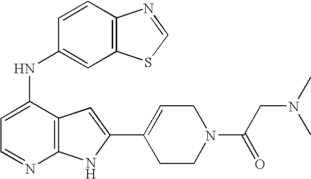 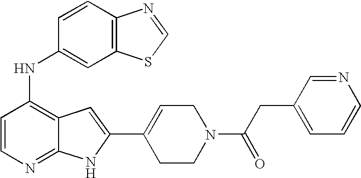 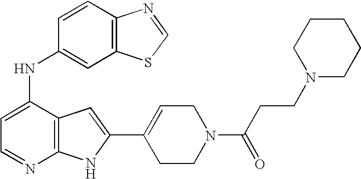 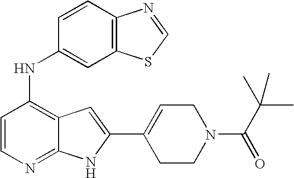 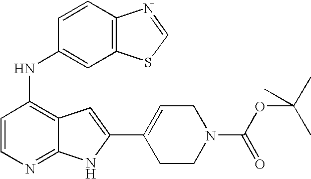 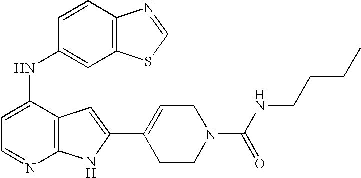 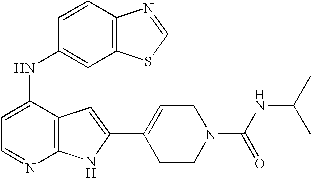 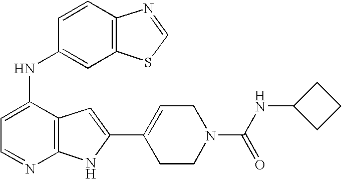 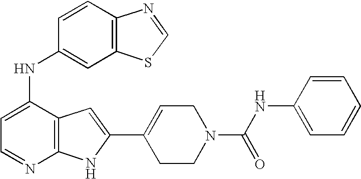 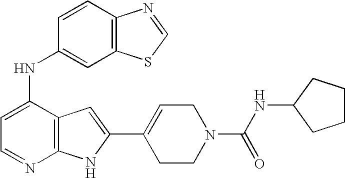 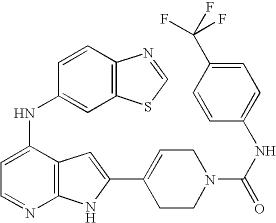 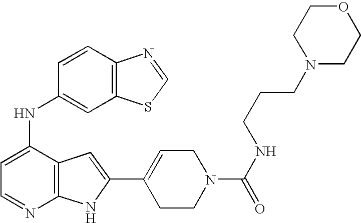 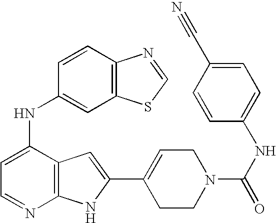 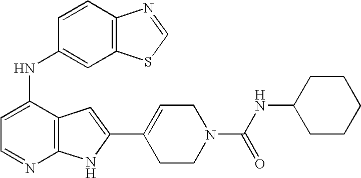 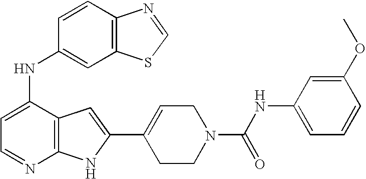 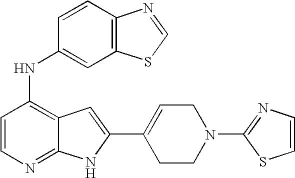 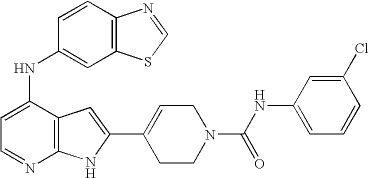 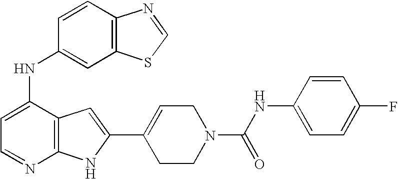 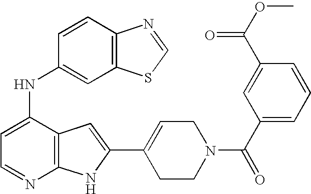 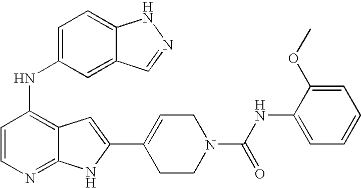 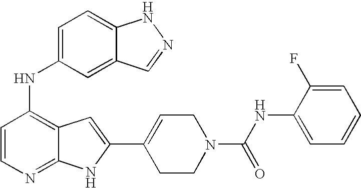 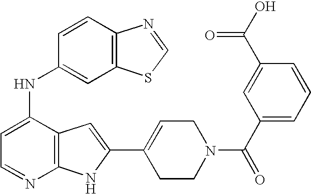 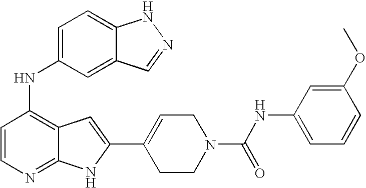 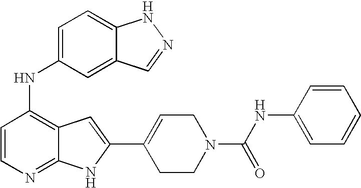 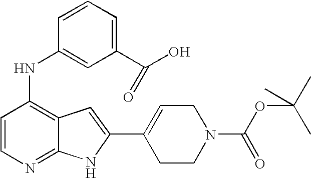 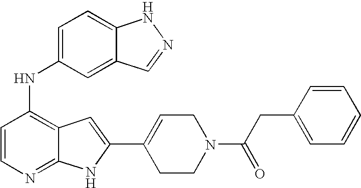 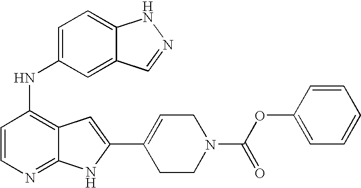 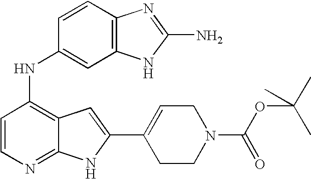 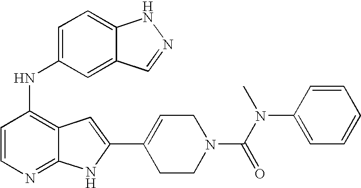 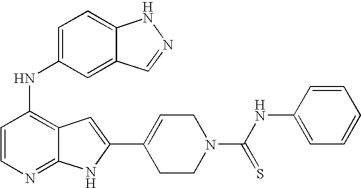 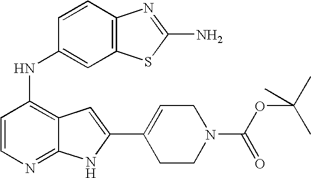 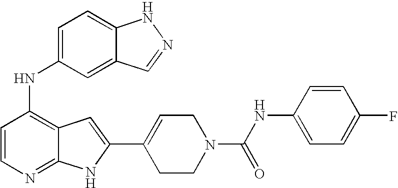 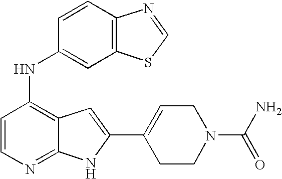 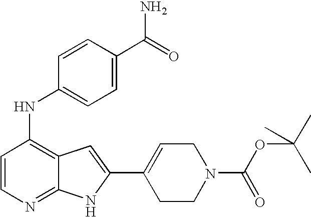 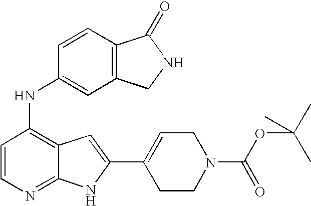 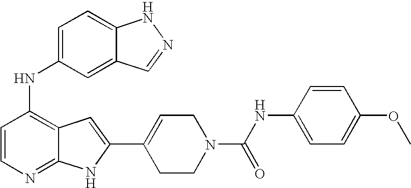 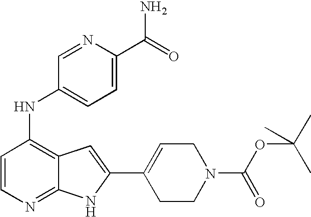 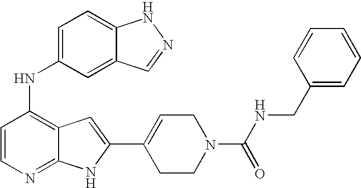 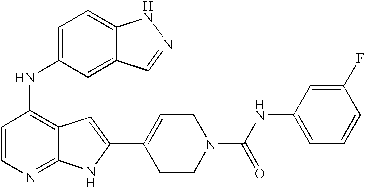 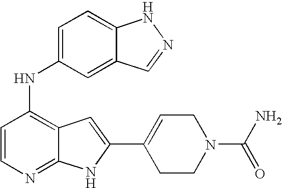 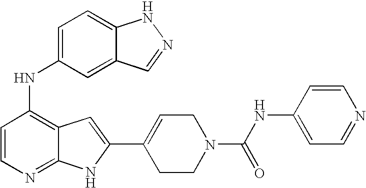 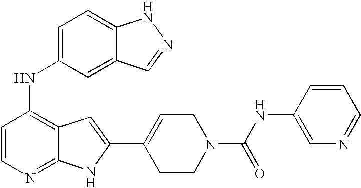 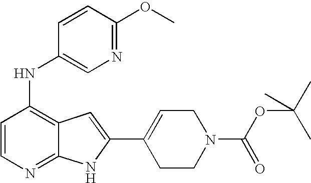 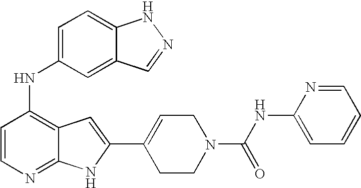 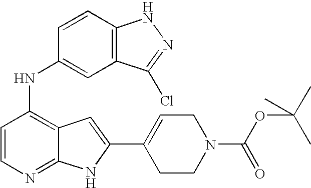 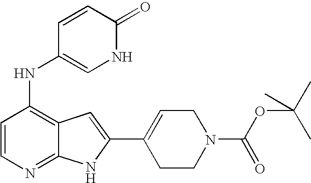 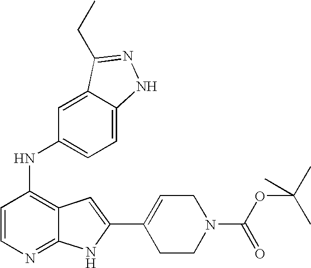 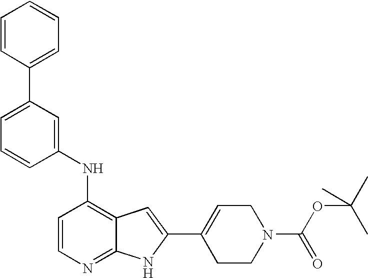 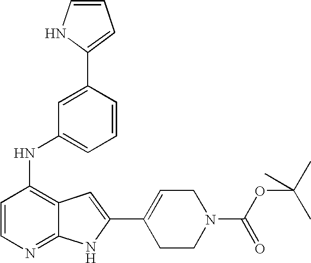 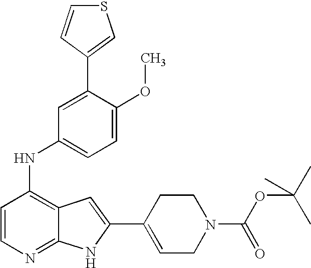 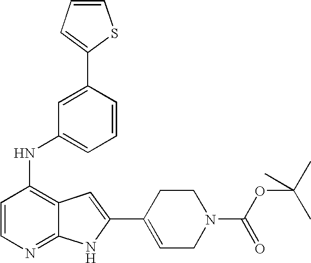 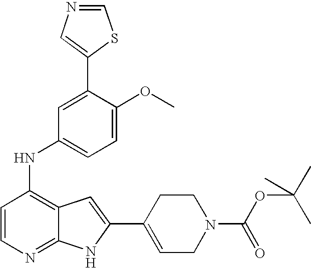 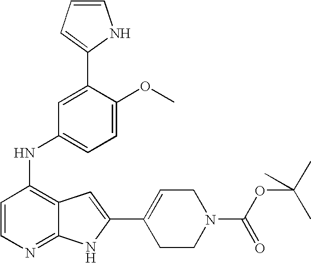 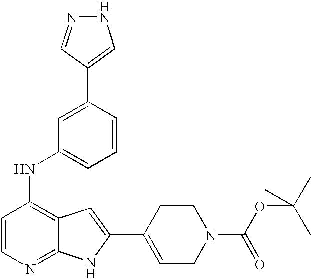 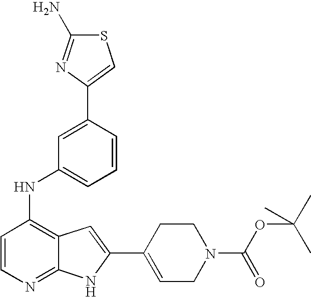 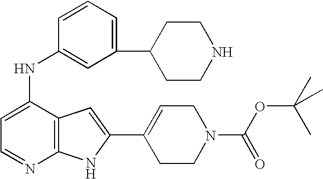 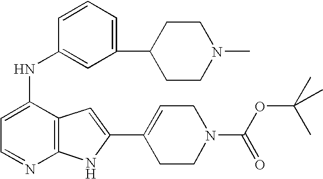 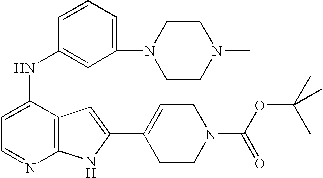 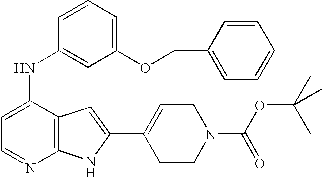 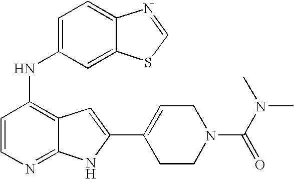 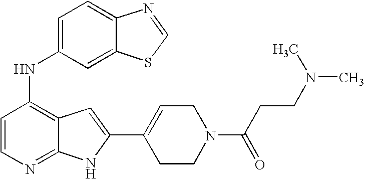 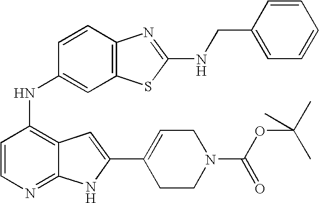 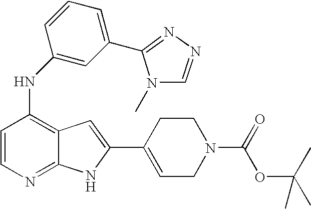 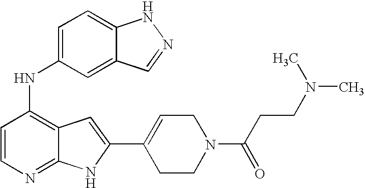 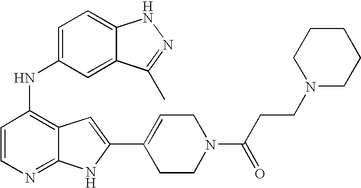 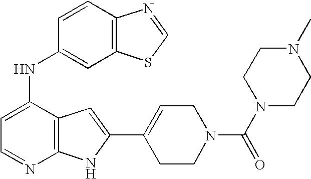 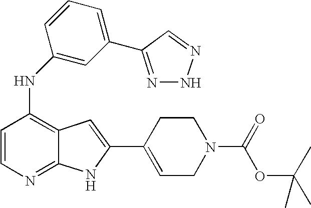 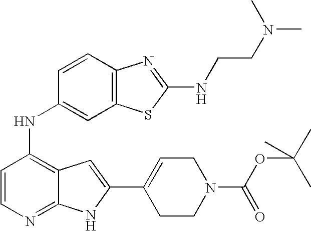 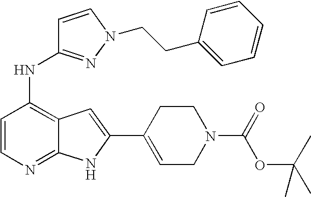 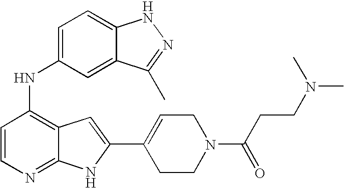 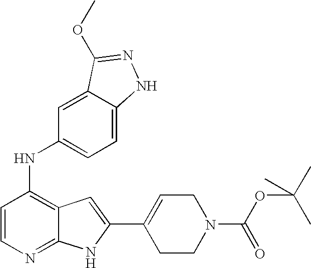 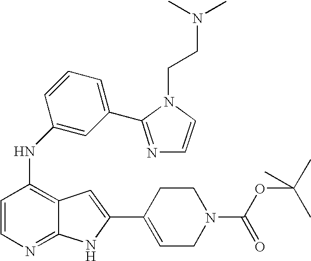 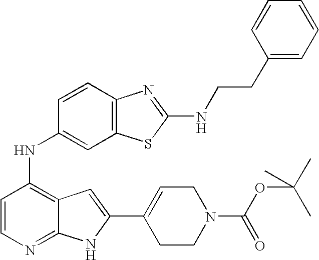 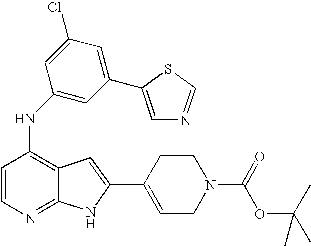 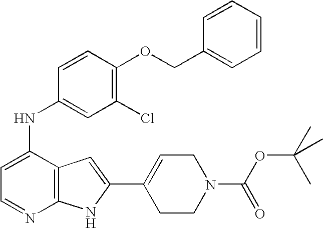 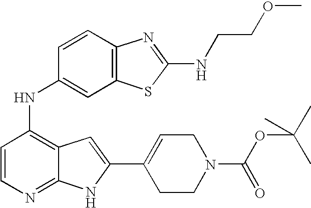 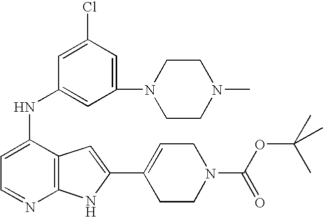 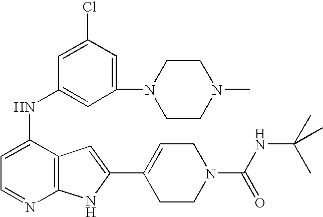 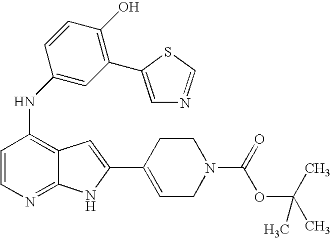 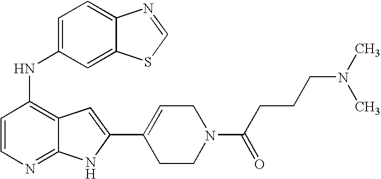 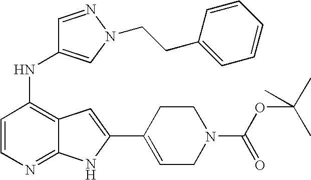 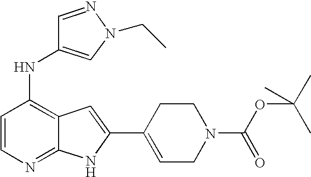 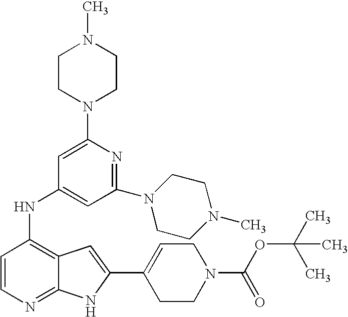 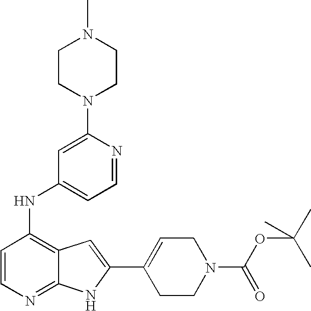 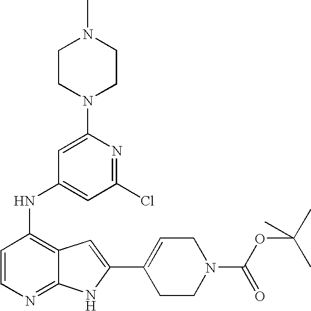 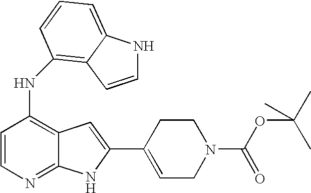 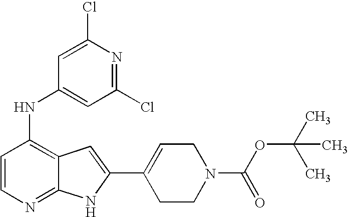 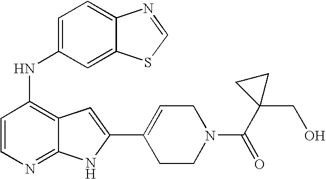 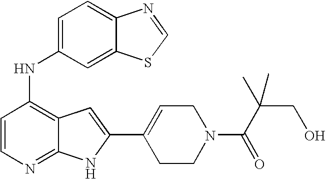 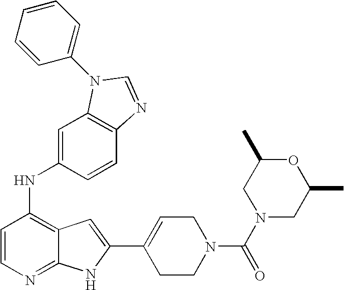 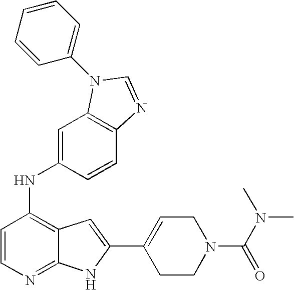 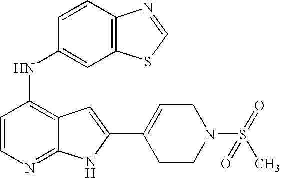 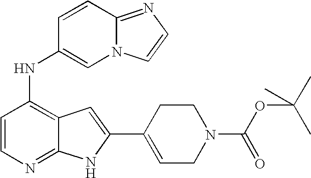 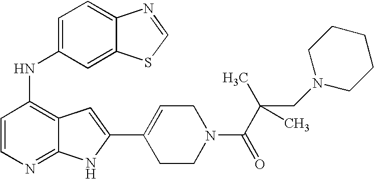 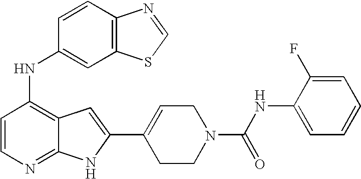 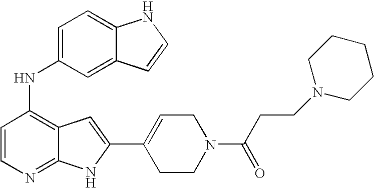 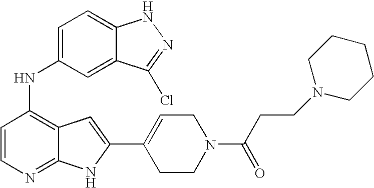 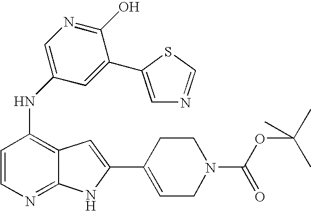 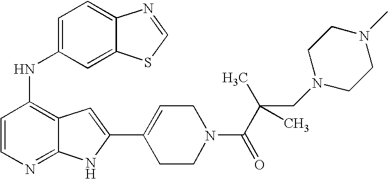 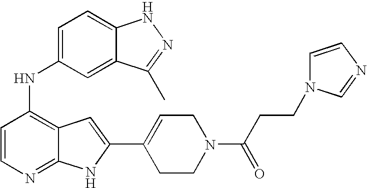 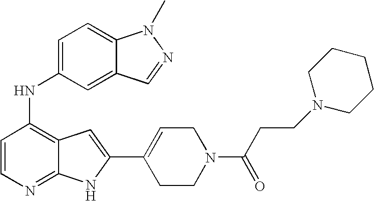 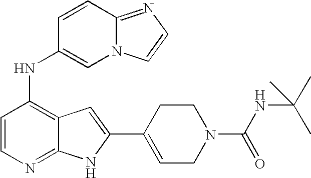 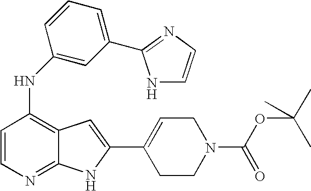 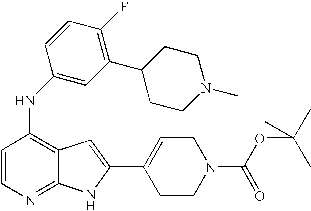 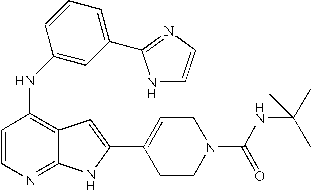 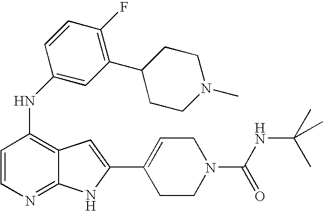 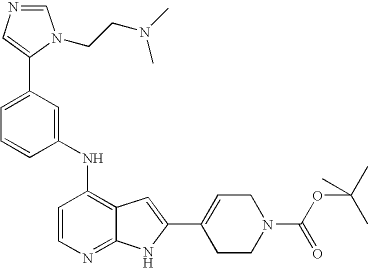 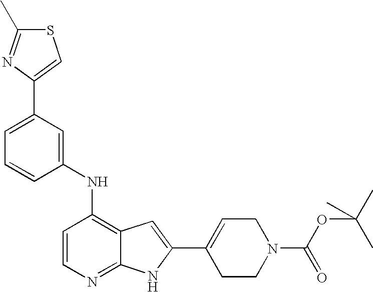 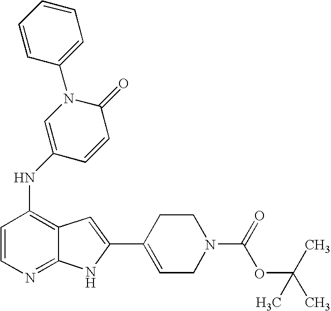 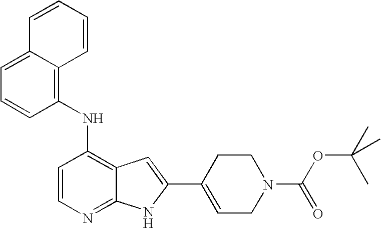 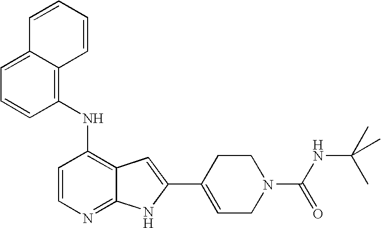 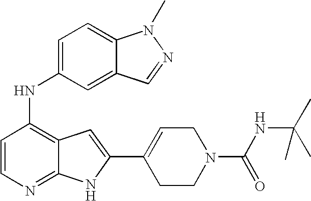 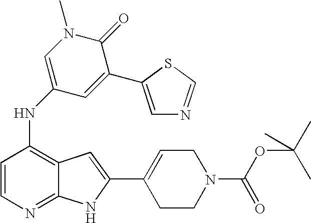 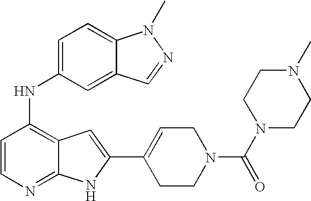 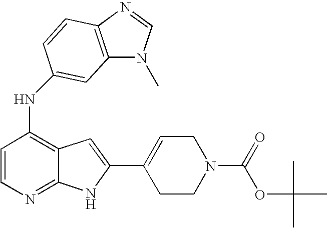 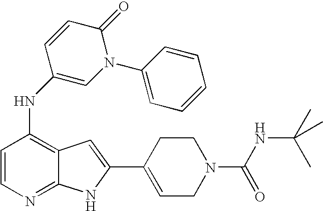 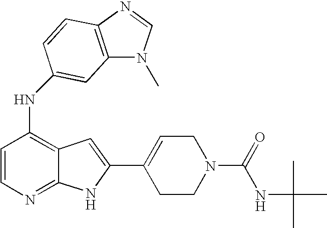 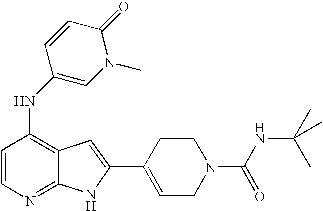 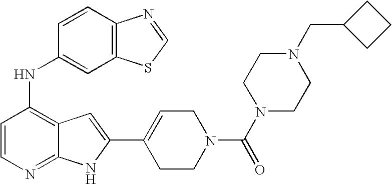 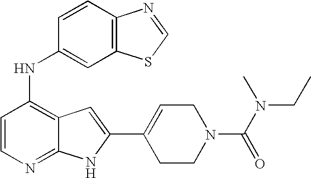 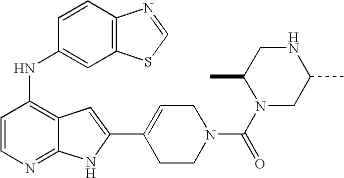 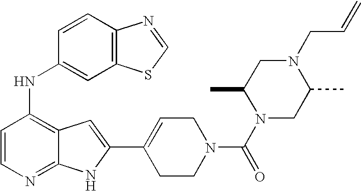 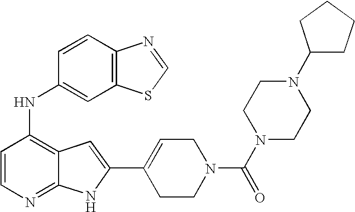 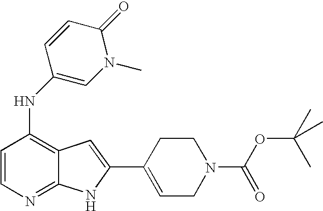 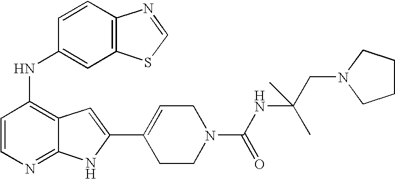 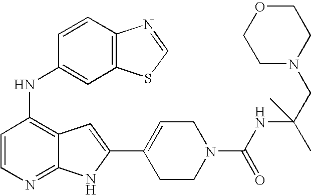 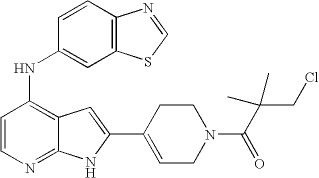 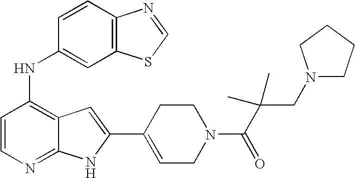 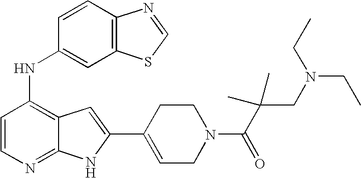 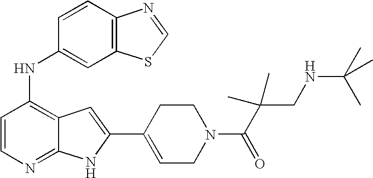 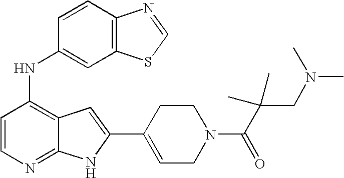 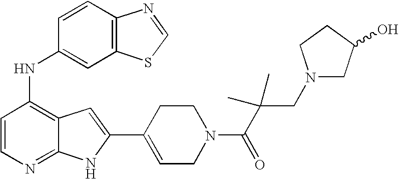 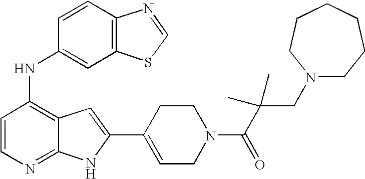 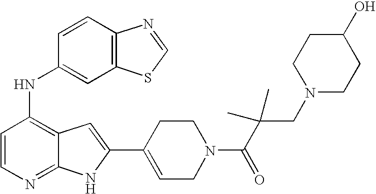 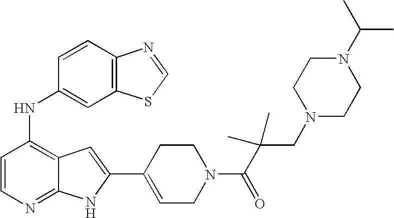 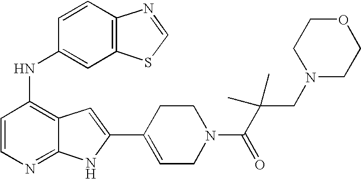 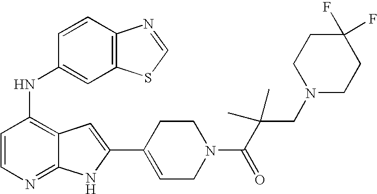 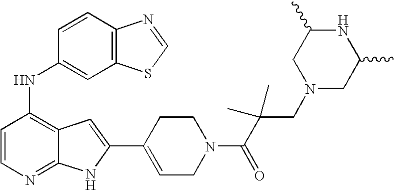 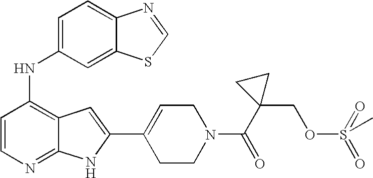 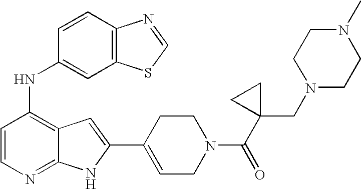 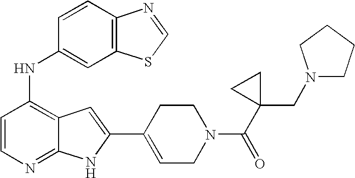 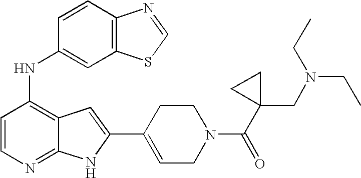 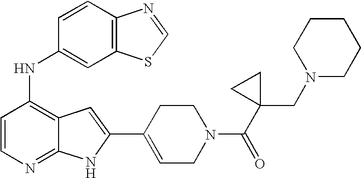 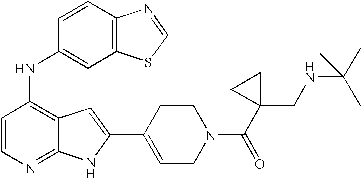 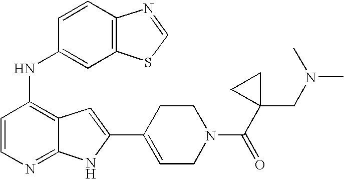 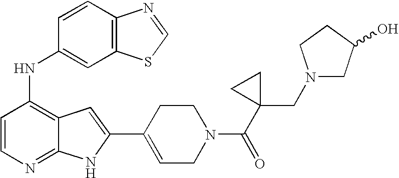 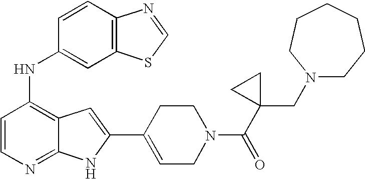 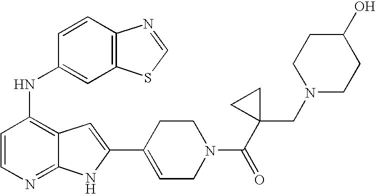 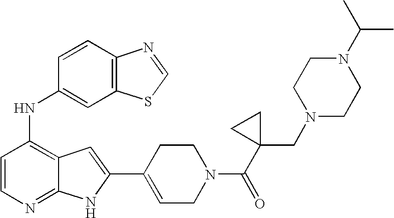 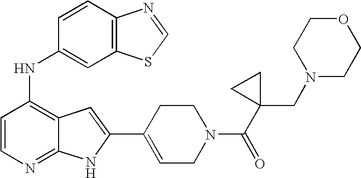 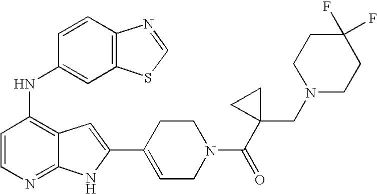 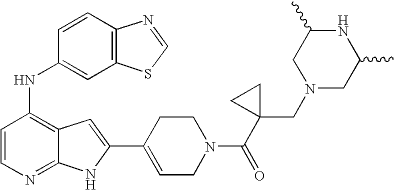 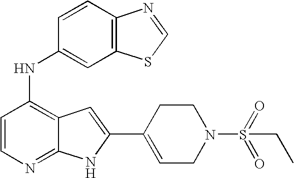 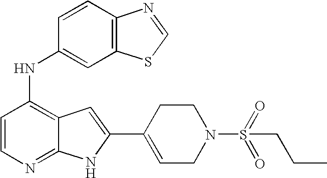 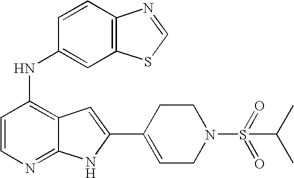 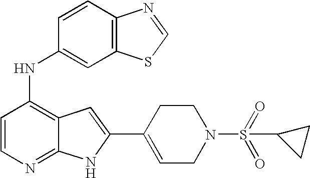 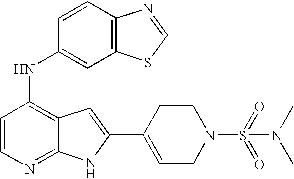 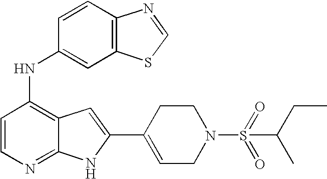 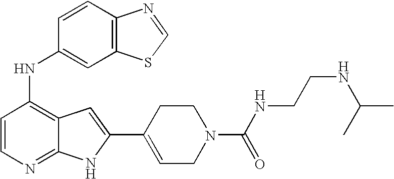 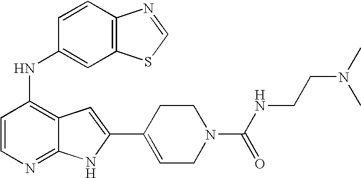 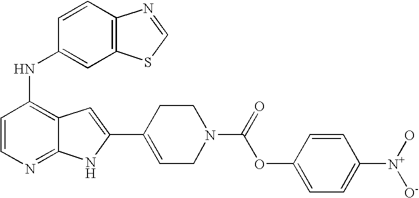 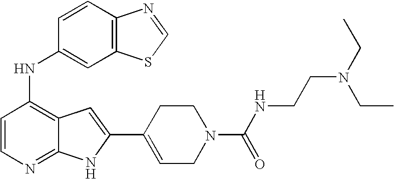 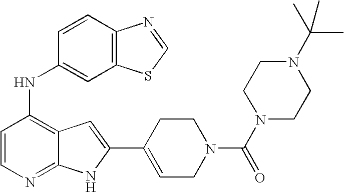 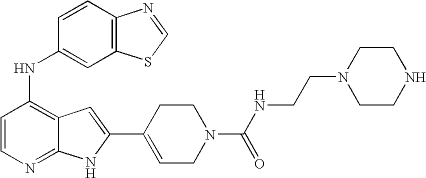 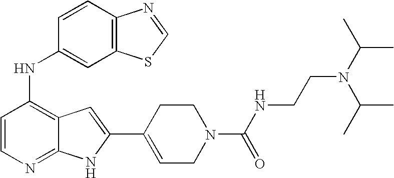 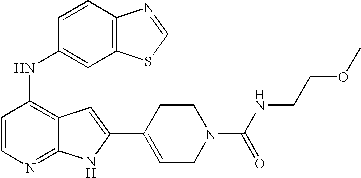 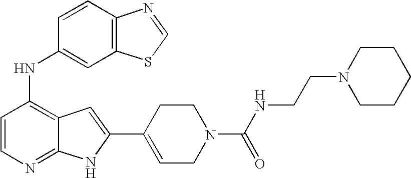 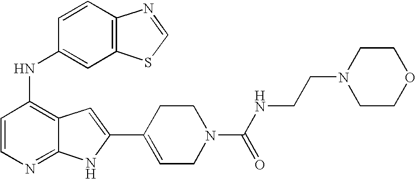 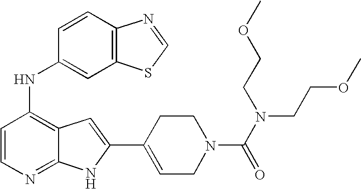 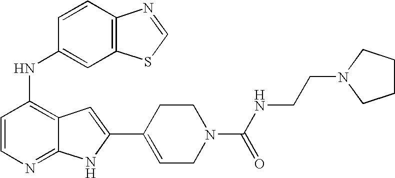 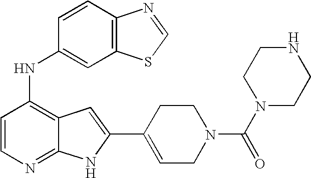 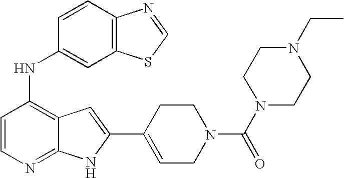 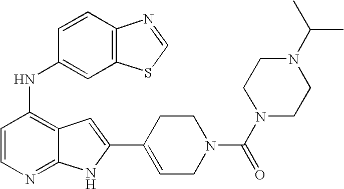 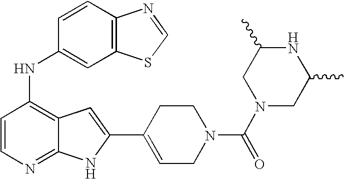 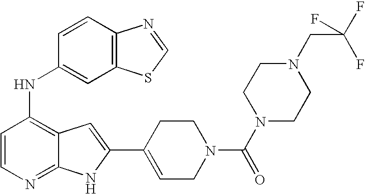 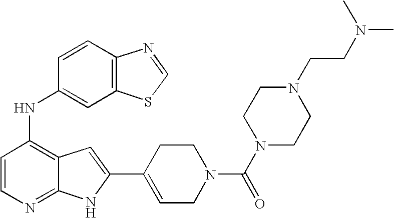 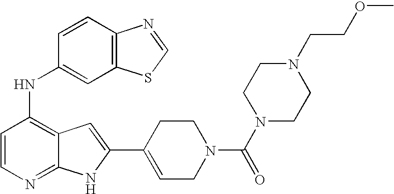 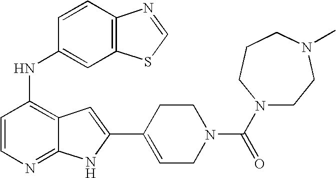 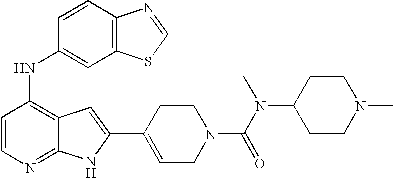 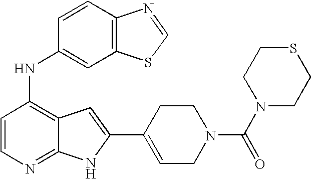 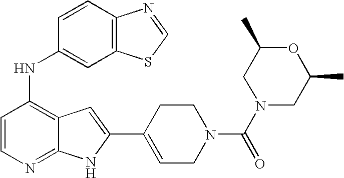 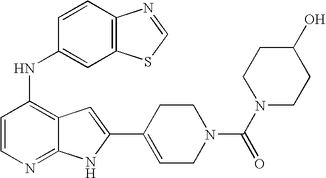 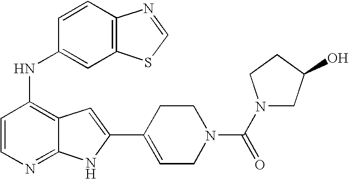 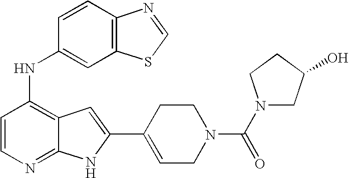 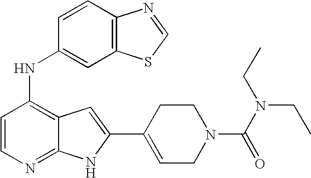 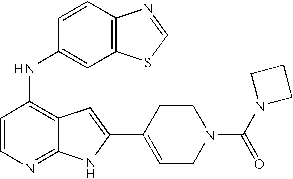 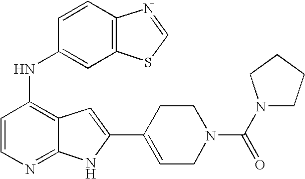 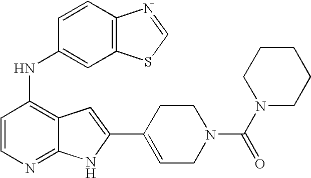 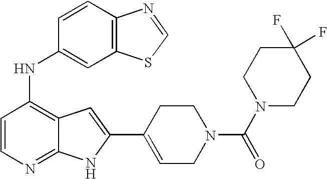 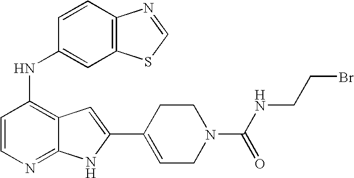 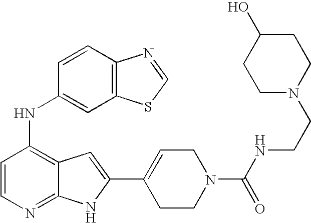 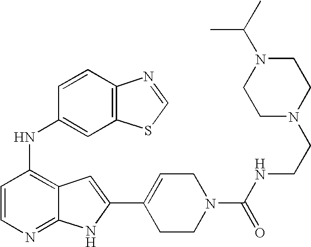 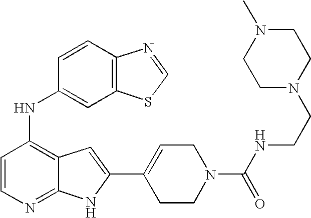 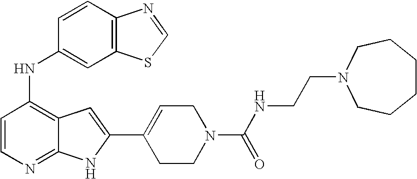 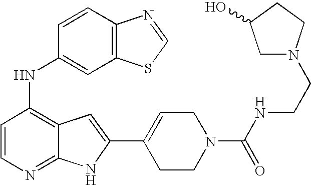 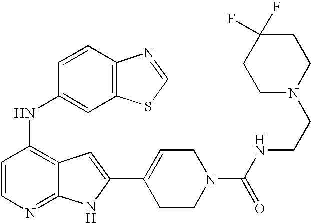 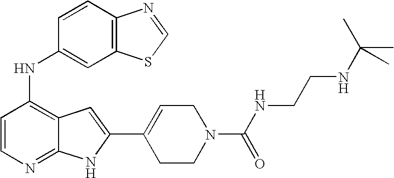 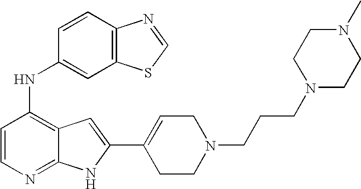 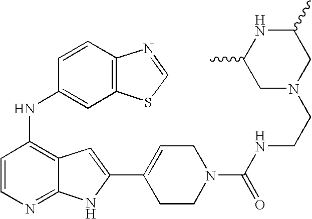 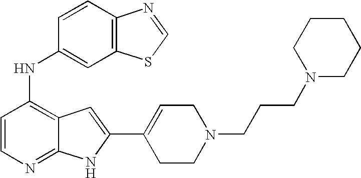 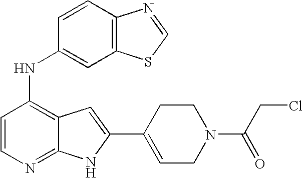 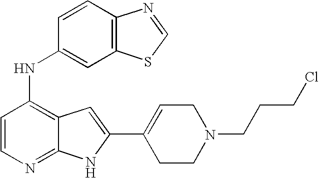 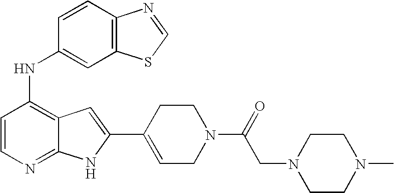 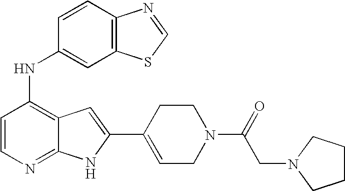 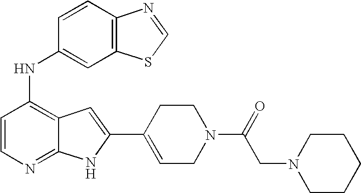 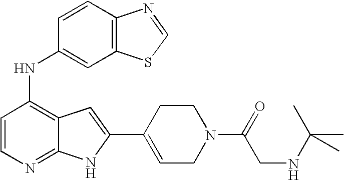 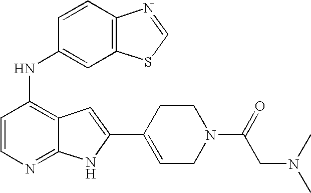 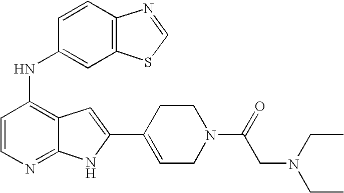 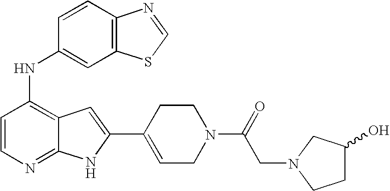 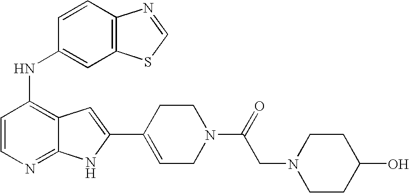 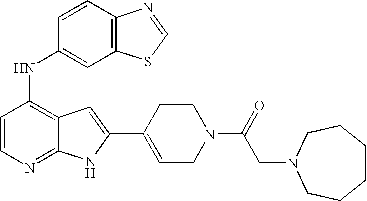 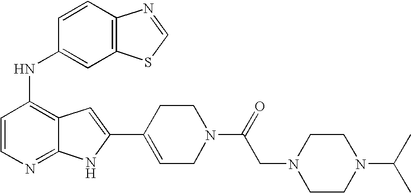 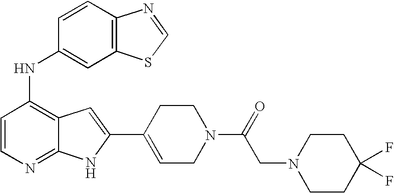 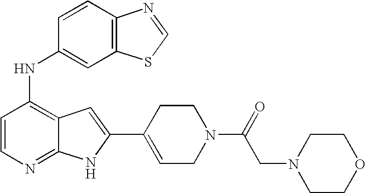 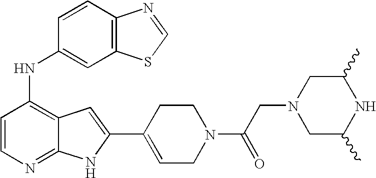 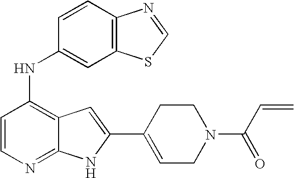 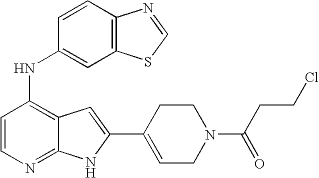 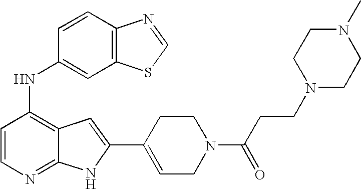 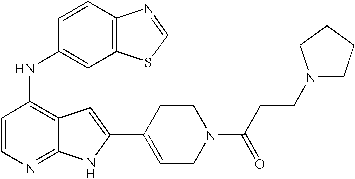 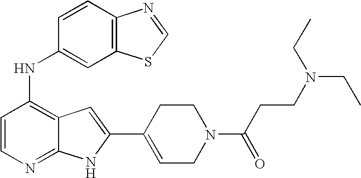 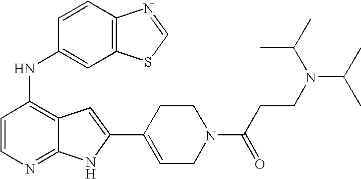 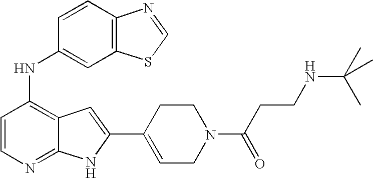 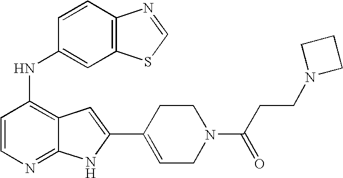 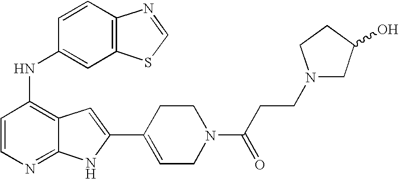 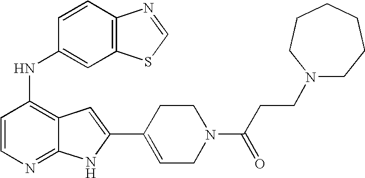 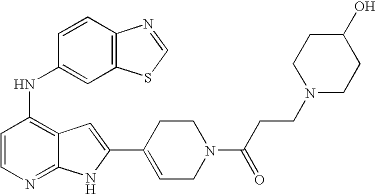 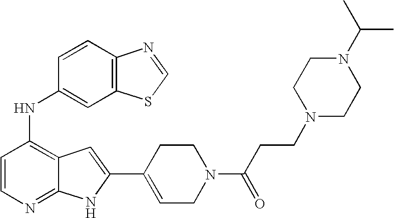 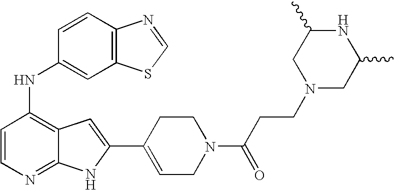 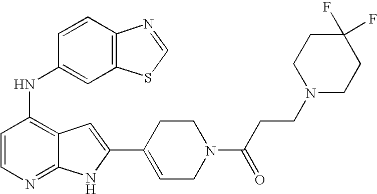 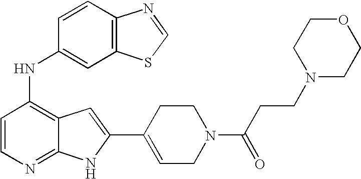 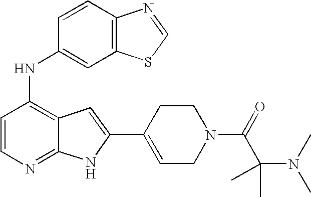 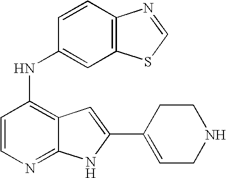 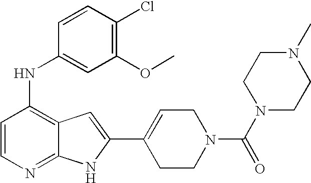 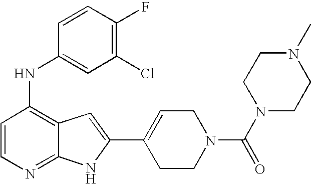 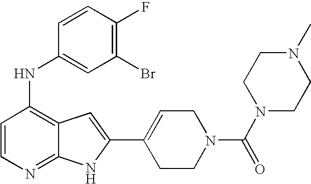 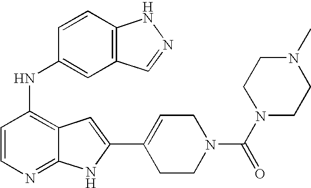 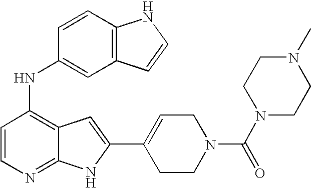 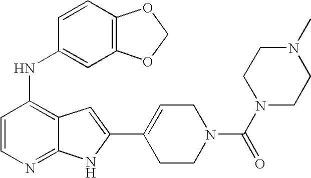 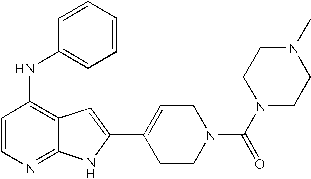 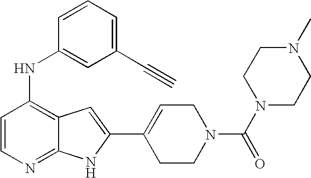 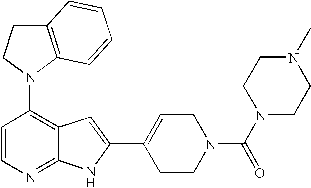 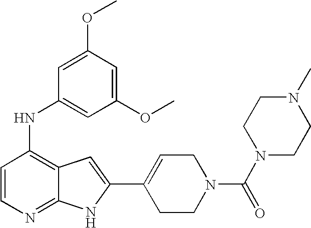 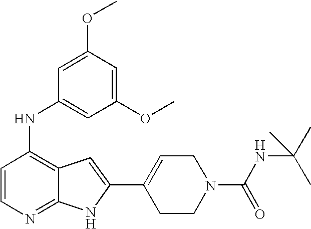 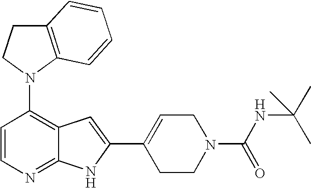 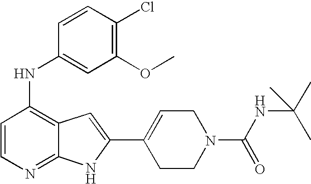 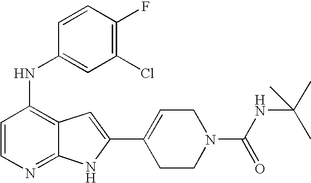 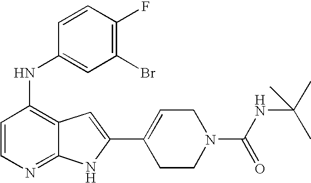 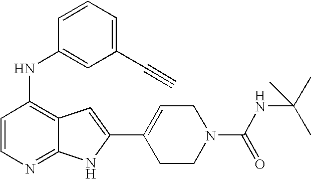 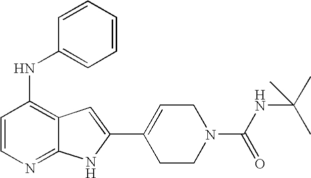 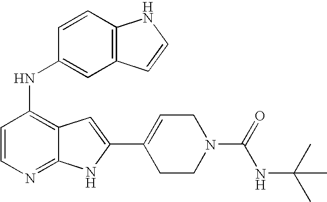 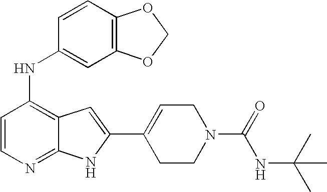 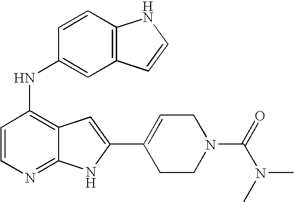 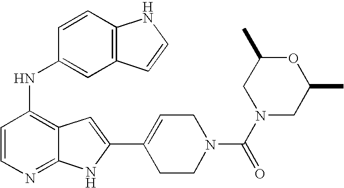 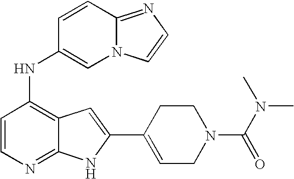 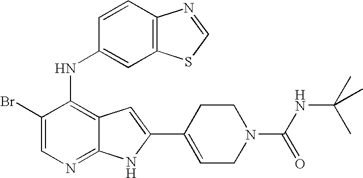 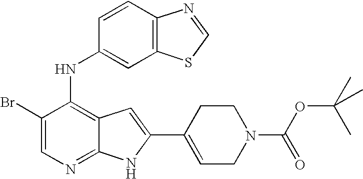 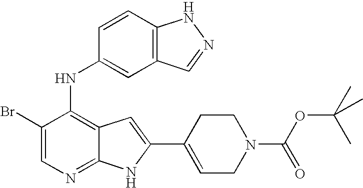 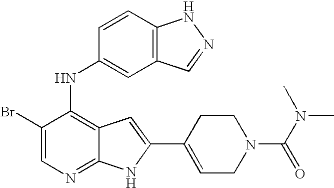 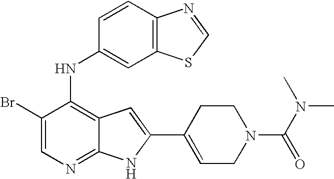 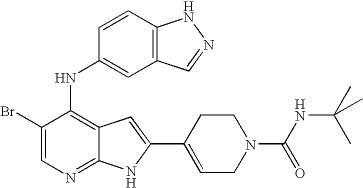 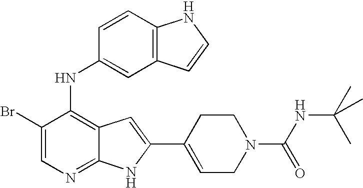 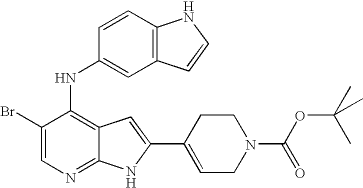 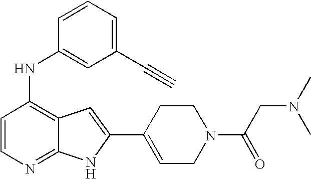 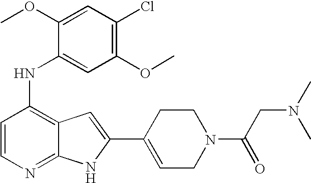 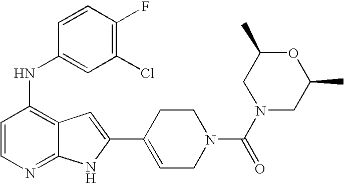 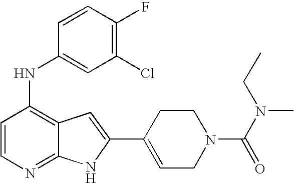 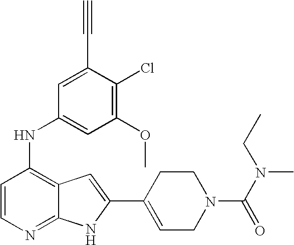 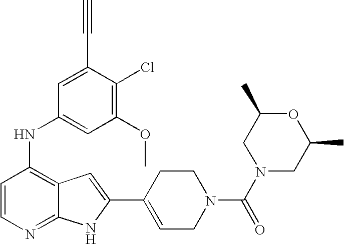 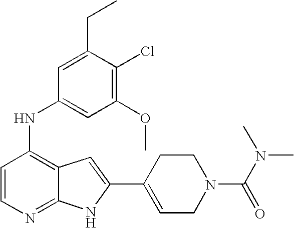 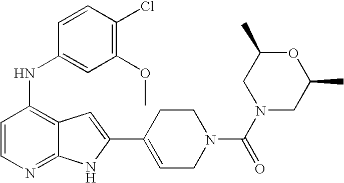 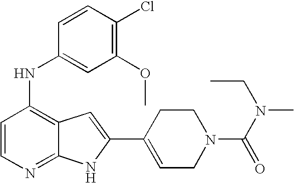 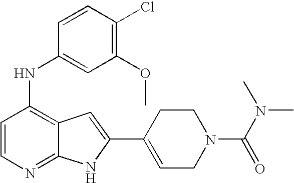 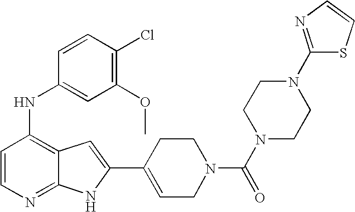 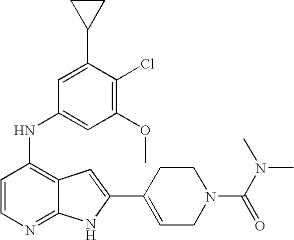 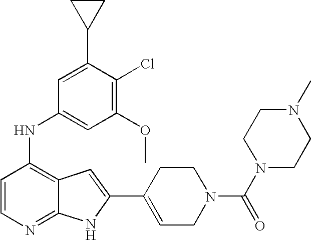 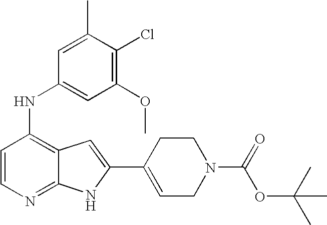 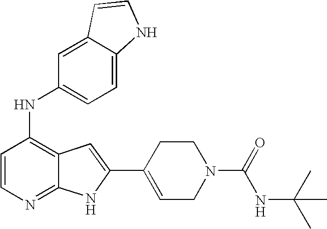 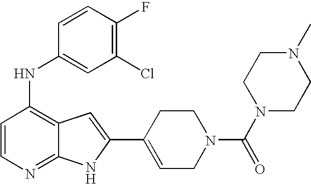 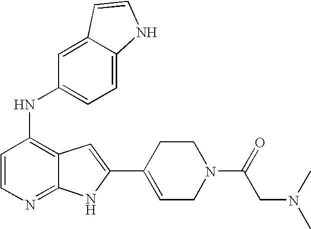 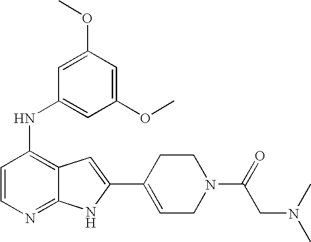 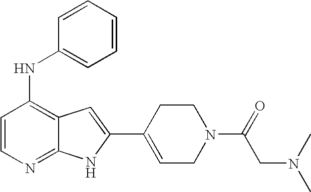 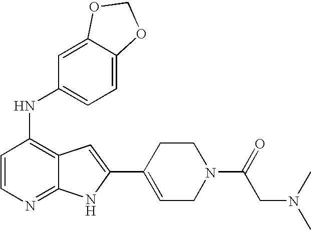 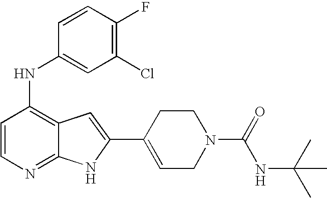 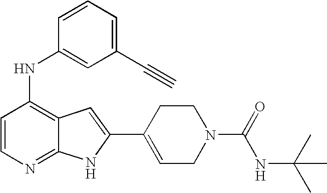 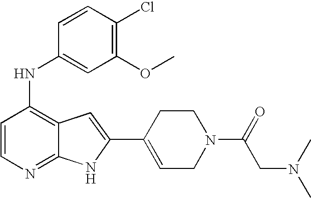 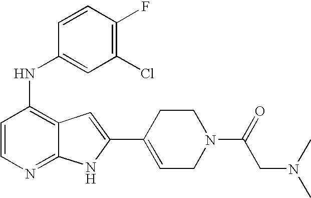 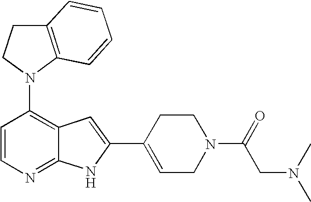 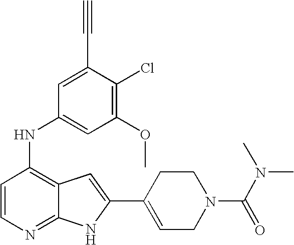 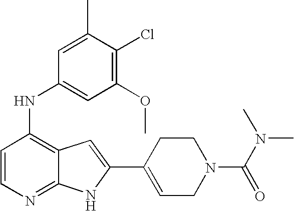 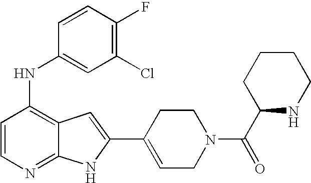 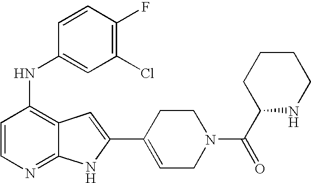 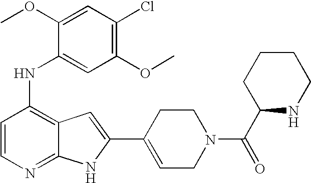 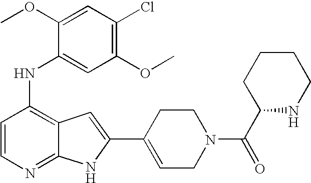 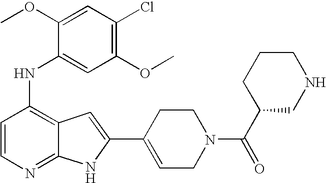 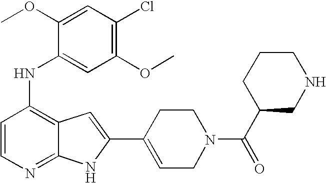 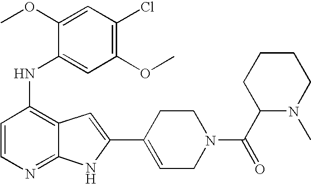 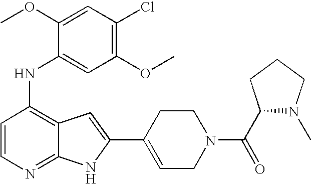 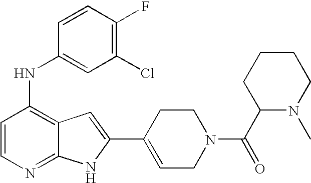 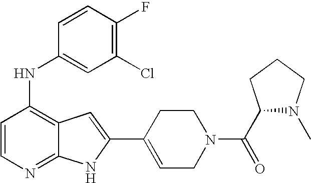 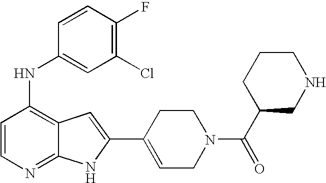 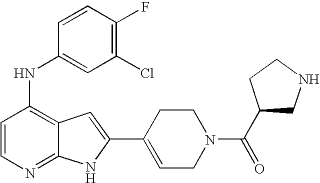 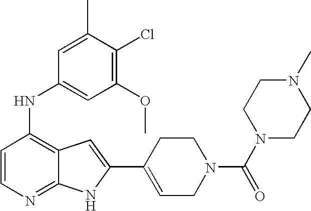 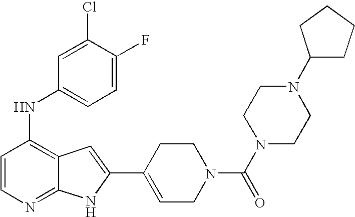 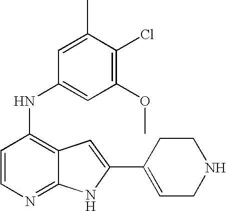
or a pharmaceutically acceptable salt thereof.

15. A compound consisting of
4-[4-(4-Fluoro-3-thiazol-5-ylphenylamino)-1H-pyrrolo[2,3-b]pyridin-2-yl]-3,6-dihydro-2H-pyridine-1-carboxylic acid tert-butyl ester;
4-[4-(4-Fluoro-3-thiazol-5-ylphenylamino)-1H-pyrrolo[2,3-b]pyridin-2-yl]-3,6-dihydro-2H-pyridine-1-carboxylic acid tert-butylamide;
4-{4-[4-Fluoro-3-(4-methylpiperazin-1-yl)-phenylamino]-1H-pyrrolo[2,3-b]pyridin-2-yl}-3,6-dihydro-2H-pyridine-1-carboxylic acid tert-butyl ester;
4-{4-[4-Fluoro-3(4-methylpiperazin-1-yl)-phenylamino]-1H-pyrrolo[2,3-b]pyridin-2-yl}-3,6-dihydro-2H-pyridine-1-carboxylic acid tert-butylamide;
4-{4-[4-Fluoro-3-(1-methylazetidin-3-ylmethyl)-phenylamino]-1H-pyrrolo[2,3-b]pyridin-2-yl}-3,6-dihydro-2H-pyridine-1-carboxylic acid tert-butyl ester;
4-{4-[4-Fluoro-3-(1-methylazetidin-3-ylmethyl)-phenylamino]-1H-pyrrolo[2,3-b]pyridin-2-yl}-3,6-dihydro-2H-pyridine-1-carboxylic acid tert-butylamide;
4-{4-[4-Fluoro-3-(1-methylazetidin-3-yloxy)-phenylamino]-1H-pyrrolo[2,3-b]pyridin-2-yl}-3,6-dihydro-2H-pyridine-1-carboxylic acid tert-butyl ester;
4-{4-[4-Fluoro-3-(1-methylazetidin-3-yloxy)-phenylamino]-1H-pyrrolo[2,3-b]pyridin-2-yl }-3,6-dihydro-2H-pyridine-1-carboxylic acid tert-butyl ester;
4-{4-[4-Fluoro-3-(4-methylpiperazin-1-ylmethyl)-phenylamino]-1H-pyrrolo[2,3-b]pyridin-2-yl}-3,6-dihydro-2H-pyridine-1-carboxylic acid tert-butyl ester;
4-{4-[4-Fluoro-3-(4-methylpiperazin-1-ylmethyl)-phenylamino]-1H-pyrrolo[2,3-b]pyridin-2-yl}-3,6-dihydro-2H-pyridine-1-carboxylic acid tert-butylamide;
4-{4-[4-Fluoro-3-(1-methyl-1,2,3,6-tetrahydropyridin-4-yl)-phenylamino]-1H-pyrrolo[2,3-b]pyridin-2-yl}-3,6-dihydro-2H-pyridine-1-carboxylic acid tert-butyl ester;
4-{4-[4-Fluoro-3-(1-methyl-1,2,3,6-tetrahydropyridin-4-yl)-phenylamino]-1H-pyrrolo[2,3-b]pyridin-2-yl}-3,6-dihydro-2H-pyridine-1-carboxylic acid tert-butyl amide;
4-{4-[4-Fluoro-3-(1-methyl-2,5-dihydro-1H-pyrrol-3-yl)-phenylamino]-1H-pyrrolo[2,3-b]pyridin-2-yl}-3,6-dihydro-2H-pyridine-1-carboxylic acid tert-butyl ester;
4-{4-[4-Fluoro-3-(1-methyl-2,5-dihydro-1H-pyrrol-3-yl)-phenylamino]-1H-pyrrolo[2,3-b]pyridin-2-yl}-3,6-dihydro-2H-pyridine-1-carboxylic acid tert-butyl amide;
(S)-4-{4-[4-Fluoro-3-(1-methylpyrrolidin-3-yl)-phenylamino]-1H-pyrrolo[2,3-b]pyridin-2-yl}-3,6-dihydro-2H-pyridine-1-carboxylic acid tert-butyl ester;
(S)-4-{4-[4-Fluoro-3-(1-methylpyrrolidin-3-yl)-phenylamino]-1H-pyrrolo[2,3-b]pyridin-2-yl}-3,6-dihydro-2H-pyridine-1-carboxylic acid tert-butyl amide;
(R)-4-{4-[4-Fluoro-3-(1-methylpyrrolidin-3-yl)-phenylamino]-1H-pyrrolo[2,3-b]pyridin-2-yl}-3,6-dihydro-2H-pyridine-1-carboxylic acid tert-butyl ester;
(R)-4-{4-[4-Fluoro-3-(1-methylpyrrolidin-3-yl)-phenylamino]-1H-pyrrolo[2,3-b]pyridin-2-yl }-3,6-dihydro-2H-pyridine-1-carboxylic acid tert-butyl amide;
{4-[4-(Benzothiazol-6-ylamino)-1H-pyrrolo[2,3-b]pyridin-2-yl]-3,6-dihydro-2H-pyridin-1-yl}-(2,2,4-trimethylpiperazin-1-yl)-methanone;
{4-[4-(Benzothiazol-6-ylamino)-1H-pyrrolo[2,3-b]pyridin-2-yl]-3,6-dihydro-2H-pyridin-1-yl }-(2,4,5-trimethylpiperazin-1-yl)-methanone;
{4-[4(-Benzothiazol-6-ylamino)-1H-pyrrolo[2,3-b]pyridin-2-yl]-3,6-dihydro-2H-pyridin-1-yl}-(3,4,5-trimethylpiperazin-1-yl)-methanone;
{4-[4-(Benzothiazol-6-ylamino)-1H-pyrrolo[2,3-b]pyridin-2-yl]-3,6-dihydro-2H-pyridin-1-y}-[4-(2,2,2-trifluoroethyl)-piperazin-1-yl]-methanone;
{4-[4-(Benzothiazol-6-ylamino)-1H-pyrrolo[2,3-b]pyridin-2-yl]-3,6-dihydro-2H-pyridin-1-yl}-(4-tert-butylpiperazin-1-yl)-methanone;
Benzothiazol-6-yl-[6-(3,6-dihydro-2H-[1,2′]bipyridinyl-4-yl)-1H-pyrrolo[2,3-b]pyridin-2-yl]-amine;
Benzothiazol-6-yl-[6-(1-thiazol-2-yl-1,2,3,6-tetrahydropyridin-4-yl)-1H-pyrrolo[2,3-b]pyridin-2-yl]-amine;
Benzothiazol-6-yl-[6-(1-oxazol-2-yl-1,2,3,6-tetrahydropyridin-4-yl)-1H-pyrrolo[2,3-b]pyridin-2-yl]-amine;
4-[4-(3-Phenyl-3H-benzimidazol-5-ylamino)-1H-pyrrolo[2,3-b]pyridin-2-yl]-3,6-dihydro-2H-pyridine-1-carboxylic acid tert-butyl ester;
4-[4-(3-Phenyl-3H-benzimidazol-5-ylamino)-1H-pyrrolo[2,3-b]pyridin-2-yl]-3,6-dihydro-2H-pyridine-1-carboxylic acid tert-butylamide;
4-{4-[3-(2-Carbamoylphenyl)-3H-benzimidazol-5-ylamino]-1H-pyrrolo[2,3-b]pyridin-2-yl}-3,6-dihydro-2H-pyridine-1-carboxylic acid tert-butylamide;
4-{-[3-(2-Carbamoylphenyl)-3H-benzimidazol-5-ylamino]-1H-pyrrolo[2,3-b]pyridin-2-yl}-3,6-dihydro-2H-pyridine-1-carboxylic acid tert-butyl ester;
4-{4-[3-(2-Aminoethyl)-3H-benzimidazol-5-ylamino]-1H-pyrrolo[2,3-b]pyridin-2-yl}-3,6-dihydro-2H-pyridine-1-carboxylic acid tert-butylamide;
4-{4-[3-(2-Aminoethyl)-3H-benzimidazol-5-ylamino]-1H-pyrrolo[2,3-b]pyridin-2-yl}-3,6-dihydro-2H-pyridine-1-carboxylic acid tert-butyl ester;
4-{4-[3-(2-Dimethylaminoethyl)-3H-benzimidazol-5-ylamino]-1H-pyrrolo[2,3-b]pyridin-2-yl}-3,6-dihydro-2H-pyridine-1-carboxylic acid tert-butylamide;
4-{4-[3-(2-Dimethylaminoethyl)-3H-benzimidazol-5-ylamino]-1H-pyrrolo[2,3-b]pyridin-2-yl}-3,6-dihydro-2H-pyridine-1-carboxylic acid tert-butyl ester;
4-{4-[3-(2-Acetylaminoethyl)-3H-benzimidazol-5-ylamino]-1H-pyrrolo[2,3-b]pyridin-2-yl}-3,6-dihydro-2H-pyridine-1-carboxylic acid tert-butylamide;
4-{4-[3-(2-Acetylaminoethyl)-3H-benzimidazol-5-ylamino]-1H-pyrrolo[2,3-b]pyridin-2-yl}-3,6-dihydro-2H-pyridine-1-carboxylic acid tert-butyl ester;
4-[4-(Imidazo[1,2-a]pyridin-6-ylamino)-1H-pyrrolo[2,3-b]pyridin-2-yl]-3,6-dihydro-2H-pyridine-1-carboxylic acid tert-butylamide;
4-[4-(Imidazo[1,2-a]pyridin-6-ylamino)-1H-pyrrolo[2,3-b]pyridin-2-yl]-3,6-dihydro-2H-pyridine-1-carboxylic acid tert-butyl ester;
4-[4-(3-Methylimidazo[1,2-a]pyridin-6-ylamino)-1H-pyrrolo[2,3-b]pyridin-2-yl]-3,6-dihydro-2H-pyridine-1-carboxylic acid tert-butylamide;
4-[4-(3-Methylimidazo[1,2-a]pyridin-6-ylamino)-1H-pyrrolo[2,3-b]pyridin-2-yl]-3,6-dihydro-2H-pyridine-1-carboxylic acid tert-butyl ester;
4-[4-(3-Phenylimidazo[1,2-a]pyridin-6-ylamino)-1H-pyrrolo[2,3-b]pyridin-2-yl]-3,6-dihydro-2H-pyridine-1-carboxylic acid tert-butylamide;
4-[4-(3-Phenylimidazo[1,2-a]pyridin-6-ylamino)-1H-pyrrolo[2,3-b]pyridin-2-yl]-3,6-dihydro-2H-pyridine-1-carboxylic acid tert-butyl ester;
4-{4-[3-(2-Carbamoylphenyl)-imidazo[1,2-a]pyridin-6-ylamino]-1H-pyrrolo[2,3-b]pyridin-2-yl}-3,6-dihydro-2H-pyridine-1-carboxylic acid tert-butylamide;
4-{4-[3-(2-Carbamoylphenyl)-imidazo[1,2-a]pyridin-6-ylamino]-1H-pyrrolo[2,3-b]pyridin-2-yl}-3,6-dihydro-2H-pyridine-1-carboxylic acid tert-butyl ester;
4-{4-[3-(2-Dimethylaminoethyl)-imidazo[1,2-a]pyridin-6-ylamino]-1H-pyrrolo[2,3-b]pyridin-2-yl}-3,6-dihydro-2H-pyridine-1-carboxylic acid tert-butylamide;
4-{4-[3-(2-Dimethylaminoethyl)-imidazo[1,2-a]pyridin-6-ylamino]-1H-pyrrolo[2,3-b]pyridin-2-yl}-3,6-dihydro-2H-pyridine-1-carboxylic acid tert-butyl ester;
4-{4-[3-(2-Acetylaminoethyl)-imidazo[1,2-a]pyridin-6-ylamino]-1H-pyrrolo[2,3-b]pyridin-2-yl}-3,6-dihydro-2H-pyridine-1-carboxylic acid tert-butylamide;
4-{4-[3-(2-Acetylaminoethyl)-imidazo[1,2-a]pyridin-6-ylamino]-1H-pyrrolo[2,3-b]pyridin-2-yl}-3,6-dihydro-2H-pyridine-1-carboxylic acid tert-butyl ester;
4-[4-(3-Dimethylaminomethylimidazo[1,2-a]pyridin-6-ylamino)-1H-pyrrolo[2,3-b]pyridin-2-yl]-3,6-dihydro-2H-pyridine-1-carboxylic acid tert-butylamide;
4-[4-(3-Dimethylaminomethylimidazo[1,2-a]pyridin-6-ylamino)-1H-pyrrolo[2,3-b]pyridin-2-yl]-3,6-dihydro-2H-pyridine-1-carboxylic acid tert-butyl ester;
4-[4-(7-Aminomethyl-1H-indazol-5-ylamino)-1H-pyrrolo[2,3-b]pyridin-2-yl]-3,6-dihydro-2H-pyridine-1-carboxylic acid tert-butylamide;
4-[4-(7-Aminomethyl-1H-indazol-5-ylamino)-1H-pyrrolo[2,3-b]pyridin-2-yl]-3,6-dihydro-2H-pyridine-1-carboxylic acid tert-butyl ester;
4-[4-(7-Aminomethyl-1-methyl-1H-indazol-5-ylamino)-1H-pyrrolo[2,3-b]pyridin-2-yl]-3,6-dihydro-2H-pyridine-1-carboxylic acid tert-butylamide;
4-[4-(7-Aminomethyl-1-methyl-1H-indazol-5-ylamino)-1H-pyrrolo[2,3-b]pyridin-2-yl]-3,6-dihydro-2H-pyridine-1-carboxylic acid tert-butyl ester;
4-[4-(7-Dimethylaminomethyl-1-methyl-1H-indazol-5-ylamino)-1H-pyrrolo[2,3-b]pyridin-2-yl]-3,6-dihydro-2H-pyridine-1-carboxylic acid tert-butylamide;
4-[4-(7-Dimethylaminomethyl-1-methyl-1H-indazol-5-ylamino)-1H-pyrrolo[2,3-b]pyridin-2-yl]-3,6-dihydro-2H-pyridine-1-carboxylic acid tert-butyl ester;
4-[4-(7-Dimethylaminomethyl-1H-indazol-5-ylamino)-1H-pyrrolo[2,3-b]pyridin-2-yl]-3,6-dihydro-2H-pyridine-1-carboxylic acid tert-butylamide;
4-[4-(7-Dimethylaminomethyl-1H-indazol-5-ylamino)-1H-pyrrolo[2,3-b]pyridin-2-yl]-3,6-dihydro-2H-pyridine-1-carboxylic acid tert-butyl ester;
4-{4-[7-(2-Dimethylaminoethyl)-1H-indazol-5-ylamino]-1H-pyrrolo[2,3-b]pyridin-2-yl}-3,6-dihydro-2H-pyridine-1-carboxylic acid tert-butylamide;
4-{4-[7-(2-Dimethylaminoethyl)-1H-indazol-5-ylamino]-1H-pyrrolo[2,3-b]pyridin-2-yl}-3,6-dihydro-2H-pyridine-1-carboxylic acid tert-butyl ester;
4-{4-[7-(2-Dimethylaminoethyl)-1-methyl-1H-indazol-5-ylamino]-1H-pyrrolo[2,3-b]pyridin-2-yl}-3,6-dihydro-2H-pyridine-1-carboxylic acid tert-butylamide;
4-{4-[7-(2-Dimethylaminoethyl)-1-methyl-1H-indazol-5-ylamino]-1H-pyrrolo[2,3-b]pyridin-2-yl}-3,6-dihydro-2H-pyridine-1-carboxylic acid tert-butyl ester;
4-[4-(Imidazo[1,5-a]pyridin-6-ylamino)-1H-pyrrolo[2,3-b]pyridin-2-yl]-3,6-dihydro-2H-pyridine-1-carboxylic acid tert-butylamide;
4-[4-(Imidazo[1,5-a]pyridin-6-ylamino)-1H-pyrrolo[2,3-b]pyridin-2-yl]-3,6-dihydro-2H-pyridine-1-carboxylic acid tert-butyl ester;
4-[4-(3-Methylimidazo[1,5-a]pyridin-6-ylamino)-1H-pyrrolo[2,3-b]pyridin-2-yl]-3,6-dihydro-2H-pyridine-1-carboxylic acid tert-butylamide;
4-[4-(3-Methylimidazo[1,5-a]pyridin-6-ylamino)-1H-pyrrolo[2,3-b]pyridin-2-yl]-3,6-dihydro-2H-pyridine-1-carboxylic acid tert-butyl ester;
4-[4-(1-Methylimidazo[1,5-a]pyridin-6-ylamino)-1H-pyrrolo[2,3-b]pyridin-2-yl]-3,6-dihydro-2H-pyridine-1-carboxylic acid tert-butylamide;
4-[4-(1-Methylimidazo[1,5-a]pyridin-6-ylamino)-1H-pyrrolo[2,3-b]pyridin-2-yl]-3,6-dihydro-2H-pyridine-1-carboxylic acid tert-butyl ester;
4-[4-(Imidazo[1,5-a]pyridin-7-ylamino)-1H-pyrrolo[2,3-b]pyridin-2-yl]-3,6-dihydro-2H-pyridine-1-carboxylic acid tert-butylamide;
4-[4-(Imidazo[1,5-a]pyridin-7-ylamino)-1H-pyrrolo[2,3-b]pyridin-2-yl]-3,6-dihydro-2H-pyridine-1-carboxylic acid tert-butyl ester;
4-[4-(3-Methylimidazo[1,5-a]pyridin-7-ylamino)-1H-pyrrolo[2,3-b]pyridin-2-yl]-3,6-dihydro-2H-pyridine-1-carboxylic acid tert-butylamide;
4-[4-(3-Methylimidazo[1,5-a]pyridin-7-ylamino)-1H-pyrrolo[2,3-b]pyridin-2-yl]-3,6-dihydro-2H-pyridine-1-carboxylic acid tert-butyl ester;
4-[4-(1-Methylimidazo[1,5-a]pyridin-7-ylamino)-1H-pyrrolo[2,3-b]pyridin-2-yl]-3,6-dihydro-2H-pyridine-1-carboxylic acid tert-butylamide;
4-[4-(1-Methylimidazo[1,5-a]pyridin-7-ylamino)-1H-pyrrolo[2,3-b]pyridin-2-yl]-3,6-dihydro-2H-pyridine-1-carboxylic acid tert-butyl ester;
4-[4-(Benzothiazol-6-ylamino)-1H-pyrrolo[2,3-b]pyridin-2-yl]-3,6-dihydro-2H-pyridine-1-carboxylic acid (2-hydroxy-1,1-dimethylethyl)-amide;
4-[4-(1-Methyl-1H-indazol-5-ylamino)-1H-pyrrolo[2,3-b]pyridin-2-yl]-3,6-dihydro-2H-pyridine-1-carboxylic acid (2-hydroxy-1,1-dimethylethyl)-amide;
4-[4-(Benzothiazol-6-ylamino)-1H-pyrrolo[2,3-b]pyridin-2-yl]-3,6-hydro-2H-pyridine-1-carboxylic acid (2-methoxy-1,1-dimethylethyl)-amide;
4-[4-(1-Methyl-1H-indazol-5-ylamino)-1H-pyrrolo[2,3-b]pyridin-2-yl]-3,6-dihydro-2H-pyridine-1-carboxylic acid (2-methoxy-1,1-dimethylethyl)-amide;
4-[4-(Benzothiazol-6-ylamino)-1H-pyrrolo[2,3-b]pyridin-2-yl]-3,6-dihydro-2H-pyridine-1-carboxylic acid (2-dimethylamino-1,1-dimethylethyl)-amide;
4-[4-(1-Methyl-1H-indazol-5-ylamino)-1H-pyrrolo[2,3-b]pyridin-2-yl]-3,6-dihydro-2H-pyridine-1-carboxylic acid (2-dimethylamino-1,1-dimethylethyl)-amide;
4-[4-(Benzothiazol-6-ylamino)-1H-pyrrolo[2,3-b]pyridin-2-yl]-3,6-dihydro-2H-pyridine-1-carboxylic acid (1,1-dimethyl-2-pyrrolidin-1-ylethyl)-amide;
4-[4-(1-Methyl-1H-indazol-5-ylamino)-1H-pyrrolo[2,3-b]pyridin-2-yl]-3,6-dihydro-2H-pyridine-1-carboxylic acid (1,1-dimethyl-2-pyrrolidin-1-ylethyl)-amide;
4-[4-(Benzothiazol-6-ylamino)-1H-pyrrolo[2,3-b]pyridin-2-yl]-3,6-dihydro-2H-pyridine-1-carboxylic acid (1,1-dimethyl-2-morpholin-4-ylethyl)-amide;
4-[4-(1-Methyl-1H-indazol-5-ylamino)-1H-pyrrolo[2,3-b]pyridin-2-yl]-3,6-dihydro-2H-pyridine-1-carboxylic acid (1,1-dimethyl-2-morpholin-4-ylethyl)-amide;
4-[4-(1H-Indol-5-ylamino)-1H-pyrrolo[2,3-b]pyridin-2-yl]-3,6-dihydro-2H-pyridine-1-carboxylic acid tert-butylamide;
4-[4-(Quinolin-6-ylamino)-1H-pyrrolo[2,3-b]pyridin-2-yl]-3,6-dihydro-2H-pyridine-1-carboxylic acid tert-butylamide;
(Hexahydropyrrolo[1,2-a]pyrazin-2-yl)-{4-[4-(imidazo[1,2-a]pyridin-6-ylamino)-1H-pyrrolo[2,3-b]pyridin-2-yl]-3,6-dihydro-2H-pyridin-1-yl}-methanone;
(Hexahydropyrrolo[1,2-a]pyrazin-2-yl)-{4-[4-(3-methyl-3H-benzoimidazol-5-ylamino)-1H-pyrrolo[2,3-b]pyridin-2-yl]-3,6-dihydro-2H-pyridin-1-yl}-methanone;
(Hexahydropyrrolo[1,2-a]pyrazin-2-yl)-{4-[4-(1-methyl-1H-indazol-5-ylamino)-1H-pyrrolo[2,3-b]pyridin-2-yl]-3,6-dihydro-2H-pyridin-1-yl}-methanone;
{4-[4-(Benzothiazol-6-ylamino)-1H-pyrrolo[2,3-b]pyridin-2-yl]-3,6-dihydro-2H-pyridin-1-yl}-(hexahydropyrrolo[1,2-a]pyrazin-2-yl)-methanone;
2-Dimethylamino-1-{4-[4-(3-ethynylphenylamino)-1H-pyrrolo[2,3-b]pyridin-2-yl]-3,6-dihydro-2H-pyridin-1-yl}-ethanone;
1-{4-[4-(4-Chloro-2,5-dimethoxyphenylamino)-1H-pyrrolo[2,3-b]pyridin-2-yl]-3,6-dihydro-2H-pyridin-1-yl}-2-dimethylaminoethanone;
{4-[4-(3-Chloro-4-fluorophenylamino)-1H-pyrrolo[2,3-b]pyridin-2-yl]-3,6-dihydro-2H-pyridin-1-yl}-(cis-2,6-dimethylmorpholin-4-yl)-methanone;
4-[4-(3-Chloro-4-fluorophenylamino)-1H-pyrrolo[2,3-b]pyridin-2-yl]-3,6-dihydro-2H-pyridine-1-carboxylic acid ethyl-methyl-amide;
4-[4-(4-Chloro-3-ethynyl-5-methoxyphenylamino)-1H-pyrrolo[2,3-b]pyridin-2-yl]-3,6-dihydro-2H-pyridine-1-carboxylic acid ethyl-methyl-amide;
{4-[4-4-Chloro-3-ethynyl-5-methoxyphenylamino)-1H-pyrrolo[2,3-b]pyridin-2-yl]-3,6-dihydro-2H-pyridin-1-yl}-(cis-2,6-dimethylmorpholin-4-yl)-methanone;
4-[4-(4-Chloro-3-ethyl-5-methoxyphenylamino)-1H-pyrrolo[2,3-b]pyridin-2-yl]-3,6-dihydro-2H-pyridine-1-carboxylic acid dimethylamide;
{4-[4-(4-Chloro-3-methoxyphenylamino)-1H-pyrrolo[2,3-b]pyridin-2-yl]-3,6-dihydro-2H-pyridin-1-yl}-(cis-2,6-dimethylmorpholin-4-yl)-methanone;
4-[4-(4-Chloro-3-methoxyphenylamino)-1H-pyrrolo[2,3-b]pyridin-2-yl]-3,6-dihydro-2H-pyridine-1-carboxylic acid ethyl-methyl-amide;
4-[4-(4-Chloro-3-methoxyphenylamino)-1H-pyrrolo[2,3-b]pyridin-2-yl]-3,6-dihydro-2H-pyridine-1-carboxylic acid dimethylamide;
{4-[4-(4-Chloro-3-methoxyphenylamino)-1H-pyrrolo[2,3-b]pyridin-2-yl]-3,6-dihydro-2H-pyridin-1-yl}-(4-thiazol-2-yl-piperazin-1-yl)-methanone;
4-[4-(4-Chloro-3-cyclopropyl-5-methoxyphenylamino)-1H-pyrrolo[2,3-b]pyridin-2-yl]-3,6-dihydro-2H -pyridine-1-carboxylicacid dimethylamide;
{4-[4-(4-Chloro-3-cyclopropyl-5-methoxyphenylamino)-1H-pyrrolo[2,3-b]pyridin-2-yl]-3,6-dihydro-2H-pyridin-1-yl}-(4-methylpiperazin-1-yl)-methanone;
4-[4-(4-Chloro-3-methoxy-5-methylphenylamino)-1H-pyrrolo[2,3-b]pyridin-2-yl]-3,6-dihydro-2H-pyridine-1-carboxylic acid tert-butyl ester;
4-[4-(1H-Indol-5-ylamino)-1H-pyrrolo[2,3-b]pyridin-2-yl]-3,6-dihydro-2H-pyridine-1-carboxylic acid tert-butylamide;
{4-[4-(1H-Indol-5-ylamino)-1H-pyrrolo[2,3-b]pyridin-2-yl]-3,6-dihydro-2H-pyridin-1-yl}-(4-methylpiperazin-1-yl)-methanone;
{4-[4-(3-Chloro-4-fluorophenylamino)-1H-pyrrolo[2,3-b]pyridin-2-yl]-3,6-dihydro-2H-yridin-1-yl}-(4-methylpiperazin-1-yl)-methanone;
2-Dimethylamino-1-4-[4-(1H-indol-5-ylamino)-1H-pyrrolo[2,3-b]pyridin-2-yl]-3,6-dihydro-2H-pyridin-1-yl-ethanone;
1-4-[4-(3,5-Dimethoxyphenylamino)-1H-pyrrolo[2,3-b]pyridin-2-yl]-3,6-dihydro-2H-pyridin-1-yl-2-dimethylaminoethanone;
2-Dimethylamino-1-[4-(4-phenylamino-1H-pyrrolo[2,3-b]pyridin-2-yl)-3,6-dihydro-2H-pyridin-1-yl]-ethanone;
4-[4-(Benzo[1,3]dioxol-5-ylamino)-1H-pyrrolo[2,3-b]pyridin-2-yl]-3,6-dihydro-2H-pyridin-1-yl-4-methylpiperazin-1-yl)-methanone;
4-[4-(3-Chloro-4-fluorophenylamino)-1H-pyrrolo[2,3-b]pyridin-2-yl]-3,6-dihydro-2H-pyridine-1-carboxylic acid t-butylamide;
4-[4-(3-Ethynylphenylamino)-1H-pyrrolo[2,3-b]pyridin-2-yl]-3,6-dihydro-2H-pyridine-1-carboxylic acid tert-butylamide;
1-4-[4-(4-Chloro-3-methoxyphenylamino)-1H-pyrrolo[2,3-b]pyridin-2-yl]-3,6-dihydro-2H-pyridin-1-yl-2-dimethylaminoethanone;
1-4-[4-(3-Chloro-4-fluorophenylamino)-1H-pyrrolo[2,3-b]pyridin-2-yl]-3,6-dihydro-2H-pyridin-1-yl-2-dimethylaminoethanone;
1-4-[4-(2,3-Dihydroindol-1-yl)-1H-pyrrolo[2,3-b]pyridin-2-yl]-3,6-dihydro-2H-pyridin-1-yl-2-dimethylaminoethanone;
4-[4-(4-Chloro-3-ethynyl-5-methoxyphenylamino)-1H-pyrrolo[2,3-b]pyridin-2-yl]-3,6-dihydro-2H-pyridine- -carboxylic acid dimethylamide;
4-[4-(4-Chloro-3-methoxy-5-methylphenylamino)-1H-pyrrolo[2,3-b]pyridin-2-yl]-3,6-dihydro-2H-pyridine-1-carboxylic acid dimethylamide;
{4-[4-(3-Chloro-4-fluorophenylamino)-1H-pyrrolo[2,3-b]pyridin-2-yl]-3,6-dihydro-2H-pyridin-1-yl}-(R)-piperidin-2-yl-methanone;
{4-[4-(4-Chloro-2,5-dimethoxyphenylamino)-1H-pyrrolo[2,3-b]pyridin-2-yl]-3,6-dihydro-2H-pyridin-1-yl}-(R)-piperidin-2-yl-methanone;
{4-[4-(4-Chloro-2,5-dimethoxyphenylamino)-1H-pyrrolo[2,3-b]pyridin-2-yl]-3,6-dihydro-2H-pyridin-1-yl}-(S)-piperidin-2-yl-methanone;
{4-[4-(4-Chloro-2,5-dimethoxyphenylamino)-1H-pyrrolo[2,3-b]pyridin-2-yl]-3,6-dihydro-2H-pyridin-1-yl}-(S)-piperidin-3-yl-methanone;
{4-[4-(4-Chloro-2,5-dimethoxyphenylamino)-1H-pyrrolo[2,3-b]pyridin-2-yl]-3,6-dihydro-2H-pyridin-1-yl}-(R)-piperidin-3-yl-methanone;
{4-[4-(4-Chloro-2,5-dimethoxyphenylamino)-1H-pyrrolo[2,3-b]pyridin-2-yl]-3,6-dihydro-2H-pyridin-1-yl}-(1-methylpiperidin-2-yl)-methanone;
{4-[4-(4-Chloro-2,5-dimethoxyphenylamino)-1H-pyrrolo[2,3-b]pyridin-2-yl]-3,6-dihydro-2H-pyridin-1-yl}-((S)-1-methylpyrrolidin-2-yl)-methanone;
{4-[4-(4-Chloro-2,5-dimethoxyphenylamino)-1H-pyrrolo[2,3-b]pyridin-2-yl]-3,6-dihydro-2H-pyridin-1-yl}-((S)-1-methylpyrrolidin-2-yl)-methanone;
{4-[4-(3-Chloro-4-fluorophenylamino)-1H-pyrrolo[2,3-b]pyridin-2-yl]-3,6-dihydro-2H-pyridin-1-yl}-((S)-1-methylpyrrolidin-2-yl)-methanone;
{4-[4-(3-Chloro-4-fluorophenylamino)-1H-pyrrolo[2,3-b]pyridin-2-yl]-3,6-dihydro-2H-pyridin-1-yl}-(S)-piperidin-3-yl-methanone;
{4-[4-3-Chloro-4-fluorophenylamino)-1H-pyrrolo[2,3-b]pyridin-2-yl]-3,6-dihydro-2H-pyridin-1-yl}-(S)-pyrrolidin-3-yl-methanone;
{4-[4-(4-Chloro-3-methoxy-5-methylphenylamino)-1H-pyrrolo[2,3-b]pyridin-2-yl]-3,6-dihydro-2H-pyridin-l -yl}-(4-methylpiperazin-1-yl)-methanone;
N-(4-Chloro-3-methoxy-5-methylphenyl)-2-(1,2,3,6-tetrahydropyridin-4-yl)-1H-pyrrolo[2,3-b]pyridin-4-amine;
or a pharmaceutically acceptable salt thereof.

16. The compound according to claim 1, wherein RI is selected from the following table, wherein the wavy bond is connected to Y: 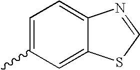 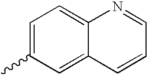 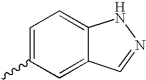 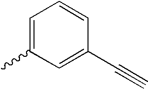 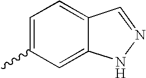 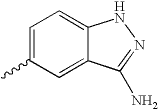 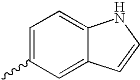 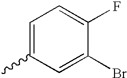 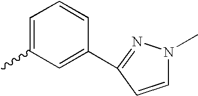 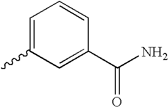 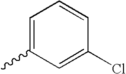 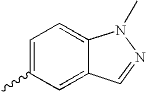 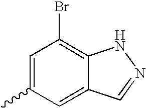 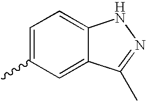 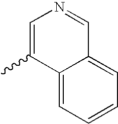 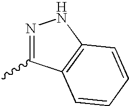 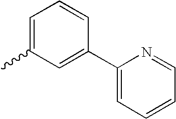 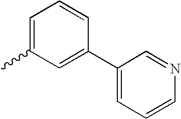 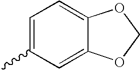 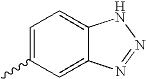 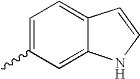 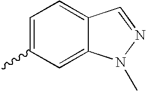 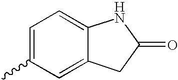 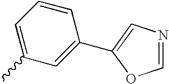 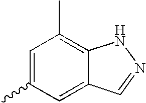 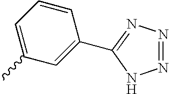 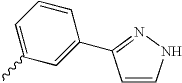 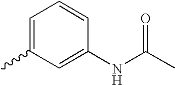 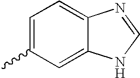 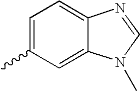 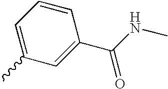 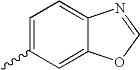 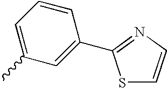 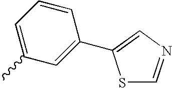 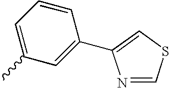 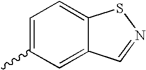 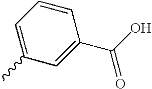 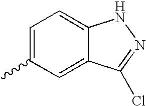 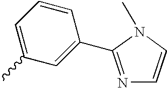 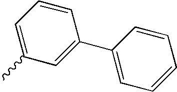 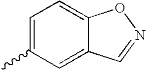 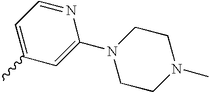 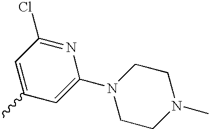 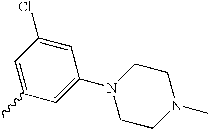 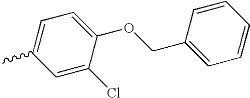 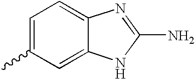 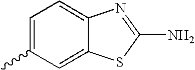 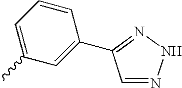 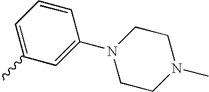 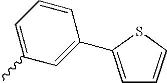 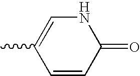 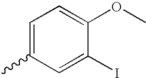 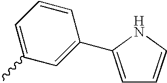 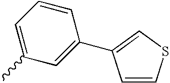 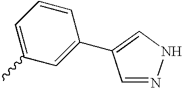 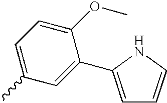 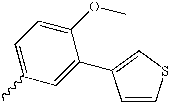 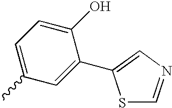 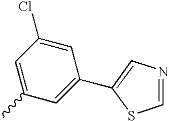 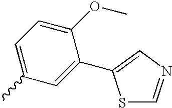 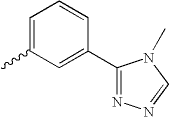 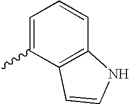 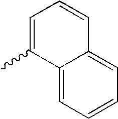 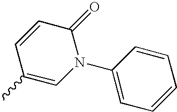 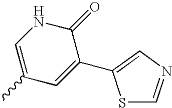 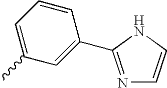 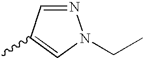 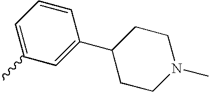 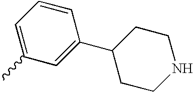 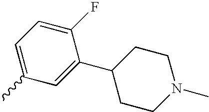 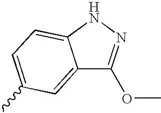 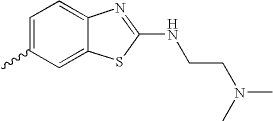 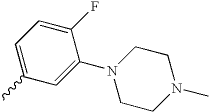 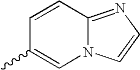 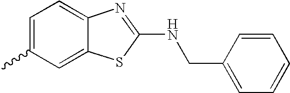 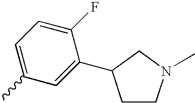 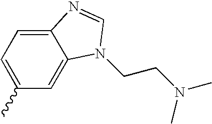 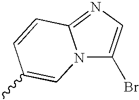 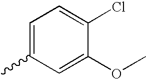 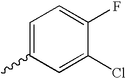 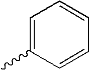 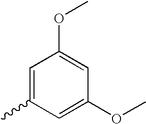 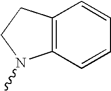 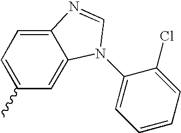 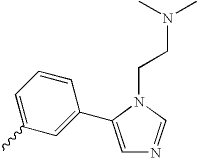 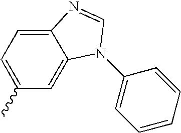 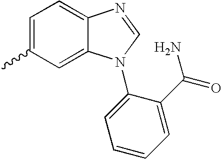 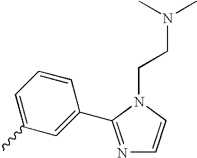 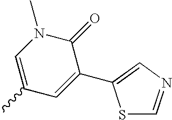 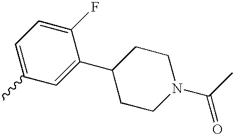 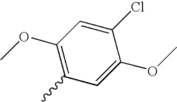 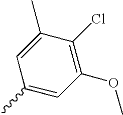 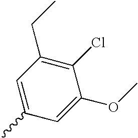 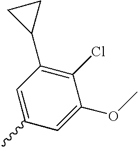 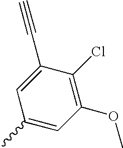 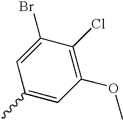
or pharmaceutically acceptable salt thereof.

17. The compound according to claim 1, wherein Z is selected from the following table, wherein the dotted line is connected to Cy: 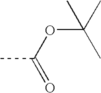 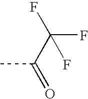 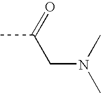 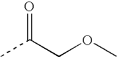 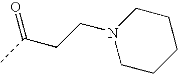 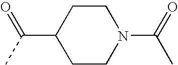 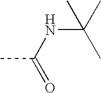 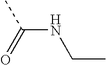 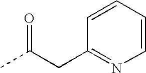 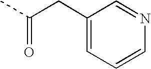 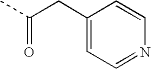 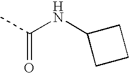 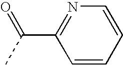 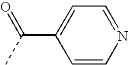 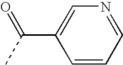 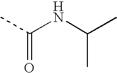 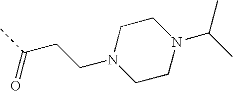 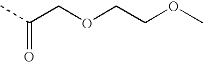 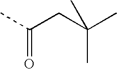 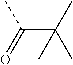 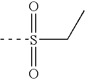 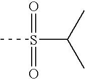 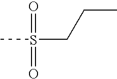 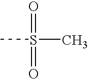 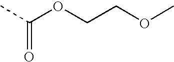 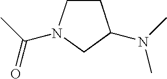 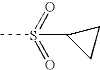 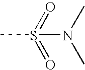 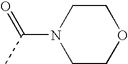 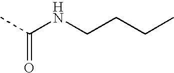 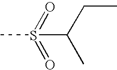 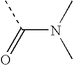 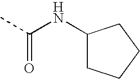 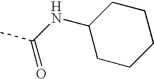 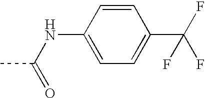 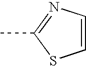 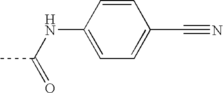 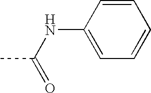 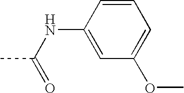 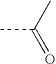 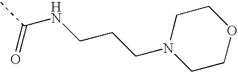 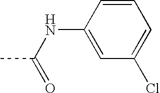 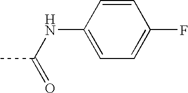 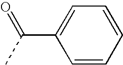 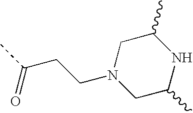 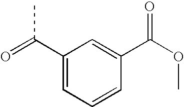 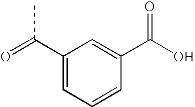 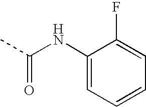 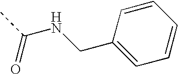 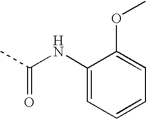 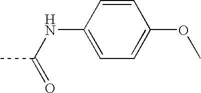 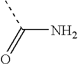 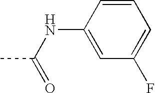 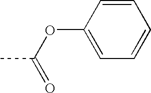 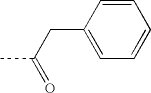 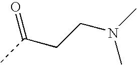 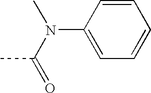 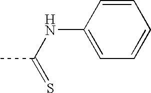 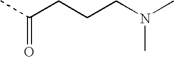 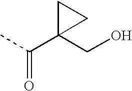 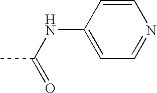 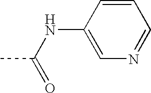 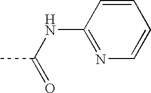 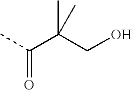 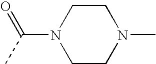 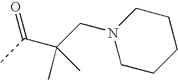 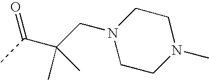 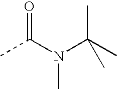 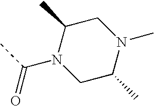 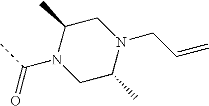 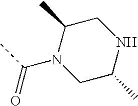 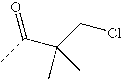 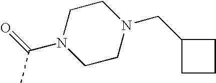 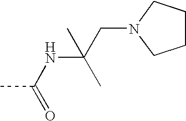 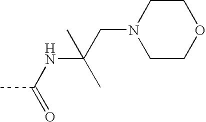 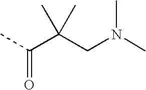 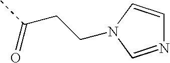 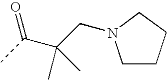 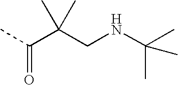 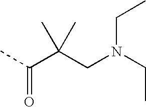 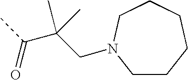 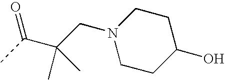 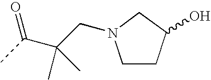 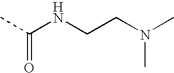 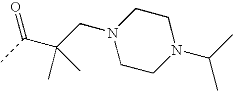 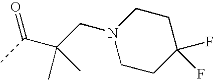 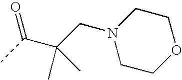 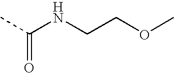 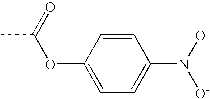 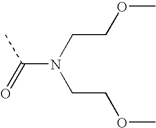 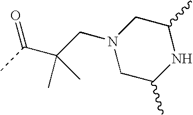 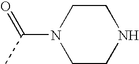 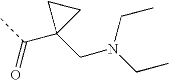 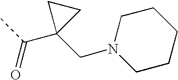 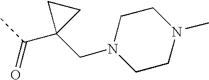 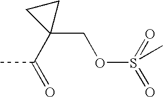 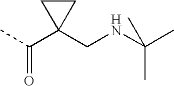 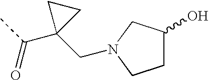 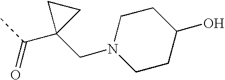 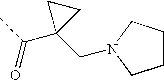 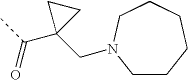 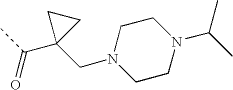 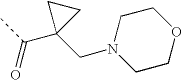 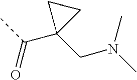 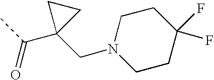 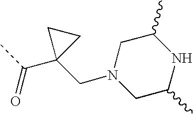 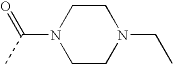 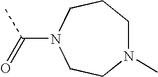 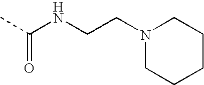 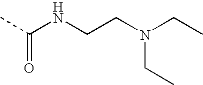 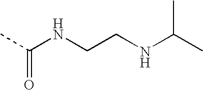 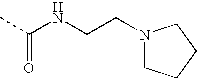 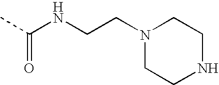 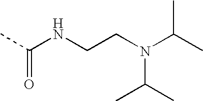 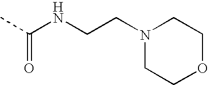 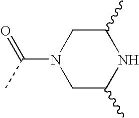 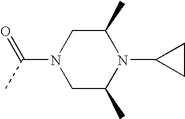 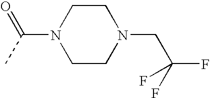 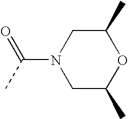 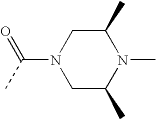 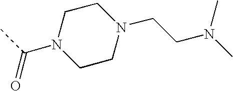 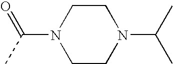 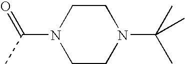 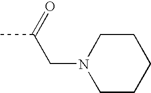 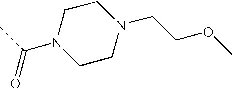 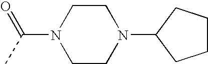 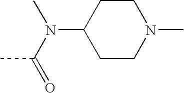 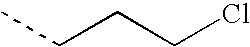 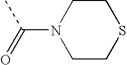 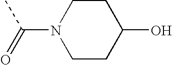 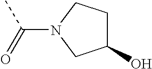 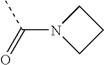 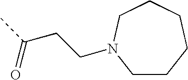 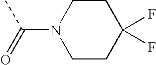 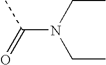 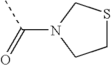 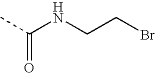 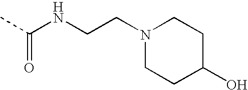 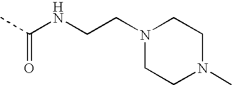 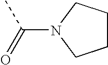 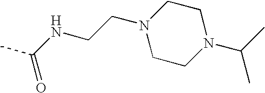 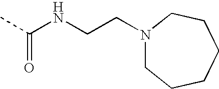 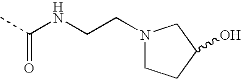 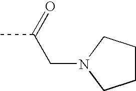 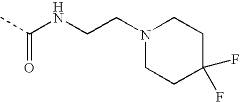 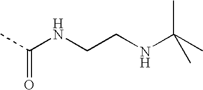 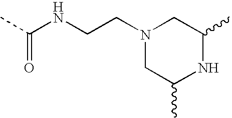 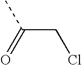 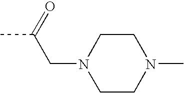 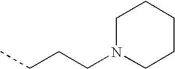 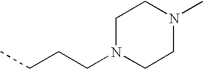 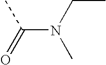 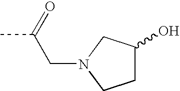 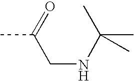 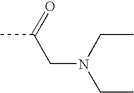 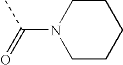 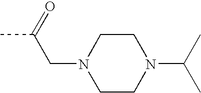 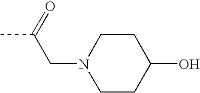 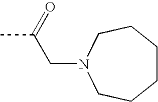 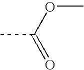 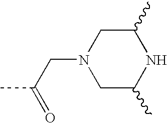 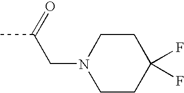 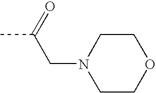 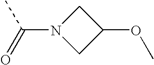 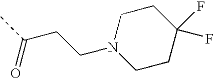 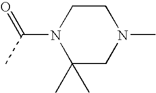 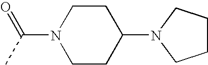 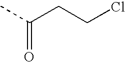 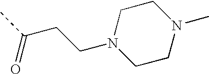 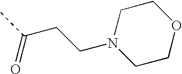 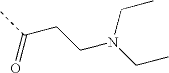 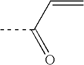 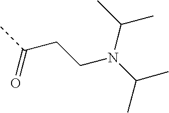 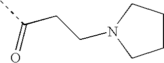 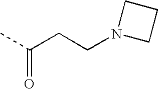 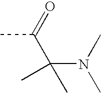 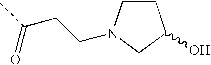 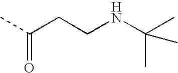 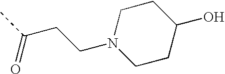 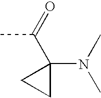 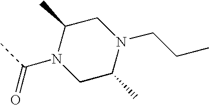 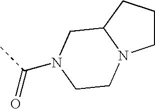 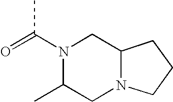 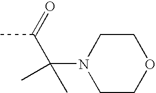 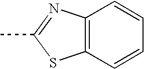 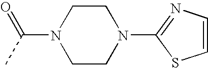 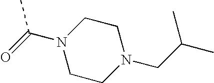 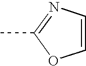 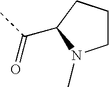 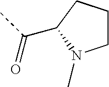 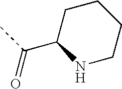 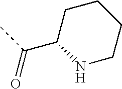 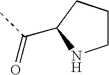 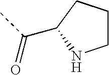 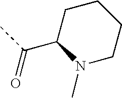 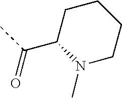 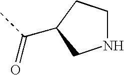 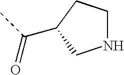 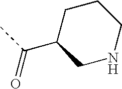 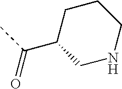 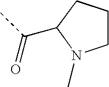 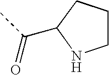 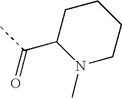 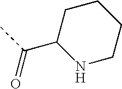 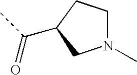 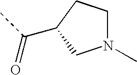 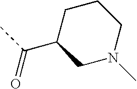 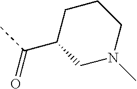 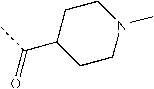 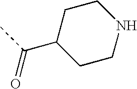
or a pharmaceutically acceptable salt thereof.

18. A composition comprising a compound according to claim 1, or a pharmaceutically acceptable salt thereof, and a pharmaceutically acceptable carrier.

19. A composition comprising a compound according to claim 1, or a pharmaceutically acceptable salt thereof, and at least one of AVASTIN, IRESSA, TARCEVA, ERBITUX, or cisplatin.

20. A method for the treatment of cancer of the lung, breast, prostate, pancreas, head, neck or blood comprising a step of administering to a subject in need thereof an effective amount of a compound according to claim 1, or a pharmaceutically acceptable salt thereof.
